(19) United States
(12) Patent Application Publication
(10) Pub. No.: US 2026/0124259 A1
(43) Pub. Date: May 7, 2026

(54) STAPHYLOCOCCUS PHAGE COMPOSITIONS AND COCKTAILS THEREOF

(71) Applicant: Locus Biosciences, Inc., Morrisville, NC (US)

(72) Inventors: David G. OUSTEROUT, Morrisville, NC (US); Hannah Hewitt TUSON, Morrisville, NC (US); Lana MCMILLAN, Morrisville, NC (US); Robert MCKEE, Morrisville, NC (US)

(73) Assignee: Locus Biosciences, Inc., Morrisville, NC (US)

(21) Appl. No.: 19/121,791

(22) PCT Filed: Oct. 16, 2023
(86) PCT No.: PCT/US2023/076994

§ 371 Date: Apr. 16, 2025

Related U.S. Application Data
(60) Provisional application No. 63/379,807, filed on Oct. 17, 2022.

(51) Int. Cl. A61K 35/76; A61P 31/02; C12N 7/00

(52) U.S. Cl. CPC A61K 35/76; A61P 31/02; C12N 7/00; C12N 2795/10032

(57) ABSTRACT

Disclosed here are phage compositions for infecting and/or killing Staphylococcus spp.

### CROSS-REFERENCE

This application claims the benefit of U.S. Provisional Application No. 63/379,807, filed on Oct. 17, 2022, which is incorporated herein by reference in its entirety.

### INCORPORATION BY REFERENCE OF SEQUENCE LISTING

The present application is being filed along with a Sequence Listing in electronic format. The Sequence Listing is provided as a file entitled 53240-754601_SL.xml, created Sep. 21, 2023, which is 4,258,034 bytes in size. The information in the electronic format of the Sequence Listing is incorporated by reference in its entirety.

### SUMMARY

In certain aspects, described herein is a composition comprising at least two bacteriophage, wherein a first bacteriophage is at least 80% identical to p1378 or p4815, and a second bacteriophage is at least 80% identical to p1494e002 or p2808. In some embodiments, the first bacteriophage is at least 80% identical to p1378, and the second bacteriophage is at least 80% identical to p1494e002. In some embodiments, the first bacteriophage is at least 80% identical to p1378, and the second bacteriophage is at least 80% identical to p2808. In some embodiments, the first bacteriophage is at least 80% identical to p4815, and the second bacteriophage is at least 80% identical to p1494e002. In some embodiments, the first bacteriophage is at least 80% identical to p4815, and the second bacteriophage is at least 80% identical to p2808. In some embodiments, the composition further comprises comprising a third bacteriophage at least 80% identical to the p4815. In some embodiments, the composition further comprises a fourth bacteriophage at least 80% identical to the p2808. In some embodiments, the composition further comprises a third bacteriophage at least 80% identical to the p4815. In some embodiments, the composition further comprises a third bacteriophage at least 80% identical to the p2808. In certain aspects, described herein is a composition comprising a plurality of bacteriophage comprising a first bacteriophage and a second bacteriophage, wherein a bacteria treated with the plurality of bacteriophage has a reduced amount of regrowth as compared to treatment with the first bacteriophage or the second bacteriophage alone, wherein the first bacteriophage comprises a Rosenblumvirus and the second bacteriophage comprises a Phietavirus or a Kayvirus. In some embodiments, the amount of regrowth is measured by optical density (OD) at a wavelength of 600 nm. In some embodiments, the amount of regrowth of the bacteria treated with the plurality of bacteriophage 12 hours after treatment is less than or equal to about 50%, 40%, 30%, 20%, or 10% of regrowth of the bacteria treated with the first bacteriophage. In some embodiments, the amount of regrowth of the bacteria treated with the plurality of bacteriophage 12 hours after treatment is less than or equal to about 50%, 40%, 30%, 20%, or 10% of regrowth of the bacteria treated with the second bacteriophage. In some embodiments, the bacteria is a Staphylococcus bacteria. In some embodiments, the bacteria comprises Staphylococcus aureus, methicillin resistant Staphylococcus aureus, Staphylococcus epidermidis, Staphylococcus salivarius, Staphylococcus argenteus, Staphylococcus hemolyticus, or Staphylococcus schweitzeri, or any combination of two or more thereof. In some embodiments, the bacteria comprises the Staphylococcus aureus.

In certain aspects, described herein is a bacteriophage engineered to render the bacteriophage lytic by removal, replacement, or inactivation of a lysogenic sequence, wherein the lysogenic sequence comprises at least 10%, 20%, 30%, 40%, 50%, 60%, 70%, 80%, 90%, or 100% of a sequence selected from SEQ ID NOS: 49-58. In some embodiments, the bacteriophage is an engineered Phictavirus. In some embodiments, described herein is a composition comprising the bacteriophage described herein, further comprising a Rosenblumvirus and/or a Kayvirus. In some embodiments, described herein is a method of treating a disease or condition related to Staphylococcus, the method comprising administering to a subject in need thereof the bacteriophage described herein, or the composition described herein. In some embodiments, Staphylococcus is causative of, and/or contributes to, the disease or condition.

In certain aspects, described herein is a bacteriophage comprising at least 70%, 75%, 80%, 85%, 90%, 95%, or 100% sequence identity with p1378 (PTA-127329). In certain aspects, described herein is a bacteriophage comprising at least 70%, 75%, 80%, 85%, 90%, 95%, or 100% sequence identity with p4815 (PTA-127331). In certain aspects, described herein is a bacteriophage comprising at least 70%, 75%, 80%, 85%, 90%, 95%, or 100% sequence identity with p1494e002 (PTA-127345). In certain aspects, described herein is a bacteriophage comprising at least 70%, 75%, 80%, 85%, 90%, 95%, or 100% sequence identity with p2808 (PTA-127332).

In certain aspects, described herein is a composition comprising at least two bacteriophage, wherein a first bacteriophage comprises at least 70%, 75%, 80%, 85%, 90%, 95%, or 100% sequence identity with p1378, and a second bacteriophage comprises at least 70%, 75%, 80%, 85%, 90%, 95%, or 100% sequence identity with p1381, p4815, p1378e062, p1378e074, p4815e037, p1378e075, p1381e017, p4815e053, p1498e001, p3693e001, p3224e002, p5593e001, p1468e003, p1478e003, p1494e002, p2808, or p1473.

In certain aspects, described herein is a composition comprising at least two bacteriophage, wherein a first bacteriophage comprises at least 70%, 75%, 80%, 85%, 90%, 95%, or 100% sequence identity with p4815, and a second bacteriophage comprises at least 70%, 75%, 80%, 85%, 90%, 95%, or 100% sequence identity with p1381, p1378, p1378e062, p1378e074, p4815e037, p1378e075, p1381e017, p4815e053, p1498e001, p3693e001, p3224e002, p5593e001, p1468e003, p1478e003, p1494e002, p2808, or p1473. In some embodiments, the second bacteriophage comprises at least at least 70%, 75%, 80%, 85%, 90%, 95% or 100% sequence identity with p4815, 01494e002, or p2808. In some embodiments, the composition further comprises a third bacteriophage, wherein the third bacteriophage comprises at least 70%, 75%, 80%, 85%, 90%, 95% or 100% sequence identity with p4815, 01494e002, or p2808. In some embodiments, the composition further comprises a fourth bacteriophage, wherein the fourth bacteriophage comprises at least 70%, 75%, 80%, 85%, 90%, 95% or 100% sequence identity with p4815, 01494e002, or p2808.

In certain aspects, described herein is a composition comprising at least two bacteriophage, wherein a first bacteriophage comprises at least 70%, 75%, 80%, 85%, 90%, 95%, or 100% sequence identity with p1494e002, and a second bacteriophage comprises at least 70%, 75%, 80%, 85%, 90%, 95%, or 100% sequence identity with p1381, p1378, p4815, p1378e062, p1378e074, p4815e037, p1378e075, p1381e017, p4815e053, p1498e001, p3693e001, p3224e002, p5593e001, p1468e003, p1478e003, p2808, or p1473.

In certain aspects, described herein is a composition comprising at least two bacteriophage, wherein a first bacteriophage comprises at least 70%, 75%, 80%, 85%, 90%, 95%, or 100% sequence identity with p2808, and a second bacteriophage comprises at least 70%, 75%, 80%, 85%, 90%, 95%, or 100% sequence identity with p1381, p1378, p4815, p1378e062, p1378e074, p4815e037, p1378e075, p1381e017, p4815e053, p1498e001, p3693e001, p3224e002, p5593e001, p1468e003, p1478e003, p1494e002, or p1473. In some embodiments, the at least two bacteriophage of the composition infect at least about 90% of a collection of at least about 30 Staphylococcus bacteria. In some embodiments, the infection of the at least about 90% is determined with a plaque assay or growth inhibition assay. In some embodiments, the at least about 90% is at least about 95%. In some embodiments, the at least about 90% is at least about 98%. In some embodiments, the at least about 90% is at least about 99%. In some embodiments, the collection of Staphylococcus bacteria comprises comprise Staphylococcus bacteria having a MLST of 8, 5, 22, 15, 1, 30, 398, 105, 45, 672, 2250, 582, 72, 97, 239, 34, 87, 101, 109, 1159, 1165, 1181, 12, 121, 152, 1750, 20, 225, 25, 291, 3628, 59, 7, 779, 88, 10, 1011, 1049, 1156, 1351, 149, 1637, 1649, 1757, 1842, 188, 1970, 2066, 256, 2867, 2945, 3149, 3182, 3510, 39, 395, 4317, 47, 4730, 50, 508, 573, 6, 630, 737, 828, 848, 923, or 93, or a combination of two or more thereof. In some embodiments, the collection of Staphylococcus bacteria comprises bacteria isolated from a bloodstream infection. In some embodiments, at least about 40% of the collection of Staphylococcus bacteria are multidrug resistant.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts the predicted lysogeny region of a Phietavirus bacteriophage p1473.

FIG. 2 depicts the dilution series of wild-type (WT) p1473 and several variants plated on a lawn of Staphylococcus aureus using the double agar overlay method. WT phage and variants Var002 and Var006 produced small hazy plaques, while variants Var009, Var010 and Var012 produced larger clearer plaques.

FIG. 3 depicts the larger plaque morphology for wild-type p1473, Var010 and Var012 in a close-up image, with red arrows indicating individual plaques.

FIG. 4 depicts the bacteriophage killing assay with strain b4063 (USA300 strain FPR3757) in LB challenged with Wild-type p1473, Var012, or Var042 as measured by bacterial counts as a function of time.

FIG. 5 depicts the growth curves of three S. aureus clinical isolates (b2991, b3022 and b3202) in LB challenged with p1473 WT or p1473 Var012.

FIG. 6 depicts the sequence of the Var010 deletion (SEQ ID NO: 30).

FIG. 7 depicts the sequence of the Var012 deletion (SEQ ID NO: 31), which is also the first deletion in Var042.

FIG. 8 depicts the second deletion in Var042 (SEQ ID NO: 32).

FIG. 9 depicts PCR screening of a Kayvirus phage engineered to contain a Type I CRISPR System IB from Listeria monocytogenes (LMIB).

FIG. 10 depicts PCR screening of the lnqO promoter into phage.

FIG. 11 depicts the concentration of b2655 inoculated with engineered and wildtype p4815 Kayvirus phage over time. Each engineered p4815 Kayvirus comprises one of three promotor variants used to drive expression of lacticin Q (lnqQ): a Pcat-lnqQ, PsarA-lnqQ, or PtmpG-lnqQ promoter.

FIG. 12 depicts an alignment of the genomes of parent Kayvirus phage with the engineered variant containing DNase I and the P_{cat} promoter.

FIG. 13A depicts the deletion in the p1494e002 phage (SEQ ID NO: 58, also referred to as e002 or var0022).

FIG. 13B depicts the concentrations of FPR3757 inoculated with engineered (p1494e002) and wildtype (p1494 WT) p1494 over time.

FIG. 13C depicts the growth curve of Staphylococcus isolate b2655 over time after infection with wildtype phage (p1498 WT), engineered phage (p1498e001) or no phage.

FIG. 14A depicts growth curves of b4735 inoculated with individual phage (p1378, p1494e002, p2808, p4815) and a cocktail (CK811).

FIG. 14B depicts growth curves of b2745 inoculated with individual phage (p1378, p1494e002, p2808, p4815) and a phage cocktail (CK811).

FIG. 14C depicts growth curves of b4681 inoculated with individual phage (p1378, p1494e002, p2808, p4815) and a phage cocktail (CK811).

FIG. 15 depicts a Venn diagram showing the different bacteria strains targeted by Phietavirus, Rosenblumvirus, and Kayvirus, and combinations of the bacteriophage.

FIG. 16A depicts growth curves of Staphylococcus isolate b4681 after infection with p1378 or no phage. FIG. 16B depicts growth curves of Staphylococcus isolate b4681 after infection with p4815 or no phage. FIG. 16C depicts growth curves of Staphylococcus isolate b4681 after infection with p2808 or no phage. FIG. 16D depicts growth curves of Staphylococcus isolate b4681 after infection with p1494e002 or no phage. FIG. 16E depicts growth curves of Staphylococcus isolate b4681 after infection with a 3 genus cocktail (CK811 comprising p1378, p1494e002, p2808 and p4815), or no phage.

FIG. 17A depicts growth curves of Staphylococcus isolate b4681 after infection with p1378 or no phage. FIG. 17B depicts growth curves of Staphylococcus isolate b4681 after infection with p2808 or no phage. FIG. 17C depicts growth curves of Staphylococcus isolate b4681 after infection with a 2 genus cocktail (CK665 comprising p1378, p2808 and p4815) or no phage. FIG. 17D depicts growth curves of Staphylococcus isolate b4681 after infection with p4815 or no phage. FIG. 17E depicts growth curves of Staphylococcus isolate b4681 after infection with p1498e001 or no phage. FIG. 17F depicts growth curves of Staphylococcus isolate b4681 after infection with a 3 genus cocktail (CK766 comprising p1378, p1498e001, p2808 and p4815) or no phage.

FIG. 18A depicts growth curves of Staphylococcus isolate b4787 after infection with p1378 or no phage. FIG. 18B depicts growth curves of Staphylococcus isolate b4787 after infection with p2808 or no phage. FIG. 18C depicts growth curves of Staphylococcus isolate b4787 after infection with p4815 or no phage. FIG. 18D depicts growth curves of Staphylococcus isolate b4787 after infection with p1498e001 or no phage. FIG. 18E depicts growth curves of Staphylococcus isolate b4787 after infection with a 2 genus cocktail (CK665) or no phage. FIG. 18F depicts growth curves of Staphylococcus isolate b4787 after infection with 3 genus cocktail (CK766), or no phage.

FIG. 19A depicts growth curves of Staphylococcus isolate b4665 after infection with p1378 or no phage. FIG. 19B depicts growth curves of Staphylococcus isolate b4665 after infection with p2808 or no phage. FIG. 19C depicts growth curves of Staphylococcus isolate b4665 after infection with a 2 genus cocktail (CK665) or no phage. FIG. 19D depicts growth curves of Staphylococcus isolate b4665 after infection with p4815 or no phage. FIG. 19E depicts growth curves of Staphylococcus isolate b4665 after infection with p1498e001, or no phage. FIG. 19F depicts growth curves of Staphylococcus isolate b4665 after infection with 3 genus cocktail (CK766), or no phage.

FIG. 20A depicts growth curves of Staphylococcus isolate b4689 after infection with p1378 or no phage. FIG. 20B depicts growth curves of Staphylococcus isolate b4689 after infection with p2808 or no phage. FIG. 20C depicts growth curves of Staphylococcus isolate b4689 after infection with a 2 genus cocktail (CK665) or no phage. FIG. 20D depicts growth curves of Staphylococcus isolate b4689 after infection with p4815 or no phage. FIG. 20E depicts growth curves of Staphylococcus isolate b4689 after infection with p1498e001 or no phage. FIG. 20F depicts growth curves of Staphylococcus isolate b4689 after infection with a 3 genus cocktail (CK766), or no phage.

### DETAILED DESCRIPTION

Disclosed herein, in certain aspects, are compositions and methods for killing and/or infecting Staphylococcus with bacteriophage. In certain aspects, described herein is a composition comprising a plurality of bacteriophage comprising a first bacteriophage and a second bacteriophage, wherein the plurality of bacteriophage infect at least about 90% of a collection of at least about 30 Staphylococcus bacteria. In certain aspects, described herein is a composition comprising a plurality of bacteriophage comprising a first bacteriophage that binds a first receptor of a Staphylococcus bacteria in a collection of Staphylococcus bacteria, and a second bacteriophage that binds a second receptor of a Staphylococcus bacteria in the collection of Staphylococcus bacteria. In some embodiments, a bacteriophage specifically binds to a first receptor if the binding between the bacteriophage and a second receptor is less than about 10% of the binding between the bacteriophage and the first receptor. In some embodiments, the first bacteriophage specifically binds a first receptor of a Staphylococcus bacteria in a collection of Staphylococcus bacteria. In some embodiments, second bacteriophage specifically binds a second receptor of a Staphylococcus bacteria in the collection of Staphylococcus bacteria.

In certain aspects, described herein is a composition comprising a plurality of bacteriophage comprising a first bacteriophage and a second bacteriophage, wherein a bacteria treated with the plurality of bacteriophage has a reduced amount of regrowth as compared to treatment with the first bacteriophage or the second bacteriophage alone.

In some embodiments, the bacteriophage described herein target or infect Staphylococcus. In some embodiments, the bacteriophage is engineered to remove lysogeny. In some embodiments, the bacteriophage comprises a CRISPR/Cas system as described herein. In some embodiments, the bacteriophage comprises an antimicrobial agent or peptide. In some embodiments, the bacteriophage does not comprise a CRISPR/Cas system. In some embodiments, the bacteriophage does not comprise an antimicrobial agent or peptide.

Disclosed herein, in certain embodiments, are compositions comprising a plurality of bacteriophage. In some cases, the plurality comprises one or more engineered bacteriophage, e.g., engineered to remove a lysogenic gene, and/or to include a payload such as a CRISPR-Cas component and/or antimicrobial peptide. In some embodiments, the plurality of bacteriophage target a wide host range. For instance, the composition targets at least about 90% of a collection of at least about 30 Staphylococcus bacteria. In some embodiments, the plurality of bacteriophage comprises a first bacteriophage that binds a first receptor of a Staphylococcus bacteria, a second bacteriophage that binds a second receptor of the Staphylococcus bacteria, wherein the plurality of bacteriophage is more resilient to resistance by the Staphylococcus bacteria than the first or second bacteriophage alone.

### Bacteriophage

Described herein, in certain aspects, are bacteriophage that target Staphylococcus spp. In some embodiments, the bacteriophage targets S. aureus. In some embodiments, the bacteriophage specifically targets Staphylococcus spp. over other bacterial species. In some embodiments, the bacteriophage targets Staphylococcus spp. in the absence of a CRISPR-Cas system.

In some embodiments, the bacteriophage targets Staphylococcus spp. In some embodiments, the bacteriophage is a Kayvirus, a Twortvirus, a Rosenblumvirus, a Phictavirus, a Triavirus, a Dubowvirus, a Beccayunavirus, a Peeveelvirus, a Coventryvirus, or a Rockefellervirus. As used herein, a Phictavirus may comprise a Phictavirus or a Dubowvirus. In some embodiments, the bacteriophage is a Kayvirus. In some embodiments, the bacteriophage is a Twortvirus. In some embodiments, the bacteriophage is a Rosenblumvirus. In some embodiments, the bacteriophage is a Phietavirus. In some embodiments, the bacteriophage is a Triavirus. In some embodiments, the bacteriophage is a Dubowvirus. In some embodiments, the bacteriophage is a Beccayunavirus. In some embodiments, the bacteriophage is a Peeveelvirus. In some embodiments, the bacteriophage is a Coventryvirus. In some embodiments, the bacteriophage is a Rockefellervirus. In some embodiments, the bacteriophage encodes a CRISPR-Cas system. In some embodiments, the bacteriophage encodes a peptide. In some embodiments, the bacteriophage does not comprise or encode a CRISPR-Cas system. In some embodiments, the bacteriophage does not comprise or encode a peptide.

In some embodiments, the bacteriophage is a Phietavirus. In some embodiments, the Phictavirus is engineered to remove, replace, or inactivate a lysogenic gene or a promoter of a lysogenic gene. As a non-limiting example, the lysogenic gene encodes for a repressor. In some embodiments, removal of the lysogenic gene comprises removing from about 1% to 100% of the lysogenic gene, or about 10 to about 1,200 base pairs of the lysogenic gene are removed. In some embodiments, the lysogenic gene encodes for an amino acid sequence at least about 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 47 or 48. In some embodiments, the bacteriophage is engineered to comprise or encode one or more components of a CRISPR-Cas system, and/or an antimicrobial peptide. In some embodiments, the bacteriophage does not comprise or encode one or more components of a CRISPR-Cas system. In some embodiments, the bacteriophage does not comprise or encode an antimicrobial peptide.

In some embodiments, the Phictavirus is p1473. In some embodiments, the bacteriophage comprises at least 70%, 75%, 80%, 85%, 90%, 95%, or 100% sequence identity with p1473. In some embodiments, the p1473 is engineered to remove, replace, or inactivate a lysogenic gene or a promoter of a lysogenic gene. In some embodiments, the Phietavirus is a p1473 comprising a nucleic acid encoding a CRISPR-Cas system disclosed herein. In some embodiments, the Phietavirus is a p1473 comprising a nucleic acid encoding a peptide disclosed herein.

In some embodiments, the Phietavirus is p1498. In some embodiments, the bacteriophage comprises at least 70%, 75%, 80%, 85%, 90%, 95%, or 100% sequence identity with p1498. In some embodiments, the p1498 is engineered to remove, replace, or inactivate a lysogenic gene or a promoter of a lysogenic gene. In some embodiments, the p1498 is engineered to remove, replace, or inactivate at least 10%, 20%, 30%, 40%, 50%, 60%, 70%, 80%, 90%, or 100% of SEQ ID NO: 52. In some embodiments, the bacteriophage comprises at least 70%, 75%, 80%, 85%, 90%, 95%, or 100% sequence identity with p1498e001 (PTA-127336). In some embodiments, the Phietavirus is a p1498 comprising a nucleic acid encoding a CRISPR-Cas system disclosed herein. In some embodiments, the Phietavirus is a p1498 comprising a nucleic acid encoding a peptide disclosed herein.

In some embodiments, the Phietavirus is p3693. In some embodiments, the bacteriophage comprises at least 70%, 75%, 80%, 85%, 90%, 95%, or 100% sequence identity with p3693. In some embodiments, the p3693 is engineered to remove, replace, or inactivate a lysogenic gene or a promoter of a lysogenic gene. In some embodiments, the p3693 is engineered to remove, replace, or inactivate at least 10%, 20%, 30%, 40%, 50%, 60%, 70%, 80%, 90%, or 100% of SEQ ID NO: 53. In some embodiments, the bacteriophage comprises at least 70%, 75%, 80%, 85%, 90%, 95%, or 100% sequence identity with p3693e001 (PTA-127337). In some embodiments, the Phietavirus is a p3693 comprising a nucleic acid encoding a CRISPR-Cas system disclosed herein. In some embodiments, the Phictavirus is a p3693 comprising a nucleic acid encoding a peptide disclosed herein.

In some embodiments, the Phietavirus is p3224. In some embodiments, the bacteriophage comprises at least 70%, 75%, 80%, 85%, 90%, 95%, or 100% sequence identity with p3224. In some embodiments, the p3224 is engineered to remove, replace, or inactivate a lysogenic gene or a promoter of a lysogenic gene. In some embodiments, the p3224 is engineered to remove, replace, or inactivate at least 10%, 20%, 30%, 40%, 50%, 60%, 70%, 80%, 90%, or 100% of SEQ ID NO: 54. In some embodiments, the bacteriophage comprises at least 70%, 75%, 80%, 85%, 90%, 95%, or 100% sequence identity with p3224e054. In some embodiments, the bacteriophage comprises at least 70%, 75%, 80%, 85%, 90%, 95%, or 100% sequence identity with p3224e002 (PTA-127341). In some embodiments, the Phictavirus is a p3224 comprising a nucleic acid encoding a CRISPR-Cas system disclosed herein. In some embodiments, the Phictavirus is a p3224 comprising a nucleic acid encoding a peptide disclosed herein.

In some embodiments, the Phictavirus is p5593. In some embodiments, the bacteriophage comprises at least 70%, 75%, 80%, 85%, 90%, 95%, or 100% sequence identity with p5593. In some embodiments, the p5593 is engineered to remove, replace, or inactivate a lysogenic gene or a promoter of a lysogenic gene. In some embodiments, the p5593 is engineered to remove, replace, or inactivate at least 10%, 20%, 30%, 40%, 50%, 60%, 70%, 80%, 90%, or 100% of SEQ ID NO: 55. In some embodiments, the bacteriophage comprises at least 70%, 75%, 80%, 85%, 90%, 95%, or 100% sequence identity with p5593e001 (PTA-127342). In some embodiments, the Phietavirus is a p5593 comprising a nucleic acid encoding a CRISPR-Cas system disclosed herein. In some embodiments, the Phictavirus is a p5593 comprising a nucleic acid encoding a peptide disclosed herein.

In some embodiments, the Phietavirus is p1468. In some embodiments, the bacteriophage comprises at least 70%, 75%, 80%, 85%, 90%, 95%, or 100% sequence identity with p1468. In some embodiments, the p1468 is engineered to remove, replace, or inactivate a lysogenic gene or a promoter of a lysogenic gene. In some embodiments, the p1468 is engineered to remove, replace, or inactivate at least 10%, 20%, 30%, 40%, 50%, 60%, 70%, 80%, 90%, or 100% of SEQ ID NO: 56. In some embodiments, the bacteriophage comprises at least 70%, 75%, 80%, 85%, 90%, 95%, or 100% sequence identity with p1468e003 (PTA-127343). In some embodiments, the Phictavirus is a p1468 comprising a nucleic acid encoding a CRISPR-Cas system disclosed herein. In some embodiments, the Phictavirus is a p1468 comprising a nucleic acid encoding a peptide disclosed herein.

In some embodiments, the Phietavirus is p1478. In some embodiments, the bacteriophage comprises at least 70%, 75%, 80%, 85%, 90%, 95%, or 100% sequence identity with p1478. In some embodiments, the p1478 is engineered to remove, replace, or inactivate a lysogenic gene or a promoter of a lysogenic gene. In some embodiments, the p1478 is engineered to remove, replace, or inactivate at least 10%, 20%, 30%, 40%, 50%, 60%, 70%, 80%, 90%, or 100% of SEQ ID NO: 57. In some embodiments, the bacteriophage comprises at least 70%, 75%, 80%, 85%, 90%, 95%, or 100% sequence identity with p1478e003 (PTA-127344). In some embodiments, the Phictavirus is a p1478 comprising a nucleic acid encoding a CRISPR-Cas system disclosed herein. In some embodiments, the Phictavirus is a p1478 comprising a nucleic acid encoding a peptide disclosed herein.

In some embodiments, the Phictavirus is p1494. In some embodiments, the bacteriophage comprises at least 70%, 75%, 80%, 85%, 90%, 95%, or 100% sequence identity with p1494. In some embodiments, the p1494 is engineered to remove, replace, or inactivate a lysogenic gene or a promoter of a lysogenic gene. In some embodiments, the p1494 is engineered to remove, replace, or inactivate at least 10%, 20%, 30%, 40%, 50%, 60%, 70%, 80%, 90%, or 100% of SEQ ID NO: 58. In some embodiments, the bacteriophage comprises at least 70%, 75%, 80%, 85%, 90%, 95%, or 100% sequence identity with p1494e002 (PTA-127345). In some embodiments, the Phictavirus is a p1494 comprising a nucleic acid encoding a CRISPR-Cas system disclosed herein. In some embodiments, the Phictavirus is a p1494 comprising a nucleic acid encoding a peptide disclosed herein.

In some embodiments, the Phietavirus is p1498. In some embodiments, the bacteriophage comprises at least 70%, 75%, 80%, 85%, 90%, 95%, or 100% sequence identity with p1498. In some embodiments, the p1498 is engineered to remove, replace, or inactivate a lysogenic gene or a promoter of a lysogenic gene. In some embodiments, the p1498 is engineered to remove, replace, or inactivate at least 10%, 20%, 30%, 40%, 50%, 60%, 70%, 80%, 90%, or 100% of SEQ ID NO: 52. In some embodiments, the bacteriophage comprises at least 70%, 75%, 80%, 85%, 90%, 95%, or 100% sequence identity with p1498e001 (PTA-127336). In some embodiments, the p1498 is engineered to remove, replace, or inactivate at least 10%, 20%, 30%, 40%, 50%, 60%, 70%, 80%, 90%, or 100% of SEQ ID NO: 52. In some embodiments, the Phietavirus is a p1498 comprising a nucleic acid encoding a CRISPR-Cas system disclosed herein. In some embodiments, the Phictavirus is a p1498 comprising a nucleic acid encoding a peptide disclosed herein.

In some embodiments, the bacteriophage is a Rosenblumvirus. In some embodiments, the Rosenblumvirus comprises a nucleic acid encoding a CRISPR-Cas system. In some embodiments, the Rosenblumvirus comprises a nucleic acid encoding a peptide. In some embodiments, the bacteriophage does not comprise or encode one or more components of a CRISPR-Cas system. In some embodiments, the bacteriophage does not comprise or encode an antimicrobial peptide.

In some embodiments, the Rosenblumvirus is p2808 (PTA-127332). In some embodiments, the bacteriophage comprises at least 70%, 75%, 80%, 85%, 90%, 95%, or 100% sequence identity with p2808. In some embodiments, the p2808 is engineered to remove, replace, or inactivate a lysogenic gene or a promoter of a lysogenic gene. In some embodiments, the Rosenblumvirus is a p2808 comprising a nucleic acid encoding a CRISPR-Cas system disclosed herein. In some embodiments, the Rosenblumvirus is a p2808 comprising a nucleic acid encoding a peptide disclosed herein.

In some embodiments, the Rosenblumvirus is Staphylococcus bacteriophage rv44AHJD. In some embodiments, the Rosenblumvirus is Staphylococcus bacteriophage pabna. In some embodiments, the Rosenblumvirus is Staphylococcus bacteriophage 66. In some embodiments, the Rosenblumvirus is selected from bacteriophage identified under NCBI taxonomy ID 690287. In some embodiments, the bacteriophage comprises at least 70%, 75%, 80%, 85%, 90%, 95%, or 100% sequence identity with a Rosenblumvirus. In some embodiments, the Rosenblumvirus is engineered to remove, replace, or inactivate a lysogenic gene or a promoter of a lysogenic gene. In some embodiments, the Rosenblumvirus comprising a nucleic acid encoding a CRISPR-Cas system disclosed herein. In some embodiments, the Rosenblumvirus comprises a nucleic acid encoding a peptide disclosed herein.

In some embodiments, the bacteriophage is a Kayvirus. In some embodiments, the Kayvirus comprises a nucleic acid encoding a CRISPR-Cas system. In some embodiments, the Kayvirus comprises a nucleic acid encoding a peptide. In some embodiments, the bacteriophage does not comprise or encode one or more components of a CRISPR-Cas system. In some embodiments, the bacteriophage does not comprise or encode an antimicrobial peptide.

In some embodiments, the Kayvirus is p1378 (PTA-127329). In some embodiments, the bacteriophage comprises at least 70%, 75%, 80%, 85%, 90%, 95%, or 100% sequence identity with p1378. In some embodiments, the bacteriophage comprises at least 70%, 75%, 80%, 85%, 90%, 95%, or 100% sequence identity with p1378e075 (PTA-127338). In some embodiments, the p1378 is engineered to remove, replace, or inactivate a lysogenic gene or a promoter of a lysogenic gene. In some embodiments, the bacteriophage comprises at least 70%, 75%, 80%, 85%, 90%, 95%, or 100% sequence identity with p1378e062 (PTA-127333). In some embodiments, the p1378 is engineered to remove, replace, or inactivate at least 10%, 20%, 30%, 40%, 50%, 60%, 70%, 80%, 90%, or 100% of SEQ ID NO: 49. In some embodiments, the bacteriophage comprises at least 70%, 75%, 80%, 85%, 90%, 95%, or 100% sequence identity with p1378e074 (PTA-127334). In some embodiments, the p1378 is engineered to remove, replace, or inactivate at least 10%, 20%, 30%, 40%, 50%, 60%, 70%, 80%, 90%, or 100% of SEQ ID NO: 50. In some embodiments, the p1378 is engineered to remove, replace, or inactivate at least 10%, 20%, 30%, 40%, 50%, 60%, 70%, 80%, 90%, or 100% of SEQ ID NO: 51. In some embodiments, the p1378 is engineered to remove, replace, or inactivate at least 10%, 20%, 30%, 40%, 50%, 60%, 70%, 80%, 90%, or 100% of SEQ ID NO: 50 and at least 10%, 20%, 30%, 40%, 50%, 60%, 70%, 80%, 90%, or 100% of SEQ ID NO: 51. In some embodiments, the bacteriophage comprises at least 70%, 75%, 80%, 85%, 90%, 95%, or 100% sequence identity with p1378e074. In some embodiments, the Kayvirus is a p1378 comprising a nucleic acid encoding a CRISPR-Cas system disclosed herein. In some embodiments, the Kayvirus is a p1378 comprising a nucleic acid encoding a peptide disclosed herein.

In some embodiments, the Kayvirus is p1381 (PTA-127330). In some embodiments, the bacteriophage comprises at least 70%, 75%, 80%, 85%, 90%, 95%, or 100% sequence identity with p1381. In some embodiments, the bacteriophage comprises at least 70%, 75%, 80%, 85%, 90%, 95%, or 100% sequence identity with p1381e017 (PTA-127339). In some embodiments, the p1381 is engineered to remove, replace, or inactivate a lysogenic gene or a promoter of a lysogenic gene. In some embodiments, the Kayvirus is a p1381 comprising a nucleic acid encoding a CRISPR-Cas system disclosed herein. In some embodiments, the Kayvirus is a p1381 comprising a nucleic acid encoding a peptide disclosed herein.

In some embodiments, the Kayvirus is p4815 (PTA-127331). In some embodiments, the bacteriophage comprises at least 70%, 75%, 80%, 85%, 90%, 95%, or 100% sequence identity with p4815. In some embodiments, the bacteriophage comprises at least 70%, 75%, 80%, 85%, 90%, 95%, or 100% sequence identity with p4815e037 (PTA-127335). In some embodiments, the bacteriophage comprises at least 70%, 75%, 80%, 85%, 90%, 95%, or 100% sequence identity with p4815e053 (PTA-127340). In some embodiments, the p4815 is engineered to remove, replace, or inactivate a lysogenic gene or a promoter of a lysogenic gene. In some embodiments, the Kayvirus is a p4815 comprising a nucleic acid encoding a CRISPR-Cas system disclosed herein. In some embodiments, the Kayvirus is a p4815 comprising a nucleic acid encoding a peptide disclosed herein.

In some embodiments, bacteriophages of interest are obtained from environmental sources or from commercial research vendors. In some embodiments, obtained bacteriophages are screened for lytic activity against a library of bacteria and their associated strains. In some embodiments, the bacteriophages are screened against a library of bacteria and their associated strains for their ability to generate primary resistance in the screened bacteria.

In some embodiments, a nucleic acid sequence is inserted into a bacteriophage, e.g., a nucleic acid sequence encoding one or more components of a CRISPR-Cas system, and/or a peptide. In some embodiments, the insertion of the nucleic acid sequence into a bacteriophage preserves the lytic activity of the bacteriophage. In some embodiments, the nucleic acid sequence is inserted into the bacteriophage genome. In some embodiments, the nucleic acid sequence is inserted into the bacteriophage genome at a transcription terminator site at the end of an operon of interest. In some embodiments, the nucleic acid sequence is inserted into the bacteriophage genome as a replacement for one or more removed non-essential genes. In some embodiments, the nucleic acid sequence is inserted into the bacteriophage genome as a replacement for one or more removed lysogenic genes. In some embodiments, the replacement of non-essential and/or lysogenic genes with the nucleic acid sequence does not affect the lytic activity of the bacteriophage. In some embodiments, the replacement of non-essential and/or lysogenic genes with the nucleic acid sequence preserves the lytic activity of the bacteriophage. In some embodiments, the replacement of non-essential and/or lysogenic genes with the nucleic acid sequence enhances the lytic activity of the bacteriophage. In some embodiments, the replacement of non-essential and/or lysogenic genes with the nucleic acid sequence renders a lysogenic bacteriophage lytic.

### Bacteriophage Cocktails

Also disclosed herein is a cocktail comprising two or more bacteriophage. In some embodiments, the two or more bacteriophage are selected from the lineage consisting of a Kayvirus, a Twortvirus, a Rosenblumvirus, a Phictavirus or a Triavirus. In some embodiments, at least one bacteriophage of the cocktail comprises a CRISPR-Cas system. In some embodiments, at least two bacteriophages of the cocktail comprise a CRISPR-Cas system. In some embodiments, at least three bacteriophage of the cocktail comprise a CRISPR-Cas system. In some embodiments, at least four bacteriophage of the cocktail comprise a CRISPR-Cas system. In some embodiments, at least one bacteriophage of the cocktail does not comprise a CRISPR-Cas system. In some embodiments, at least two bacteriophages of the cocktail do not comprise a CRISPR-Cas system. In some embodiments, at least one bacteriophage of the cocktail comprises an antimicrobial peptide as described herein. In some embodiments, at least two bacteriophages of the cocktail comprise a nucleic acid encoding an antimicrobial peptide as described herein. In some embodiments, at least one bacteriophage comprises a nucleic acid encoding a CRISPR-Cas system and at least one bacteriophage comprises a nucleic acid encoding an antimicrobial peptide. In some embodiments, at least one bacteriophage of the cocktail comprises a nucleic acid encoding a CRISPR-Cas system. In some embodiments, the bacteriophage of the cocktail do not comprise a nucleic acid encoding a CRISPR-Cas system or an antimicrobial peptide.

In some embodiments, the cocktail comprises a Phietavirus. In some cases, the Phietavirus is engineered to remove, replace, or inactivate a lysogenic gene or a promoter of a lysogenic gene. In some example cocktails, the cocktail comprises a Phietavirus and a Rosenblumvirus. In some example cocktails, the cocktail comprises a Phictavirus and a Kayvirus. In some example cocktails, the cocktail comprises a Phietavirus, Rosenblumvirus, and Kayvirus. In some cases, the Phietavirus comprises a nucleic acid encoding a CRISPR-Cas system. In some cases, the Phictavirus comprises a nucleic acid encoding an antimicrobial peptide. In some cases, the Phictavirus binds to a different bacteria receptor than another bacteriophage in the cocktail. In some cases, if the bacteria develops resistance and/or has a mutation that prevents infection with the another bacteriophage, the Phietavirus is capable of infecting the bacteria. In some such cases, the cocktail is more resilient against resistance formation by the bacteria than a single bacteriophage.

In some embodiments, the cocktail comprises a Rosenblumvirus. In some example cocktails, the cocktail comprises a Rosenblumvirus and a Phictavirus. In some cases, the Phictavirus is engineered to remove, replace, or inactivate a lysogenic gene or a promoter of a lysogenic gene. In some example cocktails, the cocktail comprises a Rosenblumvirus and a Kayvirus. In some example cocktails, the cocktail comprises a Phietavirus, Rosenblumvirus, and Kayvirus. In some cases, the Rosenblumvirus comprises a nucleic acid encoding a CRISPR-Cas system. In some cases, the Rosenblumvirus comprises a nucleic acid encoding an antimicrobial peptide. In some cases, the Rosenblumvirus binds to a different bacteria receptor than another bacteriophage in the cocktail. In some cases, if the bacteria develops resistance and/or has a mutation that prevents infection with the another bacteriophage, the Rosenblumvirus is capable of infecting the bacteria. In some such cases, the cocktail is more resilient against resistance formation by the bacteria than a single bacteriophage.

In some embodiments, the cocktail comprises a Kayvirus. In some example cocktails, the cocktail comprises a Kayvirus and a Rosenblumvirus. In some example cocktails, the cocktail comprises a Kayvirus and a Phietavirus. In some cases, the Phictavirus is engineered to remove, replace, or inactivate a lysogenic gene or a promoter of a lysogenic gene. In some example cocktails, the cocktail comprises a Phietavirus, Rosenblumvirus, and Kayvirus. In some cases, the Kayvirus comprises a nucleic acid encoding a CRISPR-Cas system. In some cases, the Kayvirus comprises a nucleic acid encoding an antimicrobial peptide. In some cases, the Kayvirus binds to a different bacteria receptor than another bacteriophage in the cocktail. In some cases, if the bacteria develops resistance and/or has a mutation that prevents infection with the another bacteriophage, the Kayvirus is capable of infecting the bacteria. In some such cases, the cocktail is more resilient against resistance formation by the bacteria than a single bacteriophage.

In some embodiments, a plurality of bacteriophages are used together. In some embodiments, the plurality of bacteriophages used together targets the same or different bacteria within a sample or subject. In some embodiments, a cocktail comprising a plurality of bacteriophages is used together. In some embodiments, the cocktail comprises at least 2, 3, 4, 5, 6, 7, 8, 9, 10, 11, 12, 13, 14, 15, 16, 17, 18, 19, 20, or more than 20 phages. In some embodiments, the cocktail comprises 2 phages. In some embodiments, the cocktail comprises 3 phages. In some embodiments, the cocktail comprises 4 phages. In some embodiments, the cocktail comprises 5 phages. In some embodiments, the cocktail comprises 6 phages. In some embodiments, at least one bacteriophage in the cocktail comprises a CRISPR array. In some embodiments, at least 2, 3, 4, 5, 6, 7, 8, 9, 10, 11, 12, 13, 14, 15, 16, 17, 18, 19, 20, or more than 20 bacteriophages present in the cocktail comprise a CRISPR array. In some embodiments, at least one bacteriophage in the cocktail comprises a nucleic acid sequence encoding a Cascade polypeptide. In some embodiments, at least 2, 3, 4, 5, 6, 7, 8, 9, 10, 11, 12, 13, 14, 15, 16, 17, 18, 19, 20, or more than 20 bacteriophages present in the cocktail comprise a nucleic acid sequence encoding a Cascade polypeptide. In some embodiments, at least one bacteriophage in the cocktail comprises a nucleic acid sequence encoding a Cas3 polypeptide. In some embodiments, at least 2, 3, 4, 5, 6, 7, 8, 9, 10, 11, 12, 13, 14, 15, 16, 17, 18, 19, 20, or more than 20 bacteriophages present in the cocktail comprise a nucleic acid sequence encoding a Cas3 polypeptide. In some embodiments, at least one bacteriophage in the cocktail comprises a nucleic acid sequence encoding an antimicrobial peptide.

In some embodiments, the cocktail comprises at least two bacteriophage, wherein the bacteriophage comprises p1378, p4815, p1494e002, or p2808. In some embodiments, the first bacteriophage comprises at least 70%, 75%, 80%, 85%, 90%, 95% or 100% sequence identity with p1378. In some embodiments, the second bacteriophage comprises at least 70%, 75%, 80%, 85%, 90%, 95% or 100% sequence identity with p4815. In some embodiments, the third bacteriophage comprises at least 70%, 75%, 80%, 85%, 90%, 95% or 100% sequence identity with p1494e002. In some embodiments, the fourth bacteriophage comprises at least 70%, 75%, 80%, 85%, 90%, 95% or 100% sequence identity with p2808. In some embodiments, at least one, two, three, or four bacteriophage comprise a CRISPR-Cas system. In some embodiments, at least one, two, three, or four bacteriophage do not comprise a CRISPR Cas system.

In some embodiments, the first bacteriophage comprises at least 70%, 75%, 70%, 85%, 90%, 95% or 100% sequence identity with p1378. In some embodiments, the cocktail comprises a second bacteriophage, wherein the second bacteriophage comprises at least 70%, 75%, 80%, 85%, 90%, 95%, or 100% sequence identity with p1381, p4815, p1378e062, p1378e074, p4815e037, p1378e075, p1381e017, p4815e053, p1498e001, p3693e001, p3224e002, p5593e001, p1468e003, p1478e003, p1494e002, p2808, or p1473. In some embodiments, the cocktail comprises a third bacteriophage, wherein the third bacteriophage comprises at least 70%, 75%, 80%, 85%, 90%, 95%, or 100% sequence identity with p1381, p4815, p1378e062, p1378e074, p4815e037, p1378e075, p1381e017, p4815e053, p1498e001, p3693e001, p3224e002, p5593e001, p1468e003, p1478e003, p1494e002, p2808, or p1473. In some embodiments, the cocktail comprises a fourth bacteriophage, wherein the fourth bacteriophage comprises at least 70%, 75%, 80%, 85%, 90%, 95%, or 100% sequence identity with p1381, p4815, p1378e062, p1378e074, p4815e037, p1378e075, p1381e017, p4815e053, p1498e001, p3693e001, p3224e002, p5593e001, p1468e003, p1478e003, p1494e002, p2808, or p1473. In some embodiments, the cocktail comprises a fifth bacteriophage, wherein the fifth bacteriophage comprises at least 70%, 75%, 80%, 85%, 90%, 95%, or 100% sequence identity with p1381, p4815, p1378e062, p1378e074, p4815e037, p1378e075, p1381e017, p4815e053, p1498e001, p3693e001, p3224e002, p5593e001, p1468e003, p1478e003, p1494e002 p2808, or p1473. In some embodiments, the cocktail comprises a sixth bacteriophage, wherein the sixth bacteriophage comprises at least 70%, 75%, 80%, 85%, 90%, 95%, or 100% sequence identity with p1381, p4815, p1378e062, p1378e074, p4815e037, p1378e075, p1381e017, p4815e053, p1498e001, p3693e001, p3224e002, p5593e001, p1468e003, p1478e003, p1494e002, p2808, or p1473.

In some embodiments, the first bacteriophage comprises at least 70%, 75%, 70%, 85%, 90%, 95% or 100% sequence identity with p4815. In some embodiments, the cocktail comprises a second bacteriophage, wherein the second bacteriophage comprises at least 70%, 75%, 80%, 85%, 90%, 95%, or 100% sequence identity with p1378, p1381, p1378e062, p1378e074, p4815e037, p1378e075, p1381e017, p4815e053, p1498e001, p3693e001, p3224e002, p5593e001, p1468e003, p1478e003, p1494e002, p2808, or p1473. In some embodiments, the cocktail comprises a third bacteriophage, wherein the third bacteriophage comprises at least 70%, 75%, 80%, 85%, 90%, 95%, or 100% sequence identity with p1378, p1381, p1378e062, p1378e074, p4815e037, p1378e075, p1381e017, p4815e053, p1498e001, p3693e001, p3224e002, p5593e001, p1468e003, p1478e003, p1494e002, p2808, or p1473. In some embodiments, the cocktail comprises a fourth bacteriophage, wherein the fourth bacteriophage comprises at least 70%, 75%, 80%, 85%, 90%, 95%, or 100% sequence identity with p1378, p1381, p1378e062, p1378e074, p4815e037, p1378e075, p1381e017, p4815e053, p1498e001, p3693e001, p3224e002, p5593e001, p1468e003, p1478e003, p1494e002, p2808, or p1473. In some embodiments, the cocktail comprises a fifth bacteriophage, wherein the fifth bacteriophage comprises at least 70%, 75%, 80%, 85%, 90%, 95%, or 100% sequence identity with p1378, p1381, p1378e062, p1378e074, p4815e037, p1378e075, p1381e017, p4815e053, p1498e001, p3693e001, p3224e002, p5593e001, p1468e003, p1478e003, p1494e002, p2808, or p1473. In some embodiments, the cocktail comprises a sixth bacteriophage, wherein the sixth bacteriophage comprises at least 70%, 75%, 80%, 85%, 90%, 95%, or 100% sequence identity with p1378, p1381, p1378e062, p1378e074, p4815e037, p1378e075, p1381e017, p4815e053, p1498e001, p3693e001, p3224e002, p5593e001, p1468e003, p1478e003, p1494e002, p2808, or p1473.

In some embodiments, the first bacteriophage comprises at least 70%, 75%, 70%, 85%, 90%, 95% or 100% sequence identity with p1494e002. In some embodiments, the cocktail comprises a second bacteriophage, wherein the second bacteriophage comprises at least 70%, 75%, 80%, 85%, 90%, 95%, or 100% sequence identity with p1378, p1381, p4815, p1378e062, p1378e074, p4815e037, p1378e075, p1381e017, p4815e053, p1498e001, p3693e001, p3224e002, p5593e001, p1468e003, p1478e003, p2808, or p1473. In some embodiments, the cocktail comprises a third bacteriophage, wherein the third bacteriophage comprises at least 70%, 75%, 80%, 85%, 90%, 95%, or 100% sequence identity with p1378, p1381, p4815, p1378e062, p1378e074, p4815e037, p1378e075, p1381e017, p4815e053, p1498e001, p3693e001, p3224e002, p5593e001, p1468e003, p1478e003, p2808, or p1473. In some embodiments, the cocktail comprises a fourth bacteriophage, wherein the fourth bacteriophage comprises at least 70%, 75%, 80%, 85%, 90%, 95%, or 100% sequence identity with p1378, p1381, p4815, p1378e062, p1378e074, p4815e037, p1378e075, p1381e017, p4815e053, p1498e001, p3693e001, p3224e002, p5593e001, p1468e003, p1478e003, p2808, or p1473. In some embodiments, the cocktail comprises a fifth bacteriophage, wherein the fifth bacteriophage comprises at least 70%, 75%, 80%, 85%, 90%, 95%, or 100% sequence identity with p1378, p1381, p4815, p1378e062, p1378e074, p4815e037, p1378e075, p1381e017, p4815e053, p1498e001, p3693e001, p3224e002, p5593e001, p1468e003, p1478e003, p2808, or p1473. In some embodiments, the cocktail comprises a sixth bacteriophage, wherein the sixth bacteriophage comprises at least 70%, 75%, 80%, 85%, 90%, 95%, or 100% sequence identity with p1378, p1381, p4815, p1378e062, p1378e074, p4815e037, p1378e075, p1381e017, p4815e053, p1498e001, p3693e001, p3224e002, p5593e001, p1468e003, p1478e003, p2808, or p1473.

In some embodiments, the first bacteriophage comprises at least 70%, 75%, 70%, 85%, 90%, 95% or 100% sequence identity with p2808. In some embodiments, the cocktail comprises a second bacteriophage, wherein the second bacteriophage comprises at least 70%, 75%, 80%, 85%, 90%, 95%, or 100% sequence identity with p1378, p1381, p4815, p1378e062, p1378e074, p4815e037, p1378e075, p1381e017, p4815e053, p1498e001, p3693e001, p3224e002, p5593e001, p1468e003, p1478e003, p1494e002, or p1473. In some embodiments, the cocktail comprises a third bacteriophage, wherein the third bacteriophage comprises at least 70%, 75%, 80%, 85%, 90%, 95%, or 100% sequence identity with p1378, p1381, p4815, p1378e062, p1378e074, p4815e037, p1378e075, p1381e017, p4815e053, p1498e001, p3693e001, p3224e002, p5593e001, p1468e003, p1478e003, p1494e002, or p1473. In some embodiments, the cocktail comprises a fourth bacteriophage, wherein the fourth bacteriophage comprises at least 70%, 75%, 80%, 85%, 90%, 95%, or 100% sequence identity with p1378, p1381, p4815, p1378e062, p1378e074, p4815e037, p1378e075, p1381e017, p4815e053, p1498e001, p3693e001, p3224e002, p5593e001, p1468e003, p1478e003, p1494e002, or p1473. In some embodiments, the cocktail comprises a fifth bacteriophage, wherein the fifth bacteriophage comprises at least 70%, 75%, 80%, 85%, 90%, 95%, or 100% sequence identity with p1378, p1381, p4815, p1378e062, p1378e074, p4815e037, p1378e075, p1381e017, p4815e053, p1498e001, p3693e001, p3224e002, p5593e001, p1468e003, p1478e003, p1494e002, or p1473. In some embodiments, the cocktail comprises a sixth bacteriophage, wherein the sixth bacteriophage comprises at least 70%, 75%, 80%, 85%, 90%, 95%, or 100% sequence identity with p1378, p1381, p4815, p1378e062, p1378e074, p4815e037, p1378e075, p1381e017, p4815e053, p1498e001, p3693e001, p3224e002, p5593e001, p1468e003, p1478e003, p1494e002 or p1473.

In some embodiments, the first bacteriophage comprises at least 70%, 75%, 70%, 85%, 90%, 95% or 100% sequence identity with p1378, p1381, p4815, p1378e062, p1378e074, p4815e037, p1378e075, p1381e017, p4815e053, p1498e001, p3693e001, p3224e002, p5593e001, p1468e003, p1478e003, p1494e002 p2808, or p1473. In some embodiments, the cocktail comprises a second bacteriophage, wherein the second bacteriophage comprises at least 70%, 75%, 80%, 85%, 90%, 95%, or 100% sequence identity with p1378, p1381, p4815, p1378e062, p1378e074, p4815e037, p1378e075, p1381e017, p4815e053, p1498e001, p3693e001, p3224e002, p5593e001, p1468e003, p1478e003, p1494e002, p2808, or p1473. In some embodiments, the cocktail comprises a third bacteriophage, wherein the third bacteriophage comprises at least 70%, 75%, 80%, 85%, 90%, 95%, or 100% sequence identity with p1378, p1381, p4815, p1378e062, p1378e074, p4815e037, p1378e075, p1381e017, p4815e053, p1498e001, p3693e001, p3224e002, p5593e001, p1468e003, p1478e003, p1494e002, p2808, or p1473. In some embodiments, the cocktail comprises a fourth bacteriophage, wherein the fourth bacteriophage comprises at least 70%, 75%, 80%, 85%, 90%, 95%, or 100% sequence identity with p1378, p1381, p4815, p1378e062, p1378e074, p4815e037, p1378e075, p1381e017, p4815e053, p1498e001, p3693e001, p3224e002, p5593e001, p1468e003, p1478e003, p1494e002, p2808, or p1473. In some embodiments, the cocktail comprises a fifth bacteriophage, wherein the fifth bacteriophage comprises at least 70%, 75%, 80%, 85%, 90%, 95%, or 100% sequence identity with p1378, p1381, p4815, p1378e062, p1378e074, p4815e037, p1378e075, p1381e017, p4815e053, p1498e001, p3693e001, p3224e002, p5593e001, p1468e003, p1478e003, p1494e002, p2808, or p1473. In some embodiments, the cocktail comprises a sixth bacteriophage, wherein the sixth bacteriophage comprises at least 70%, 75%, 80%, 85%, 90%, 95%, or 100% sequence identity with p1378, p1381, p4815, p1378e062, p1378e074, p4815e037, p1378e075, p1381e017, p4815e053, p1498e001, p3693e001, p3224e002, p5593e001, p1468e003, p1478e003, p1494e002, p2808, or p1473.

In some embodiments, the bacteriophage cocktail has a host range greater than that of an individual bacteriophage. The increased host range may allow for targeting a large number of strains of S. aureus. In some embodiments, the bacteriophage cocktail targets at least 70%, 75%, 80%, 85%, 90%, 95%, 97.5%, 99% or more than 99% of strains of S. aureus. In some embodiments, the strains of S. aureus include: b004604, b004605, b004606, b004607, b004608, b004609, b004610, b004611, b004612, b004613, b004614, b004615, b004616, b004617, b004618, b004619, b004620, b004621, b004622, b004623, b004624, b004625, b004626, b004627, b004628, b004629, b004630, b004631, b004632, b004633, b004634, b004635, b004636, b004637, b004638, b004639, b004640, b004641, b004642, b004643, b004644, b004645, b004646, b004647, b004648, b004649, b004650, b004651, b004652, b004653, b004654, b004655, b004656, b004657, b004658, b004659, b004660, b004661, b004662, b004663, b004664, b004665, b004666, b004667, b004668, b004669, b004670, b004671, b004672, b004673, b004674, b004675, b004676, b004677, b004678, b004679, b004680, b004681, b004682, b004683, b004684, b004685, b004686, b004687, b004688, b004689, b004690, b004691, b004692, b004693, b004694, b004695, b004696, b004697, b004698, b004699, b004700, b004701, b004702, b004703, b004704, b004705, b004706, b004707, b004708, b004709, b004710, b004711, b004712, b004713, b004714, b004715, b004716, b004717, b004718, b004719, b004720, b004721, b004722, b004723, b004724, b004725, b004726, b004727, b004728, b004729, b004730, b004731, b004732, b004733, b004734, b004735, b004736, b004737, b004738, b004739, b004740, b004741, b004742, b004743, b004744, b004745, b004746, b004747, b004748, b004749, b004750, b004751, b004752, b004753, b004754, b004755, b004756, b004757, b004758, b004759, b004760, b004761, b004762, b004763, b004764, b004765, b004766, b004767, b004768, b004769, b004770, b004771, b004772, b004773, b004774, b004775, b004776, b004777, b004778, b004779, b004780, b004781, b004782, b004783, b004784, b004785, b004786, b004787, b004788, b004789, b004790, b004791, b004792, b004793, b004794, b004795, b004796, b004797, b004798, b004799, b004800, b004801, b004802, b004803, b004804, b004805, b004806, b004807, b004808, b004809, b004810, b004811, b004812, b004813, b004814, b004815, b004816, b004817, b004818, b004819, b004820, b004821, b004822, b004823, b004824, b004825, b004826, b004827, b004828, b004829, b004830, b004831, b004832, b004833, b004834, b004835, b004836, b004837, b004838, b004839, b004840, b004841, b004842, b004843, b004844, b004845, b004846, b004847, b004848, b004849, b004850, b004851, b004852, b004853, b004854, b004855, b004856, b004857, b004858, b004859, b004860, b004861, b004862, b004863, b004864, b004865, b004866, b004867, b004868, b004869, b004870, b004871, b004872, b004873, b004874, b004875, b004876, b004877, b004878, b004879, b004880, b004881, b004882, b004883, b004884, b004885, b004886, b004887, b004888, b004889, b004890, b004891, b004892, b004893, b004894, b004895, b004896, b004897, b004898, b004899, b004900, b004901, b004902, b004903, b004904, b004905, b004906, b004907, b004908, b004909, b004910, and b004911. In some embodiments, the strains of S. aureus include comprise Staphylococcus bacteria having a MLST of 8, 5, 22, 15, 1, 30, 398, 105, 45, 672, 2250, 582, 72, 97, 239, 34, 87, 101, 109, 1159, 1165, 1181, 12, 121, 152, 1750, 20, 225, 25, 291, 3628, 59, 7, 779, 88, 10, 1011, 1049, 1156, 1351, 149, 1637, 1649, 1757, 1842, 188, 1970, 2066, 256, 2867, 2945, 3149, 3182, 3510, 39, 395, 4317, 47, 4730, 50, 508, 573, 6, 630, 737, 828, 848, 923, or 93. In some embodiments, the Staphylococcus bacteria comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 different MLST. In some embodiments, the Staphylococcus bacteria are isolated from a blood stream infection. In some embodiments, at least about 10%, 20%, 30%, 40%, 50%, 60%, 70%, 80%, or 90% of the Staphylococcus strains are multidrug resistant. In some embodiments, the bacteriophage cocktail targets S. aureus specifically and does not target other species of bacteria. In some embodiments, the bacteriophage cocktail targets S. aureus and does not target other Staphylococcus species. The increased host range may allow for targeting a large number of strains of Staphylococcus spp. In some embodiments, the bacteriophage cocktail targets at least 70%, 75%, 80%, 85%, 90%, 95%, 97.5%, 99% or more than 99% of strains of Staphylococcus spp. In some embodiments, the bacteriophage cocktail targets Staphylococcus spp. specifically and does not target other species of bacteria. In some embodiments, the bacteriophage cocktail targets Staphylococcus spp. and does not target non Staphylococcus spp. In some embodiments, the bacteriophage cocktail comprises two or more of: Phietavirus, Rosenblumvirus, and Kayvirus.

In some embodiments, the bacteriophage in the cocktail are selected to minimize the ability of the target bacteria to evolve resistance. In some embodiments, the cocktail comprises a first bacteriophage that binds a first receptor of a Staphylococcus bacteria, and a second bacteriophage that binds for a second receptor of the Staphylococcus bacteria, wherein the plurality of bacteriophage is more resilient to resistance by the Staphylococcus bacteria than the first or second bacteriophage alone. For instance, if the Staphylococcus bacteria develops resistance to the first bacteriophage, the bacteria is still susceptible to infection by the second bacteriophage, and vice versa. In some embodiments, at least two bacteriophage from different genus target at least 70%, 75%, 80%, 85%, 90%, 95%, 97.5%, 99% or more than 99% of strains of S. aureus. In some embodiments, the cocktail comprises at least two bacteriophage, wherein the first bacteriophage binds a first receptor on Staphylococcus and the second bacteriophage binds a second receptor on Staphylococcus. In some embodiments, the bacteriophage cocktail comprises two or more of: Phietavirus, Rosenblumvirus, and Kayvirus.

### Staphylococcus

In some embodiments, the bacterium comprises one or more species of Staphylococcus. In some embodiments, the bacterium comprises one or more strains of Staphylococcus. In some embodiments, the target bacterium is Staphylococcus aureus. In some embodiments, the target bacterium is Staphylococcus aureus, methicillin-resistant Staphylococcus aureus (MRSA), Staphylococcus epidermidis, Staphylococcus salivarius, Staphylococcus argenteus, Staphylococcus hemolyticus, Staphylococcus schweitzeri or any combination thereof.

In some embodiments, the target bacterium causes an infection or disease. In some embodiments, the infection or disease is acute or chronic. In some embodiments, the infection or disease is localized or systemic. In some embodiments, infection or disease is idiopathic. In some embodiments, the infection or disease is acquired through means including, but not limited to, respiratory inhalation, ingestion, skin and wound infections, bone infections, blood stream infections, middle-ear infections, gastrointestinal tract infections, peritoneal membrane infections, urinary tract infections, urogenital tract infections, oral soft tissue infections, intra-abdominal infections, epidermal or mucosal absorption, eye infections (including contact lens contamination), endocarditis, infections in cystic fibrosis, non-cystic fibrosis bronchiectasis (NCFB), infections of indwelling medical devices such as joint prostheses, dental implants, catheters and cardiac implants, sexual contact, and/or hospital-acquired and ventilator-associated bacterial pneumonias. In some embodiments, the target bacterium causes urinary tract infection. In some embodiments, the target bacterium causes and/or exacerbates an inflammatory disease. In some embodiments, the target bacterium causes and/or exacerbates an autoimmune disease. In some embodiments, the target bacterium causes and/or exacerbates eczema. In some embodiments, the target bacterium causes and/or exacerbates Atopic Dermatitis. In some embodiments, the target bacterium causes and/or exacerbates inflammatory bowel disease (IBD). In some embodiments, the target bacterium causes and/or exacerbates psoriasis. In some embodiments, the target bacterium causes and/or exacerbates psoriatic arthritis (PA). In some embodiments, the target bacterium causes and/or exacerbates rheumatoid arthritis (RA). In some embodiments, the target bacterium causes and/or exacerbates systemic lupus erythematosus (SLE). In some embodiments, the target bacterium causes and/or exacerbates multiple sclerosis (MS). In some embodiments, the target bacterium causes and/or exacerbates Graves' disease. In some embodiments, the target bacterium causes and/or exacerbates Hashimoto's thyroiditis. In some embodiments, the target bacterium causes and/or exacerbates Myasthenia gravis. In some embodiments, the target bacterium causes and/or exacerbates vasculitis. In some embodiments, the target bacterium causes and/or exacerbates cancer. In some embodiments, the target bacterium causes and/or exacerbates cancer progression. In some embodiments, the target bacterium causes and/or exacerbates cancer metastasis. In some embodiments, the target bacterium causes and/or exacerbates resistance to cancer therapy. In some embodiments, the therapy used to address cancer includes, but is not limited to, chemotherapy, immunotherapy, hormone therapy, targeted drug therapy, and/or radiation therapy. In some embodiments, the cancer develops in organs including, but not limited to the, anus, bladder, blood and blood components, bone, bone marrow, brain, breast, cervix uteri, colon and rectum, esophagus, kidney, larynx, lymphatic system, muscle (i.e., soft tissue), oral cavity and pharynx, ovary, pancreas, prostate, skin, small intestine, stomach, testis, thyroid, uterus, and/or vulva. In some embodiments, the target bacterium causes and/or exacerbates disorders of the central nervous system (CNS). In some embodiments, the target bacterium causes and/or exacerbates attention deficit/hyperactivity disorder (ADHD). In some embodiments, the target bacterium causes and/or exacerbates autism. In some embodiments, the target bacterium causes and/or exacerbates bipolar disorder. In some embodiments, the target bacterium causes and/or exacerbates major depressive disorder. In some embodiments, the target bacterium causes and/or exacerbates epilepsy. In some embodiments, the target bacterium causes and/or exacerbates neurodegenerative disorders including, but not limited to, Alzheimer's disease, Huntington's disease, and/or Parkinson's disease.

In some embodiments, the target bacteria comprises a Staphylococcus selected from one or more of: b004604, b004605, b004606, b004607, b004608, b004609, b004610, b004611, b004612, b004613, b004614, b004615, b004616, b004617, b004618, b004619, b004620, b004621, b004622, b004623, b004624, b004625, b004626, b004627, b004628, b004629, b004630, b004631, b004632, b004633, b004634, b004635, b004636, b004637, b004638, b004639, b004640, b004641, b004642, b004643, b004644, b004645, b004646, b004647, b004648, b004649, b004650, b004651, b004652, b004653, b004654, b004655, b004656, b004657, b004658, b004659, b004660, b004661, b004662, b004663, b004664, b004665, b004666, b004667, b004668, b004669, b004670, b004671, b004672, b004673, b004674, b004675, b004676, b004677, b004678, b004679, b004680, b004681, b004682, b004683, b004684, b004685, b004686, b004687, b004688, b004689, b004690, b004691, b004692, b004693, b004694, b004695, b004696, b004697, b004698, b004699, b004700, b004701, b004702, b004703, b004704, b004705, b004706, b004707, b004708, b004709, b004710, b004711, b004712, b004713, b004714, b004715, b004716, b004717, b004718, b004719, b004720, b004721, b004722, b004723, b004724, b004725, b004726, b004727, b004728, b004729, b004730, b004731, b004732, b004733, b004734, b004735, b004736, b004737, b004738, b004739, b004740, b004741, b004742, b004743, b004744, b004745, b004746, b004747, b004748, b004749, b004750, b004751, b004752, b004753, b004754, b004755, b004756, b004757, b004758, b004759, b004760, b004761, b004762, b004763, b004764, b004765, b004766, b004767, b004768, b004769, b004770, b004771, b004772, b004773, b004774, b004775, b004776, b004777, b004778, b004779, b004780, b004781, b004782, b004783, b004784, b004785, b004786, b004787, b004788, b004789, b004790, b004791, b004792, b004793, b004794, b004795, b004796, b004797, b004798, b004799, b004800, b004801, b004802, b004803, b004804, b004805, b004806, b004807, b004808, b004809, b004810, b004811, b004812, b004813, b004814, b004815, b004816, b004817, b004818, b004819, b004820, b004821, b004822, b004823, b004824, b004825, b004826, b004827, b004828, b004829, b004830, b004831, b004832, b004833, b004834, b004835, b004836, b004837, b004838, b004839, b004840, b004841, b004842, b004843, b004844, b004845, b004846, b004847, b004848, b004849, b004850, b004851, b004852, b004853, b004854, b004855, b004856, b004857, b004858, b004859, b004860, b004861, b004862, b004863, b004864, b004865, b004866, b004867, b004868, b004869, b004870, b004871, b004872, b004873, b004874, b004875, b004876, b004877, b004878, b004879, b004880, b004881, b004882, b004883, b004884, b004885, b004886, b004887, b004888, b004889, b004890, b004891, b004892, b004893, b004894, b004895, b004896, b004897, b004898, b004899, b004900, b004901, b004902, b004903, b004904, b004905, b004906, b004907, b004908, b004909, b004910, and b004911. In some embodiments, the Staphylococcus bacteria comprise Staphylococcus bacteria having a MLST of 8, 5, 22, 15, 1, 30, 398, 105, 45, 672, 2250, 582, 72, 97, 239, 34, 87, 101, 109, 1159, 1165, 1181, 12, 121, 152, 1750, 20, 225, 25, 291, 3628, 59, 7, 779, 88, 10, 1011, 1049, 1156, 1351, 149, 1637, 1649, 1757, 1842, 188, 1970, 2066, 256, 2867, 2945, 3149, 3182, 3510, 39, 395, 4317, 47, 4730, 50, 508, 573, 6, 630, 737, 828, 848, 923, or 93. In some embodiments, the Staphylococcus bacteria comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 different MLST. In some embodiments, the Staphylococcus bacteria are isolated from a blood stream infection.

### Lysogeny Removal

Disclosed herein, in certain embodiments, are bacteriophage engineered to remove, replace, or inactivate a lysogenic gene or a promoter of a lysogenic gene. As a non-limiting example, the lysogenic gene encodes for a repressor. In some embodiments, the bacteriophage is further engineered to comprise one or more components of a CRISPR-Cas system, and/or an antimicrobial peptide. In some embodiments, the lysogenic gene encodes for a repressor. In some embodiments, removal of the lysogenic gene comprises removing from about 1% to 100% of the lysogenic gene, or about 10 to about 1,200 base pairs of the lysogenic gene are removed.

In some embodiments, the bacteriophage is an obligate lytic bacteriophage. In some embodiments, the bacteriophage is a temperate bacteriophage with retained lysogeny genes. In some embodiments, the bacteriophage is a temperate bacteriophage with some lysogeny genes removed, replaced, or inactivated. In some embodiments, the bacteriophage is a temperate bacteriophage with a lysogeny gene removed, replaced, or inactivated, thereby rendering the bacteriophage lytic. In some embodiments, the bacteriophage is rendered lytic by removal of at least a portion of a lysogenic gene, or a promoter of a lysogenic gene. In some embodiments, the portion is at least about 1% to 100% of the nucleotides of the lysogenic gene. In some embodiments, the portion is less than about 1%. In some embodiments, the portion removed is a single base. In some embodiments, the portion is about 10 base pairs to all of the lysogenic gene. For example, the portion removed is about 10-1200, 10-1100, 10-1000, 10-900, 10-800, 10-700, 10-600, 10-500, 10-400, 10-300, 10-200, 10-100, 50-1200, 50-1100, 50-1000, 50-900, 50-800, 50-700, 50-600, 50-500, 50-400, 50-300, 50-200, 50-100, 100-1200, 100-1100, 100-1000, 100-900, 100-800, 100-700, 100-600, 100-500, 100-400, 100-300, or 100-200 base pairs. In some embodiments, the lysogenic gene encodes for an amino acid sequence at least about 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 47 or 48. In some embodiments, the lysogenic region comprises a sequence at least about 80%, 81%, 82%, 83%, 84%, 85%, 86%, 87%, 88%, 89%, 90%, 91%, 92%, 93%, 94%, 95%, 96%, 97%, 98%, 99% or 100% identical to any one of SEQ ID NOS: 49-58. In some embodiments, the lysogenic region comprises a sequence at least about 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 49. In some embodiments, the lysogenic region comprises a sequence at least about 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 50. In some embodiments, the lysogenic region comprises a sequence at least about 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 51. In some embodiments, the lysogenic region comprises a sequence at least about 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 52. In some embodiments, the lysogenic region comprises a sequence at least about 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 53. In some embodiments, the lysogenic region comprises a sequence at least about 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 54. In some embodiments, the lysogenic region comprises a sequence at least about 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 55. In some embodiments, the lysogenic region comprises a sequence at least about 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 56. In some embodiments, the lysogenic region comprises a sequence at least about 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 57. In some embodiments, the lysogenic region comprises a sequence at least about 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 58. In some embodiments, at least a portion of any one of SEQ ID NOS: 49-58 is removed. For instance, at least a portion is at least 1%, 10%, 20%, 30%, 40%, 50%, 60%, 70%, 80%, or 90% of any one of SEQ ID NOS: 49-58.

In some embodiments, the nucleic acid sequence is introduced into the bacteriophage genome at a first location while one or more non-essential and/or lysogenic genes are separately removed and/or inactivated from the bacteriophage genome at a separate location. In some embodiments, the nucleic acid sequence is introduced into the bacteriophage at a first location while one or more non-essential and/or lysogenic genes are separately removed and/or inactivated from the bacteriophage genome at multiple separate locations. In some embodiments, the removal and/or inactivation of one or more non-essential and/or lysogenic genes does not affect the lytic activity of the bacteriophage. In some embodiments, the removal and/or inactivation of one or more non-essential and/or lysogenic genes preserves the lytic activity of the bacteriophage. In some embodiments, the removal of one or more non-essential and/or lysogenic genes renders a lysogenic bacteriophage into a lytic bacteriophage.

In some embodiments, the bacteriophage is a temperate bacteriophage which has been rendered lytic by any of the aforementioned means. In some embodiments, a temperate bacteriophage is rendered lytic by the removal, replacement, or inactivation of one or more lysogenic genes. In some embodiments, the lytic activity of the bacteriophage is due to the removal, replacement, or inactivation of at least one lysogeny gene. In some embodiments, the lysogenic gene plays a role in the maintenance of lysogenic cycle in the bacteriophage. In some embodiments, the lysogenic gene plays a role in establishing the lysogenic cycle in the bacteriophage. In some embodiments, the lysogenic gene plays a role in both establishing the lysogenic cycle and in the maintenance of the lysogenic cycle in the bacteriophage.

In some embodiments, the bacteriophage is rendered lytic by removal of a sequence at least about 80%, 81%, 82%, 83%, 84%, 85%, 86%, 87%, 88%, 89%, 90%, 91%, 92%, 93%, 94%, 95%, 96%, 97%, 98%, 99% or 100% identical to any one of SEQ ID NOS: 49-58. In some embodiments, at least a portion of any one of SEQ ID NOS: 49-58 is removed. For instance, at least a portion is at least 1%, 10%, 20%, 30%, 40%, 50%, 60%, 70%, 80%, or 90% of any one of SEQ ID NOS: 49-58.

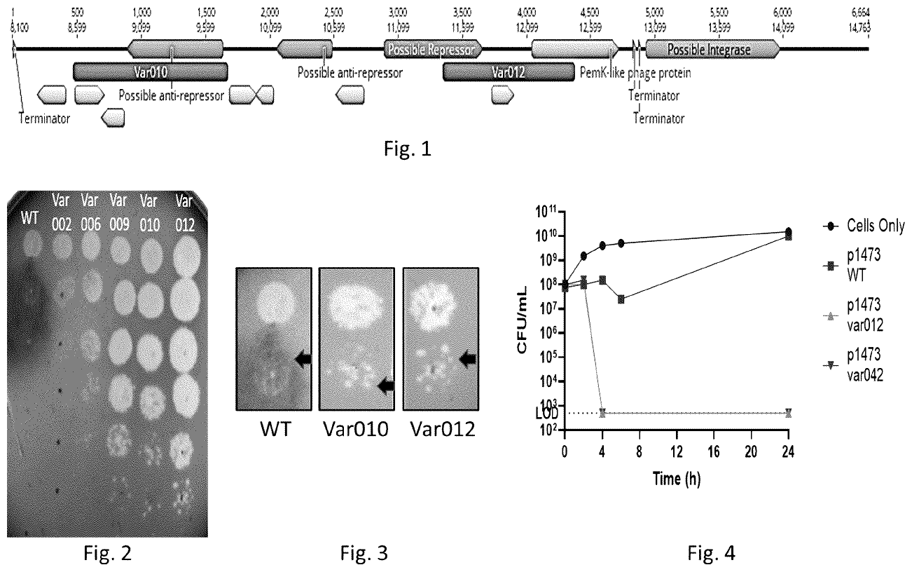

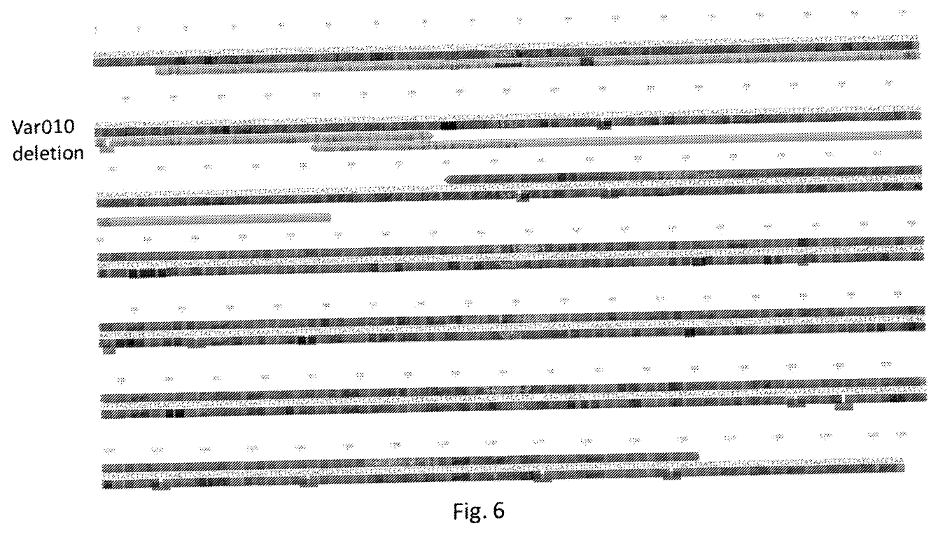

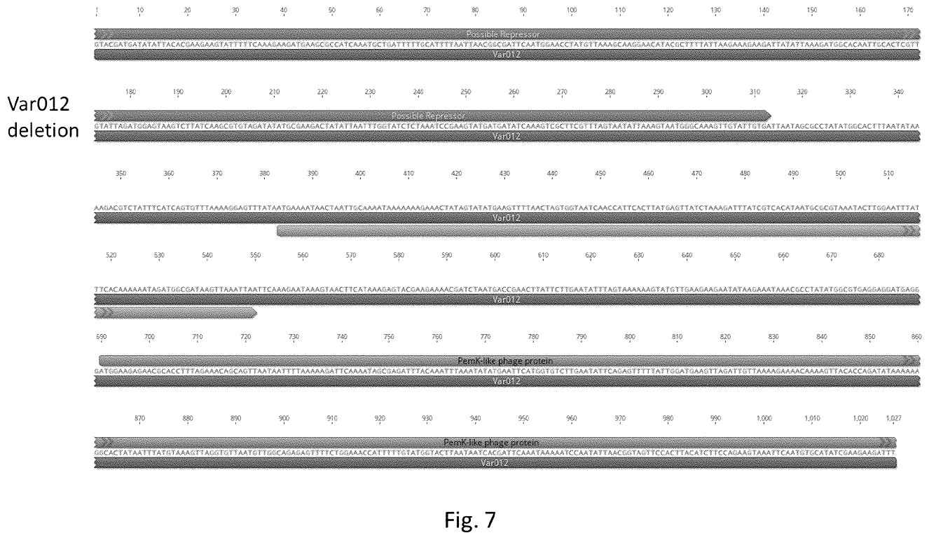

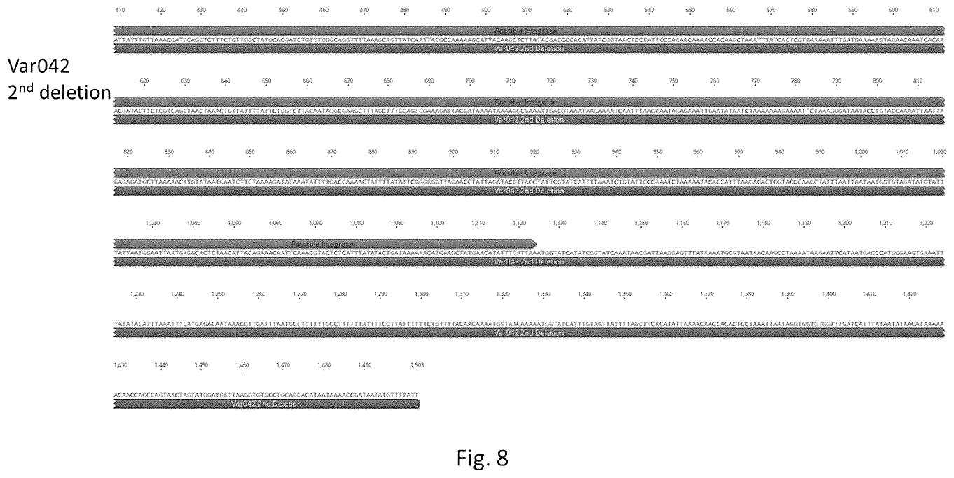

In some embodiments, the lysogenic gene is a repressor gene. In some embodiments, the bacteriophage is rendered lytic by removal of the “var010” region annotated as “var010” deletion in FIG. 6 (SEQ ID NO: 30). In some embodiments, the bacteriophage is rendered lytic by removal of at least 100, 200, 300, 400, 500, 600, 700, 800, 900, 1000, 1100 or 1200 nucleotides of the var010 region. In some embodiments, the bacteriophage is rendered lytic by removal of the “var012” region annotated as “var012” deletion in FIG. 7 (SEQ ID NO: 31). In some embodiments, the bacteriophage is rendered lytic by removal of at least 100, 200, 300, 400, 500, 600, 700, 800, 900, or 1000 nucleotides of the var012 region. In some embodiments, the bacteriophage is rendered lytic by removal of at least In some embodiments, the bacteriophage is rendered lytic by removal of the “var042” region annotated as “var042” deletion in FIG. 8 (SEQ ID NO: 32). In some embodiments, the bacteriophage is rendered lytic by removal of at least 100, 200, 300, 400, 500, 600, 700, 800, 900, 1000, 1100, 1200, 1300, 1400, or 1500 nucleotides of the var042 region. In some embodiments, the bacteriophage is rendered lytic by removal of the “var009” deletion (SEQ ID NO: 30). In some embodiments, the bacteriophage is rendered lytic by removal of at least 100, 200, 300, 400, 500, 600, 700, 800, 900, 1000, 1100, 1200, 1300, 1400, or 1500 nucleotides of the var009 region. In some embodiments, the bacteriophage is rendered lytic by removal of at least 10, 20, 30, or 40 nucleotides of the var0022 deletion (SEQ ID NO: 58). In some embodiments, the bacteriophage is rendered lytic by removal of at least 10, 20, 30, 40, 50, 60, 70, 80, 90, 100, 200, 300, 400, 500, 600, 700, 800, 900, 1000, 1100, 1200, 1300, 1400, or 1500 nucleotides encoding the repressor having an amino acid sequence at least about 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 47 or 48. In some embodiments, the bacteriophage is rendered lytic by the deletion of an antirepressor gene integrase, Pem-K like phage protein gene, or a combination thereof. In some embodiments, the bacteriophage is rendered lytic by the deletion of a gene depicted in FIG. 1. In some embodiments, the bacteriophage is rendered lytic by the deletion of an antirepressor gene, integrase, Pem-K like phage protein gene, or a combination thereof, wherein the gene is depicted in FIG. 1. In some embodiments, the bacteriophage is rendered lytic by deletion of at least 10-2000, 20-2000, 30-2000, 40-2000, 50-2000, 60-2000, 70-2000, 80-2000, 90-2000, 100-2000, 200-2000, 300-2000, 400-2000, 500-2000, 600-2000, 700-2000, 80-2000, 900-2000, 1000-2000, 1100-2000, 1200-2000, 1300-2000, 1400-2000, 1500-2000, 1600-2000,1700-2000, 1800-2000 or 1900-2000 base pairs of an antirepressor gene. In some embodiments, the bacteriophage is rendered lytic by deletion of at least 5%, 10%, 15%, 20%, 25%, 30%, 35%, 40%, 45%, 50%, 55%, 60%, 65%, 70%, 75%, 80%, 85%, 90% or 95% of an antirepressor gene. In some embodiments, the lysogenic gene is cl repressor gene. In some embodiments, the lysogenic gene is an activator gene. In some embodiments, the lysogenic gene is cell gene. In some embodiments, the lysogenic gene is Cro gene. In some embodiments, the lysogenic gene is int (integrase) gene. In some embodiments, two or more lysogeny genes are removed, replaced, or inactivated to cause arrest of a bacteriophage lysogeny cycle and/or induction of a lytic cycle. In some embodiments, a temperate bacteriophage is rendered lytic by the insertion of one or more lytic genes. In some embodiments the lytic gene encodes a holin, an endolysin, an endopeptidase, a pinholin, a spanin, or an Rz/Rz1-like protein. In some embodiments, the lytic gene encodes a holin. In some embodiments, the lytic gene encodes an endolysin. In some embodiments, the lytic gene encodes an endopeptidase. In some embodiments, the lytic gene encodes a pinholin. In some embodiments, the lytic gene encodes a spanin. In some embodiments, the lytic gene encodes an Rz/Rz-1 like protein. In some embodiments, a temperate bacteriophage is rendered lytic by the insertion of one or more genes that contribute to the induction of a lytic cycle. In some embodiments, a temperate bacteriophage is rendered lytic by altering the expression of one or more genes that contribute to the induction of a lytic cycle. In some embodiments, a temperate bacteriophage phenotypically changes from a lysogenic bacteriophage to a lytic bacteriophage. In some embodiments, a temperate bacteriophage is rendered lytic by environmental alterations. In some embodiments, environmental alterations include, but are not limited to, alterations in temperature, pH, or nutrients, exposure to antibiotics, hydrogen peroxide, foreign DNA, or DNA damaging agents, presence of organic carbon, and presence of heavy metal (e.g. in the form of chromium (VI). In some embodiments, a temperate bacteriophage that is rendered lytic is prevented from reverting to lysogenic state. In some embodiments, a temperate bacteriophage that is rendered lytic is prevented from reverting back to lysogenic state by way the self-targeting activity of the first introduced CRISPR array. In some embodiments, a temperate bacteriophage that is rendered lytic is prevented from reverting back to lysogenic state by way of introducing an additional CRISPR array. In some embodiments, the bacteriophage does not confer any new properties onto the target bacterium beyond cellular death caused by lytic activity of the bacteriophage and/or the activity of the first or second CRISPR array.

In some embodiments, the replacement, removal, inactivation, or any combination thereof, of one or more non-essential and/or lysogenic genes is achieved by chemical, biochemical, and/or any suitable method. In some embodiments, the insertion of one or more lytic genes is achieved by any suitable chemical, biochemical, and/or physical method by homologous recombination.

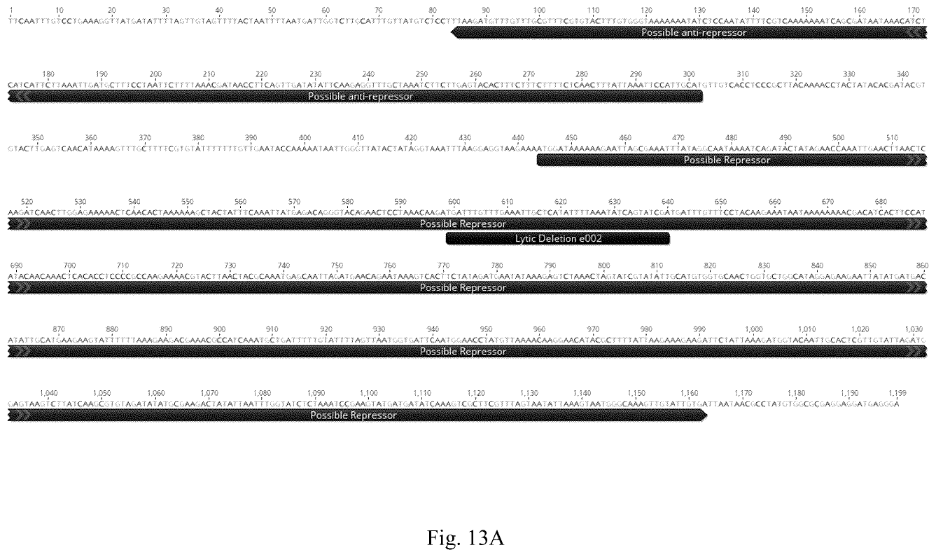

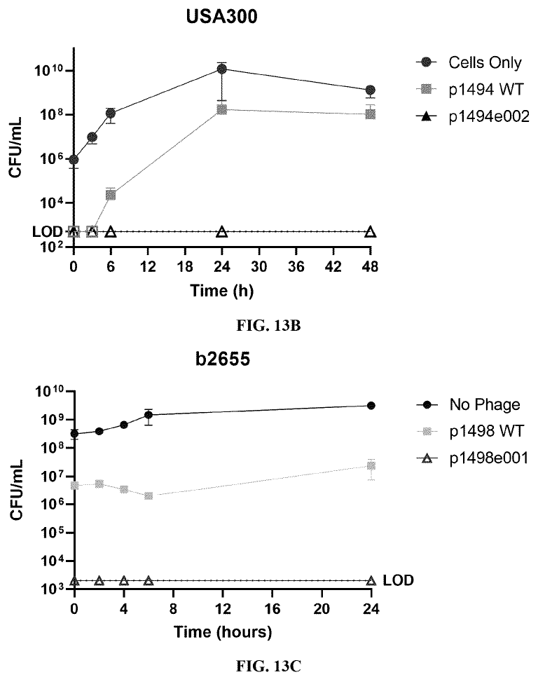

Further disclosed herein, in some embodiments, are temperate bacteriophages comprising a first nucleic acid sequence encoding a first spacer sequence or a crRNA transcribed therefrom, wherein the first spacer sequence is complementary to a target nucleotide sequence from a target gene in a target bacterium, provided that the bacteriophage is rendered lytic by removal of the region annotated as “var010” in FIG. 6, “var012” in FIG. 7, “var042” in FIG. 8, “e002” or “var0022” in FIG. 13A (SEQ ID NO: 58), at least a portion of SEQ ID NO: 47 or 48, or combinations thereof. Further disclosed herein, in some embodiments, are temperate bacteriophages comprising a first nucleic acid sequence encoding a first spacer sequence or a crRNA transcribed therefrom, wherein the first spacer sequence is complementary to a target nucleotide sequence from a target gene in a target bacterium, provided that the bacteriophage is rendered lytic by removal of a sequence at least about 80%, 81%, 82%, 83%, 84%, 85%, 86%, 87%, 88%, 89%, 90%, 91%, 92%, 93%, 94%, 95%, 96%, 97%, 98%, 99% or 100% identical to any one of SEQ ID NOS: 49-58. In some embodiments, at least a portion of any one of SEQ ID NOS: 49-58 is removed. For instance, at least a portion is at least 1%, 10%, 20%, 30%, 40%, 50%, 60%, 70%, 80%, or 90% of any one of SEQ ID NOS: 49-58. In some embodiments, the bacteriophage infects multiple bacterial strains. In some embodiments, the target nucleotide sequence comprises all or a part of a promoter sequence for the target gene. In some embodiments, the target nucleotide sequence comprises all or a part of a nucleotide sequence located on a coding strand of a transcribed region of the target gene. In some embodiments, the target nucleotide sequence comprises at least a portion of an essential gene that is needed for survival of the target bacterium. In some embodiments, the essential gene is Tsf, acpP, gapA, infA, secY, csrA, trmD, fisA, fusA, glyQ, eno, nusG, dnaA, pheS, rplB, gltX, hisS, rplC, aspS, gyrB, dnaE, rpoA, rpoB, pheT, infB, rpsC, rplF, alaS, leuS, serS, rplD, gyrA, glmS, fus, adk, rpsK, rplR, ctrA, parC, tRNA-Ser, tRNA-Asn, or metK. In some embodiments, the target nucleotide sequence is in a non-essential gene or other genomic locus. In some embodiments, the target nucleotide sequence is in a non-essential gene. In some embodiments, the target nucleotide sequence is in a non-essential genomic locus. In some embodiments, the target nucleotide sequence is a noncoding sequence. In some embodiments, the noncoding sequence is an intergenic sequence. In some embodiments, the spacer sequence is complementary to a target nucleotide sequence of a highly conserved sequence in a target bacterium. In some embodiments, the spacer sequence is complementary to a target nucleotide sequence of a sequence present in the target bacterium. In some embodiments, the spacer sequence is complementary to a target nucleotide sequence that comprises all or a part of a promoter sequence of the essential gene. In some embodiments, the first nucleic acid sequence comprises a first CRISPR array further comprising at least one repeat sequence. In some embodiments, the at least one repeat sequence is operably linked to the first spacer sequence at either its 5′ end or its 3′ end. In some embodiments, the target bacterium is S. aureus.

### Antimicrobial Agents and Peptides

In some embodiments, a bacteriophage disclosed herein is further genetically modified to express an antibacterial peptide, a functional fragment of an antibacterial peptide, and/or a lytic gene. In some embodiments, a bacteriophage disclosed herein express at least one antimicrobial agent or peptide disclosed herein. In some embodiments, the bacteriophage comprises a nucleic acid that encodes a peptide that prevents phage degradation or a peptide that assists in breaking down or degrading biofilm matrix.

In some embodiments, a bacteriophage described herein comprises a nucleic acid that encodes a peptide that prevents phage degradation or enables escape of the phage from the host defenses. In some embodiments, a bacteriophage disclosed herein comprises a nucleic acid sequence that encodes an enzybiotic where the protein product of the nucleic acid sequence targets phage resistant bacteria. In some embodiments, the peptide comprises TreA (e.g., a sequence at least 80% identical to SEQ ID NO: 10). In some embodiments, the peptide comprises Ipi (e.g., a sequence at least 80% identical to SEQ ID NO: 11).

In some embodiments, the bacteriophage comprises nucleic acids which encode enzymes which assist in breaking down or degrading biofilm matrix. In some embodiments, a bacteriophage disclosed herein comprises nucleic acids encoding Dispersin D aminopeptidase, amylase, carbohydrase, carboxypeptidase, catalase, cellulase, chitinase, cutinase, cyclodextrin glycosyltransferase, deoxyribonuclease, esterase, alpha-galactosidase, beta-galactosidase, glucoamylase, alpha-glucosidase, beta-glucosidase, haloperoxidase, invertase, laccase, lipase, mannosidase, oxidase, pectinolytic enzyme, peptidoglutaminase, peroxidase, phytase, polyphenoloxidase, proteolytic enzyme, ribonuclease, transglutaminase, xylanase or lyase. In some embodiments, the enzyme is selected from the group consisting of cellulases, such as glycosyl hydroxylase family of cellulases, such as glycosyl hydroxylase 5 family of enzymes also called cellulase A; polyglucosamine (PGA) depolymerases; and colonic acid depolymerases, such as 1,4-L-fucodise hydrolase, colanic acid, depolymerazing alginase, DNase I, or combinations thereof. In some embodiments, a bacteriophage disclosed herein secretes an enzyme disclosed herein. In some embodiments, the peptide disrupts quorum sensing and biofilm formation. In some embodiments, the peptide increases the sensitivity of a bacterial cell to an antibiotic. In some embodiments, the enzyme comprises DNAse I (e.g., a sequence at least 80% identical to SEQ ID NO: 9). In some embodiments, the enzyme comprises RIP (e.g., a sequence at least 80% identical to SEQ ID NO: 14). In some embodiments, the enzyme comprises FS3 (e.g., a sequence at least 80% identical to SEQ ID NO: 12).

In some embodiments, an antimicrobial agent or peptide is expressed and/or secreted by a bacteriophage disclosed herein. In some embodiments, the antimicrobial agent or peptide comprises PLNC8a. In some embodiments, the antimicrobial agent or peptide comprises PLNC8B. In some embodiments, the antimicrobial agent or peptide comprises LytM. In some embodiments, the antimicrobial agent or peptide comprises an anti-restriction modification enzyme. In some embodiments, the antimicrobial agent or peptide comprises lacticin Q (LnqQ, e.g., a sequence at least 80% identical to SEQ ID NO: 13 or 15). In some embodiments, the LnqQ peptide is at least 80%, 85%, 90%, 91%, 92%, 93%, 94%, 95%, 96%, 97%, 98%, 99%, or 100% identical to SEQ ID NO: 13 or 15. In some embodiments, the LnqQ peptide is at least 80%, 85%, 90%, 91%, 92%, 93%, 94%, 95%, 96%, 97%, 98%, 99%, or 100% identical to SEQ ID NO: 13. In some embodiments, the LnqQ peptide is at least 80%, 85%, 90%, 91%, 92%, 93%, 94%, 95%, 96%, 97%, 98%, 99%, or 100% identical to SEQ ID NO: 15.

In some embodiments, an antimicrobial agent or peptide is expressed and/or secreted by a bacteriophage disclosed herein. In some embodiments, a bacteriophage disclosed herein secretes and expresses an antibiotic such as ampicillin, penicillin, penicillin derivatives, cephalosporins, monobactams, carbapenems, ofloxacin, ciprofloxacin, levofloxacin, gatifloxacin, norfloxacin, lomefloxacin, trovafloxacin, moxifloxacin, sparfloxacin, gemifloxacin, pazufloxacin or any antibiotic disclosed herein. In some embodiments, a bacteriophage disclosed herein comprises a nucleic acid sequence encoding an antibacterial peptide, expresses an antibacterial peptide, or secretes a peptide that aids or enhances killing of a target bacterium. In some embodiments, a bacteriophage disclosed herein comprises a nucleic acid sequence encoding a peptide, a nucleic acid sequence encoding an antibacterial peptide, expresses an antibacterial peptide, or secretes a peptide that aids or enhances the activity of the first and/or the second Type I CRISPR-Cas system.

In some embodiments, the antimicrobial agent or peptide is encoded by a sequence comprising at least or about 70%, 80%, 85%, 90%, 91%, 92%, 93%, 94%, 95%, 96%, 97%, 98%, 99%, or 100% sequence identity to SEQ ID NO. 1. In some embodiments, the antimicrobial agent or peptide is encoded by a sequence comprising at least or about 70%, 80%, 85%, 90%, 91%, 92%, 93%, 94%, 95%, 96%, 97%, 98%, 99%, or 100% sequence identity to SEQ ID NO. 2. In some embodiments, the antimicrobial agent or peptide is encoded by a sequence comprising at least or about 70%, 80%, 85%, 90%, 91%, 92%, 93%, 94%, 95%, 96%, 97%, 98%, 99%, or 100% sequence identity to SEQ ID NO. 3. In some embodiments, the antimicrobial agent or peptide is encoded by a sequence comprising at least or about 70%, 80%, 85%, 90%, 91%, 92%, 93%, 94%, 95%, 96%, 97%, 98%, 99%, or 100% sequence identity to SEQ ID NO. 4. In some embodiments, the antimicrobial agent or peptide is encoded by a sequence comprising at least or about 70%, 80%, 85%, 90%, 91%, 92%, 93%, 94%, 95%, 96%, 97%, 98%, 99%, or 100% sequence identity to SEQ ID NO. 5. In some embodiments, the antimicrobial agent or peptide is encoded by a sequence comprising at least or about 70%, 80%, 85%, 90%, 91%, 92%, 93%, 94%, 95%, 96%, 97%, 98%, 99%, or 100% sequence identity to SEQ ID NO. 7.

In some embodiments, the antimicrobial agent or peptide comprises at least or about 70%, 80%, 85%, 90%, 91%, 92%, 93%, 94%, 95%, 96%, 97%, 98%, 99%, or 100% sequence identity to SEQ ID NO. 9. In some embodiments, the antimicrobial agent or peptide comprises at least or about 70%, 80%, 85%, 90%, 91%, 92%, 93%, 94%, 95%, 96%, 97%, 98%, 99%, or 100% sequence identity to SEQ ID NO. 10. In some embodiments, the antimicrobial agent or peptide comprises at least or about 70%, 80%, 85%, 90%, 91%, 92%, 93%, 94%, 95%, 96%, 97%, 98%, 99%, or 100% sequence identity to SEQ ID NO. 11. In some embodiments, the antimicrobial agent or peptide comprises at least or about 70%, 80%, 85%, 90%, 91%, 92%, 93%, 94%, 95%, 96%, 97%, 98%, 99%, or 100% sequence identity to SEQ ID NO. 12. In some embodiments, the antimicrobial agent or peptide comprises at least or about 70%, 80%, 85%, 90%, 91%, 92%, 93%, 94%, 95%, 96%, 97%, 98%, 99%, or 100% sequence identity to SEQ ID NO. 13. In some embodiments, the antimicrobial agent or peptide comprises at least or about 70%, 80%, 85%, 90%, 91%, 92%, 93%, 94%, 95%, 96%, 97%, 98%, 99%, or 100% sequence identity to SEQ ID NO. 14. In some embodiments, the antimicrobial agent or peptide comprises at least or about 70%, 80%, 85%, 90%, 91%, 92%, 93%, 94%, 95%, 96%, 97%, 98%, 99%, or 100% sequence identity to SEQ ID NO. 15.

### CRISPR/CAS Systems

CRISPR-Cas systems are naturally adaptive immune systems found in bacteria and archaea. The CRISPR system is a nuclease system involved in defense against invading phages and plasmids that provides a form of acquired immunity. There is a diversity of CRISPR-Cas systems based on the set of cas genes and their phylogenetic relationship. There are at least six different types (I through VI) where Type I represents over 50% of all identified systems in both bacteria and archaea. In some embodiments, a Type I, Type II, Type III, Type IV, Type V, or Type VI CRISPR-Cas system is used herein.

Type I systems are divided into seven subtypes including: Type I-A, Type I-B, Type I-C, Type I-D, Type I-E, Type I-F, and Type I-U. Type I CRISPR-Cas systems include a multi-subunit complex called Cascade (for complex associated with antiviral defense), Cas3 (a protein with nuclease, helicase, and exonuclease activity that is responsible for degradation of the target DNA), and CRISPR array encoding crRNA (stabilizes Cascade complex and directs Cascade and Cas3 to DNA target). Cascade forms a complex with the crRNA, and the protein-RNA pair recognizes its genomic target by complementary base pairing between the 5′ end of the crRNA sequence and a predefined protospacer. This complex is directed to homologous loci of pathogen DNA via regions encoded within the crRNA and protospacer-adjacent motifs (PAMs) within the pathogen genome. Base pairing occurs between the crRNA and the target DNA sequence leading to a conformational change. In the Type I-E system, the PAM is recognized by the CasA protein within Cascade, which then unwinds the flanking DNA to evaluate the extent of base pairing between the target and the spacer portion of the crRNA. Sufficient recognition leads Cascade to recruit and activate Cas3. Cas3 then nicks the non-target strand and begins degrading the strand in a 3′-to-5′ direction.

In the Type I-C system, the proteins Cas5, Cas8c, and Cas7 form the Cascade effector complex. Cas5 processes the pre-crRNA (which can take the form of a multi-spacer array, or a single spacer between two repeats) to produce individual crRNA(s) made up of a hairpin structure formed from the remaining repeat sequence and a linear spacer. The effector complex then binds to the processed crRNA and scans DNA to identify PAM sites. In the Type I-C system, the PAM is recognized by the Cas8c protein, which then acts to unwind the DNA duplex. If the sequence 3′ of the PAM matches the crRNA spacer that is bound to effector complex, a conformational change in the complex occurs and Cas3 is recruited to the site. Cas3 then nicks the non-target strand and begins degrading the DNA.

In the Type I-B system, the proteins Cas8b1, Cas7, and Cas5 form the Cascade effector complex. Cas5 processes the pre-crRNA (which can take the form of a multi-spacer array, or a single spacer between two repeats) to produce individual crRNA(s) made up of a hairpin structure formed from the remaining repeat sequence and a linear spacer. The effector complex then binds to the processed crRNA and scans DNA to identify PAM sites. In the Type I-B system, the PAM is recognized by the Cas8b1 protein, which then acts to unwind the DNA duplex. If the sequence 3′ of the PAM matches the crRNA spacer that is bound to effector complex, a conformational change in the complex occurs and Cas3 is recruited to the site. Cas3 then nicks the non-target strand and begins degrading the DNA. In some embodiments, the Type I-B system is from Listeria monocytogenes (LMIB) (e.g., SEQ ID NO: 22). In some embodiments, the Type I-B system is modified from Listeria monocytogenes (LMIB) (e.g., SEQ ID NO: 23). In some embodiments, the Type I-B system comprises a sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOS: 25-29.

In some embodiments, the CRISPR-Cas system is endogenous to the target bacterium. In some embodiments, when a CRISPR-Cas system is endogenous to the target bacterium, the target bacterium comprises at least one gene encoding a Cas polypeptide. In some embodiments, when a CRISPR-Cas system is endogenous to the target bacterium, the target bacterium comprises a nucleic acid encoding a Cas3 polypeptide. In some embodiments, when a CRISPR-Cas system is endogenous to the target bacterium, the target bacterium comprises a nucleic acid encoding a CASCADE complex. In some embodiments, when a CRISPR-Cas system is endogenous to the target bacterium, the target bacterium comprises a nucleic acid sequence encoding a Cas3 polypeptide and a CASCADE complex.

In some embodiments, the CRISPR-Cas system is exogenous to the target bacterium. In some embodiments, when a CRISPR-Cas system is exogenous to the target bacterium, the bacteriophage comprises at least one gene encoding a Cas polypeptide. In some embodiments, when a CRISPR-Cas system is exogenous to the target bacterium, the bacteriophage comprises a nucleic acid encoding a Cas3 polypeptide. In some embodiments, when a CRISPR-Cas system is exogenous to the target bacterium, the bacteriophage comprises a nucleic acid encoding a CASCADE complex. In some embodiments, when a CRISPR-Cas system is exogenous to the target bacterium, the bacteriophage comprises a nucleic acid sequence encoding a Cas3 polypeptide and a CASCADE complex. In some embodiments, when a CRISPR-Cas system is exogenous to the target bacterium, the target bacterium does not comprise a nucleic acid encoding a Cas3 polypeptide. In some embodiments, when a CRISPR-Cas system is exogenous to the target bacterium, the target bacterium does not express a Cas3 polypeptide. In some embodiments, when a CRISPR-Cas system is exogenous to the target bacterium, the target bacterium does not comprise a nucleic acid encoding a CASCADE complex. In some embodiments, when a CRISPR-Cas system is exogenous to the target bacterium, the target bacterium does not express a CASCADE complex. In some embodiments, when a CRISPR-Cas system is exogenous to the target bacterium, the target bacterium does not comprise a nucleic acid sequence encoding a Cas3 polypeptide and a CASCADE complex. In some embodiments, when a CRISPR-Cas system is exogenous to the target bacterium, the target bacterium does not express a Cas3 polypeptide and a CASCADE complex.

In some embodiments, the CRISPR-Cas system is a Type I CRISPR-Cas system. In some embodiments, the CRISPR-Cas system is a Type I-A CRISPR-Cas system. In some embodiments, the CRISPR-Cas system is a Type I-B CRISPR-Cas system. In some embodiments, the CRISPR-Cas system is a Type I-B CRISPR-Cas system derived from Listeria monocytogenes. In some embodiments, the CRISPR-Cas system is a Type I-C CRISPR-Cas system. In some embodiments, the CRISPR-Cas system is a Type I-D CRISPR-Cas system. In some embodiments, the CRISPR-Cas system is a Type I-E CRISPR-Cas system. In some embodiments, the CRISPR-Cas system is a Type I-F CRISPR-Cas system. In some embodiments, the CRISPR-Cas system is a Type I-U CRISPR-Cas system. In some embodiments, the CRISPR-Cas system is a Type II CRISPR-Cas system. In some embodiments, the CRISPR-Cas system is a Type III CRISPR-Cas system.

In some embodiments, processing of a CRISPR-array disclosed herein includes, but is not limited to, the following processes: 1) transcription of the nucleic acid encoding a pre-crRNA; 2) recognition of the pre-crRNA by Cascade and/or specific members of Cascade, such as Cas6, and (3) processing of the pre-crRNA by Cascade or members of Cascade, such as Cas6, into mature crRNAs. In some embodiments, the mode of action for a Type I CRISPR system includes, but is not limited to, the following processes: 4) mature crRNA complexation with Cascade; 5) target recognition by the complexed mature crRNA/Cascade complex; and 6) nuclease activity at the target leading to DNA degradation.

In some embodiments, the Type I CRISPR-Cas system is a Type I-A system, Type I-B system, Type I-C system, Type I-D system, Type I-E system, Type I-F system, or Type I-U system. In some embodiments, the Type I CRISPR-Cas system is a Type I-A system. In some embodiments, the Type I CRISPR-Cas system is a Type I-B system. In some embodiments, the Type I CRISPR-Cas system is a Type I-C system. In some embodiments, the Type I CRISPR-Cas system is a Type I-D system. In some embodiments, the Type I CRISPR-Cas system is a Type I-E system. In some embodiments, the Type I CRISPR-Cas system is a Type I-F system. In some embodiments, the Type I CRISPR-Cas system is a Type I-U system. In some embodiments, the Type I CRISPR-Cas system comprises Cascade polypeptides. Type I Cascade polypeptides process CRISPR arrays to produce a processed RNA that is then used to bind the complex to a target sequence that is complementary to the spacer in the processed RNA. In some embodiments, the Type I Cascade complex is a Type I-A Cascade polypeptides, a Type I-B Cascade polypeptides, a Type I-C Cascade polypeptides, a Type I-D Cascade polypeptides, a Type I-E Cascade polypeptides, a Type I-F Cascade polypeptides, or a Type I-U Cascade polypeptides.

In some embodiments, the Type I Cascade complex comprises: (a) a nucleotide sequence encoding a Cas6b polypeptide, a nucleotide sequence encoding a Cas8b (Csh1) polypeptide, a nucleotide sequence encoding a Cas7 (Csh2) polypeptide, and a nucleotide sequence encoding a Cas5 polypeptide (Type I-B); (b) a nucleotide sequence encoding a Cas5d polypeptide, a nucleotide sequence encoding a Cas8c (Csd1) polypeptide, and a nucleotide sequence encoding a Cas7 (Csd2) polypeptide (Type I-C); (c) a nucleotide sequence encoding a Cse1 (CasA) polypeptide, a nucleotide sequence encoding a Cse2 (CasB) polypeptide, a nucleotide sequence encoding a Cas7 (CasC) polypeptide, a nucleotide sequence encoding a Cas5 (CasD) polypeptide, and a nucleotide sequence encoding a Cas6c (CasE) polypeptide (Type I-E); (d) a nucleotide sequence encoding a Cys1 polypeptide, a nucleotide sequence encoding a Cys2 polypeptide, a nucleotide sequence encoding a Cas7 (Cys3) polypeptide, and a nucleotide sequence encoding a Cas6f polypeptide (Type I-F); (e) a nucleotide sequence encoding a Cas7 (Csa2) polypeptide, a nucleotide sequence encoding a Cas8a1 (Csx13) polypeptide or a Cas8a2 (Csx9) polypeptide, a nucleotide sequence encoding a Cas5 polypeptide, a nucleotide sequence encoding a Csa5 polypeptide, a nucleotide sequence encoding a Cas6a polypeptide, a nucleotide sequence encoding a Cas3′ polypeptide, and a nucleotide sequence encoding a Cas3″ polypeptide having no nuclease activity (Type I-A); (f) a nucleotide sequence encoding a Cas1 Od (Csc3) polypeptide, a nucleotide sequence encoding a Csc2 polypeptide, a nucleotide sequence encoding a Csc1 polypeptide, and a nucleotide sequence encoding a Cas6d polypeptide (Type I-D); and/or (g) Cas8u2 polypeptide, a Cas7 polypeptide, and a fused Cas5-Cas6 polypeptide (Type I-U). In some embodiments, the Type I Cascade complex comprises a Cascade polypeptide disclosed herein

In some embodiments, the Type I CRISPR-Cas system comprises Cascade polypeptides. Type I Cascade polypeptides process CRISPR arrays to produce a processed RNA that is then used to bind the complex to a target sequence that is complementary to the spacer in the processed RNA. In some embodiments, the Type I Cascade complex is a Type I-A Cascade polypeptides, a Type I-B Cascade polypeptides, a Type I-C Cascade polypeptides, a Type I-D Cascade polypeptides, a Type I-E Cascade polypeptides, a Type I-F Cascade polypeptides, or a Type I-U Cascade polypeptides. In some embodiments, the CRISPR-Cas system is a Type I-B CRISPR-Cas system from Listeria monocytogenes (LMIB).

In some embodiments, the CRISPR-Cas system is encoded by a sequence comprising at least or about 70%, 80%, 85%, 90%, 91%, 92%, 93%, 94%, 95%, 96%, 97%, 98%, 99%, or 100% sequence identity to SEQ ID NO: 22. In some instances, the CRISPR-Cas system is encoded by a sequence comprising at least a portion having at least or about 3, 4, 5, 6, 7, 8, 9, 10, 12, 14, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more than 50 nucleotides of SEQ ID NO. 22. In some instances, the CRISPR-Cas system is encoded by a sequence comprising at least a portion having at least or about 50, 55, 60, 65, 70, 75, 80, 85, 90, 95, 100, 105, 110, 115 120, 125, 130, 135, 140, 145, 150, 155, 160, 165, 170, 175, 180, 185, 190, 195, 200, 205, 210, 215, or more than 215 nucleotides of SEQ ID NO. 22. In some embodiments, the CRISPR-Cas system is encoded by a sequence comprising at least or about 70%, 80%, 85%, 90%, 91%, 92%, 93%, 94%, 95%, 96%, 97%, 98%, 99%, or 100% sequence identity to SEQ ID NO: 23. In some instances, the CRISPR-Cas system is encoded by a sequence comprising at least a portion having at least or about 3, 4, 5, 6, 7, 8, 9, 10, 12, 14, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more than 50 nucleotides of SEQ ID NO. 23. In some instances, the CRISPR-Cas system is encoded by a sequence comprising at least a portion having at least or about 50, 55, 60, 65, 70, 75, 80, 85, 90, 95, 100, 105, 110, 115 120, 125, 130, 135, 140, 145, 150, 155, 160, 165, 170, 175, 180, 185, 190, 195, 200, 205, 210, 215, or more than 215 nucleotides of SEQ ID NO. 23.

In some embodiments, the CRISPR-Cas system comprises at least or about 70%, 80%, 85%, 90%, 91%, 92%, 93%, 94%, 95%, 96%, 97%, 98%, 99%, or 100% sequence identity to SEQ ID NO: 25 (e.g., Cas6). In some embodiments, the CRISPR-Cas system comprises at least or about 70%, 80%, 85%, 90%, 91%, 92%, 93%, 94%, 95%, 96%, 97%, 98%, 99%, or 100% sequence identity to SEQ ID NO: 29 (e.g., Cas8). In some embodiments, the CRISPR-Cas system comprises at least or about 70%, 80%, 85%, 90%, 91%, 92%, 93%, 94%, 95%, 96%, 97%, 98%, 99%, or 100% sequence identity to SEQ ID NO: 26 (e.g., Cas7). In some embodiments, the CRISPR-Cas system comprises at least or about 70%, 80%, 85%, 90%, 91%, 92%, 93%, 94%, 95%, 96%, 97%, 98%, 99%, or 100% sequence identity to SEQ ID NO: 27 (e.g., Cas5). In some embodiments, the CRISPR-Cas system comprises at least or about 70%, 80%, 85%, 90%, 91%, 92%, 93%, 94%, 95%, 96%, 97%, 98%, 99%, or 100% sequence identity to SEQ ID NO: 28 (e.g., Cas3). In some instances, the CRISPR-Cas system comprises at least or about 95% homology to any one of SEQ ID NOS: 25-29. In some instances, the CRISPR-Cas system comprises at least or about 97% homology to any one of SEQ ID NOS: 25-29. In some instances, the CRISPR-Cas system comprises at least or about 99% homology to any one of SEQ ID NOS: 25-29. In some instances, the CRISPR-Cas system comprises 100% homology to any one of SEQ ID NOS: 25-29. In some instances, the CRISPR-Cas system comprises at least a portion having at least or about 3, 4, 5, 6, 7, 8, 9, 10, 12, 14, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more than 50 amino acids of any one of SEQ ID NOS: 25-29. In some instances, the CRISPR-Cas system comprises at least a portion having at least or about 50, 55, 60, 65, 70, 75, 80, 85, 90, 95, 100, 105, 110, 115 120, 125, 130, 135, 140, 145, 150, 155, 160, 165, 170, 175, 180, 185, 190, 195, 200, 205, 210, 215, or more than 215 amino acids of any one of SEQ ID NOS: 25-29.

In some embodiments, the CRISPR-Cas system comprises a Cas6 polypeptide encoded by a sequence with at least or about 70%, 80%, 85%, 90%, 91%, 92%, 93%, 94%, 95%, 96%, 97%, 98%, 99%, or 100% sequence identity to SEQ ID NO: 33. In some embodiments, the CRISPR-Cas system comprises a Cas8 polypeptide encoded by a sequence with at least or about 70%, 80%, 85%, 90%, 91%, 92%, 93%, 94%, 95%, 96%, 97%, 98%, 99%, or 100% sequence identity to SEQ ID NO: 34. In some embodiments, the CRISPR-Cas system comprises a Cas7 polypeptide encoded by a sequence with at least or about 70%, 80%, 85%, 90%, 91%, 92%, 93%, 94%, 95%, 96%, 97%, 98%, 99%, or 100% sequence identity to SEQ ID NO: 35. In some embodiments, the CRISPR-Cas system comprises a Cas5 polypeptide encoded by a sequence with at least or about 70%, 80%, 85%, 90%, 91%, 92%, 93%, 94%, 95%, 96%, 97%, 98%, 99%, or 100% sequence identity to SEQ ID NO: 36. In some embodiments, the CRISPR-Cas system comprises a Cas3 polypeptide encoded by a sequence with at least or about 70%, 80%, 85%, 90%, 91%, 92%, 93%, 94%, 95%, 96%, 97%, 98%, 99%, or 100% sequence identity to SEQ ID NO: 37.

In some embodiments, the CRISPR-Cas system comprises a Cas6 polypeptide encoded by a sequence with at least or about 70%, 80%, 85%, 90%, 91%, 92%, 93%, 94%, 95%, 96%, 97%, 98%, 99%, or 100% sequence identity to SEQ ID NO: 38. In some embodiments, the CRISPR-Cas system comprises a Cas8 polypeptide encoded by a sequence with at least or about 70%, 80%, 85%, 90%, 91%, 92%, 93%, 94%, 95%, 96%, 97%, 98%, 99%, or 100% sequence identity to SEQ ID NO: 39. In some embodiments, the CRISPR-Cas system comprises a Cas7 polypeptide encoded by a sequence with at least or about 70%, 80%, 85%, 90%, 91%, 92%, 93%, 94%, 95%, 96%, 97%, 98%, 99%, or 100% sequence identity to SEQ ID NO: 40. In some embodiments, the CRISPR-Cas system comprises a Cas5 polypeptide encoded by a sequence with at least or about 70%, 80%, 85%, 90%, 91%, 92%, 93%, 94%, 95%, 96%, 97%, 98%, 99%, or 100% sequence identity to SEQ ID NO: 41. In some embodiments, the CRISPR-Cas system comprises a Cas3 polypeptide encoded by a sequence with at least or about 70%, 80%, 85%, 90%, 91%, 92%, 93%, 94%, 95%, 96%, 97%, 98%, 99%, or 100% sequence identity to SEQ ID NO: 42.

### CRISPR Array

In some embodiments, described herein is a CRISPR array (crArray) comprising a spacer sequence and at least one repeat sequence. In some embodiments, the CRISPR array encodes a processed, mature crRNA. In some embodiments, the mature crRNA is introduced into a phage or a target bacterium described herein. In some embodiments, the phage comprises a nucleic acid that encodes a processed, mature crRNA. In some embodiments, an endogenous or exogenous Cas6 processes the CRISPR array into mature crRNA. In some embodiments, an exogenous Cas6 is introduced into the phage. In some embodiments, the phage comprises an exogenous Cas6. In some embodiments, an exogenous Cas6 is introduced into a target bacterium.

In some embodiments, processing of a CRISPR-array disclosed herein includes, but is not limited to, the following processes: 1) transcription of the nucleic acid encoding a pre-crRNA; 2) recognition of the pre-crRNA by Cascade and/or specific members of Cascade, such as Cas6, and (3) processing of the pre-crRNA by Cascade or members of Cascade, such as Cas6, into mature crRNAs. In some embodiments, the mode of action for a Type I CRISPR system includes, but is not limited to, the following processes: 4) mature crRNA complexation with Cascade; 5) target recognition by the complexed mature crRNA/Cascade complex; and 6) nuclease activity at the target leading to DNA degradation.

In some embodiments, the CRISPR array comprises a spacer sequence. In some embodiments, the CRISPR array further comprises at least one repeat sequence. In some embodiments, the at least one repeat sequence is operably linked to the spacer sequence at either its 5′ end or its 3′ end. In some embodiments, a CRISPR array is of any length and comprises any number of spacer nucleotide sequences alternating with repeat nucleotide sequences necessary to achieve the desired level of killing of a target bacterium by targeting one or more target sequences. In some embodiments, the CRISPR array comprises, consists essentially of, or consists of 1 to about 100 spacer nucleotide sequences, each linked on its 5′ end and its 3′ end to a repeat nucleotide sequence. In some embodiments, the CRISPR array as disclosed herein, comprises essentially of, or consists of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more, spacer nucleotide sequences. In some embodiments, the CRISPR array comprises at least or about 70%, 80%, 85%, 90%, 91%, 92%, 93%, 94%, 95%, 96%, 97%, 98%, 99%, or 100% sequence identity to SEQ ID NO. 24. In some embodiments, the CRISPR array comprises at least or about 70%, 80%, 85%, 90%, 91%, 92%, 93%, 94%, 95%, 96%, 97%, 98%, 99%, or 100% sequence identity to amino acids 41 to 267 of SEQ ID NO. 24. In some embodiments, the repeat sequence comprises at least or about 70%, 80%, 85%, 90%, 91%, 92%, 93%, 94%, 95%, 96%, 97%, 98%, 99%, or 100% sequence identity to SEQ ID NO. 43. In some embodiments, the spacer sequence comprises at least or about 70%, 80%, 85%, 90%, 91%, 92%, 93%, 94%, 95%, 96%, 97%, 98%, 99%, or 100% sequence identity to any one of SEQ ID NO. 44-46. In some embodiments, the spacer sequence comprises at least or about 70%, 80%, 85%, 90%, 91%, 92%, 93%, 94%, 95%, 96%, 97%, 98%, 99%, or 100% sequence identity to SEQ ID NO. 44. In some embodiments, the spacer sequence comprises at least or about 70%, 80%, 85%, 90%, 91%, 92%, 93%, 94%, 95%, 96%, 97%, 98%, 99%, or 100% sequence identity to SEQ ID NO. 45. In some embodiments, the spacer sequence comprises at least or about 70%, 80%, 85%, 90%, 91%, 92%, 93%, 94%, 95%, 96%, 97%, 98%, 99%, or 100% sequence identity to SEQ ID NO. 46.

### Spacer Sequence

In some embodiments, the spacer sequence is complementary to a target nucleotide sequence in a target bacterium. In some embodiments, the target nucleotide sequence is a coding region. In some embodiments, the coding region is an essential gene. In some embodiments, the coding region is a nonessential gene. In some embodiments, the target nucleotide sequence is a noncoding sequence. In some embodiments, the noncoding sequence is an intergenic sequence. In some embodiments, the spacer sequence is complementary to a target nucleotide sequence of a highly conserved sequence in a target bacterium. In some embodiments, the spacer sequence is complementary to a target nucleotide sequence of a sequence present in the target bacterium. In some embodiments, the spacer sequence is complementary to a target nucleotide sequence that comprises all or a part of a promoter sequence of the essential gene. In some embodiments, the spacer sequence comprises one, two, three, four, or five mismatches as compared to the target nucleotide sequence. In some embodiments, the mismatches are contiguous. In some embodiments, the mismatches are noncontiguous. In some embodiments, the spacer sequence has 70% complementarity to a target nucleotide sequence. In some embodiments, the spacer sequence has 80% complementarity to a target nucleotide sequence. In some embodiments, the spacer sequence is 85%, 90%, 95%, 96%, 97%, 98%, 99% or 100% complementarity to a target nucleotide sequence. In some embodiments, the spacer sequence has 100% complementarity to the target nucleotide sequence. In some embodiments, the spacer sequence has complete complementarity or substantial complementarity over a region of a target nucleotide sequence that are at least about 8 nucleotides to about 150 nucleotides in length. In some embodiments, a spacer sequence has complete complementarity or substantial complementarity over a region of a target nucleotide sequence that is at least about 20 nucleotides to about 100 nucleotides in length. In some embodiments, the 5′ region of the spacer sequence is 100% complementary to a target nucleotide sequence while the 3′ region of the spacer is substantially complementary to the target nucleotide sequence and therefore the overall complementarity of the spacer sequence to the target nucleotide sequence is less than 100%. For example, in some embodiments, the first 7, 8, 9, 10, 11, 12, 13, 14, 15, 16 nucleotides in the 3′ region of a 20 nucleotide spacer sequence (seed region) is 100% complementary to the target nucleotide sequence, while the remaining nucleotides in the 5′ region of the spacer sequence are substantially complementary (e.g., at least about 70% complementary) to the target nucleotide sequence. In some embodiments, the first 7 to 12 nucleotides of the 3′ end of the spacer sequence is 100% complementary to the target nucleotide sequence, while the remaining nucleotides in the 5′ region of the spacer sequence are substantially complementary (e.g., at least about 50% complementary (e.g., 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to the target nucleotide sequence. In some embodiments, the first 7 to 10 nucleotides in the 3′ end of the spacer sequence is 75%-99% complementary to the target nucleotide sequence, while the remaining nucleotides in the 5′ region of the spacer sequence are at least about 50% to about 99% complementary to the target nucleotide sequence. In some embodiments, the first 7 to 10 nucleotides in the 3′ end of the spacer sequence is 100% complementary to the target nucleotide sequence, while the remaining nucleotides in the 5′ region of the spacer sequence are substantially complementary (e.g., at least about 70% complementary) to the target nucleotide sequence. In some embodiments, the first 10 nucleotides (within the seed region) of the spacer sequence is 100% complementary to the target nucleotide sequence, while the remaining nucleotides in the 5′ region of the spacer sequence are substantially complementary (e.g., at least about 70% complementary) to the target nucleotide sequence. In some embodiment, the 5′ region of a spacer sequence (e.g., the first 8 nucleotides at the 5′ end, the first 10 nucleotides at the 5′ end, the first 15 nucleotides at the 5′ end, the first 20 nucleotides at the 5′ end) have about 75% complementarity or more (75% to about 100% complementarity) to the target nucleotide sequence, while the remainder of the spacer sequence have about 50% or more complementarity to the target nucleotide sequence. In some embodiments, the first 8 nucleotides at the 5′ end of the spacer sequence have 100% complementarity to the target nucleotide sequence or have one or two mutations and therefore is about 88% complementary or about 75% complementary to the target nucleotide sequence, respectively, while the remainder of the spacer nucleotide sequence is at least about 50% or more complementary to the target nucleotide sequence.

In some embodiments, the spacer sequence is about 15 nucleotides to about 150 nucleotides in length. In some embodiments, the spacer nucleotide sequence is about 15 nucleotides to about 100 nucleotides in length (e.g., about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 nucleotides or more). In some embodiments, the spacer nucleotide sequence is a length of about 8 to about 150 nucleotides, about 8 to about 100 nucleotides, about 8 to about 50 nucleotides, about 8 to about 40 nucleotides, about 8 to about 30 nucleotides, about 8 to about 25 nucleotides, about 8 to about 20 nucleotides, about 10 to about 150 nucleotides, about 10 to about 100 nucleotides, about 10 to about 80 nucleotides, about 10 to about 50 nucleotides, about 10 to about 40, about 10 to about 30, about 10 to about 25, about 10 to about 20, about 15 to about 150, about 15 to about 100, about 15 to about 50, about 15 to about 40, about 15 to about 30, about 20 to about 150 nucleotides, about 20 to about 100 nucleotides, about 20 to about 80 nucleotides, about 20 to about 50 nucleotides, about 20 to about 40, about 20 to about 30, about 20 to about 25, at least about 8, at least about 10, at least about 15, at least about 20, at least about 25, at least about 30, at least about 32, at least about 35, at least about 40, at least about 44, at least about 50, at least about 60, at least about 70, at least about 80, at least about 90, at least about 100, at least about 110, at least about 120, at least about 130, at least about 140, at least about 150 nucleotides in length, or more, and any value or range therein. In some embodiments, the Listeria monocytogenes Type I-B Cas system has a spacer length of about 30 to 39 nucleotides, about 31 to about 38 nucleotides, about 32 to about 37 nucleotides, about 36 to about 37 nucleotides, or about 37 nucleotides. In some embodiments, the Listeria monocytogenes Type I-B system has a spacer length of about 37 nucleotides. In some embodiments, the Listeria monocytogenes Type I-B Cas system has a spacer length of at least about 10, at least about 15, at least about 20, at least about 21, at least about 22, at least about 23, at least about 24, at least about 25, at least about 26, at least about 27, at least about 29, at least about 29, at least about 30, at least about 31, at least about 32, at least about 33, at least about 34, at least about, at least about 35, at least about 36, at least about 37, at least about 38, at least about 39, at least about 20, at least about 41, at least about 42, at least about 43, at least about 44, at least about 45, or more than about 45 nucleotides.

In some embodiments, the identity of two or more spacer sequences of the CRISPR array is the same. In some embodiments, the identity of two or more spacer sequences of the CRISPR array is different. In some embodiments, the identity of two or more spacer sequences of the CRISPR array is different but are complementary to one or more target nucleotide sequences. In some embodiments, the identity of two or more spacer sequences of the CRISPR array is different and are complementary to one or more target nucleotide sequences that are overlapping sequences. In some embodiments, the identity of two or more spacer sequences of the CRISPR array is different and are complementary to one or more target nucleotide sequences that are not overlapping sequences. In some embodiments, the target nucleotide sequence is about 10 to about 40 consecutive nucleotides in length located immediately adjacent to a PAM sequence (PAM sequence located immediately 3′ of the target region) in the genome of the organism. In some embodiments, a target nucleotide sequence is located adjacent to or flanked by a PAM (protospacer adjacent motif).

The PAM sequence is found in the target gene next to the region to which a spacer sequence binds as a result of being complementary to that region and identifies the point at which base pairing with the spacer nucleotide sequence begins. The exact PAM sequence that is required varies between each different CRISPR-Cas system and is identified through established bioinformatics and experimental procedures. Non-limiting examples of PAMs include CCA, CCT, CCG, TTC, AAG, AGG, ATG, GAG, and/or CC. For Type I systems, the PAM is located immediately 5′ to the sequence that matches the spacer, and thus is 3′ to the sequence that base pairs with the spacer nucleotide sequence, and is directly recognized by Cascade. Once a protospacer is recognized, Cascade generally recruits the endonuclease Cas3, which cleaves and degrades the target DNA. For Type II systems, the PAM is required for a Cas9/sgRNA to form an R-loop to interrogate a specific DNA sequence through Watson-Crick pairing of its guide RNA with the genome. The PAM specificity is a function of the DNA-binding specificity of the Cas9 protein (e.g., a -protospacer adjacent motif recognition domain at the C-terminus of Cas9)

In some embodiments, the target nucleotide sequence in the bacterium to be killed is any essential target nucleotide sequence of interest. In some embodiments, the target nucleotide sequence is a non-essential sequence. In some embodiments, a target nucleotide sequence comprises, consists essentially of or consist of all or a part of a nucleotide sequence encoding a promoter, or a complement thereof, of the essential gene. In some embodiments, the spacer nucleotide sequence is complementary to a promoter, or a part thereof, of the essential gene. In some embodiments, the target nucleotide sequence comprises all or a part of a nucleotide sequence located on a coding or a non-coding strand of the essential gene. In some embodiments, the target nucleotide sequence comprises all or a part of a nucleotide sequence located on a coding of a transcribed region of the essential gene.

In some embodiments, the essential gene is any gene of an organism that is critical for its survival. However, being essential is highly dependent on the circumstances in which an organism lives. For instance, a gene required to digest starch is only essential if starch is the only source of energy. In some embodiments, the target nucleotide sequence comprises all or a part of a promoter sequence for the target gene. In some embodiments, the target nucleotide sequence comprises all or a part of a nucleotide sequence located on a coding strand of a transcribed region of the target gene. In some embodiments, the target nucleotide sequence comprises at least a portion of an essential gene that is needed for survival of the target bacterium. In some embodiments, the essential gene is Tsf, acpP, gapA, infA, secY, csrA, trmD, ftsA, fusA, glyQ, eno, nusG, dnaA, pheS, rplB, gltX, hisS, rplC, aspS, gyrB, dnaE, rpoA, rpoB, pheT, infB, rpsC, rplF, alaS, leuS, serS, rplD, gyrA, glmS, fus, adk, rpsK, rplR, ctrA, parC, tRNA-Ser, tRNA-Asn, or metK. In some embodiments, a non-essential gene is any gene of an organism that is not critical for survival. However, being non-essential is highly dependent on the circumstances in which an organism lives.

In some embodiments, non-limiting examples of the target nucleotide sequence of interest includes a target nucleotide sequence encoding a transcriptional regulator, a translational regulator, a polymerase gene, a metabolic enzyme, a transporter, an RNase, a protease, a DNA replication enzyme, a DNA modifying or degrading enzyme, a regulatory RNA, a transfer RNA, or a ribosomal RNA. In some embodiments, the target nucleotide sequence is from a gene involved in cell-division, cell structure, metabolism, motility, pathogenicity, virulence, or antibiotic resistance. In some embodiments, the target nucleotide sequence is from a hypothetical gene whose function is not yet characterized. Thus, for example, these genes are any genes from any bacterium.

The appropriate spacer sequences for a full-construct phage may be identified by locating a search set of representative genomes, searching the genomes with relevant parameters, and determining the quality of a spacer for use in a CRISPR engineered phage.

First, a suitable search set of representative genomes is located and acquired for the organism/species/target of interest. The set of representative genomes may be found in a variety of databases, including without limitations the NCBI GenBank or the PATRIC database. NCBI GenBank is one of the largest databases available and contains a mixture of reference and submitted genomes for nearly every organism sequenced to date. Specifically, for pathogenic prokaryotes, the PATRIC (Pathosystems Resource Integration Center) database provides an additional comprehensive resource of genomes and provides a focus on clinically relevant strains and genomes relevant to a drug product. Both of the above databases allow for bulk downloading of genomes via FTP (File Transfer Protocol) servers, enabling rapid and programmatic dataset acquisition

Next, the genomes are searched with relevant parameters to locate suitable spacer sequences. Genomes may be read from start to end, in both the forward and reverse complement orientations, to locate contiguous stretches of DNA that contain a PAM (Protospacer Adjacent Motif) site. The spacer sequence will be the N-length DNA sequence 3′ or 5′ adjacent to the PAM site (depending on the CRISPR system type), where N is specific to the Cas system of interest and is generally known ahead of time. Characterizing the PAM sequence and spacer sequences may be performed during the discovery and initial research of a Cas system. Every observed PAM-adjacent spacer may be saved to a file and/or database for downstream use. The exact PAM sequence that is required varies between each different CRISPR-Cas system and is identified through established bioinformatics and experimental procedures.

Next, the quality of a spacer for use in a CRISPR engineered phage is determined. Each observed spacer may be evaluated to determine how many of the evaluated genomes they are present in. The observed spacers may be evaluated to see how many times they may occur in each given genome. Spacers that occur in more than one location per genome may be advantageous because the Cas system may not be able to recognize the target site if a mutation occurs, and each additional “backup” site increases the likelihood that a suitable, non-mutated target location will be present. The observed spacers may be evaluated to determine whether they occur in functionally annotated regions of the genome. If such information is available, the functional annotations may be further evaluated to determine whether those regions of the genome are “essential” for the survival and function of the organism. By focusing on spacers that occur in all, or nearly all, evaluated genomes of interest (>=99%), the spacer selection may be broadly applicable to many targeted genomes. Provided a large selection pool of conserved spacers exists, preference may be given to spacers that occur in regions of the genome that have known function, with higher preference given if those genomic regions are “essential” for survival and occur more than 1 time per genome.

In some embodiments, the spacer comprises at least or about 70%, 80%, 85%, 90%, 91%, 92%, 93%, 94%, 95%, 96%, 97%, 98%, 99%, or 100% sequence identity to any one of SEQ ID NOS. 44-46. In some embodiments, the spacer sequence comprises at least or about 70%, 80%, 85%, 90%, 91%, 92%, 93%, 94%, 95%, 96%, 97%, 98%, 99%, or 100% sequence identity to SEQ ID NO. 44. In some embodiments, the spacer sequence comprises at least or about 70%, 80%, 85%, 90%, 91%, 92%, 93%, 94%, 95%, 96%, 97%, 98%, 99%, or 100% sequence identity to SEQ ID NO. 45. In some embodiments, the spacer sequence comprises at least or about 70%, 80%, 85%, 90%, 91%, 92%, 93%, 94%, 95%, 96%, 97%, 98%, 99%, or 100% sequence identity to SEQ ID NO. 46.

### Repeat Nucleotide Sequences

In some embodiments, a repeat nucleotide sequence of the CRISPR array comprises a nucleotide sequence of any known repeat nucleotide sequence of a CRISPR-Cas system. In some embodiment, the CRISPR-Cas system is a Type I CRISPR-Cas system. In some embodiment, a repeat nucleotide sequence is of a synthetic sequence comprising the secondary structure of a native repeat from a Type I CRISPR-Cas system (e.g., an internal hairpin). In some embodiments, the repeat nucleotide sequences are distinct from one another based on the known repeat nucleotide sequences of a CRISPR-Cas system. In some embodiments, the repeat nucleotide sequences are each composed of distinct secondary structures of a native repeat from a CRISPR-Cas system (e.g., an internal hairpin). In some embodiments, the repeat nucleotide sequences are a combination of distinct repeat nucleotide sequences operable with a CRISPR-Cas system.

In some embodiments, the spacer sequence is linked at its 5′ end to the 3′ end of a repeat sequence. In some embodiments, the spacer sequence is linked at its 5′ end to about 1 to about 8, about 1 to about 10, or about 1 to about 15 nucleotides of the 3′ end of a repeat sequence. In some embodiments, the about 1 to about 8, about 1 to about 10, about 1 to about 15 nucleotides of the repeat sequence are a portion of the 3′ end of a repeat sequence. In some embodiments, the spacer nucleotide sequence is linked at its 3′ end to the 5′ end of a repeat sequence. In some embodiments, the spacer is linked at its 3′ end to about 1 to about 8, about 1 to about 10, or about 1 to about 15 nucleotides of the 5′ end of a repeat sequence. In some embodiments, the about 1 to about 8, about 1 to about 10, about 1 to about 15 nucleotides of the repeat sequence are a portion of the 5′ end of a repeat sequence.

In some embodiments, the spacer nucleotide sequence is linked at its 5′ end to a first repeat sequence and linked at its 3′ end to a second repeat sequence to form a repeat-spacer-repeat sequence. In some embodiments, the spacer sequence is linked at its 5′ end to the 3′ end of a first repeat sequence and is linked at its 3′ end to the 5′ of a second repeat sequence where the spacer sequence and the second repeat sequence are repeated to form a repeat-(spacer-repeat) n sequence such that n is any integer from 1 to 100. In some embodiments, a repeat-(spacer-repeat) n sequence comprises, consists essentially of, or consists of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more, spacer nucleotide sequences.

In some embodiments, the repeat sequence is identical to or substantially identical to a repeat sequence from a wild-type CRISPR loci. In some embodiments, the repeat sequence is a sequence described herein. In some embodiments, the repeat sequence comprises a portion of a wild type repeat sequence (e.g., 1, 2, 3, 4, 5, 6, 7, 8, 9, 10, 11, 12, 13, 14, 15 or more contiguous nucleotides of a wild type repeat sequence). In some embodiments, the repeat sequence comprises, consists essentially of, or consists of at least one nucleotide (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, or more nucleotides, or any range therein). In some embodiments, the repeat sequence comprises, consists essentially of, or consists of no more than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, or 100 nucleotides. In some embodiments, the repeat sequence comprises about 20 to 40, 21 to 40, 22 to 40 23 to 40, 24 to 40, 25 to 40, 26 to 40, 27 to 40, 28 to 40, 29 to 40, 30 to 30, 31 to 40, 32 to 40, 33 to 40, 34 to 40, 35 to 40, 36 to 40, 37 to 40, 38 to 40, 39 to 40, 20 to 39, 20 to 38, 20 to 37, 20 to 36, 20 to 35, 20 to 34, 20 to 33, 20 to 32, 20 to 31, 20 to 30, 20 to 29, 20 to 28, 20 to 26, 20 to 25, 20 to 24, 20 to 23, 20 to 22, or 20 to 21 nucleotides. In some embodiments, the repeat sequence comprises about 20 to 35, 21 to 35, 22 to 35 23 to 35, 24 to 35, 25 to 35, 26 to 35, 27 to 35, 28 to 35, 29 to 35, 30 to 30, 31 to 35, 32 to 35, 33 to 35, 34 to 35, 25 to 40, 25 to 39, 25 to 38, 25 to 37, 25 to 36, 25 to 35, 25 to 34, 25 to 33, 25 to 32, 25 to 31, 25 to 30, 25 to 29, 25 to 28, 25 to 26 nucleotides. In some embodiments, the system is a L. monocytogenes Type I-B Cas system. In some embodiments, the L. monocytogenes Type I-B Cas system has a repeat length of about 25 to 38 nucleotides.

In some embodiments, the repeat comprises at least or about 70%, 80%, 85%, 90%, 91%, 92%, 93%, 94%, 95%, 96%, 97%, 98%, 99%, or 100% sequence identity to SEQ ID NO. 43.

### Transcriptional Activators

In some embodiments, the nucleic acid sequence further comprises a transcriptional activator. In some embodiments, the transcriptional activator encoded regulates the expression of genes of interest within the Staphylococcus species. In some embodiments, the transcriptional activator activates the expression of genes of interest within the Staphylococcus species whether exogenous or endogenous. In some embodiments, the transcriptional activator activates the expression genes of interest within the Staphylococcus species by disrupting the activity of one or more inhibitory elements within the Staphylococcus species. In some embodiments, the inhibitory element comprises a transcriptional repressor. In some embodiments, the inhibitory element comprises a global transcriptional repressor. In some embodiments the inhibitory element is a histone-like nucleoid-structuring (H-NS) protein or homologue or functional fragment thereof. In some embodiments, the inhibitory element is a leucine responsive regulatory protein (LRP). In some embodiments, the inhibitory element is a CodY protein.

In some bacteria, the CRISPR-Cas system is poorly expressed and considered silent under most environmental conditions. In these bacteria, the regulation of the CRISPR-Cas system is the result of the activity of transcriptional regulators, for example histone-like nucleoid-structuring (H-NS) protein which is widely involved in transcriptional regulation of the host genome. H-NS exerts control over host transcriptional regulation by multimerization along AT-rich sites resulting in DNA bending.

Similarly, in some bacteria, the repression of the CRISPR-Cas system is controlled by an inhibitory element, for example the leucine responsive regulatory protein (LRP). LRP has been implicated in binding to upstream and downstream regions of the transcriptional start sites. Notably, the activity of LRP in regulating expression of the CRISPR-Cas system varies from bacteria to bacteria. Unlike, H-NS which has broad inter-species repression activity, LRP has been shown to differentially regulate the expression of the host CRISPR-Cas system. As such, in some instances, LRP reflects a host-specific means of regulating CRISPR-Cas system expression in different bacteria.

In some instances, the repression of CRISPR-Cas system is also controlled by inhibitory element CodY. CodY is a GTP-sensing transcriptional repressor that acts through DNA binding. The intracellular concentration of GTP acts as an indicator for the environmental nutritional status. Under normal culture conditions, GTP is abundant and binds with CodY to repress transcriptional activity. However, as GTP concentrations decreases, CodY becomes less active in binding DNA, thereby allowing transcription of the formerly repressed genes to occur. As such, CodY acts as a stringent global transcriptional repressor.

In some embodiments, the transcriptional activator is a LeuO polypeptide, any homolog or functional fragment thereof, a leuO coding sequence, or an agent that upregulates LeuO. In some embodiments, the transcriptional activator comprises any ortholog or functional equivalent of LeuO. In some bacteria, LeuO acts in opposition to H-NS by acting as a global transcriptional regulator that responds to environmental nutritional status of a bacterium. Under normal conditions, LeuO is poorly expressed. However, under amino acid starvation and/or reaching of the stationary phase in the bacterial life cycle, LeuO is upregulated. Increased expression of LeuO leads to it antagonizing H-NS at overlapping promoter regions to effect gene expression. Overexpression of LeuO upregulates the expression of the CRISPR-Cas system.

In some embodiments, the expression of LeuO leads to disruption of an inhibitory element. In some embodiments, the disruption of an inhibitory element due to expression of LeuO removes the transcriptional repression of a CRISPR-Cas system. In some embodiments, the expression of LeuO removes transcriptional repression of a CRISPR-Cas system due to activity of H-NS. In some embodiments, the disruption of an inhibitory element due to the expression of LeuO causes an increase in the expression of a CRISPR-Cas system. In some embodiments, the increase in the expression of a CRISPR-Cas system due to the disruption of an inhibitory element caused by the expression of LeuO causes an increase in the CRISPR-Cas processing of a nucleic acid sequence comprising a CRISPR array. In some embodiments, the increase in the expression of a CRISPR-Cas system due to the disruption of an inhibitory element by the expression of LeuO causes an increase in the CRISPR-Cas processing of a nucleic acid sequence comprising a CRISPR array so as to increase the level of lethality of the CRISPR array against a bacterium. In some embodiments, transcriptional activator causes increase activity of a bacteriophage and/or the CRISPR-Cas system.

### Regulatory Elements

In some embodiments, the nucleic acid sequences are operatively associated with a variety of promoters, terminators and other regulatory elements for expression in various organisms or cells. In some embodiments, the nucleic acid sequence further comprises a leader sequence. In some embodiments, the nucleic acid sequence further comprises a promoter sequence. In some embodiments, at least one promoter and/or terminator is operably linked the CRISPR array. Any promoter useful with this disclosure is used and includes, for example, promoters functional with the organism of interest as well as constitutive, inducible, developmental regulated, tissue-specific/preferred-promoters, and the like, as disclosed herein. A regulatory element as used herein is endogenous or heterologous. In some embodiments, an endogenous regulatory element derived from the subject organism is inserted into a genetic context in which it does not naturally occur (e.g. a different position in the genome than as found in nature), thereby producing a recombinant or non-native nucleic acid.

In some embodiments, expression of the nucleic acid sequence is constitutive, inducible, temporally regulated, developmentally regulated, or chemically regulated. In some embodiments, the expression of the nucleic acid sequence is made constitutive, inducible, temporally regulated, developmentally regulated, or chemically regulated by operatively linking the nucleic acid sequence to a promoter functional in an organism of interest. In some embodiments, repression is made reversible by operatively linking the nucleic acid sequence to an inducible promoter that is functional in an organism of interest. The choice of promoter disclosed herein varies depending on the quantitative, temporal and spatial requirements for expression, and also depending on the host cell to be transformed.

Exemplary promoters for use with the methods, bacteriophages and compositions disclosed herein include promoters that are functional in bacteria. For example, L-arabinose inducible (araBAD, P_{BAD}) promoter, any lac promoter, L-rhamnose inducible (rhaPBAD) promoter, T7 RNA polymerase promoter, tre promoter, tac promoter, lambda phage promoter (p_{L}p_{L}-9G-50), anhydrotetracycline-inducible (tetA) promoter, trp, Ipp, phoA, recA, proU, cst-1, cadA, nar, Ipp-lac, cspA, 11-lac operator, T3-lac operator, T4 gene 32, T5-lac operator, nprM-lac operator, Vhb, Protein A, corynebacterial-E. coli like promoters, thr, horn, diphtheria toxin promoter, sig A, sig B, nusG, SoxS, katb, a-amylase (Pamy), Ptms, P43 (comprised of two overlapping RNA polymerase σ factor recognition sites, σA, σB), Ptms, P43, rplK-rplA, ferredoxin promoter, and/or xylose promoter. In some embodiments, the promoter is a BBa_J23102 promoter. In some embodiments, the promoter works in a broad range of bacteria, such as BBa_J23104, BBa_J23109. In some embodiments the promoter is derived from the target bacterium, such as endogenous CRISPR promoter, endogenous Cas operon promoter or the promoter from sarA, lipA, ptsH or cap1 of S. aureus.

In some embodiments, the promoter comprises at least or about 70%, 80%, 85%, 90%, 91%, 92%, 93%, 94%, 95%, 96%, 97%, 98%, 99%, or 100% sequence identity to any one of SEQ ID NOs: 16-19. In some instances, the promoter comprises at least a portion having at least or about 3, 4, 5, 6, 7, 8, 9, 10, 12, 14, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more than 50 nucleotides of any one of SEQ ID NOS: 16-19. In some instances, the promoter comprises at least a portion having at least or about 50, 55, 60, 65, 70, 75, 80, 85, 90, 95, 100, 105, 110, 115 120, 125, 130, 135, 140, 145, 150, 155, 160, 165, 170, 175, 180, 185, 190, 195, 200, 205, 210, 215, or more than 215 nucleotides of any one of SEQ ID NOS: 16-19.

In some embodiments, the nucleic acid comprises a ribosomal binding site (RBS) downstream of a promoter, e.g., a promoter described herein. In some cases, the RBS is native to the gene that it is operably associated with. In some cases, the RBS is not native to the gene that it is operably associated with. In some cases, the RBS is native to the promoter that it is operably associated with. In some cases, the RBS is not native to the promoter that it is operably associated with. As a non-limiting example, the RBS for the sarA promoter is the RBS from sodB, rather than sarA. For instance, the RBS used with a promoter for a translation of a protein herein comprises SEQ ID NO: 21. In some cases, the promoter is sarA.

### Expression Cassette

In some embodiments, the nucleic acid sequence is an expression cassette or in an expression cassette. In some embodiments, the expression cassettes are designed to express the nucleic acid sequence disclosed herein. In some embodiments, the nucleic acid sequence is an expression cassette encoding components of a CRISPR-Cas system and/or peptide. In some embodiments, the nucleic acid sequence is an expression cassette encoding components of a Type I CRISPR-Cas system. In some embodiments, the nucleic acid sequence is an expression cassette encoding an operable CRISPR-Cas system. In some embodiments, the nucleic acid sequence is an expression cassette encoding the operable components of a Type I CRISPR-Cas system, including Cascade and Cas3. In some embodiments, the nucleic acid sequence is an expression cassette encoding the operable components of a Type I CRISPR-Cas system, including a crRNA, Cascade and Cas3. In some embodiments, the nucleic acid sequence is an expression cassette encoding a peptide (e.g., antimicrobial peptide).

In some embodiments, an expression cassette comprising a nucleic acid sequence of interest is chimeric, meaning that at least one of its components is heterologous with respect to at least one of its other components. In some embodiments, an expression cassette is naturally occurring but has been obtained in a recombinant form useful for heterologous expression.

In some embodiments, an expression cassette includes a transcriptional and/or translational termination region (i.e. termination region) that is functional in the selected host cell. In some embodiments, termination regions are responsible for the termination of transcription beyond the heterologous nucleic acid sequence of interest and for correct mRNA polyadenylation. In some embodiments, the termination region is native to the transcriptional initiation region, is native to the operably linked nucleic acid sequence of interest, is native to the host cell, or is derived from another source (i.e., foreign or heterologous to the promoter, to the nucleic acid sequence of interest, to the host, or any combination thereof). In some embodiments, terminators are operably linked to the nucleic acid sequence disclosed herein.

In some embodiments, an expression cassette includes a nucleotide sequence for a selectable marker. In some embodiments, the nucleotide sequence encodes either a selectable or a screenable marker, depending on whether the marker confers a trait that is selected for by chemical means, such as by using a selective agent (e.g. an antibiotic), or on whether the marker is simply a trait that one identifies through observation or testing, such as by screening (e.g., fluorescence).

### Vectors

In addition to expression cassettes, the nucleic acid sequences disclosed herein (e.g. nucleic acid sequence comprising a CRISPR array, CRISPR-Cas, peptide, antimicrobial agent) are used in connection with vectors. A vector comprises a nucleic acid molecule comprising the nucleotide sequence(s) to be transferred, delivered or introduced. Non-limiting examples of general classes of vectors include, but are not limited to, a viral vector, a plasmid vector, a phage vector, a phagemid vector, a cosmid vector, a fosmid vector, a bacteriophage, an artificial chromosome, or an agrobacterium binary vector in double or single stranded linear or circular form which may or may not be self-transmissible or mobilizable. A vector transforms prokaryotic or eukaryotic host either by integration into the cellular genome or exist extrachromosomally (e.g. autonomous replicating plasmid with an origin of replication). Additionally, included are shuttle vectors by which is meant a DNA vehicle capable, naturally or by design, of replication in two different host organisms. In some embodiments, a shuttle vector replicates in actinomycetes and bacteria and/or eukaryotes. In some embodiments, the nucleic acid in the vector are under the control of, and operably linked to, an appropriate promoter or other regulatory elements for transcription in a host cell. In some embodiments, the vector is a bi-functional expression vector which functions in multiple hosts.

### Sequence Optimization

In some embodiments, the nucleic acid sequence encoding a payload (e.g. the nucleic acid insert) is optimized for stable expression in a phage genome. In some embodiments, the insert is stable through at least 2, 3, 4, 5, 6, 7, 8, 9, or 10 generations of passaging. In some embodiments, the nucleic acid sequence is optimized by optimizing the insertion site, modifying secondary structures, modifying DNA modification sites, modifying restriction enzyme motifs, codon optimization, GC % optimization, or a combination thereof. In some embodiments, the insertion site of the nucleic acid sequence is optimized. In some embodiments, the nucleic acid sequence is modified to remove secondary structures.

In certain embodiments, the bacteriophage comprises a nucleic acid insert modified from an exogenous nucleic acid described herein, wherein the nucleic acid comprises a first plurality of codons encoding for a first protein, and the nucleic acid insert comprises a second plurality of codons encoding for a second protein, wherein the first protein and the second protein have at least 90% amino acid sequence identity, and wherein at least 50% of the second plurality of codons are high frequency codons in the bacteriophage genome. In certain aspects, described herein is a method of inserting an exogenous sequence comprising a plurality of codons encoding a first protein into a bacteriophage, the method comprising substituting one or more of the plurality of codons with a codon native to the bacteriophage to generate a nucleic acid insert encoding a second protein, wherein the first protein and the second protein have at least 90% amino acid sequence identity. In certain aspects, described herein is a nucleic acid insert modified from an exogenous nucleic acid, wherein the nucleic acid comprises a first plurality of codons encoding for a first protein, and the nucleic acid insert comprises a second plurality of codons encoding for a second protein, wherein the first protein and the second protein have at least 90% amino acid sequence identity, and wherein at least 50% of the second plurality of codons are high frequency codons in the bacteriophage genome. In some embodiments, the first protein and the second protein have at least 95%, 97.5%, 99% or 99.5% sequence identity. In some embodiments, at least 50%<60%, 70%, 80%, 90% or more than 90% of the second plurality of codons are high frequency codons in the bacteriophage genome. In some embodiments, the second plurality of codons match the profile of codons in the bacteriophage genome.

In some embodiments, the nucleic acid sequence is modified to remove DNA modification sites. In some embodiments, the DNA modification sites comprise DNA methylation sites.

In some embodiments, the nucleic acid sequence is modified to remove restriction enzyme motifs. In some embodiments, the nucleic acid sequence is modified to remove restriction enzyme motifs for a restriction enzyme derived from a bacterial species described herein. In some embodiments, the nucleic acid insert does not comprise, or comprises fewer than 10 sites recognized by a bacterial enzyme.

In some embodiments, the nucleic acid sequence is codon optimized for expression in any species of interest. Codon optimization involves modification of a nucleotide sequence for codon usage bias using species-specific codon usage tables. The codon usage tables are generated based on a sequence analysis of the most highly expressed genes for the species of interest. When the nucleotide sequences are to be expressed in the nucleus, the codon usage tables are generated based on a sequence analysis of highly expressed nuclear genes for the species of interest. The modifications of the nucleotide sequences are determined by comparing the species-specific codon usage table with the codons present in the native polynucleotide sequences. Codon optimization of a nucleotide sequence results in a nucleotide sequence having less than 100% identity (e.g., 50%, 60%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and the like) to the native nucleotide sequence but which still encodes a polypeptide having the same function as that encoded by the original nucleotide sequence. In some embodiments, the nucleic acid sequences of this disclosure are codon optimized for expression in the organism/species of interest.

In some embodiments, the nucleic acid sequence is modified to optimize the percent GC content. In some embodiments, the percent GC content is modified so that at least 10%, 20%, 30%, 40%, 50%, 60%, 70%, 80% 90% or more than 90% of the nucleotides comprises guanine or cytosine. In some embodiments, the percent GC content is modified so that no more than 10%, 20%, 30%, 40%, 50%, 60%, 70%, 80% 90% or more than 90% of the nucleotides comprises guanine or cytosine.

In some embodiments, the exogenous nucleic acid is a bacterial nucleic acid. In some embodiments, the nucleic acid insert and the bacterial nucleic acid have less than 100%, 95%, 90%, 80%, 70%, 60%, or 50% sequence identity. In some embodiments, the first bacterial protein is a CRISPR-Cas protein as described herein. In some embodiments, the first bacterial protein is an antimicrobial agent and/or peptide as described herein.

### Transformation

In some embodiments, the nucleic acid sequence, and/or expression cassettes disclosed herein are expressed transiently and/or stably incorporated into the genome of a host organism. In some embodiments, a the nucleic acid sequence and/or expression cassettes disclosed herein is introduced into a cell by any method known to those of skill in the art. Exemplary methods of transformation include transformation via electroporation of competent cells, passive uptake by competent cells, chemical transformation of competent cells, as well as any other electrical, chemical, physical (mechanical) and/or biological mechanism that results in the introduction of nucleic acid into a cell, including any combination thereof. In some embodiments, transformation of a cell comprises nuclear transformation. In some embodiments, transformation of a cell comprises plasmid transformation and conjugation.

In some embodiments, when more than one nucleic acid sequence is introduced, the nucleotide sequences are assembled as part of a single nucleic acid construct, or as separate nucleic acid constructs, and are located on the same or different nucleic acid constructs. In some embodiments, nucleotide sequences are introduced into the cell of interest in a single transformation event, or in separate transformation events.

### Methods of Use

Disclosed herein, in certain embodiments, are methods of killing a target bacterium comprising contacting or introducing into a target bacterium any of the bacteriophages disclosed herein. In some embodiments, the target bacterium is a Staphylococcus spp.

Further disclosed herein, in certain embodiments, are methods of modifying a mixed population of bacterial cells having a first bacterial species that comprises a target nucleotide sequence in the essential gene and a second bacterial species that does not comprise a target nucleotide sequence in the essential gene, the method comprising introducing into the mixed population of bacterial cells any of the bacteriophages disclosed herein.

Also disclosed herein, in certain embodiments, are methods of treating a disease in an individual in need thereof, the method comprising administering to the individual any of the bacteriophages disclosed herein.

In some embodiments, the target bacterium is killed solely by the lytic activity of the bacteriophage. In some embodiments, the target bacterium is Staphylococcus. In some embodiments, a population of Staphylococcus bacteria is targeted by the bacteriophage. In some embodiments, a population of Staphylococcus bacteria is killed by the bacteriophage. In some embodiments, the bacteriophage cocktail described herein targets a large number of Staphylococcus strains. In some embodiments, the bacteriophage cocktail targets at least 70%, 75%, 80%, 85%, 90%, 95%, 97.5%, 99% or more than 99% of strains of Staphylococcus. In some embodiments, the bacteriophage cocktail targets at least 70%, 75%, 80%, 85%, 90%, 95%, 97.5%, 99% or more than 99% of strains of S. aureus. In some embodiments, the bacteriophage cocktail kills at least 70%, 75%, 80%, 85%, 90%, 95%, 97.5%, 99% or more than 99% of strains of Staphylococcus. In some embodiments, the bacteriophage cocktail kills at least 70%, 75%, 80%, 85%, 90%, 95%, 97.5%, 99% or more than 99% of strains of S. aureus. In some embodiments, the bacteriophage cocktail targets Staphylococcus spp. specifically and does not target other species of bacteria. In some embodiments, the bacteriophage cocktail targets Staphylococcus spp. and does not target non Staphylococcus spp. In some embodiments, the bacteriophage cocktail comprises two or more of: Phictavirus, Rosenblumvirus, and Kayvirus.

In some embodiments, the Staphylococcus bacterium is killed solely by lytic activity of the bacteriophage. In some embodiments, the Staphylococcus bacterium is killed solely by activity of the CRISPR-Cas system. In some embodiments, the Staphylococcus bacterium is killed by the processing of the CRISPR array by a CRISPR-Cas system to produce a processed crRNA capable of directing CRISPR-Cas based endonuclease activity and/or cleavage at the target nucleotide sequence in the target gene of the bacterium. In some embodiments, the Staphylococcus bacterium is killed solely by the antimicrobial peptide.

In some embodiments, the Staphylococcus bacterium is killed by lytic activity of the bacteriophage in combination with activity of the Type I CRISPR-Cas system. In some embodiments, the Staphylococcus bacterium is killed by the activity of the Type I CRISPR-Cas system, independently of the lytic activity of the bacteriophage. In some embodiments, the activity of the Type I CRISPR-Cas system supplements or enhances the lytic activity of the bacteriophage. In some embodiments, the activity of the Type I CRISPR-Cas system and the lytic activity of the bacteriophage are additive.

In some embodiments, the Staphylococcus bacterium is killed by lytic activity of the bacteriophage in combination with activity of the Type I CRISPR-Cas system and the anti-microbial peptide. In some embodiments, the Staphylococcus bacterium is killed by the activity of the Type I CRISPR-Cas system, independently of the lytic activity of the bacteriophage and the anti-microbial peptide. In some embodiments, the activity of the Type I CRISPR-Cas system supplements or enhances the lytic activity of the bacteriophage and the anti-microbial peptide. In some embodiments, the activity of the Type I CRISPR-Cas system, the lytic activity of the bacteriophage, and the activity of the antimicrobial peptide are additive.

In some embodiments, the lytic activity of the bacteriophage and the activity of the Type I CRISPR-Cas system is synergistic. In some embodiments, a synergistic activity is defined as an activity resulting in a greater level of phage kill than the additive combination of the lytic activity of the bacteriophage and the Type I CRISPR-Cas system. In some embodiments, the lytic activity of the bacteriophage is modulated by a concentration of the bacteriophage. In some embodiments, the activity of the Type I CRISPR-Cas system is modulated by a concentration of the bacteriophage.

In some embodiments, the synergistic killing of the bacterium is modulated to favor killing by the lytic activity of the bacteriophage over the activity of the CRISPR-Cas system by increasing the concentration of bacteriophage administered to the bacterium. In some embodiments, the synergistic killing of the bacterium is modulated to disfavor killing by the lytic activity of the bacteriophage over the activity of the CRISPR-Cas system by decreasing the concentration of bacteriophage administered to the bacterium. In some embodiments, at low concentrations, lytic replication allows for amplification and killing of the target bacteria. In some embodiments, at high concentrations, amplification of a phage is not required. In some embodiments, the synergistic killing of the bacterium is modulated to favor killing by the activity of the CRISPR-Cas system over the lytic activity of the bacteriophage by altering the number, the length, the composition, the identity, or any combination thereof, of the spacers so as to increase the lethality of the CRISPR array. In some embodiments, the synergistic killing of the bacterium is modulated to disfavor killing by the activity of the CRISPR-Cas system over the lytic activity of the bacteriophage by altering the number, the length, the composition, the identity, or any combination thereof, of the spacers so as to decrease the lethality of the CRISPR array.

In some embodiments, the lytic activity of the bacteriophage, the activity of the Type I CRISPR-Cas system, and the activity of the antimicrobial peptide is synergistic. In some embodiments, a synergistic activity is defined as an activity resulting in a greater level of phage kill than the additive combination of the lytic activity of the bacteriophage, the Type I CRISPR-Cas system, and the antimicrobial peptide. In some embodiments, the lytic activity of the bacteriophage is modulated by a concentration of the bacteriophage and the antimicrobial peptide. In some embodiments, the activity of the Type I CRISPR-Cas system is modulated by a concentration of the bacteriophage.

### Administration Routes and Dosage

Dose and duration of the administration of a composition disclosed herein will depend on a variety of factors, including the subject's age, subject's weight, and tolerance of the phage. In some embodiments, a bacteriophage disclosed herein is administered to patients intra-arterially, intravenously, intraurethrally, intramuscularly, orally, subcutaneously, by inhalation, or any combination thereof. In some embodiments, a bacteriophage disclosed herein is administered to patients by oral administration. In some embodiments, a bacteriophage disclosed herein is administered to patients by topical, cutaneous, transdermal, transmucosal, implantation, sublingual, buccal, rectal, vaginal, ocular, otic, or nasal administration. In some embodiments, a bacteriophage disclosed herein is administered to patients by any combination of the aforementioned routes of administration.

In some embodiments, the compositions (bacteriophage) disclosed herein are administered before, during, or after the occurrence of a disease or condition. In some embodiment, the timing of administering the composition containing the bacteriophage varies. In some embodiments, the pharmaceutical compositions are used as a prophylactic and are administered continuously to subjects with a propensity to conditions or diseases in order to prevent the occurrence of the disease or condition. In some embodiments, pharmaceutical compositions are administered to a subject during or as soon as possible after the onset of the symptoms. In some embodiments, the administration of the compositions is initiated within the first 48 hours of the onset of the symptoms, within the first 24 hours of the onset of the symptoms, within the first 6 hours of the onset of the symptoms, or within 3 hours of the onset of the symptoms. In some embodiments, the initial administration of the composition is via any route practical, such as by any route described herein using any formulation described herein. In some embodiments, the compositions is administered as soon as is practicable after the onset of a disease or condition is detected or suspected, and for a length of time necessary for the treatment of the disease, such as, for example, from about 1 month to about 3 months. In some embodiments, the length of treatment will vary for each subject.

### Bacterial Infections

Disclosed herein, in certain embodiments, are methods of treating bacterial infections. In some embodiments, the bacteriophages disclosed herein treat or prevent diseases or conditions mediated or caused by bacteria as disclosed herein in a human or animal subject. In some embodiments, the bacteriophages disclosed herein treat or prevent diseases or conditions caused or exacerbated by bacteria as disclosed herein in a human or animal subject. Such bacteria are typically in contact with tissue of the subject including: gut, oral cavity, lung, armpit, ocular, vaginal, anal, car, nose or throat tissue. In some embodiments, a bacterial infection is treated by modulating the activity of the bacteria and/or by directly killing of the bacteria.

In some embodiments, the bacterium is Staphylococcus spp. In some embodiments, the bacterium is S. aureus.

In some embodiments, one or more Staphylococcus species present in a bacterial population are pathogenic.

In some embodiments, the bacteriophages disclosed herein are used to treat an infection, a disease, or a condition, in the gastrointestinal tract of a subject. In some embodiments, the bacteriophages are used to modulate and/or kill target bacteria within the microbiome or gut flora of a subject. In some embodiments, the bacteriophages are used to selectively modulate and/or kill one or more target bacteria from a plurality of bacteria within the microbiome or gut flora of a subject. In some embodiments, the bacteriophages are used to selectively modulate and/or kill one or more target enteropathogenic bacteria from a plurality of bacteria within the microbiome or gut flora of a subject.

In some embodiments, the bacteriophages disclosed herein are used to treat an infection, a disease, or a condition, in the urinary tract of a subject. In some embodiments, the bacteriophages are used to modulate and/or kill target bacteria within the urinary tract flora of a subject. The urinary tract flora includes, but is not limited, to Staphylococcus epidermidis, Enterococcus faecalis, and some alpha-hemolytic Streptococci. In some embodiments, the bacteriophages are used to selectively modulate and/or kill one or more target uropathogenic bacteria from a plurality of bacteria within the urinary tract flora of a subject.

In some embodiments, the bacteriophages disclosed herein are used to treat an infection, a disease, or a condition, on the skin of a subject. In some embodiments, the bacteriophages are used to modulate and/or kill target bacteria on the skin of a subject.

In some embodiments, the bacteriophages disclosed herein are used to treat an infection, a disease, or a condition, on a mucosal membrane of a subject. In some embodiments, the bacteriophages are used to modulate and/or kill target bacteria on the mucosal membrane of a subject.

In some embodiments, the pathogenic bacteria are antibiotic resistant. In some embodiments, the pathogenic bacteria is methicillin resistant. In some embodiments, the pathogenic bacteria is methicillin resistant Staphylococcus aureus.

In some embodiments, the one or more target bacteria present in the bacterial population form a biofilm. In some embodiments, the biofilm comprises pathogenic bacteria. In some embodiments, the bacteriophage disclosed herein is used to treat a biofilm.

In some embodiments, the bacterium includes Staphylococcus spp. In some embodiments, the bacterium is Staphylococcus aureus.

In some embodiments, the bacteriophage treats acne and other related skin infections.

In some embodiments, the Staphylococcus species is a multiple drug resistant (MDR) bacteria strain. An MDR strain is a bacteria strain that is resistant to at least one antibiotic. In some embodiments, a bacteria strain is resistant to an antibiotic class such as a cephalosporin, a fluoroquinolone, a carbapenem, a colistin, an aminoglycoside, vancomycin, streptomycin, and methicillin. In some embodiments, the bacteria strain is Staphylococcus aureus. In some embodiments, the pathogenic bacteria is methicillin resistant Staphylococcus aureus.

In some embodiments, the bacterium is S. aureus. In some embodiments, the methods and compositions disclosed herein are for use in veterinary and medical applications as well as research applications.

### Microbiome

“Microbiome”, “microbiota”, and “microbial habitat” are used interchangeably hereinafter and refer to the ecological community of microorganisms that live on or in a subject's bodily surfaces, cavities, and fluids. Non-limiting examples of habitats of microbiome include: gut, colon, skin, skin surfaces, skin pores, vaginal cavity, umbilical regions, conjunctival regions, intestinal regions, stomach, nasal cavities and passages, gastrointestinal tract, urogenital tracts, saliva, mucus, and feces. In some embodiments, the microbiome comprises microbial material including, but not limited to, bacteria, archaea, protists, fungi, and viruses. In some embodiments, the microbial material comprises a gram-negative bacterium. In some embodiments, the microbial material comprises a gram-positive bacterium. In some embodiments, the microbial material comprises Proteobacteria, Actinobacteria, Bacteroidetes, or Firmicutes.

In some embodiments, the bacteriophages as disclosed herein are used to modulate or kill target bacteria within the microbiome of a subject. In some embodiments, the bacteriophages are used to modulate and/or kill target bacteria within the microbiome by the CRISPR-Cas system, lytic activity, or a combination thereof. In some embodiments, the bacteriophages are used to modulate and/or kill target bacteria within the microbiome of a subject. In some embodiments, the bacteriophages are used to selectively modulate and/or kill one or more target bacteria from a plurality of bacteria within the microbiome of a subject.

In some embodiments, the bacteriophages are used to modulate or kill target single or plurality of bacteria within the microbiome or gut flora of the gastrointestinal tract of a subject. Modification (e.g., dysbiosis) of the microbiome or gut flora increases the risk for health conditions such as diabetes, mental disorders, ulcerative colitis, colorectal cancer, autoimmune disorders, obesity, diabetes, diseases of the central nervous system and inflammatory bowel disease. An exemplary bacteria associated with diseases and conditions of gastrointestinal tract and are being modulated or killed by the bacteriophages include strains, sub-strains, and enterotypes of S. aureus.

In some embodiments, the bacteriophages are used to modulate or kill target single or plurality of bacteria within the microbiome or gut flora of the gastrointestinal tract of a subject. Modification (e.g., dysbiosis) of the microbiome or gut flora increases the risk for health conditions such as diabetes, mental disorders, ulcerative colitis, colorectal cancer, autoimmune disorders, obesity, diabetes, diseases of the central nervous system and inflammatory bowel disease. An exemplary list of the bacteria associated with diseases and conditions of gastrointestinal tract and are being modulated or killed by the bacteriophages include strains, sub-strains, and enterotypes of Enterobacteriaceae, Pasteurellaceae, Fusobacteriaceae, Neisseriaceae, Veillonellaceae, Gemellaceae, Bacteroidales, Clostridiales, Erysipelotrichaceae, Bifidobacteriaceae, Bacteroides, Faccalibacterium, Roseburia, Blautia, Ruminococcus, Coprococcus, Streptococcus, Dorea, Blautia, Ruminococcus, Lactobacillus, Enterococcus, Streptococcus, Actinomyces, Lactococcus, Roseburia, Blautia, Dialister, Desulfovibrio, Escherichia, Lactobacillus, Coprococcus, Clostridium, Bifidobacterium, Klebsiella, Granulicatella, Eubacterium, Anaerostipes, Parabacteroides, Coprobacillus, Gordonibacter, Collinsella, Bacteroide, Faecalibacterium, Anaerotruncus, Alistipes, Haemophilus, Anaerococcus, Veillonella, Arevotella, Akkermansia, Bilophila, Sutterella, Eggerthella, Holdemania, Gemella, Peptoniphilus, Rothia, Pediococcus, Citrobacter, Odoribacter, Enterobacteria, Fusobacterium, Proteus, Escherichia coli, Fusobacterium nucleatum, Haemophilus parainfluenzae (Pasteurellaceae), Veillonella parvula, Eikenella corrodens (Neisseriaceae), Gemella moribillum, Bacteroides vulgatus, Bacteroides caccae, Bifidobacterium bifidum, Bifidobacterium longum, Bifidobacterium adolescentis, Bifidobacterium dentum, Blautia hansenii, Ruminococcus gnavus, Clostridium nexile, Faecalibacterium prausnitzii, Ruminoccus torques, Clostridium bolteae, Eubacterium rectale, Roseburia intestinalis, and Coprococcus iomes.

In some embodiments, the bacteriophages are used to modulate or kill target single or plurality of bacteria within the microbiome or flora of the epidermis of a subject. Modification (e.g., dysbiosis) of the microbiome or skin flora increases the risk for health conditions such as eczema or Atopic Dermatitis.

In some embodiments, a bacteriophage disclosed herein is administered to a subject to promote a healthy microbiome. In some embodiments, a bacteriophage disclosed herein is administered to a subject to restore a subject's microbiome to a microbiome composition that promotes health. In some embodiments, a composition comprising a bacteriophage disclosed herein comprises a prebiotic or a third agent. In some embodiment, microbiome related disease or disorder is treated by a bacteriophage disclosed herein.

### Environmental Therapy

In some embodiments, bacteriophages disclosed herein are further used for food and agriculture sanitation (including meats, fruits and vegetable sanitation), hospital sanitation, home sanitation, vehicle and equipment sanitation, industrial sanitation, etc. In some embodiments, bacteriophages disclosed herein are used for the removal of antibiotic-resistant or other undesirable pathogens from medical, veterinary, animal husbandry, or any additional environments bacteria are passed to humans or animals.

Environmental applications of phage in health care institutions are for equipment such as endoscopes and environments such as ICUs which are potential sources of nosocomial infection due to pathogens that are difficult or impossible to disinfect. In some embodiments, a phage disclosed herein is used to treat equipment or environments inhabited by bacterial genera which become resistant to commonly used disinfectants. In some embodiments, phage compositions disclosed herein are used to disinfect inanimate objects. In some embodiments, an environment disclosed herein is sprayed, painted, or poured onto with aqueous solutions with phage titers. In some embodiments, a bacteriophage disclosed herein is applied by aerosolizing agents that include dry dispersants to facilitate distribution of the bacteriophage into the environment. In some embodiments, objects are immersed in a solution containing bacteriophage disclosed herein.

### Sanitation

In some embodiments, bacteriophages disclosed herein are used as sanitation agents in a variety of fields. Although the terms “phage” or “bacteriophage” may be used, it should be noted that, where appropriate, this term should be broadly construed to include a single bacteriophage, multiple bacteriophages, such as a bacteriophage mixtures and mixtures of a bacteriophage with an agent, such as a disinfectant, a detergent, a surfactant, water, etc.

In some embodiments, bacteriophages are used to sanitize hospital facilities, including operating rooms, patient rooms, waiting rooms, lab rooms, or other miscellaneous hospital equipment. In some embodiments, this equipment includes electrocardiographs, respirators, cardiovascular assist devices, intraaortic balloon pumps, infusion devices, other patient care devices, televisions, monitors, remote controls, telephones, beds, etc. In some situations, the bacteriophage is applied through an aerosol canister. In some embodiments, bacteriophage is applied by wiping the phage on the object with a transfer vehicle.

In some embodiments, a bacteriophage described herein is used in conjunction with patient care devices. In some embodiment, bacteriophage is used in conjunction with a conventional ventilator or respiratory therapy device to clean the internal and external surfaces between patients. Examples of ventilators include devices to support ventilation during surgery, devices to support ventilation of incapacitated patients, and similar equipment. In some embodiments, the conventional therapy includes automatic or motorized devices, or manual bag-type devices such as are commonly found in emergency rooms and ambulances. In some embodiments, respiratory therapy includes inhalers to introduce medications such as bronchodilators as commonly used with chronic obstructive pulmonary disease or asthma, or devices to maintain airway patency such as continuous positive airway pressure devices.

In some embodiment, a bacteriophage described herein is used to cleanse surfaces and treat colonized people in an area where highly contagious bacterial diseases, such as meningitis or enteric infections are present.

In some embodiments, water supplies are treated with a composition disclosed herein. In some embodiments, bacteriophage disclosed herein is used to treat contaminated water, water found in cisterns, wells, reservoirs, holding tanks, aqueducts, conduits, and similar water distribution devices. In some embodiments, the bacteriophage is applied to industrial holding tanks where water, oil, cooling fluids, and other liquids accumulate in collection pools. In some embodiments, a bacteriophage disclosed herein is periodically introduced to the industrial holding tanks in order to reduce bacterial growth.

In some embodiments, bacteriophages disclosed herein are used to sanitize a living area, such as a house, apartment, condominium, dormitory, or any living area. In some embodiments, the bacteriophage is used to sanitize public areas, such as theaters, concert halls, museums, train stations, airports, pet areas, such as pet beds, or litter boxes. In this capacity, the bacteriophage is dispensed from conventional devices, including pump sprayers, aerosol containers, squirt bottles, pre-moistened towelettes, etc, applied directly to (e.g., sprayed onto) the area to be sanitized, or be transferred to the area via a transfer vehicle, such as a towel, sponge, etc. In some embodiments, a phage disclosed herein is applied to various rooms of a house, including the kitchen, bedrooms, bathrooms, garage, basement, etc. In some embodiments, a phage disclosed herein is in the same manner as conventional cleaners. In some embodiments, the phage is applied in conjunction with (before, after, or simultaneously with) conventional cleaners provided that the conventional cleaner is formulated so as to preserve adequate bacteriophage biologic activity.

In some embodiments, a bacteriophage disclosed herein is added to a component of paper products, either during processing or after completion of processing of the paper products. Paper products to which a bacteriophage disclosed herein is added include, but are not limited to, paper towels, toilet paper, moist paper wipes.

### Food Safety

In some embodiments, a bacteriophage described herein is used in any food product or nutritional supplement, for preventing contamination. Examples for food or pharmaceuticals products are milk, yoghurt, curd, cheese, fermented milks, milk based fermented products, ice-creams, fermented cereal based products, milk based powders, infant formulae or tablets, liquid suspensions, dried oral supplement, wet oral supplement, or dry-tube-feeding.

The broad concept of bacteriophage sanitation is applicable to other agricultural applications and organisms. Produce, including fruits and vegetables, dairy products, and other agricultural products. For example, freshly cut produce frequently arrive at the processing plant contaminated with pathogenic bacteria. This has led to outbreaks of food-borne illness traceable to produce. In some embodiments, the application of bacteriophage preparations to agricultural produce substantially reduces or eliminate the possibility of food-borne illness through application of a single phage or phage mixture with specificity toward species of bacteria associated with food-borne illness. In some embodiments, bacteriophages are applied at various stages of production and processing to reduce bacterial contamination at that point or to protect against contamination at subsequent points.

In some embodiments, specific bacteriophages are applied to produce in restaurants, grocery stores, produce distribution centers. In some embodiments, bacteriophages disclosed herein are periodically or continuously applied to the fruit and vegetable contents of a salad bar. In some embodiments, the application of bacteriophages to a salad bar or to sanitize the exterior of a food item is a misting or spraying process or a washing process.

In some embodiments, a bacteriophage described herein is used in matrices or support media containing with packaging containing meat, produce, cut fruits and vegetables, and other foodstuffs. In some embodiments, polymers that are suitable for packaging are impregnated with a bacteriophage preparation.

In some embodiments, a bacteriophage described herein is used in farm houses and livestock feed. In some embodiments, on a farm raising livestock, the livestock is provided with bacteriophage in their drinking water, food, or both. In some embodiments, a bacteriophage described herein is sprayed onto the carcasses and used to disinfect the slaughter area.

The use of specific bacteriophages as biocontrol agents on produce provides many advantages. For example, bacteriophages are natural, non-toxic products that will not disturb the ecological balance of the natural microflora in the way the common chemical sanitizers do, but will specifically lyse the targeted food-borne pathogens. Because bacteriophages, unlike chemical sanitizers, are natural products that evolve along with their host bacteria, new phages that are active against recently emerged, resistant bacteria are rapidly identified when required, whereas identification of a new effective sanitizer is a much longer process, several years.

### Pharmaceutical Compositions

Disclosed herein, in certain embodiments, are pharmaceutical compositions comprising (a) the nucleic acid sequences as disclosed herein; and (b) a pharmaceutically acceptable excipient. Also disclosed herein, in certain embodiments, are pharmaceutical compositions comprising (a) the bacteriophages as disclosed herein; and (b) a pharmaceutically acceptable excipient. Further disclosed herein, in certain embodiments, are pharmaceutical compositions comprising (a) the compositions as disclosed herein; and (b) a pharmaceutically acceptable excipient.

In some embodiments, the disclosure provides pharmaceutical compositions and methods of administering the same to treat bacterial, archacal infections or to disinfect an area. In some embodiments, the pharmaceutical composition comprises any of the reagents discussed above in a pharmaceutically acceptable carrier. In some embodiments, a pharmaceutical composition or method disclosed herein treats bloodstream infections (BSI) and/or inflammatory diseases (e.g. atopic dermatitis (AD)). In some embodiments, a pharmaceutical composition or method disclosed herein treats eczema. In some embodiments, a pharmaceutical composition or method disclosed herein treats atopic dermatitis.

In some embodiments, compositions disclosed herein comprise medicinal agents, pharmaceutical agents, carriers, adjuvants, dispersing agents, diluents, and the like.

In some embodiments, the bacteriophages disclosed herein are formulated for administration in a pharmaceutical carrier in accordance with suitable methods. In some embodiments, the manufacture of a pharmaceutical composition according to the disclosure, the bacteriophage is admixed with, inter alia, an acceptable carrier. In some embodiments, the carrier is a solid (including a powder) or a liquid, or both, and is preferably formulated as a unit-dose composition. In some embodiments, one or more bacteriophages are incorporated in the compositions disclosed herein, which are prepared by any suitable method of a pharmacy.

In some embodiment, a method of treating subject's in-vivo, comprising administering to a subject a pharmaceutical composition comprising a bacteriophage disclosed herein in a pharmaceutically acceptable carrier, wherein the pharmaceutical composition is administered in a therapeutically effective amount. In some embodiments, the administration of the bacteriophage to a human subject or an animal in need thereof are by any means known in the art.

In some embodiments, bacteriophages disclosed herein are for oral administration. In some embodiments, the bacteriophages are administered in solid dosage forms, such as capsules, tablets, and powders, or in liquid dosage forms, such as elixirs, syrups, and suspensions. In some embodiments, compositions and methods suitable for buccal (sublingual) administration include lozenges comprising the bacteriophages in a flavored base, usually sucrose and acacia or tragacanth; and pastilles comprising the bacteriophages in an inert base such as gelatin and glycerin or sucrose and acacia.

In some embodiments, methods and compositions of the present disclosure are suitable for parenteral administration comprising sterile aqueous and non-aqueous injection solutions of the bacteriophage. In some embodiments, these preparations are isotonic with the blood of the intended recipient. In some embodiments, these preparations comprise antioxidants, buffers, bacteriostals and solutes which render the composition isotonic with the blood of the intended recipient. In some embodiments, aqueous and non-aqueous sterile suspensions include suspending agents and thickening agents. In some embodiments, compositions disclosed herein are presented in unit\dose or multi-dose containers, for example sealed ampoules and vials, and are stored in a freeze-dried (lyophilized) condition requiring only the addition of the sterile liquid carrier, for example, saline or water for injection on immediately prior to use.

In some embodiment, methods and compositions suitable for rectal administration are presented as unit dose suppositories. In some embodiments, these are prepared by admixing the bacteriophage with one or more conventional solid carriers, for example, cocoa butter, and then shaping the resulting mixture. In some embodiments, methods and compositions suitable for topical application to the skin are in the form of an ointment, cream, lotion, paste, gel, spray, aerosol, or oil. In some embodiments, carriers which are used include petroleum jelly, lanoline, polyethylene glycols, alcohols, transdermal enhancers, and combinations of two or more thereof.

In some embodiments, methods and compositions suitable for transdermal administration are presented as discrete patches adapted to remain in intimate contact with the epidermis of the recipient for a prolonged period of time.

In some embodiments, methods and compositions suitable for nasal administration or otherwise administered to the lungs of a subject include any suitable means, e.g., administered by an aerosol suspension of respirable particles comprising the bacteriophage compositions, which the subject inhales. In some embodiments, the respirable particles are liquid or solid. As used herein, “aerosol” includes any gas-borne suspended phase, which is capable of being inhaled into the bronchioles or nasal passages. In some embodiments, aerosols of liquid particles are produced by any suitable means, such as with a pressure-driven aerosol nebulizer or an ultrasonic nebulizer. In some embodiments, aerosols of solid particles comprising the composition is produced with any solid particulate medicament aerosol generator, by techniques known in the pharmaceutical art.

In some embodiment, methods and compositions suitable for administering bacteriophages disclosed herein to a surface of an object or subject includes aqueous solutions. In some embodiments, such aqueous solutions are sprayed onto the surface of an object or subject. In some embodiment, the aqueous solutions are used to irrigate and clean a physical wound of a subject form foreign debris including bacteria.

In some embodiments, the bacteriophages disclosed herein are administered to the subject in a therapeutically effective amount. In some embodiments, at least one bacteriophage composition disclosed herein is formulated as a pharmaceutical formulation. In some embodiments, a pharmaceutical formulation comprises 1, 2, 3, 4, 5, 6, 7, 8, 9, 10, 11, 12, 13, 14, 15, 16, 17, 18, 19, 20 or more bacteriophage disclosed herein. In some instances, a pharmaceutical formulation comprises a bacteriophage described herein and at least one of: an excipient, a diluent, or a carrier.

In some embodiments, a pharmaceutical formulation comprises an excipient. Excipients are described in the Handbook of Pharmaceutical Excipients, American Pharmaceutical Association (1986) and includes but are not limited to solvents, dispersion media, diluents, or other liquid vehicles, dispersion or suspension aids, surface active agents, isotonic agents, thickening or emulsifying agents, preservatives, solid binders, and lubricants.

Non-limiting examples of suitable excipients include but is not limited to a buffering agent, a preservative, a stabilizer, a binder, a compaction agent, a lubricant, a chelator, a dispersion enhancer, a disintegration agent, a flavoring agent, a sweetener, a coloring agent.

In some embodiments, an excipient is a buffering agent. Non-limiting examples of suitable buffering agents include but is not limited to sodium citrate, magnesium carbonate, magnesium bicarbonate, calcium carbonate, and calcium bicarbonate. In some embodiments, a pharmaceutical formulation comprises any one or more buffering agent listed: sodium bicarbonate, potassium bicarbonate, magnesium hydroxide, magnesium lactate, magnesium glucomate, aluminum hydroxide, sodium citrate, sodium tartrate, sodium acetate, sodium carbonate, sodium polyphosphate, potassium polyphosphate, sodium pyrophosphate, potassium pyrophosphate, disodium hydrogen phosphate, dipotassium hydrogen phosphate, trisodium phosphate, tripotassium phosphate, potassium metaphosphate, magnesium oxide, magnesium hydroxide, magnesium carbonate, magnesium silicate, calcium acetate, calcium glycerophosphate, calcium chloride, calcium hydroxide and other calcium salts.

In some embodiments an excipient is a preservative. Non-limiting examples of suitable preservatives include but is not limited to antioxidants, such as alpha-tocopherol and ascorbate, and antimicrobials, such as parabens, chlorobutanol, and phenol. In some embodiments, antioxidants include but not limited to Ethylenediaminetetraacetic acid (EDTA), citric acid, ascorbic acid, butylated hydroxytoluene (BHT), butylated hydroxy anisole (BHA), sodium sulfite, p-amino benzoic acid, glutathione, propyl gallate, cysteine, methionine, ethanol and N-acetyl cysteine. In some embodiments, preservatives include validamycin A, TL-3, sodium ortho vanadate, sodium fluoride, N-a-tosyl-Phe-chloromethylketone, N-a-tosyl-Lys-chloromethylketone, aprotinin, phenylmethylsulfonyl fluoride, diisopropylfluorophosphate, protease inhibitor, reducing agent, alkylating agent, antimicrobial agent, oxidase inhibitor, or other inhibitor.

In some embodiments, a pharmaceutical formulation comprises a binder as an excipient. Non-limiting examples of suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, and combinations thereof.

In some embodiments, the binders that are used in a pharmaceutical formulation are selected from starches such as potato starch, corn starch, wheat starch; sugars such as sucrose, glucose, dextrose, lactose, maltodextrin; natural and synthetic gums; gelatine; cellulose derivatives such as microcrystalline cellulose, hydroxypropyl cellulose, hydroxyethyl cellulose, hydroxypropyl methyl cellulose, carboxymethyl cellulose, methyl cellulose, ethyl cellulose; polyvinylpyrrolidone (povidone); polyethylene glycol (PEG); waxes; calcium carbonate; calcium phosphate; alcohols such as sorbitol, xylitol, mannitol and water or a combination thereof.

In some embodiments, a pharmaceutical formulation comprises a lubricant as an excipient. Non-limiting examples of suitable lubricants include magnesium stearate, calcium stearate, zinc stearate, hydrogenated vegetable oils, sterotex, polyoxyethylene monostearate, talc, polyethylene glycol, sodium benzoate, sodium lauryl sulfate, magnesium lauryl sulfate, and light mineral oil. In some embodiments, lubricants that are in a pharmaceutical formulation are selected from metallic stearates (such as magnesium stearate, calcium stearate, aluminum stearate), fatty acid esters (such as sodium stearyl fumarate), fatty acids (such as stearic acid), fatty alcohols, glyceryl behenate, mineral oil, paraffins, hydrogenated vegetable oils, leucine, polyethylene glycols (PEG), metallic lauryl sulphates (such as sodium lauryl sulphate, magnesium lauryl sulphate), sodium chloride, sodium benzoate, sodium acetate and talc or a combination thereof.

In some embodiments, an excipient comprises a flavoring agent. In some embodiments, flavoring agents includes natural oils; extracts from plants, leaves, flowers, and fruits; and combinations thereof.

In some embodiments, an excipient comprises a sweetener. Non-limiting examples of suitable sweeteners include glucose (corn syrup), dextrose, invert sugar, fructose, and mixtures thereof (when not used as a carrier); saccharin and its various salts such as a sodium salt; dipeptide sweeteners such as aspartame; dihydrochalcone compounds, glycyrrhizin; Stevia rebaudiana (Stevioside); chloro derivatives of sucrose such as sucralose; and sugar alcohols such as sorbitol, mannitol, xylitol, and the like.

In some instances, a pharmaceutical formulation comprises a coloring agent. Non-limiting examples of suitable color agents include food, drug and cosmetic colors (FD&C), drug and cosmetic colors (D&C), and external drug and cosmetic colors (Ext. D&C).

In some embodiments, the pharmaceutical formulation disclosed herein comprises a chelator. In some embodiments, a chelator includes ethylenediamine-N,N,N′,N′-tetraacetic acid (EDTA); a disodium, trisodium, tetrasodium, dipotassium, tripotassium, dilithium and diammonium salt of EDTA; a barium, calcium, cobalt, copper, dysprosium, europium, iron, indium, lanthanum, magnesium, manganese, nickel, samarium, strontium, or zinc chelate of EDTA.

In some instances, a pharmaceutical formulation comprises a diluent. Non-limiting examples of diluents include water, glycerol, methanol, ethanol, and other similar biocompatible diluents. In some embodiments, a diluent is an aqueous acid such as acetic acid, citric acid, maleic acid, hydrochloric acid, phosphoric acid, nitric acid, sulfuric acid, or similar.

In some embodiments, a pharmaceutical formulation comprises a surfactant. In some embodiments, surfactants are be selected from, but not limited to, polyoxyethylene sorbitan fatty acid esters (polysorbates), sodium lauryl sulphate, sodium stearyl fumarate, polyoxyethylene alkyl ethers, sorbitan fatty acid esters, polyethylene glycols (PEG), polyoxyethylene castor oil derivatives, docusate sodium, quaternary ammonium compounds, amino acids such as L-leucine, sugar esters of fatty acids, glycerides of fatty acids or a combination thereof.

In some instances, a pharmaceutical formulation comprises an additional pharmaceutical agent. In some embodiments, an additional pharmaceutical agent is an antibiotic agent. In some embodiments, an antibiotic agent is of the group consisting of aminoglycosides, ansamycins, carbacephem, carbapenems, cephalosporins (including first, second, third, fourth and fifth generation cephalosporins), lincosamides, macrolides, monobactams, nitrofurans, quinolones, penicillin, sulfonamides, polypeptides or tetracycline.

In some embodiments, an antibiotic agent described herein is an aminoglycoside such as Amikacin, Gentamicin, Kanamycin, Neomycin, Netilmicin, Tobramycin or Paromomycin. In some embodiments, an antibiotic agent described herein is an Ansamycin such as Geldanamycin or Herbimycin.

In some embodiments, an antibiotic agent described herein is a carbacephem such as Loracarbef. In some embodiments, an antibiotic agent described herein is a carbapenem such as Ertapenem, Doripenem, Imipenem/Cilastatin or Meropenem.

In some embodiments, an antibiotic agent described herein is a cephalosporins (first generation) such as Cefadroxil, Cefazolin, Cefalexin, Cefalotin or Cefalothin, or alternatively a Cephalosporins (second generation) such as Cefaclor, Cefamandole, Cefoxitin, Cefprozil or Cefuroxime. In some embodiments, an antibiotic agent is a Cephalosporins (third generation) such as Cefixime, Cefdinir, Cefditoren, Cefoperazone, Cefotaxime, Cefpodoxime, Ceftibuten, Ceftizoxime and Ceftriaxone or a Cephalosporins (fourth generation) such as Cefepime or Ceftobiprole.

In some embodiments, an antibiotic agent described herein is a lincosamide such as Clindamycin and Azithromycin, or a macrolide such as Azithromycin, Clarithromycin, Dirithromycin, Erythromycin, Roxithromycin, Troleandomycin, Telithromycin and Spectinomycin.

In some embodiments, an antibiotic agent described herein is a monobactams such as Aztreonam, or a nitrofuran such as Furazolidone or Nitrofurantoin.

In some embodiments, an antibiotic agent described herein is a penicillin such as Amoxicillin, Ampicillin, Azlocillin, Carbenicillin, Cloxacillin, Dicloxacillin, Flucloxacillin, Mezlocillin, Nafcillin, Oxacillin, Penicillin G or V, Piperacillin, Temocillin and Ticarcillin.

In some embodiments, an antibiotic agent described herein is a sulfonamide such as Mafenide, Sulfonamidochrysoidine, Sulfacetamide, Sulfadiazine, Silver sulfadiazine, Sulfamethizole, Sulfamethoxazole, Sulfanilimide, Sulfasalazine, Sulfisoxazole, Trimethoprim, or Trimethoprim-Sulfamethoxazole (Co-trimoxazole) (TMP-SMX).

In some embodiments, an antibiotic agent described herein is a quinolone such as Ciprofloxacin, Enoxacin, Gatifloxacin, Levofloxacin, Lomefloxacin, Moxifloxacin, Nalidixic acid, Norfloxacin, Ofloxacin, Trovafloxacin, Grepafloxacin, Sparfloxacin and Temafloxacin.

In some embodiments, an antibiotic agent described herein is a polypeptide such as Bacitracin, Colistin or Polymyxin B.

In some embodiments, an antibiotic agent described herein is a tetracycline such as Demeclocycline, Doxycycline, Minocycline or Oxytetracycline.

### Embodiments

1. A bacteriophage derived from a temperate bacteriophage that has been rendered lytic, wherein the bacteriophage has been rendered lytic by removal, replacement, or inactivation of a lysogenic gene.
2. The bacteriophage of embodiment 1, wherein the bacteriophage has been rendered lytic by the removal of any one of SEQ ID NOS: 30-32, 47-58.
3. The bacteriophage of embodiment 1, wherein the bacteriophage has been rendered lytic by removal of at least 10%, 20%, 30%, 40%, 50%, 60%, 70%, 80%, 90% or 100% of any one of SEQ ID NOS: 30-32, 47-58.
4. The bacteriophage of embodiment 1, wherein the bacteriophage has been rendered lytic by the removal of a regulatory element of a lysogeny gene.
5. The bacteriophage of embodiment 1, wherein the bacteriophage has been rendered lytic by the removal, alteration or replacement of a promoter of a lysogeny gene.
6. The bacteriophage of embodiment 1, wherein the bacteriophage has been rendered lytic by the removal of a functional element of a lysogeny gene.
7. A composition comprising a Phietavirus bacteriophage and Rosenblumvirus bacteriophage, Phictavirus bacteriophage and Kayvirus bacteriophage, Rosenblumvirus bacteriophage and Kayvirus bacteriophage, or Phietavirus bacteriophage, Rosenblumvirus bacteriophage, and Kayvirus bacteriophage.
8. The composition of embodiment 7, comprising the Phietavirus bacteriophage, wherein the Phietavirus bacteriophage is engineered to remove, replace, or inactivate a lysogenic gene or a promoter of a lysogenic gene.
9. The composition of embodiment 8, wherein the lysogenic gene encodes for a repressor.
10. The composition of any one of embodiments 7-9, comprising the Phietavirus bacteriophage and Rosenblumvirus bacteriophage.
11. The composition of any one of embodiments 7-9, comprising the Phietavirus bacteriophage and Kayvirus bacteriophage.
12. The composition of any one of embodiments 7-9, comprising the Rosenblumvirus bacteriophage and Kayvirus bacteriophage.
13. The composition of any one of embodiments 7-9, comprising the Phictavirus bacteriophage, Rosenblumvirus, and Kayvirus bacteriophage.
14. The composition of any one of embodiments 7-13, comprising the Kayvirus bacteriophage, wherein the Kayvirus bacteriophage comprises two or more Kayvirus bacteriophage.
15. The composition of any one of embodiments 7-14, wherein the bacteriophage of the composition infect at least about 90% of a collection of at least about 30 Staphylococcus bacteria.
16. A composition comprising a plurality of bacteriophage comprising a first bacteriophage and a second bacteriophage, wherein the plurality of bacteriophage infect at least about 90% of a collection of at least about 30 Staphylococcus bacteria.
17. The composition of embodiment 16, wherein the Staphylococcus bacteria are selected from b004604, b004605, b004606, b004607, b004608, b004609, b004610, b004611, b004612, b004613, b004614, b004615, b004616, b004617, b004618, b004619, b004620, b004621, b004622, b004623, b004624, b004625, b004626, b004627, b004628, b004629, b004630, b004631, b004632, b004633, b004634, b004635, b004636, b004637, b004638, b004639, b004640, b004641, b004642, b004643, b004644, b004645, b004646, b004647, b004648, b004649, b004650, b004651, b004652, b004653, b004654, b004655, b004656, b004657, b004658, b004659, b004660, b004661, b004662, b004663, b004664, b004665, b004666, b004667, b004668, b004669, b004670, b004671, b004672, b004673, b004674, b004675, b004676, b004677, b004678, b004679, b004680, b004681, b004682, b004683, b004684, b004685, b004686, b004687, b004688, b004689, b004690, b004691, b004692, b004693, b004694, b004695, b004696, b004697, b004698, b004699, b004700, b004701, b004702, b004703, b004704, b004705, b004706, b004707, b004708, b004709, b004710, b004711, b004712, b004713, b004714, b004715, b004716, b004717, b004718, b004719, b004720, b004721, b004722, b004723, b004724, b004725, b004726, b004727, b004728, b004729, b004730, b004731, b004732, b004733, b004734, b004735, b004736, b004737, b004738, b004739, b004740, b004741, b004742, b004743, b004744, b004745, b004746, b004747, b004748, b004749, b004750, b004751, b004752, b004753, b004754, b004755, b004756, b004757, b004758, b004759, b004760, b004761, b004762, b004763, b004764, b004765, b004766, b004767, b004768, b004769, b004770, b004771, b004772, b004773, b004774, b004775, b004776, b004777, b004778, b004779, b004780, b004781, b004782, b004783, b004784, b004785, b004786, b004787, b004788, b004789, b004790, b004791, b004792, b004793, b004794, b004795, b004796, b004797, b004798, b004799, b004800, b004801, b004802, b004803, b004804, b004805, b004806, b004807, b004808, b004809, b004810, b004811, b004812, b004813, b004814, b004815, b004816, b004817, b004818, b004819, b004820, b004821, b004822, b004823, b004824, b004825, b004826, b004827, b004828, b004829, b004830, b004831, b004832, b004833, b004834, b004835, b004836, b004837, b004838, b004839, b004840, b004841, b004842, b004843, b004844, b004845, b004846, b004847, b004848, b004849, b004850, b004851, b004852, b004853, b004854, b004855, b004856, b004857, b004858, b004859, b004860, b004861, b004862, b004863, b004864, b004865, b004866, b004867, b004868, b004869, b004870, b004871, b004872, b004873, b004874, b004875, b004876, b004877, b004878, b004879, b004880, b004881, b004882, b004883, b004884, b004885, b004886, b004887, b004888, b004889, b004890, b004891, b004892, b004893, b004894, b004895, b004896, b004897, b004898, b004899, b004900, b004901, b004902, b004903, b004904, b004905, b004906, b004907, b004908, b004909, b004910, and b004911.
18. The composition of embodiment 16 or 17, wherein the collection of Staphylococcus bacteria comprises comprise Staphylococcus bacteria having a MLST of 8, 5, 22, 15, 1, 30, 398, 105, 45, 672, 2250, 582, 72, 97, 239, 34, 87, 101, 109, 1159, 1165, 1181, 12, 121, 152, 1750, 20, 225, 25, 291, 3628, 59, 7, 779, 88, 10, 1011, 1049, 1156, 1351, 149, 1637, 1649, 1757, 1842, 188, 1970, 2066, 256, 2867, 2945, 3149, 3182, 3510, 39, 395, 4317, 47, 4730, 50, 508, 573, 6, 630, 737, 828, 848, 923, or 93.
19. The composition of any one of embodiments 16-18, wherein the collection of Staphylococcus bacteria comprise bacteria isolated from a bloodstream infection. 20. The composition of any one of embodiments 16-19, wherein at least about 40% of the collection of Staphylococcus bacteria are multidrug resistant.
21. The composition of any one of embodiments 16-20, wherein the Staphylococcus bacteria comprises Staphylococcus aureus.
22. The composition of any one of embodiments 16-21, wherein infection is determined with a plaque assay or growth inhibition assay.
23. The composition of any one of embodiments 16-22, wherein the at least about 90% is at least about 95%.
24. The composition of any one of embodiments 16-22, wherein the at least about 90% is at least about 98%.
25. The composition of any one of embodiments 16-22, wherein the at least about 90% is at least about 99%.
26. The composition of any one of embodiments 16-25, wherein the at least about 30 Staphylococcus bacteria comprises b004604, b004605, b004606, b004607, b004608, b004609, b004610, b004611, b004612, b004613, b004614, b004615, b004616, b004617, b004618, b004619, b004620, b004621, b004622, b004623, b004624, b004625, b004626, b004627, b004628, b004629, b004630, b004631, b004632, b004633, b004634, b004635, b004636, b004637, b004638, b004639, b004640, b004641, b004642, b004643, b004644, b004645, b004646, b004647, b004648, b004649, b004650, b004651, b004652, b004653, b004654, b004655, b004656, b004657, b004658, b004659, b004660, b004661, b004662, b004663, b004664, b004665, b004666, b004667, b004668, b004669, b004670, b004671, b004672, b004673, b004674, b004675, b004676, b004677, b004678, b004679, b004680, b004681, b004682, b004683, b004684, b004685, b004686, b004687, b004688, b004689, b004690, b004691, b004692, b004693, b004694, b004695, b004696, b004697, b004698, b004699, b004700, b004701, b004702, b004703, b004704, b004705, b004706, b004707, b004708, b004709, b004710, b004711, b004712, b004713, b004714, b004715, b004716, b004717, b004718, b004719, b004720, b004721, b004722, b004723, b004724, b004725, b004726, b004727, b004728, b004729, b004730, b004731, b004732, b004733, b004734, b004735, b004736, b004737, b004738, b004739, b004740, b004741, b004742, b004743, b004744, b004745, b004746, b004747, b004748, b004749, b004750, b004751, b004752, b004753, b004754, b004755, b004756, b004757, b004758, b004759, b004760, b004761, b004762, b004763, b004764, b004765, b004766, b004767, b004768, b004769, b004770, b004771, b004772, b004773, b004774, b004775, b004776, b004777, b004778, b004779, b004780, b004781, b004782, b004783, b004784, b004785, b004786, b004787, b004788, b004789, b004790, b004791, b004792, b004793, b004794, b004795, b004796, b004797, b004798, b004799, b004800, b004801, b004802, b004803, b004804, b004805, b004806, b004807, b004808, b004809, b004810, b004811, b004812, b004813, b004814, b004815, b004816, b004817, b004818, b004819, b004820, b004821, b004822, b004823, b004824, b004825, b004826, b004827, b004828, b004829, b004830, b004831, b004832, b004833, b004834, b004835, b004836, b004837, b004838, b004839, b004840, b004841, b004842, b004843, b004844, b004845, b004846, b004847, b004848, b004849, b004850, b004851, b004852, b004853, b004854, b004855, b004856, b004857, b004858, b004859, b004860, b004861, b004862, b004863, b004864, b004865, b004866, b004867, b004868, b004869, b004870, b004871, b004872, b004873, b004874, b004875, b004876, b004877, b004878, b004879, b004880, b004881, b004882, b004883, b004884, b004885, b004886, b004887, b004888, b004889, b004890, b004891, b004892, b004893, b004894, b004895, b004896, b004897, b004898, b004899, b004900, b004901, b004902, b004903, b004904, b004905, b004906, b004907, b004908, b004909, b004910, and b004911.
27. The composition of any one of embodiments 16-25, wherein at least about 30 Staphylococcus bacteria comprises Staphylococcus bacteria having a MLST of 8, 5, 22, 15, 1, 30, 398, 105, 45, 672, 2250, 582, 72, 97, 239, 34, 87, 101, 109, 1159, 1165, 1181, 12, 121, 152, 1750, 20, 225, 25, 291, 3628, 59, 7, 779, 88, 10, 1011, 1049, 1156, 1351, 149, 1637, 1649, 1757, 1842, 188, 1970, 2066, 256, 2867, 2945, 3149, 3182, 3510, 39, 395, 4317, 47, 4730, 50, 508, 573, 6, 630, 737, 828, 848, 923, or 93.
28. The composition of any one of embodiments 16-27, wherein at least about 30 Staphylococcus bacteria comprises bacteria isolated from a bloodstream infection.
29. The composition of any one of embodiments 16-28, wherein at least about 40% of the at least about 30 Staphylococcus bacteria are multidrug resistant.
30. The composition of any one of embodiments 16-25, wherein the first bacteriophage and the second bacteriophage are of different genera.
31. The composition of any one of embodiments 16-30, wherein the plurality of bacteriophage comprises a Phictavirus bacteriophage and Rosenblumvirus bacteriophage, Phietavirus bacteriophage and Kayvirus bacteriophage, Rosenblumvirus bacteriophage and Kayvirus bacteriophage, or Phietavirus bacteriophage, Rosenblumvirus bacteriophage, and Kayvirus bacteriophage.
32. The composition of any one of embodiments 16-31, wherein the plurality of bacteriophage comprises a Phietavirus.
33. The composition of embodiment 32, wherein the Phietavirus is engineered to remove, replace, or inactivate a lysogenic gene or a promoter of a lysogenic gene.
34. The composition of embodiment 33, wherein the lysogenic gene encodes for a repressor.
35. A composition comprising a plurality of bacteriophage comprising a first bacteriophage that binds a first receptor of a Staphylococcus bacteria, a second bacteriophage that binds a second receptor of the Staphylococcus bacteria, wherein the plurality of bacteriophage is more resilient to resistance by the Staphylococcus bacteria than the first or second bacteriophage alone.
36. The composition of embodiment 35, wherein the first receptor and the second receptor are different.
37. The composition of embodiment 35 or embodiment 36, wherein the first bacteriophage comprises a Phietavirus.
38. The composition of embodiment 35 or embodiment 36, wherein the first bacteriophage comprises a Phietavirus and a Rosenblumvirus.
39. The composition of any one of embodiments 35-38, wherein the second bacteriophage comprises a Kayvirus.
40. The composition of embodiment 35 or embodiment 36, wherein the plurality of bacteriophage comprises: a Phietavirus bacteriophage and Rosenblumvirus bacteriophage, Phietavirus bacteriophage and Kayvirus bacteriophage, Rosenblumvirus bacteriophage and Kayvirus bacteriophage, or Phietavirus bacteriophage, Rosenblumvirus bacteriophage, and Kayvirus bacteriophage.
41. The composition of any one of embodiments 35-40, wherein the plurality of bacteriophage is more resilient to Staphylococcus resistance than the first bacteriophage or the second bacteriophage alone.
42. The composition of any one of embodiments 35-41, wherein the plurality of bacteriophage of the composition infects at least about 90% of a collection of at least about 30 Staphylococcus bacteria.
43. The composition of any one of embodiments 35-42, wherein the first bacteriophage and the second bacteriophage are of different genera.
44. The composition of embodiment 43, wherein the first bacteriophage and the second bacteriophage are capable of independently infecting at least 90% of the collection of Staphylococcus bacteria.
45. The composition of any one of embodiments 35-44, wherein the first bacteriophage comprises a Phietavirus, Rosenblumvirus, or Kayvirus.
46. The composition of any one of embodiments 35-45, wherein the second bacteriophage comprises a Phietavirus, Rosenblumvirus, or Kayvirus.
47. The composition of any one of embodiments 35-46, wherein the plurality of bacteriophage comprises: a Phietavirus bacteriophage and Rosenblumvirus bacteriophage, Phietavirus bacteriophage and Kayvirus bacteriophage, Rosenblumvirus bacteriophage and Kayvirus bacteriophage, or Phietavirus bacteriophage, Rosenblumvirus bacteriophage, and Kayvirus bacteriophage.
48. The composition of any one of embodiments 35-47, wherein the plurality of bacteriophage infects at least about 90% of the collection of Staphylococcus bacteria.
49. The composition of embodiment 48, wherein the Staphylococcus bacteria comprises Staphylococcus aureus.
50. The composition of any one of embodiments 48-49, wherein infection of the at least about 90% is determined with a plaque assay or growth inhibition assay.
51. The composition of embodiment 50, wherein the at least about 90% is at least about 95%.
52. The composition of embodiment 50, wherein the at least about 90% is at least about 98%.
53. The composition of embodiment 50, wherein the at least about 90% is at least about 99%.
54. The composition of any one of embodiments 35-53, wherein the collection of Staphylococcus bacteria comprise Staphylococcus bacteria having a MLST of 8, 5, 22, 15, 1, 30, 398, 105, 45, 672, 2250, 582, 72, 97, 239, 34, 87, 101, 109, 1159, 1165, 1181, 12, 121, 152, 1750, 20, 225, 25, 291, 3628, 59, 7, 779, 88, 10, 1011, 1049, 1156, 1351, 149, 1637, 1649, 1757, 1842, 188, 1970, 2066, 256, 2867, 2945, 3149, 3182, 3510, 39, 395, 4317, 47, 4730, 50, 508, 573, 6, 630, 737, 828, 848, 923, or 93, or a combination of two or more thereof.
55. The composition of any one of embodiments 35-54, wherein the collection of Staphylococcus bacteria comprises bacteria isolated from a bloodstream infection.
56. The composition of any one of embodiments 35-55, wherein at least about 40% of the collection of Staphylococcus bacteria are multidrug resistant.
57. A Phietavirus bacteriophage engineered to remove, replace, or inactivate a lysogenic gene or a promoter of a lysogenic gene.
58. The bacteriophage of embodiment 57, wherein the lysogenic gene encodes for a repressor.
59. The bacteriophage of embodiment 58, wherein the repressor comprises an amino acid sequence at least about 80% identical to SEQ ID NO: 47 or 48.
60. The bacteriophage of any one of embodiments 57-59, wherein removal of the lysogenic gene comprises removing from about 1% to 100% of the lysogenic gene.
61. The bacteriophage of embodiment 60, wherein about 10 to about 1,200 base pairs of the lysogenic gene are removed.
62. The bacteriophage of any one of embodiments 57-61, wherein the lysogenic gene is removed, replaced, or inactivated.
63. The bacteriophage of embodiment 62, wherein the lysogenic gene is removed.
64. The bacteriophage of any one of embodiments 57-63, wherein the promoter of the lysogenic gene is removed, replaced, or inactivated.
65. The bacteriophage of any one of embodiments 57-62, further comprising a Rosenblumvirus.
66. The bacteriophage of any one of embodiments 57-65, further comprising a Kayvirus.
67. The bacteriophage of any one of embodiments 57-66, comprising a plurality of bacteriophage comprising the Phietavirus bacteriophage, wherein the plurality of bacteriophage infects at least about 90% of a collection of at least about 30 Staphylococcus bacteria.
68. The composition of any one of embodiments 7-44, or the bacteriophage of any one of embodiments 57-68, comprising a nucleic acid encoding for an exogenous peptide selected from TreA, Ipi, DNAsel, RIP, FS3, PLNC8a, PLNC8B, LytM, LnqO, Dispersin D aminopeptidase, amylase, carbohydrase, carboxypeptidase, catalase, cellulase, chitinase, cutinase, cyclodextrin glycosyltransferase, deoxyribonuclease, esterase, alpha-galactosidase, beta-galactosidase, glucoamylase, alpha-glucosidase, beta-glucosidase, haloperoxidase, invertase, laccase, lipase, mannosidase, oxidase, pectinolytic enzyme, peptidoglutaminase, peroxidase, phytase, polyphenoloxidase, proteolytic enzyme, ribonuclease, transglutaminase, xylanase, lyase, glycosyl hydroxylase, polyglucosamine (PGA) depolymerases, colonic acid depolymerase, 1,4-L-fucodise hydrolase, colanic acid, or depolymerazing alginase.
69. The composition of any one of embodiments 7-44, or the bacteriophage of any one of embodiments 57-68, comprising one or more components of a CRISPR-Cas system.
70. The composition or bacteriophage of embodiment 69, wherein the CRISPR-Cas system is a Type IB CRISPR-Cas system from Listeria monocytogenes (LMIB).
71. The composition of any one of embodiments 7-44, or the bacteriophage of any one of embodiments 57-68, comprising a spacer sequence or a crRNA transcribed therefrom, wherein the spacer sequence is complementary to a target nucleic acid sequence from a target gene in a target bacteria.
72. The composition or bacteriophage of embodiment 71, wherein the target bacteria comprise a Staphylococcus bacteria.
73. A method of killing a bacteria in a subject infected with the bacteria, the method comprising administering to the subject a plurality of bacteriophage comprising a Rosenblumvirus.
74. The method of embodiment 73, wherein the plurality of bacteriophage comprises a Phietavirus.
75. The method of embodiment 73 or embodiment 74, wherein the plurality of bacteriophage comprises a Kayvirus.
76. A method of killing a bacteria in a subject infected with the bacteria, the method comprising administering to the subject a plurality of bacteriophage comprising a Phietavirus.
77. The method of embodiment 76, wherein the plurality of bacteriophage comprises a Rosenblumvirus.
78. The method of embodiment 76 or embodiment 77, wherein the plurality of bacteriophage comprises a Kayvirus.
79. A method of killing a bacteria in a subject infected with the bacteria, the method comprising administering to the subject a plurality of bacteriophage comprising a Kayvirus.
80. The method of embodiment 78, wherein the plurality of bacteriophage comprises a Rosenblumvirus.
81. The method of embodiment 78 or embodiment 79, wherein the plurality of bacteriophage comprises a Phietavirus.
82. A bacteriophage comprising at least 70%, 75%, 80%, 85%, 90%, 95%, or 100% sequence identity with p1378 (PTA-127329).
83. A bacteriophage comprising at least 70%, 75%, 80%, 85%, 90%, 95%, or 100% sequence identity with p1381 (PTA-127330).
84. A bacteriophage comprising at least 70%, 75%, 80%, 85%, 90%, 95%, or 100% sequence identity with p4815 (PTA-127331).
85. A bacteriophage comprising at least 70%, 75%, 80%, 85%, 90%, 95%, or 100% sequence identity with p1378e062 (PTA-127333).
86. A bacteriophage comprising at least 70%, 75%, 80%, 85%, 90%, 95%, or 100% sequence identity with p1378e074 (PTA-127334).
87. A bacteriophage comprising at least 70%, 75%, 80%, 85%, 90%, 95%, or 100% sequence identity with p4815e037 (PTA-127335).
88. A bacteriophage comprising at least 70%, 75%, 80%, 85%, 90%, 95%, or 100% sequence identity with p1378e075 (PTA-127338).
89. A bacteriophage comprising at least 70%, 75%, 80%, 85%, 90%, 95%, or 100% sequence identity with p1381e017 (PTA-127339).
90. A bacteriophage comprising at least 70%, 75%, 80%, 85%, 90%, 95%, or 100% sequence identity with p4815e053 (PTA-127340).
91. A bacteriophage comprising at least 70%, 75%, 80%, 85%, 90%, 95%, or 100% sequence identity with p1498e001 (PTA-127336).
92. A bacteriophage comprising at least 70%, 75%, 80%, 85%, 90%, 95%, or 100% sequence identity with p3693e001 (PTA-127337).
93. A bacteriophage comprising at least 70%, 75%, 80%, 85%, 90%, 95%, or 100% sequence identity with p3224e002 (PTA-127341).
94. A bacteriophage comprising at least 70%, 75%, 80%, 85%, 90%, 95%, or 100% sequence identity with p5593e001 (PTA-127342).
95. A bacteriophage comprising at least 70%, 75%, 80%, 85%, 90%, 95%, or 100% sequence identity p3224e002 (PTA-127341).
96. A bacteriophage comprising at least 70%, 75%, 80%, 85%, 90%, 95%, or 100% sequence identity with p1468e003 (PTA-127343).
97. A bacteriophage comprising at least 70%, 75%, 80%, 85%, 90%, 95%, or 100% sequence identity with p1478e003 (TA-127344).
98. A bacteriophage comprising at least 70%, 75%, 80%, 85%, 90%, 95%, or 100% sequence identity with p1494e002 (PTA-127345).
99. A bacteriophage comprising at least 70%, 75%, 80%, 85%, 90%, 95%, or 100% sequence identity with p2808 (PTA-127332).
100. A bacteriophage comprising at least 70%, 75%, 80%, 85%, 90%, 95%, or 100% sequence identity with p1473.
101. The composition of any one of embodiments 7-43, or 55-59 or the bacteriophage of any one of embodiments 1-6, 44-54, or 82-100 for use in the treatment of a Staphylococcus infection.
102. A composition for use in the treatment of a Staphylococcus infection comprising at least two bacteriophage, wherein the first bacteriophage comprises at least 70%, 75%, 80%, 85%, 90%, 95%, or 100% sequence identity with p1378, and the second bacteriophage comprises at least 70%, 75%, 80%, 85%, 90%, 95%, or 100% sequence identity with p1381, p4815, p1378e062, p1378e074, p4815e037, p1378e075, p1381e017, p4815e053, p1498e001, p3693e001, p3224e002, p5593e001, p1468e003, p1478e003, p1494e002, p2808, or p1473.
103. A composition for use in the treatment of a Staphylococcus infection comprising at least two bacteriophage, wherein the first bacteriophage comprises at least 70%, 75%, 80%, 85%, 90%, 95%, or 100% sequence identity with p4815, and the second bacteriophage comprises at least 70%, 75%, 80%, 85%, 90%, 95%, or 100% sequence identity with p1381, p1378, p1378e062, p1378e074, p4815e037, p1378e075, p1381e017, p4815e053, p1498e001, p3693e001, p3224e002, p5593e001, p1468e003, p1478e003, p1494e002, p2808, or p1473.
104. A composition for use in the treatment of a Staphylococcus infection comprising at least two bacteriophage, wherein the first bacteriophage comprises at least 70%, 75%, 80%, 85%, 90%, 95%, or 100% sequence identity with p1494e002, and the second bacteriophage comprises at least 70%, 75%, 80%, 85%, 90%, 95%, or 100% sequence identity with p1381, p1378, p4815, p1378e062, p1378e074, p4815e037, p1378e075, p1381e017, p4815e053, p1498e001, p3693e001, p3224e002, p5593e001, p1468e003, p1478e003, p2808, or p1473.
105. A composition for use in the treatment of a Staphylococcus infection comprising at least two bacteriophage, wherein the first bacteriophage comprises at least 70%, 75%, 80%, 85%, 90%, 95%, or 100% sequence identity with p2808, and the second bacteriophage comprises at least 70%, 75%, 80%, 85%, 90%, 95%, or 100% sequence identity with p1381, p1378, p4815, p1378e062, p1378e074, p4815e037, p1378e075, p1381e017, p4815e053, p1498e001, p3693e001, p3224e002, p5593e001, p1468e003, p1478e003, p1494e002, or p1473.
106. The composition of any one of embodiments 7-43, 55-59, or 101-105, or the bacteriophage of any one of embodiments 1-6, 44-54, or 82-100, for use in the manufacture of a medicament to treat a Staphylococcus infection.
107. A composition for use in the manufacture of a medicament to treat a Staphylococcus infection comprising at least two bacteriophage, wherein the first bacteriophage comprises at least 70%, 75%, 80%, 85%, 90%, 95%, or 100% sequence identity with p1378, and the second bacteriophage comprises at least 70%, 75%, 80%, 85%, 90%, 95%, or 100% sequence identity with p1381, p4815, p1378e062, p1378e074, p4815e037, p1378e075, p1381e017, p4815e053, p1498e001, p3693e001, p3224e002, p5593e001, p1468e003, p1478e003, p1494e002, p2808, or p1473.
108. A composition for use in the manufacture of a medicament to treat a Staphylococcus infection comprising at least two bacteriophage, wherein the first bacteriophage comprises at least 70%, 75%, 80%, 85%, 90%, 95%, or 100% sequence identity with p4815, and the second bacteriophage comprises at least 70%, 75%, 80%, 85%, 90%, 95%, or 100% sequence identity with p1381, p1378, p1378e062, p1378e074, p4815e037, p1378e075, p1381e017, p4815e053, p1498e001, p3693e001, p3224e002, p5593e001, p1468e003, p1478e003, p1494e002, p2808, or p1473.
109. A composition for use in the manufacture of a medicament to treat a Staphylococcus infection comprising at least two bacteriophage, wherein the first bacteriophage comprises at least 70%, 75%, 80%, 85%, 90%, 95%, or 100% sequence identity with p1494e002, and the second bacteriophage comprises at least 70%, 75%, 80%, 85%, 90%, 95%, or 100% sequence identity with p1381, p1378, p4815, p1378e062, p1378e074, p4815e037, p1378e075, p1381e017, p4815e053, p1498e001, p3693e001, p3224e002, p5593e001, p1468e003, p1478e003, p2808, or p1473.
110. A composition for use in the manufacture of a medicament to treat a Staphylococcus infection comprising at least two bacteriophage, wherein the first bacteriophage comprises at least 70%, 75%, 80%, 85%, 90%, 95%, or 100% sequence identity with p2808, and the second bacteriophage comprises at least 70%, 75%, 80%, 85%, 90%, 95%, or 100% sequence identity with p1381, p1378, p4815, p1378e062, p1378e074, p4815e037, p1378e075, p1381e017, p4815e053, p1498e001, p3693e001, p3224e002, p5593e001, p1468e003, p1478e003, p1494e002, or p1473.
111. A composition comprising a plurality of bacteriophage comprising a first bacteriophage and a second bacteriophage, wherein a bacteria treated with the plurality of bacteriophage has a reduced amount of regrowth as compared to treatment with the first bacteriophage or the second bacteriophage alone.
112. The composition of embodiment 111, wherein the first bacteriophage comprises a Phictavirus and the second bacteriophage comprises a Rosenblumvirus or a Kayvirus; the first bacteriophage comprises a Rosenblumvirus and the second bacteriophage comprises a Phictavirus or a Kayvirus; or the first bacteriophage comprises a Kayvirus and the second bacteriophage comprises a Phictavirus or a Rosenblumvirus.

### Definitions

Unless otherwise defined, all technical and scientific terms used herein have the same meaning as commonly understood by one of ordinary skill in the art to which this disclosure belongs. The terminology used in the description of the disclosure herein is for the purpose of describing particular embodiments only and is not intended to be limiting of the disclosure.

Unless the context indicates otherwise, it is specifically intended that the various features of the disclosure described herein are able of being used in any combination. Moreover, the present disclosure also contemplates that in some embodiments, any feature or combination of features set forth herein are excluded or omitted. To illustrate, if the specification states that a composition comprises components A, B and C, it is specifically intended that any of A, B or C, or a combination thereof, are omitted and disclaimed singularly or in any combination.

One of skill in the art will understand the interchangeability of terms designating the various CRISPR-Cas systems and their components due to a lack of consistency in the literature and an ongoing effort in the art to unify such terminology. Likewise, one of skill in the art will also understand the interchangeability of terms designating the various anti-CRISPR proteins due to a lack of consistency in the literature and an ongoing effort in the art to unify such terminology.

As used in the description and the appended claims, the singular forms “a,” “an” and “the” are intended to include the plural forms as well, unless the context clearly indicates otherwise. Also as used herein, “and/or” refers to and encompasses any and all possible combinations of one or more of the associated listed items, as well as the lack of combinations when interpreted in the alternative (“or”).

The term “about” as used herein when referring to a measurable value such as a dosage or time period and the like refers to variations of ±20%, ±10%, ±5%, ±1%, ±0.5%, or even ±0.1% of the specified amount. As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

The term “comprise”, “comprises”, and “comprising”, “includes”, “including”, “have” and “having”, as used herein, specify the presence of the stated features, steps, operations, elements, and/or components, but do not preclude the presence or addition of one or more other features, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed disclosure. Thus, the term “consisting essentially of” when used in a claim of this disclosure is not intended to be interpreted to be equivalent to “comprising.”

The term “consists of” and “consisting of”, as used herein, excludes any features, steps, operations, elements, and/or components not otherwise directly stated. The use of “consisting of” limits only the features, steps, operations, elements, and/or components set forth in that clause and does exclude other features, steps, operations, elements, and/or components from the claim as a whole.

As used herein, “chimeric” refers to a nucleic acid molecule or a polypeptide in which at least two components are derived from different sources (e.g., different organisms, different coding regions).

“Complement” as used herein means 100% complementarity or identity with the comparator nucleotide sequence or it means less than 100% complementarity (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and the like, complementarity). Complement or complementable may also be used in terms of a “complement” to or “complementing” a mutation.

The terms “complementary” or “complementarity”, as used herein, refer to the natural binding of polynucleotides under permissive salt and temperature conditions by base-pairing. For example, the sequence “A-G-T” binds to the complementary sequence “T-C-A.” Complementarity between two single-stranded molecules is “partial,” in which only some of the nucleotides bind, or it is complete when total complementarity exists between the single stranded molecules. The degree of complementarity between nucleic acid strands has significant effects on the efficiency and strength of hybridization between nucleic acid strands.

As used herein, the term “gene” refers to a nucleic acid molecule capable of being used to produce mRNA, tRNA, rRNA, miRNA, anti-microRNA, regulatory RNA, and the like. Genes may or may not be capable of being used to produce a functional protein or gene product. Genes include both coding and non-coding regions (e.g., introns, regulatory elements, functional elements, promoters, enhancers, termination sequences and/or 5′ and 3′ untranslated regions). A gene is “isolated” by which is meant a nucleic acid that is substantially or essentially free from components normally found in association with the nucleic acid in its natural state. Such components include other cellular material, culture medium from recombinant production, and/or various chemicals used in chemically synthesizing the nucleic acid.

As used herein, a “target nucleotide sequence” refers to the portion of a target gene that is complementary to the spacer sequence of the recombinant CRISPR array.

As used herein, a “target nucleotide sequence” refers to the portion of a target gene (i.e., target region in the genome or the “protospacer sequence,” which is adjacent to a protospacer adjacent motif (PAM) sequence) that is fully complementary or substantially complementary (e.g., at least 70% complementary (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to a spacer sequence in a CRISPR array.

As used herein, the term “protospacer adjacent motif” or “PAM” refers to a DNA sequence present on the target DNA molecule adjacent to the nucleotide sequence matching the spacer sequence. This motif is found in the target gene next to the region to which a spacer sequence binds as a result of being complementary to that region and identifies the point at which base pairing with the spacer nucleotide sequence begins. The exact PAM sequence that is required varies between each different CRISPR-Cas system. Non-limiting examples of PAMs include CCA, CCT, CCG, TTC, AAG, AGG, ATG, GAG, and/or CC. In some instances, in Type I systems, the PAM is located immediately 5′ to the sequence that matches the spacer, and thus is 3′ to the sequence that base pairs with the spacer nucleotide sequence, and is directly recognized by Cascade. In some instances, for B. halodurans Type I-C systems, the PAM is YYC, where Y can be either T or C. In some instances, for the L. monocytogenes Type I-B system, the PAM is CCW, where W can be A or T. Once a cognate protospacer and PAM are recognized, Cas3 is recruited, which then cleaves and degrades the target DNA. For Type II systems, the PAM is required for a Cas9/sgRNA to form an R-loop to interrogate a specific DNA sequence through Watson-Crick pairing of its guide RNA with the genome. The PAM specificity is a function of the DNA-binding specificity of the Cas9 protein (e.g., a -protospacer adjacent motif recognition domain at the C-terminus of Cas9).

As used herein, type I Clustered Regularly Interspaced Short Palindromic Repeats (CRISPR)-associated complex for antiviral defense (Cascade) refers to a complex of polypeptides involved in processing of pre-crRNAs and subsequent binding to the target DNA in type I CRISPR-Cas systems. These polypeptides include, but are not limited to, the Cascade polypeptides of type I subtypes I-A, I-B, I-C, I-D, I-E, I-F, and I-U. Non-limiting examples of type I-A polypeptides include Cas7 (Csa2), Cas8a1 (Csx13), Cas8a2 (Csx9), Cas5, Csa5, Cas6a, Cas3′ and/or a Cas3″. Non-limiting examples of type I-B polypeptides include Cas6b, Cas8b (Csh1), Cas7 (Csh2) and/or Cas5. Non-limiting examples of type I-C polypeptides include Cas5d, Cas8c (Csd1), and/or Cas7 (Csd2). Non-limiting examples of type I-D polypeptides include Cas10d (Csc3), Csc2, Csc1, and/or Cas6d. Non-limiting examples of type I-E polypeptides include Cse1 (CasA), Cse2 (CasB), Cas7 (CasC), Cas5 (CasD) and/or Cas6e (CasE). Non-limiting examples of type I-F polypeptides include Cys1, Cys2, Cas7 (Cys3) and/or Cas6f (Csy4). Non-limiting examples of type I-F polypeptides include Cas8u2, Cas7, and/or fused Cas5-Cas6 polypeptide. Non-limiting examples of type I-U polypeptides include Cas8a1 (Cst1), Cas7 (Cst2), Cas5 (Cst5t), and Cas3. In some embodiments, a recombinant nucleic acid described herein comprises, consists essentially of, or consists of, a nucleotide sequence encoding a subset of type-I Cascade polypeptides that function to process a CRISPR array and subsequently bind to a target DNA using the spacer of the processed CRISPR RNA as a guide.

A “CRISPR array” as used herein means a nucleic acid molecule that comprises at least two repeat sequences, or a portion of each of said repeat sequences, and at least one spacer sequence. One of the two repeat sequences, or a portion thereof, is linked to the 5′ end of the spacer sequence and the other of the two repeat sequences, or portion thereof, is linked to the 3′ end of the spacer sequence. In a recombinant CRISPR array, the combination of repeat sequences and spacer sequences is synthetic, made by man and not found in nature. In some embodiments, a “CRISPR array” refers to a nucleic acid construct that comprises from 5′ to 3′ at least one repeat-spacer sequences (e.g., about 1, 2, 3, 4, 5, 6, 7, 8, 9, 10, 11, 12, 13, 14, 15, 16, 17, 18, 19, 20, 21, 22, 23, 24, 25 or more repeat-spacer sequences, and any range or value therein), wherein the 3′ end of the 3′ most repeat-spacer sequence of the array are linked to a repeat sequence, thereby all spacers in said array are flanked on both the 5′ end and the 3′ end by a repeat sequence.

As used herein, “spacer sequence” or “spacer” refers to a nucleotide sequence that is complementary to a target DNA (i.e., target region in the genome or the “protospacer sequence,” which is adjacent to a protospacer adjacent motif (PAM) sequence). The spacer sequence is fully complementary or substantially complementary (e.g., at least about 70% complementary (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to a target DNA.

A “repeat sequence” as used herein, refers to, for example, any repeat sequence of a wild-type CRISPR locus or a repeat sequence of a synthetic CRISPR array that are separated by “spacer sequences” (e.g., a repeat-spacer-repeat sequence). A repeat sequence useful with this disclosure is any known or later identified repeat sequence of a CRISPR locus or it is a synthetic repeat designed to function in a CRISPR system, for example CRISPR Type I system.

As used herein, the term “CRISPR phage”, “CRISPR enhanced phage”, and “crPhage” refers to a bacteriophage particle comprising bacteriophage DNA comprising at least one heterologous polynucleotide that encodes at least one component of a CRISPR-Cas system (e.g., CRISPR array, crRNA; e.g., PI bacteriophage comprising an insertion of a targeting crRNA). In some embodiments, the polynucleotide encodes at least one transcriptional activator of a CRISPR-Cas system. In some embodiments, the polynucleotide encodes at least one component of an anti-CRISPR polypeptide of a CRISPR-Cas system.

As used herein, the phrase “substantially identical,” or “substantial identity” in the context of two nucleic acid molecules, nucleotide sequences or protein sequences, refers to two or more sequences or subsequences that have at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and/or 100% nucleotide or amino acid residue identity, when compared and aligned for maximum correspondence, as measured using one of the following sequence comparison algorithms or by visual inspection. In some embodiments, substantial identity refer to two or more sequences or subsequences that have at least about 70%, at least about 75%, at least about 80%, at least about 85%, at least about 90%, at least about 95, 96, 97, 98, or 99% identity. For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., San Diego, CA). An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. The comparison of one or more polynucleotide sequences is to a full-length polynucleotide sequence or to a portion thereof, or to a longer polynucleotide sequence. In some instances, “Percent identity” is determined using BLASTX version 2.0 for translated nucleotide sequences and BLASTN version 2.0 for polynucleotide sequences.

In some embodiments, the recombinant nucleic acid molecules, nucleotide sequences and polypeptides disclosed herein are “isolated.” An “isolated” nucleic acid molecule, an “isolated” nucleotide sequence or an “isolated” polypeptide is a nucleic acid molecule, nucleotide sequence or polypeptide that exists apart from its native environment. In some instances, an isolated nucleic acid molecule, nucleotide sequence or polypeptide exists in a purified form that is at least partially separated from at least some of the other components of the naturally occurring organism or virus, for example, the cell or viral structural components or other polypeptides or nucleic acids commonly found associated with the polynucleotide. In representative embodiments, the isolated nucleic acid molecule, the isolated nucleotide sequence and/or the isolated polypeptide is at least about 1%, 5%, 10%, 20%, 30%, 40%, 50%, 60%, 70%, 80%, 90%, 95% pure, or purer.

By the terms “treat,” “treating,” or “treatment,” it is intended that the severity of the subject's condition is reduced or at least partially improved or modified and that some alleviation, mitigation or decrease in at least one clinical symptom is achieved, and/or there is a delay in the progression of the disease or condition, and/or delay of the onset of a disease or illness. With respect to an infection, a disease or a condition, the term refers to a decrease in the symptoms or other manifestations of the infection, disease or condition. In some embodiments, treatment provides a reduction in symptoms or other manifestations of the infection, disease or condition by at least about 5%, e.g., about 10%, 15%, 20%, 25%, 30%, 35%, 40%, 45%, 50% or more.

The terms with respect to an “infection”, “a disease”, or “a condition”, used herein, refer to any adverse, negative, or harmful physiological condition in a subject. In some embodiments, the source of an “infection”, “a disease”, or “a condition”, is the presence of a target bacterial population in and/or on a subject. In some embodiments, the bacterial population comprises one or more target bacterial species. In some embodiments, the one or more bacteria species in the bacterial population comprise one or more strains of one or more bacteria. In some embodiments, the target bacterial population causes an “infection”, “a disease”, or “a condition” that is acute or chronic. In some embodiments, the target bacterial population causes an “infection”, “a disease”, or “a condition” that is localized or systemic. In some embodiments, the target bacterial population causes an “infection”, “a disease”, or “a condition” that is idiopathic. In some embodiments, the target bacterial population causes an “infection”, “a disease”, or “a condition” that is acquired through means, including but not limited to, respiratory inhalation, ingestion, skin and wound infections, blood stream infections, middle-car infections, gastrointestinal tract infections, peritoneal membrane infections, urinary tract infections, urogenital tract infections, oral soft tissue infections, intra-abdominal infections, epidermal or mucosal absorption, eye infections (including contact lens contamination), endocarditis, infections in cystic fibrosis, non-cystic fibrosis bronchiectasis (NCFB), infections of indwelling medical devices such as joint prostheses, dental implants, catheters and cardiac implants, sexual contact, and/or hospital-acquired and ventilator-associated bacterial pneumonias.

As used herein the term “biofilm” means an accumulation of microorganisms embedded in a matrix of polysaccharide. Biofilms form on solid biological or non-biological surfaces, as well as at liquid-air interfaces, and are medically important, accounting for over 80 percent of microbial infections in the body.

The terms “prevent,” “preventing,” and “prevention” (and grammatical variations thereof) refer to prevention and/or delay of the onset of an infection, disease, condition and/or a clinical symptom(s) in a subject and/or a reduction in the severity of the onset of the infection, disease, condition and/or clinical symptom(s) relative to what would occur in the absence of carrying out the methods disclosed herein prior to the onset of the disease, disorder and/or clinical symptom(s). Thus, in some embodiments, to prevent infection, food, surfaces, medical tools and devices are treated with compositions and by methods disclosed herein.

The terms “individual”, or “subject” as used herein includes any animal that has or is susceptible to an infection, disease or condition involving bacteria. Thus, in some embodiments, subjects are mammals, avians, reptiles, amphibians, fish, crustaceans, and mollusks. Mammalian subjects include but are not limited to humans, non-human primates (e.g., gorilla, monkey, baboon, and chimpanzee, etc.), dogs, cats, goats, horses, pigs, cattle, sheep, and the like, and laboratory animals (e.g., rats, guinea pigs, mice, gerbils, hamsters, and the like). Avian subjects include but are not limited to chickens, ducks, turkeys, geese, quail, pheasants, and birds kept as pets (e.g., parakeets, parrots, macaws, cockatoos, canaries, and the like). Fish subjects include but are not limited to species used in aquaculture (e.g., tuna, salmon, tilapia, catfish, carp, trout, cod, bass, perch, snapper, and the like). Crustacean subjects include but are not limited to species used in aquaculture (e.g., shrimp, prawn, lobster, crayfish, crab). Mollusk subjects include but are not limited to species used in aquaculture (e.g., abalone, mussel, oyster, clams, scallop). In some embodiments, suitable subjects include both males and females and subjects of any age, including embryonic (e.g., in-utero or in-ovo), infant, juvenile, adolescent, adult and geriatric subjects. In some embodiments, a subject is a human.”

As used herein, the term “isolated: in context of a nucleic acid sequence is a nucleic acid sequence that exists apart from its native environment.

As used herein, “pharmaceutically acceptable” means a material that is not biologically or otherwise undesirable, i.e., the materials are administered to a subject without causing any undesirable biological effects such as toxicity.

As used herein, the term “in vivo” is used to describe an event that takes place in a subject's body.

As used herein, the term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

The terms “bacteriophage” and “phage” are used interchangeably herein.

### EXAMPLES

The following examples are included for illustrative purposes only and are not intended to limit the scope of the invention.

### Example 1: Engineered Phage Used in this Application

Recombinant bacteriophage were engineered to contain a genetic deletion in the predicted lysogeny module region. FIG. 1 depicts the predicted lysogeny region of phage p1473. Further, FIG. 1 depicts open reading frames encoding putative repressor and antirepressor proteins and an open reading frame encoding a putative integrase. The first recombinant phage contains a genetic deletion that was introduced into the predicted lysogeny module region of the bacteriophage genome in the region including a putative antirepressor, and two clones were isolated and designated Var009 and Var010. A second recombinant phage contains a genetic deletion that was introduced into the predicted lysogeny module region of the bacteriophage genome in the region including a putative repressor and a single clone was isolated and designated Var012. A final recombinant phage (Var042) was isolated that contains the deletion from Var012 and a second deletion removing the predicted integrase gene. Two additional control variants were generated with different deletions outside of the lysogeny region as controls (not shown), isolated and designated Var002 and Var006, respectively. FIG. 6 depicts the sequence of the Var010 deletion. FIG. 7 depicts the sequence of the Var012 deletion, which is also the first deletion in Var042. FIG. 8 depicts the second deletion in Var042. Additional phage used in this application are described in Table 1.

TABLE 1
Phage used in this application

| | | | | Locus Genus | |
|---|---|---|---|---|---|
| | ATCC | | | (Literature | |
| | Accession | | Sequence | Genus if | Nearest NCBI |
| Phage | No. | Engineered | deleted | different) | Phage (Genus) |
| p1378 | PTA- | wildtype | — | Kayvirus | Staphylococcus |
| | 127329 | | | | phage MSA6 |
| | | | | | (Kayvirus) |
| p1381 | PTA- | wildtype | — | Kayvirus | Staphylococcus |
| | 127330 | | | | phage PhiSA039 |
| | | | | | (Kayvirus) |
| p4815 | PTA- | wildtype | — | Kayvirus | Staphylococcus |
| | 127331 | | | | Phage K |
| | | | | | (Kayvirus) |
| p2808 | PTA- | wildtype | — | Rosenblum | Staphylococcus |
| | 127332 | | | | Phage phiP68 |
| | | | | | (Rosenblumvirus) |
| p3693e001 | PTA- | Lysogeny | SEQ ID | Phietavirus | Staphylococcus |
| | 127337 | knockout | NO: 53 | | Phage WBG8381 |
| | | | | | (Phietavirus) |
| p1498e001 | PTA- | Lysogeny | SEQ ID | Phietavirus | Staphylococcus |
| | 127336 | knockout | NO: 52 | (Dubowvirus) | Phage SA97 |
| | | | | | (Dubowvirus) |
| p3224e002 | PTA- | Lysogeny | SEQ ID | Phietavirus | Staphylococcus |
| | 127341 | knockout | NO: 54 | (Dubowvirus) | Phage SA12 |
| | | | | | (Dubowvirus) |
| p5593e001 | PTA- | Lysogeny | SEQ ID | Phietavirus | Staphylococcus |
| | 127342 | knockout | NO: 55 | (Dubowvirus) | Phage 69 |
| | | | | | (Dubowvirus) |
| p1468e003 | PTA- | Lysogeny | SEQ ID | Phietavirus | Staphylococcus |
| | 127343 | knockout | NO: 56 | (Dubowvirus) | Phage SAP-26 |
| | | | | | (Dubowvirus) |
| p1478e003 | PTA- | Lysogeny | SEQ ID | Phietavirus | Staphylococcus |
| | 127344 | knockout | No: 57 | (Dubowvirus) | Phage ECel- |
| | | | | | 2020a |
| | | | | | (Dubowvirus) |
| p1494e002 | PTA- | Lysogeny | SEQ ID | Phietavirus | Staphylococcus |
| | 127345 | knockout | NO: 58 | (Dubowvirus) | Phage SA12 |
| | | | | | (Dubowvirus) |
| p1473 | | wildtype | — | Phietavirus | Phage |
| | | | | (Dubowvirus) | vB_SauS_713 |
| | | | | | (Dubowvirus) |
| p4815e053 | PTA- | RIP insert | | Kayvirus | Staphylococcus |
| | 127340 | (SEQ ID | | | Phage K |
| | | NO: 14) | | | (Kayvirus) |
| p4815e037 | PTA- | Ipi insert | | Kayvirus | Staphylococcus |
| | 127335 | (SEQ ID | | | Phage K |
| | | NO: 11) | | | (Kayvirus) |
| p1378e062 | PTA- | DNaseI insert | SEQ ID | Kayvirus | Staphylococcus |
| | 127333 | (SEQ ID NO: | NO: 49 | | phage MSA6 |
| | | 9) Lysogeny | | | (Kayvirus) |
| | | knockout | | | |
| p1378e074 | PTA- | TreA insert | SEQ ID | Kayvirus | Staphylococcus |
| | 127334 | (SEQ ID NO: | NO: 50, | | phage MSA6 |
| | | 10) Lysogeny | 51 | | (Kayvirus) |
| | | knockout | | | |
| p1378e075 | PTA- | FS3 insert | | Kayvirus | Staphylococcus |
| | 127338 | (SEQ ID | | | phage MSA6 |
| | | NO: 12) | | | (Kayvirus) |
| p1381e017 | PTA- | FS3 insert | | Kayvirus | Staphylococcus |
| | 127339 | (SEQ ID | | | phage PhiSA039 |
| | | NO: 12) | | | (Kayvirus) |

### Example 2: Engineered Phage Variants Exhibit Lytic Phenotype in S. aureus

FIG. 2 depicts the dilution series of wild-type (WT) p1473 and several variants plated on a lawn of S. aureus using the double agar overlay method. As depicted, the WT phage and variants Var002 and Var006 produced small hazy plaques, while variants Var009, Var010 and Var012 produced larger clearer plaques. Variants Var002 and Var006 contain mutations outside of the lysogeny region. The variants with mutants outside the lysogeny region do not show changes in plaque morphology indicating that these phage variants remain temperate. Var009, Var010 and Var012, with mutations in the lysogeny region show clearer plaques and higher efficiency of plaquing on the strain shown. These data indicate that Var009, Var010 and Var012 were successfully converted from lysogenic to lytic phenotype, as clear plaque morphology is a hallmark of lytic bacteriophages in S. aureus (References Mitarai et al J. Bacteriol. 2016. doi: 10.1128/JB.00965-15, Garcia et al. Appl. Environ. Microbiol. 2009. doi: 10.1128/AEM.01864-09) and is a known phenomenon in other examples of lysogenic phages that have been genetically converted to obligately lytic. The ability of Var010 and Var012 to plaque at higher efficiency (i.e. lower down the plate, when the phage is more dilute) may indicate that the modified phage is no longer susceptible to interference from the endogenous prophage present in the bacterial strain, as previously observed for other phages following conversion from temperate to obligate lytic (Reference: Zhang et al. Microbiol. 2013. doi: 10.1099/mic.0.067116-0). FIG. 3 depicts a close-up image of FIG. 2, showing the larger plaque morphology for wild-type p1473, Var010 and Var012. In FIG. 3, the arrows point to individual phage plaques. As depicted, Var010 and Var012 plaques display a clearer morphology compared to wild-type. Additionally, the zone of clearing with too many plaques to count is clearer in Var010 and Var012 than the WT phage.

### Example 3: Bacteriophage Killing Assay Confirms Lysogenic to Lytic Phenotype Conversion

FIG. 4 depicts the bacteriophage killing assay with strain b4063 (USA300 strain FPR3757) in LB challenged with Wild-type p1473, Var012, or Var042 as measured by bacterial counts as a function of time. For the bacteriophage killing assay with strain b4063 (USA300 strain FPR3757), bacteria were inoculated into LB in triplicate. For cells only, nothing else was added. For the other phage, Wild-type p1473, Var012 or Var042 were added at an MOI of 1. Bacterial CFUs were enumerated at 0, 2, 4, 6 and 24 hours post inoculation. As depicted in FIG. 4, at the 24 hour time point, the bacterial counts had recovered in the p1473 WT treated bacteria, but the two variants showed sustained killing to limit of detection (LOD). Temperate phages often show quick rebound due to the outgrowth of lysogens which are resistant to infection by the phage. The lack of rebound in Var012 and Var042 is consistent with the inability of these variants to form lysogens.

### Example 4: Comparison of Rebound on S. aureus Clinical Isolates

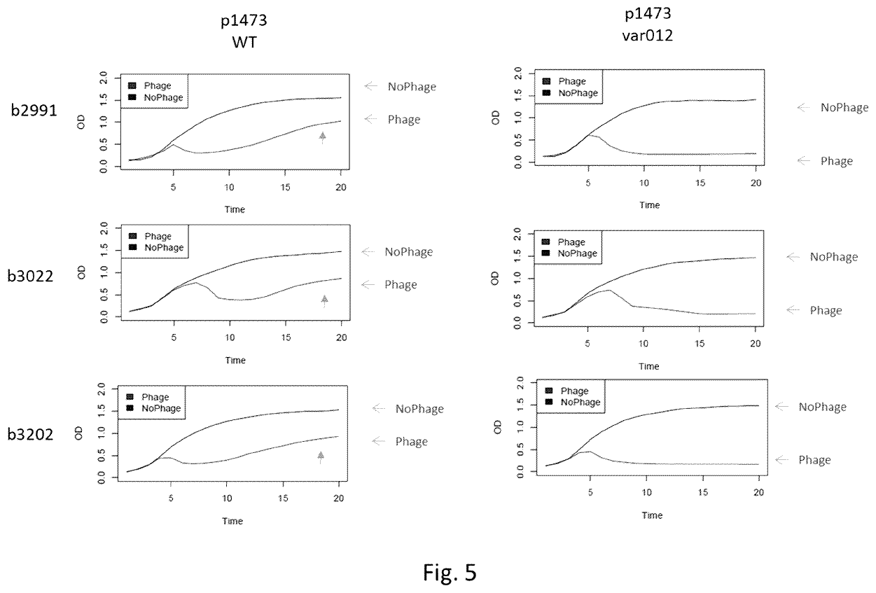

FIG. 5 depicts growth curves of three S. aureus clinical isolates (b2991, b3022 and b3202) in LB challenged with p1473 WT or p1473 Var012. Growth of the clinical isolates in LB was monitored by taking the OD at 600 nm periodically over a 24 hour time course. Control growth curves are uninfected culture controls for each strain, while test growth curves are for cultures challenged with WT or Var012 at an MOI of 1. Arrows indicate regrowth of the clinical isolates in the WT challenged cultures while the Var012 challenged cultures remain suppressed out to 24 hours. Overall, the experimental comparison of the three S. aureus clinical isolates (b2991, b3022 and b3202) in LB challenged with p1473 WT or p1473 Var012 shows more regrowth in strains challenged with WT phage, with b2991 showing the best recovery over a 24 hour time course.

### Example 5. Phage Lytic Activity when Engineered with CRISPR-Cas Full Construct

Top agar overlays are prepared by mixing 100 μL of a saturated overnight culture of b4063 with 6 mL of 0.375% agar in LB containing 10 mM MgCl₂ and 10 mM CaCl₂. After the top agar solidified, 2 μL drops of serial 10-fold dilution series of p1473 wt (wild type) and p1473-Cas (Cas system only) and p1473-crArray (targeting CRISPR Array+Cas system) are spotted onto the surface of the top agar. Plates are incubated at 37° C. for ˜18 h, then imaged using a Keyence BZ-X800 microscope at 4× and 10× magnification.

### Example 6: Engineering of a Lytic Bacteriophage

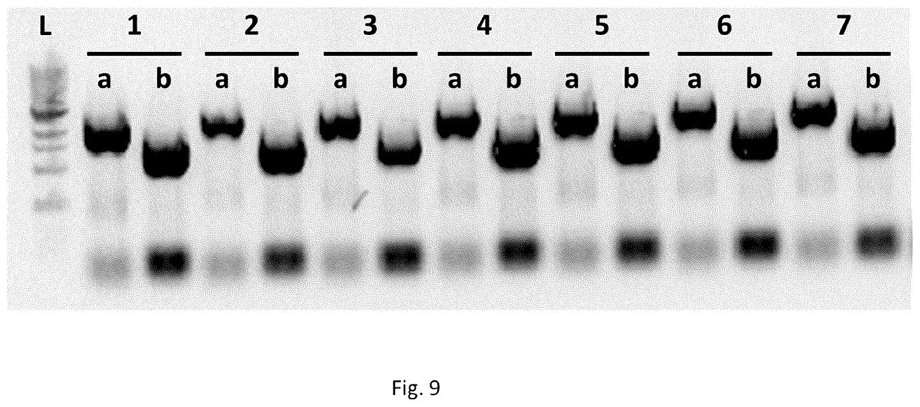

Bacteriophage was engineered with a Type IB CRISPR-Cas system (LMIB) and a CRISPR Array (SEQ ID NO: 24). The engineered phage lysate was spotted onto a bacterial overlay in order to obtain isolated (clonal) plaques. Seven plaques were picked and screened by PCR for the presence of the desired insert. Each plaque was screened using two pairs of PCR primers, with one pair covering the upstream engineering junction (i.e., the site where the wild type phage genome meets the engineered insert) and a significant portion of the insert and one pair covering the downstream engineering junction and a significant portion of the insert. Since one primer from each primer pair binds within the insert, un-engineered phages will produce no PCR bands. In FIG. 9, L designates a DNA size ladder, the numbers 1-7 indicate individual clonal plaques being screened, and a and b indicate the two primer pairs. Both primer pairs produced bands of the expected sizes for all plaques, indicating that all plaques were successfully engineered with the LMIB insert (SEQ ID NO: 23).

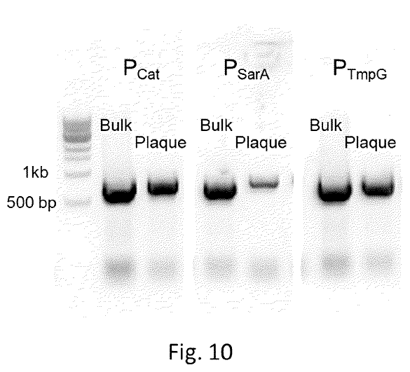

After engineering of the three promoter variants of lnqQ (SEQ NO: 7), P_{cat}-lnqQ (SEQ ID NOS: 16, 7), P_{SarA}-lnqQ (SEQ ID NOS: 17, 7), or P_{TmpG}-lnqQ (SEQ ID NOS: 18, 7), into Kayvirus phage p4815, polymerase chain reaction (PCR) was used to confirm insertion of the promoter+lnqQ into the phages. The SarA promoter used a SodB RBS (SEQ ID NO: 21). This PCR reaction used one primer upstream of the insertion site and one primer in the inserted sequence. The results are depicted in FIG. 10. Sequencing of the phage DNA confirmed insertion of the variants in the phage.

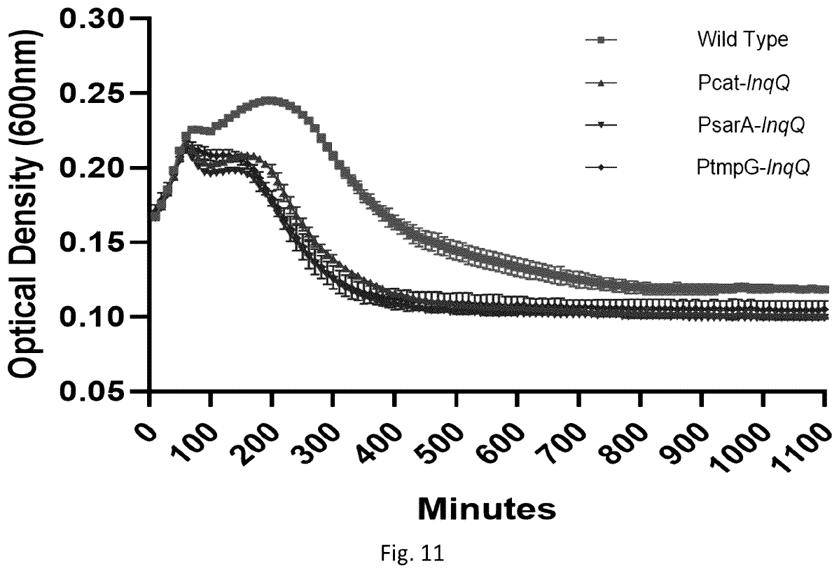

Clinical isolate b2655 was inoculated with either wild-type (WT) phage (squares), P_{cat}-lnqQ, P_{SarA}-lnqQ, or P_{TmpG}-lnqQ. For each sample, three bacterial replicates were inoculated with phage at a multiplicity of infection of 1. Cultures were grown at 37° C. in a shaking plate reader with optical density at 600 nm measured every 10 minutes for 18 hours. The results are depicted in FIG. 11. Phages with lnqQ reduced the growth of the bacteria compared to WT phage starting at around 80 minutes and persisting throughout the experiment.

### Example 7: An Engineered Bacteriophage Comprising DNAse I

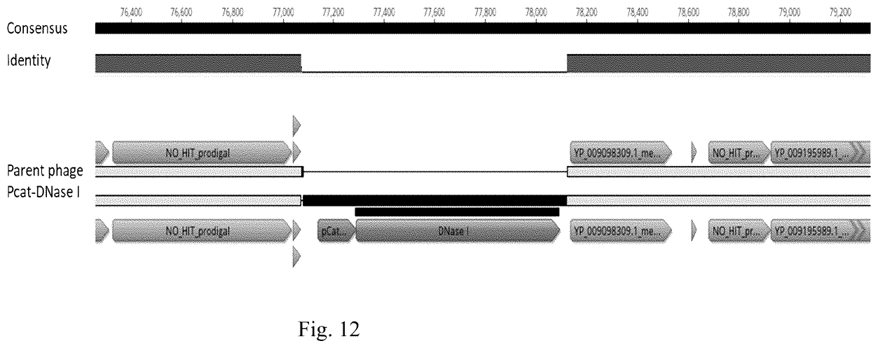

A p1378 Kayvirus bacteriophage was engineered to contain DNAse I (SEQ NO: 1) and the Pcat promoter (SEQ ID NO: 16). The DNA sequence encoding mature human DNase was modified to optimize codon usage and remove sequences not tolerated by the phage while maintaining the amino acid identity. This sequence was placed downstream of the Pcat promoter. The Pcat-DNase I sequence was engineered into the phage and the sequence was confirmed by Illumina next generation sequencing of the engineered phage genome. The results are depicted in FIG. 12.

### Example 8: Engineering of a p1494 Phage

A Phictavirus phage p1494 was isolated. Region e002 (var0022) was deleted as depicted in FIG. 13A. FIG. 13B depicts the bacteriophage killing assay with strain b4063 (USA300 strain FPR3757) in LB challenged with with-type and engineered phage as measured by bacterial counts as a function of time. Growth curves of USA300 Strain FPR3757 either with no phages/cells only (circles), wild-type p1494 (squares) or lytic p1494e002 (triangles) at a multiplicity of 1. At 0, 3, 6 and 24h post infection, cultures were diluted and plated on LB agar to enumerate the bacterial survivors. Data shows the mean and standard error of the mean for 3 replicates. Open shapes represent time points where no bacteria were recovered plotted at the limit of detection (LOD) of the assay. Closed shapes represent time points where bacterial colonies were counted. No bacteria were recovered for any time point in the lytic p1494e002 phage.

FIG. 13C depicts growth curves of Staphylococcus isolate b2655 either with no phages/cells only (circles), wild-type p1498 (squares) or lytic p1498e001 (triangles) at a multiplicity of infection of 1. At 0, 2, 4, 8 and 24h post infection, cultures were diluted and plated on LB agar to enumerate the bacterial survivors. Data shows the mean and standard error of the mean for 3 replicates. Open shapes represent time points where no bacteria were recovered plotted at the limit of detection (LOD) of the assay. Closed shapes represent time points where bacterial colonies were counted.

### Example 9: Bacteriophage Killing Assay for the Cocktail

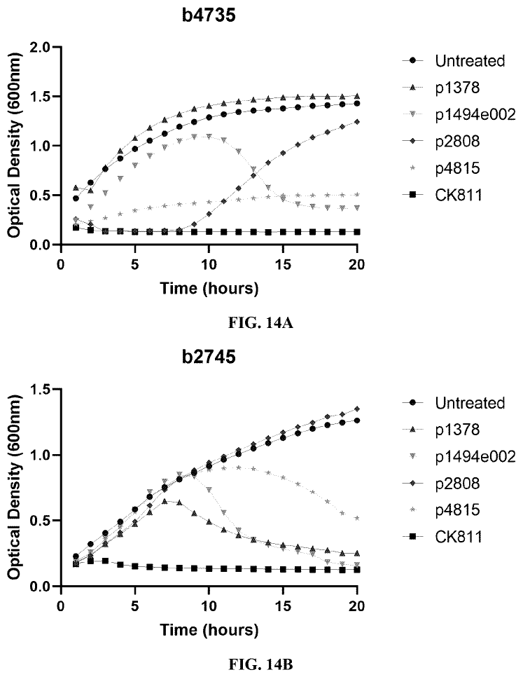

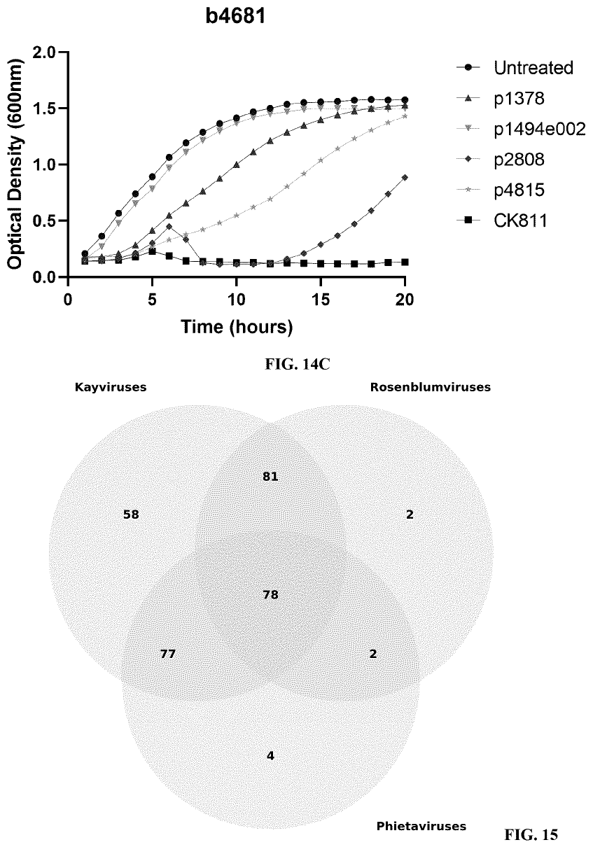

Growth curves from treatment of bacterial cultures with individual phages or CK811, a four phage cocktail comprising p1378, p1494e002, p2808 and p4815. Cultures were grown at 37° C. with aeration and optical density at 600 nm was measured hourly to monitor bacterial growth throughout the 20 hour experiment. Results are depicted in FIGS. 14A-14C. In strain b4735, an isolate from a bloodstream infection, three of the individual phages, p1494e002 (upside down triangles), p2808 (diamonds) and p4815 (stars) show reduction in bacterial growth, but sustained reduction is only seen for CK811 (squares). In strain b2655, an isolate from a respiratory infection, three of the individual phages, p1378 (triangles), p1494e002 (green upside down triangles), and p4815 (stars) show reduction in bacterial growth. Sustained repression of bacterial growth is only seen in the sample treated with the 4 phage cocktail, CK811, (squares). In strain b4681, an isolate from a bloodstream infection, three of the individual phages, p1378 (red triangles), p2808 (purple diamonds) and p4815 (orange stars) show reduction in bacterial growth, but sustained reduction is only seen for CK811 (black squares).

### Example 10: Host Range of a Bacteriophage Cocktail

A 308 strain panel of clinical isolates of S. aureus from a bloodstream infection was used to test the host range of a bacteriophage cocktail. 136 (44%) of the isolated strains were multi-drug resistant. The strains included at least 69 different multi-locus sequencing types (MLST) as depicted in Table 2. MLST is based on sequence analysis of fragments from seven S. aureus housekeeping genes, i.e., arcC, aroE, glpF, gmk, pta, tpi and yqiL. The S. aureus strains were typed using the method described in Deurenberg R. H., Vink C., Kalenić S., Friedrich A. W., Bruggeman C. A., Stobberingh E. E. The molecular evolution of methicillin-resistant Staphylococcus aureus. Clin. Microbiol. Infect. 2007; 13:222-235. doi: 10.1111/j.1469-0691.2006.01573.x. The S. aureus strains used were b004604, b004605, b004606, b004607, b004608, b004609, b004610, b004611, b004612, b004613, b004614, b004615, b004616, b004617, b004618, b004619, b004620, b004621, b004622, b004623, b004624, b004625, b004626, b004627, b004628, b004629, b004630, b004631, b004632, b004633, b004634, b004635, b004636, b004637, b004638, b004639, b004640, b004641, b004642, b004643, b004644, b004645, b004646, b004647, b004648, b004649, b004650, b004651, b004652, b004653, b004654, b004655, b004656, b004657, b004658, b004659, b004660, b004661, b004662, b004663, b004664, b004665, b004666, b004667, b004668, b004669, b004670, b004671, b004672, b004673, b004674, b004675, b004676, b004677, b004678, b004679, b004680, b004681, b004682, b004683, b004684, b004685, b004686, b004687, b004688, b004689, b004690, b004691, b004692, b004693, b004694, b004695, b004696, b004697, b004698, b004699, b004700, b004701, b004702, b004703, b004704, b004705, b004706, b004707, b004708, b004709, b004710, b004711, b004712, b004713, b004714, b004715, b004716, b004717, b004718, b004719, b004720, b004721, b004722, b004723, b004724, b004725, b004726, b004727, b004728, b004729, b004730, b004731, b004732, b004733, b004734, b004735, b004736, b004737, b004738, b004739, b004740, b004741, b004742, b004743, b004744, b004745, b004746, b004747, b004748, b004749, b004750, b004751, b004752, b004753, b004754, b004755, b004756, b004757, b004758, b004759, b004760, b004761, b004762, b004763, b004764, b004765, b004766, b004767, b004768, b004769, b004770, b004771, b004772, b004773, b004774, b004775, b004776, b004777, b004778, b004779, b004780, b004781, b004782, b004783, b004784, b004785, b004786, b004787, b004788, b004789, b004790, b004791, b004792, b004793, b004794, b004795, b004796, b004797, b004798, b004799, b004800, b004801, b004802, b004803, b004804, b004805, b004806, b004807, b004808, b004809, b004810, b004811, b004812, b004813, b004814, b004815, b004816, b004817, b004818, b004819, b004820, b004821, b004822, b004823, b004824, b004825, b004826, b004827, b004828, b004829, b004830, b004831, b004832, b004833, b004834, b004835, b004836, b004837, b004838, b004839, b004840, b004841, b004842, b004843, b004844, b004845, b004846, b004847, b004848, b004849, b004850, b004851, b004852, b004853, b004854, b004855, b004856, b004857, b004858, b004859, b004860, b004861, b004862, b004863, b004864, b004865, b004866, b004867, b004868, b004869, b004870, b004871, b004872, b004873, b004874, b004875, b004876, b004877, b004878, b004879, b004880, b004881, b004882, b004883, b004884, b004885, b004886, b004887, b004888, b004889, b004890, b004891, b004892, b004893, b004894, b004895, b004896, b004897, b004898, b004899, b004900, b004901, b004902, b004903, b004904, b004905, b004906, b004907, b004908, b004909, b004910, b004911.

TABLE 2
Multi-locus sequence typing of panel of S. aureus bacteria

| Sequencing | | |
|---|---|---|
| Type (ST) | # of Strains | % Strains |
| 8 | 57 | 18.51 |
| 5 | 42 | 13.64 |
| 22 | 20 | 6.49 |
| 15 | 10 | 3.25 |
| 1 | 9 | 2.92 |
| 30 | 8 | 2.60 |
| 398 | 8 | 2.60 |
| 105 | 6 | 1.95 |
| 45 | 6 | 1.95 |
| 672 | 5 | 1.62 |
| 2250 | 4 | 1.30 |
| 582 | 4 | 1.30 |
| 72 | 4 | 1.30 |
| 97 | 4 | 1.30 |
| 239 | 3 | 0.97 |
| 34 | 3 | 0.97 |
| 87 | 3 | 0.97 |
| 101 | 2 | 0.65 |
| 109 | 2 | 0.65 |
| 1159 | 2 | 0.65 |
| 1165 | 2 | 0.65 |
| 1181 | 2 | 0.65 |
| 12 | 2 | 0.65 |
| 121 | 2 | 0.65 |
| 152 | 2 | 0.65 |
| 1750 | 2 | 0.65 |
| 20 | 2 | 0.65 |
| 225 | 2 | 0.65 |
| 25 | 2 | 0.65 |
| 291 | 2 | 0.65 |
| 3628 | 2 | 0.65 |
| 59 | 2 | 0.65 |
| 7 | 2 | 0.65 |
| 779 | 2 | 0.65 |
| 88 | 2 | 0.65 |
| 10 | 1 | 0.32 |
| 1011 | 1 | 0.32 |
| 1049 | 1 | 0.32 |
| 1156 | 1 | 0.32 |
| 1351 | 1 | 0.32 |
| 149 | 1 | 0.32 |
| 1637 | 1 | 0.32 |
| 1649 | 1 | 0.32 |
| 1757 | 1 | 0.32 |
| 1842 | 1 | 0.32 |
| 188 | 1 | 0.32 |
| 1970 | 1 | 0.32 |
| 2066 | 1 | 0.32 |
| 256 | 1 | 0.32 |
| 2867 | 1 | 0.32 |
| 2945 | 1 | 0.32 |
| 3149 | 1 | 0.32 |
| 3182 | 1 | 0.32 |
| 3510 | 1 | 0.32 |
| 39 | 1 | 0.32 |
| 395 | 1 | 0.32 |
| 4317 | 1 | 0.32 |
| 47 | 1 | 0.32 |
| 4730 | 1 | 0.32 |
| 50 | 1 | 0.32 |
| 508 | 1 | 0.32 |
| 573 | 1 | 0.32 |
| 6 | 1 | 0.32 |
| 630 | 1 | 0.32 |
| 737 | 1 | 0.32 |
| 828 | 1 | 0.32 |
| 848 | 1 | 0.32 |
| 923 | 1 | 0.32 |
| 93 | 1 | 0.32 |
| undetermined | 42 | 13.64 |

Results are depicted in Table 3. The ability of Kayviruses, a Phietavirus, and a Rosenblumvirus to target the 308 strains of the panel was tested. As can be seen in FIG. 15, for a majority of strains tested, at least two phage infect a particular target bacteria, reducing the likelihood that a target bacteria would be able to evolve resistance against a particular phage and evade infection by another bacteriophage.

TABLE 3
Results of Host Range Assay

| Strain | Phietavirus | Rosenblumvirus | Kayviruses |
|---|---|---|---|
| Total | 52% | 53% | 95% |

### Example 11: Comparison of Rebound on S. aureus Clinical Isolates

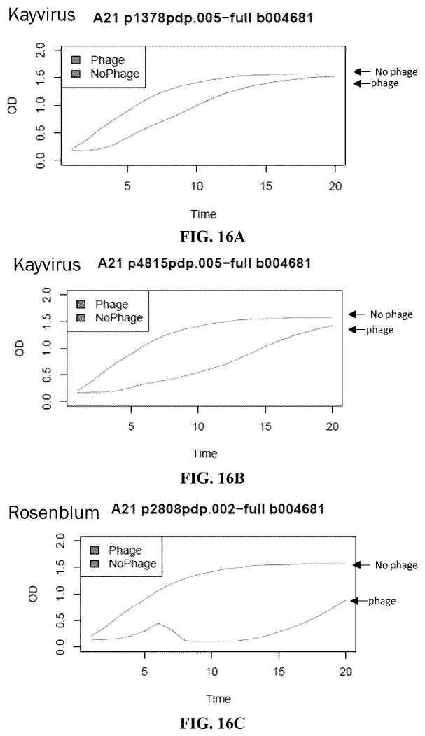

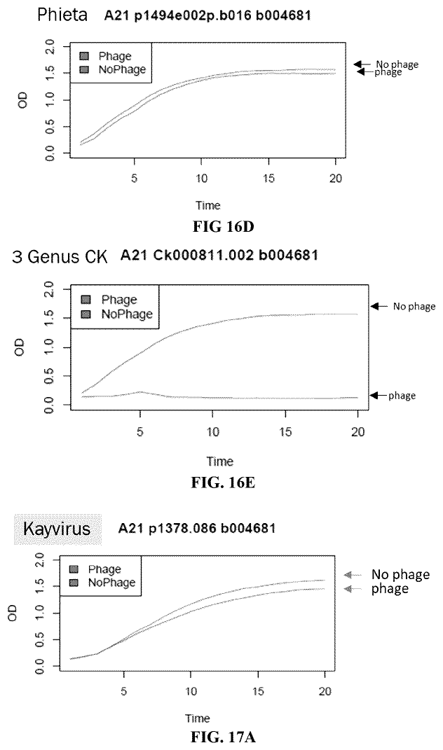

Growth of the clinical isolates b4681 in LB was monitored by taking the OD at 600 nm periodically over a 24 hr. time course. Control growth curves are uninfected culture controls for each strain, while test growth curves are for cultures challenged with phage at an MOI of 1. FIGS. 16A-16E depicts growth curves of S. aureus clinical isolates b4681 in LB challenged with p1378, p4815, p2808, p1494e002 or a 3 genus cocktail. B4681 shows rebound or no hit in all single phage treatments, but not in the 3 genus phage cocktail.

### Example 12: Comparison of Rebound on S. aureus Clinical Isolates

Growth of the clinical isolates b4681, b4787, b4655, and b4689 in LB was monitored by taking the OD at 600 nm periodically over a 24 hr. time course. Control growth curves are uninfected culture controls for each strain, while test growth curves are for cultures challenged with phage at an MOI between 0.1 and 1.

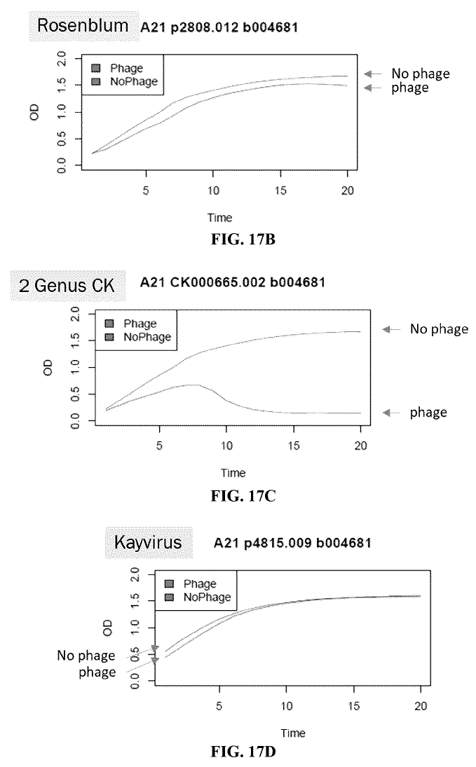

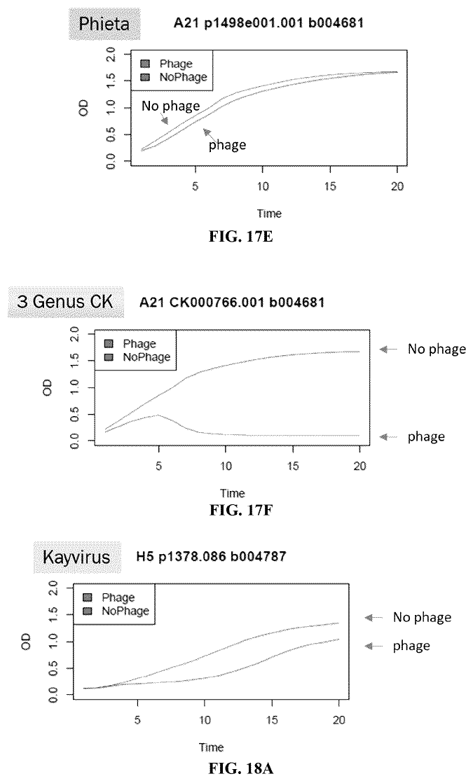

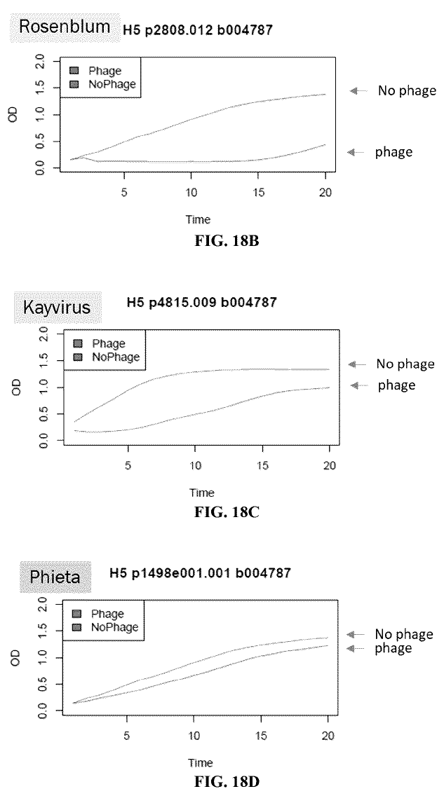

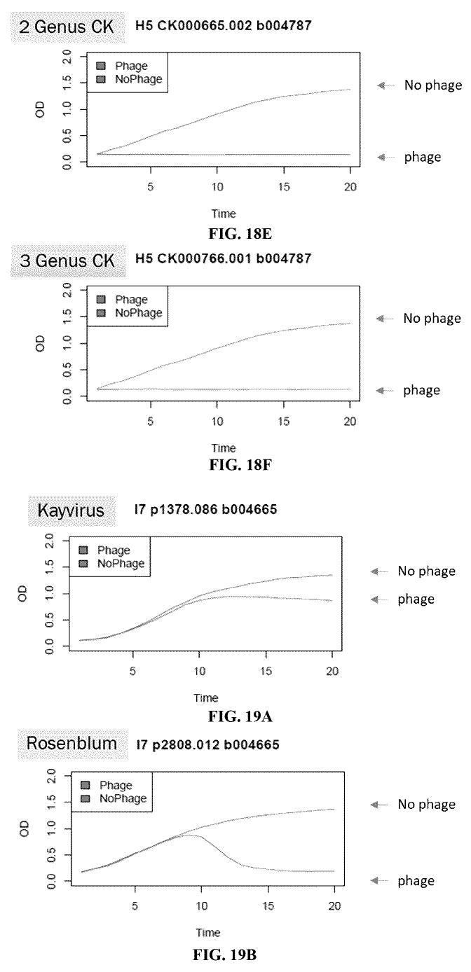

FIGS. 17A-17F depict growth curves of b4681. FIGS. 18A-18F depict growth curves of b4787. Treatment with p1378 or p2808 resulted in b4681 or b4787 growth over time (FIGS. 17A-17B, 18A-18B). Treatment with a two genus cocktail (p1378, p2808) resulted in a suppression of bacterial growth (FIG. 17C, 18C). Treatment with p4815 or p1498e001 also permitted growth of b4681 or b4787, while treatment with a 3 genus cocktail (p1378, p2808, p4815, p1498e001) resulted in reduced resistance outgrowth (FIGS. 17D-17F, 18D-18F).

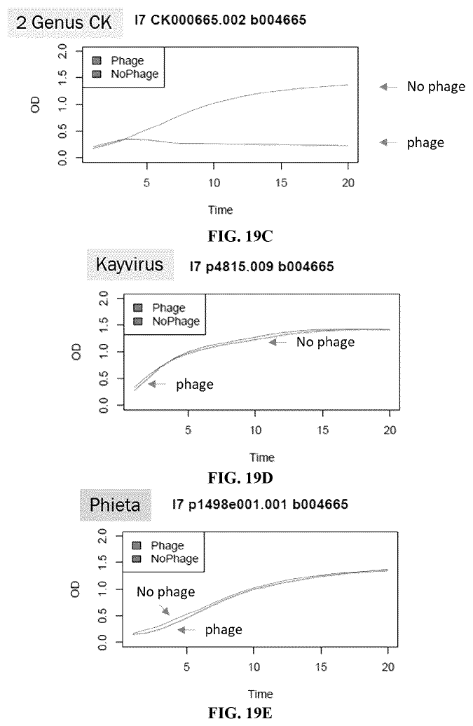

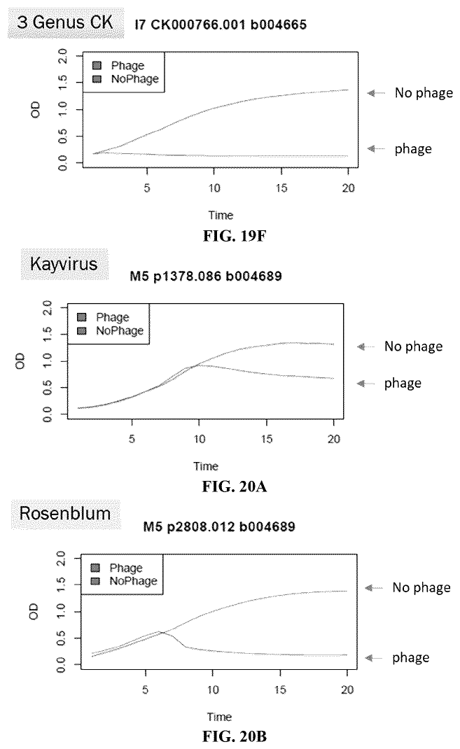

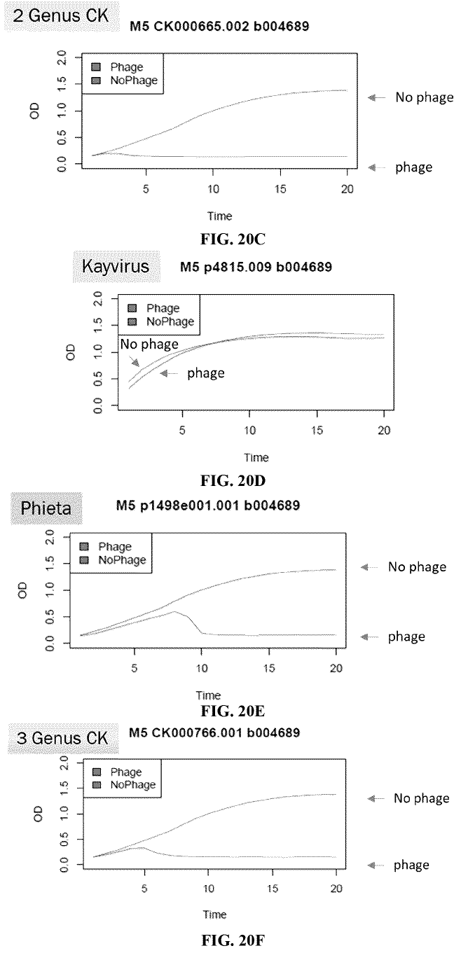

Growth of the clinical isolates b4655 and b4689 show the additive effect of the cocktail. Growth curves of b4655 are depicted in FIGS. 19A-19F and growth curves of b4689 are depicted in FIGS. 20A-20F. Treatment with p1378 or 02808 reduced growth of the clinical isolates (FIGS. 19A-19B, 20A-20B) while treatment with the two genus cocktail was more quickly able to suppress growth (FIGS. 19C, 20C). Treatment of b4655 with p4815 or p1498e001 or b4655 with p4815 also resulted in growth, although treatment with p1498e001 was able to inhibit growth of b4689 (FIGS. 19D-19E, 20D-20E), while treatment with the 3 genus cocktail quickly suppressed growth (FIGS. 19F, 20F).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Sequences

| SEQ ID NO | Description |
|---|---|
| 1-5, 7 | DNA sequences for alternative payloads |
| 9-15 | AA sequences for alternative payloads |
| 16-18 | Promoters for payloads |
| 19 | Promoter for CRISPR array |
| 21 | RBS (ribosome binding site) |
| 22 | LMIB (natural) |
| 23 | LMIB (optimized) |
| 24 | crArray with promoter |
| 25-29 | CRISPR-Cas LMIB amino acid sequences |
| 33-37 | CRISPR-Cas natural LMIB nucleotide sequences |
| 38-42 | CRISPR-Cas optimized LMIB nucleotide sequences |
| 43 | Repeat |
| 44-46 | Spacer sequences |
| 30-32, 47-58 | Phage deletions |

### Example Payload and Promoter Sequences

| SEQ | | |
|---|---|---|
| ID NO | Description | Sequence (5′-3′) |
| 1 | hDNaseI | ATGTTAAAGATTGCTGCTTTTAACATTCAAACATTTGGTGAAA |
| | | CAAAAATGAGTAATGCTACATTAGTATCATATATTGTTCAAAT |
| | | TTTATCTAGATATGATATTGCTTTAGTTCAAGAAGTTAGAGAT |
| | | AGTCATTTAACAGCTGTAGGTAAATTATTAGATAATTTAAATC |
| | | AAGATGCTCCTGATACATACCATTACGTAGTAAGTGAACCTTT |
| | | AGGTAGAAATTCATATAAAGAAAGATACTTATTTGTATATAG |
| | | ACCTGACCAAGTAAGTGCTGTAGATTCATATTATTATGATGAT |
| | | GGTTGTGAACCTTGTGGTAATGATACATTTAATAGAGAACCTG |
| | | CTATTGTTAGATTCTTTTCAAGATTTACAGAAGTAAGAGAATT |
| | | TGCTATTGTACCTTTACATGCTGCTCCTGGTGATGCTGTAGCT |
| | | GAAATTGATGCTTTATATGATGTATATTTAGATGTACAAGAAA |
| | | AATGGGGTTTAGAAGATGTAATGTTAATGGGTGATTTTAATGC |
| | | TGGTTGTAGTTATGTAAGACCTTCACAATGGTCTAGTATTAGA |
| | | TTATGGACAAGTCCTACATTTCAATGGTTAATTCCTGATTCAG |
| | | CTGATACAACAGCTACACCTACACATTGTGCTTATGATAGAAT |
| | | TGTAGTAGCTGGTATGTTATTAAGAGGTGCTGTAGTACCTGAT |
| | | TCTGCTTTACCTTTTAATTTTCAAGCTGCTTATGGTTTAAGTGA |
| | | CCAATTAGCTCAAGCTATTTCAGACCATTATCCTGTAGAAGTA |
| | | ATGTTAAAACATCATCACCATCATCACTAA |
| 2 | TreA | ATGATTGATATATACTTAGGAGAAGGTTATAATAAAGAATAC |
| | | TTGTCTAAAGCACTCAGATTAATTAATGACCATGCTCCTAGGG |
| | | AGTTAAGTTATGATTTTAATAATGTAGAAGCGGATGTTAATAT |
| | | TCACACAATGTTATATGTTAAACCTGAAGATAGGTATGTATAT |
| | | AAGGATATATCTTAT[T]ACTTCCCAGGTGATTTAATTATATGT |
| | | ATAGTTGAAGATGATGCTATTGTGTATCACCAAGGTAAACAG |
| | | GTTTCAGGTATTAGTATTTTAAGAATAATAGAAGAGATATTTT |
| | | AA |
| 3 | Ipi | ATGAAAACTTTCAAAGAATTTACTTCTACGACTACCCCGGTTT |
| | | CTACTATTACTGAAGCTACTCTTACTTCTGAAGTTATTAAAGC |
| | | AAATAAAGGACGAGAAGGTAAACCGATGATTAGTCTGGTTGA |
| | | TGGTGAAGAAATCAAAGGTACTGTTTACCTAGGTGATGGGTG |
| | | GTCTGCTAAAAAGGATGGTGCTACAATCGTTATCTCTCCTGCT |
| | | GAAGAAACTGCGTTGTTTAAAGCTAAACACATTTCTGCGGCA |
| | | CATCTCAAGATTATTGCTAAAAATCTTTTGTAA |
| 4 | FS3 | ATGTATGCTCCTTGGACAAATTTTTAA |
| 5 | RIP | ATGTATAGTCCTTGGACAAATTTTTAA |
| 7 | LnqQ | ATGATTGTTGCAAAGAGAAAGCGTGTTAAAATGGCAGGGTTT |
| | | TTAAAAGTAGTTCAATTACTAGCTAAATATGGTTCTAAAGCTG |
| | | TACAATGGGCTTGGGCAAACAAGGGTAAGATTTTAGATTGGC |
| | | TTAATGCAGGTCAGGCTATTGATTGGGTAGTTTCGAAAATTAA |
| | | GCAAATTTTAGGTATTAAGTAA |
| 9 | DNaseI | MLKIAAFNIQTFGETKMSNATLVSYIVQILSRYDIALVQEVRDSH |
| | | LTAVGKLLDNLNQDAPDTYHYVVSEPLGRNSYKERYLFVYRPD |
| | | QVSAVDSYYYDDGCEPCGNDTFNREPAIVRFFSRFTEVREFAIVP |
| | | LHAAPGDAVAEIDALYDVYLDVQEKWGLEDVMLMGDFNAGCS |
| | | YVRPSQWSSIRLWTSPTFQWLIPDSADTTATPTHCAYDRIVVAG |
| | | MLLRGAVVPDSALPFNFQAAYGLSDQLAQAISDHYPVEVMLKH |
| | | HHHHH* |
| 10 | TreA | MIDIYLGEGYNKEYLSKALRLINDHAPRELSYDENNVEADVNIHT |
| | | MLYVKPEDRYVYKDIS[Y]YFPGDLIICIVEDDAIVYHQGKQVSGI |
| | | SILRIIEEIF |
| 11 | Ipi | MKTFKEFTSTTTPVSTITEATLTSEVIKANKGREGKPMISLVDGEE |
| | | IKGTVYLGDGWSAKKDGATIVISPAEETALFKAKHISAAHLKIIA |
| | | KNLL* |
| 12 | FS3 | MYAPWTNF* |
| 14 | RIP | MYSPWTNF* |
| 13 | LnqQ | MIVAKRKRVKMAGFLKVVQLLAKYGSKAVQWAWANKGKILD |
| | | WLNAGQAIDWVVSKIKQILGIK* |
| 15 | LnqQ | MAAFMKLIQFLATKGQKYVSLAWKHKGTILKWINAGQSFEWIY |
| | | KQIKKLWA |
| 16 | pCat | AAACGAAAATTGGATAAAGTGGGATATTTTTAAAATATATAT |
| | | TTATGTTACAGTAATATTGACTTTTAAAAAAGGATTGATTCTA |
| | | ATGAAGAAAGCAGACAAGTAAGCCTCCTAAATTCACTTTAGA |
| | | TAAAAATTTAGGAGGCATATCAA |
| 17 | pSarA | TTTACTTCTCATTTTTAATTAGTTATAAT |
| 18 | pTmpG | TAATAAATAAGTTGACAGAAAGTTAATAATATGGTATACTTAT |
| | | AAAGTAATATTTAGTGGGTATACCATGTTATATTAATAAAG |
| 19 | pCap1 | AGAGTTTGCAAAATATACAGGGGATTATATATAATGGAAA |
| 21 | SodB (RBS) | GGTACCTTAGGAGGATGATTATTT |

### Example CRISPR-Cas Systems and CRISPR-Cas Components

| | | |
|---|---|---|
| 22 | LMIB (natural) | ATGAGATTAAAGATAAATTGTGATTTTGATTCTAAAATAATTTC |
| | | AAAAGATTTTCAAAGTAAAGTTGTTAGTCTGTTCAAAGCGGGC |
| | | ATTATGAAATCGAGTCCAGAGAGATATGAGAATTTATTTGGCG |
| | | GGAATAAGCATAAGCAATATACATTTTCAGTGTACCTTCCCAA |
| | | ACCTCAAAATAAAGGTGCGGAAATTCAGTTAAATGAAGCAAA |
| | | TTGTATTATTAATTTTTCAACGGGAGATGCGGAAACGGGAATC |
| | | ATTTTTTATAATGCATTGATGAGTTTGAGAGGTAGCAAAGTTTT |
| | | ATTTGGAGCTGGGAATCATATTACGGTAAAAAACATTCAAATA |
| | | GTCCCGGAGAAAAAAATTATCGGCAAACGAACAATTTTGAGA |
| | | ACGCTTTCACCTGTGGTTAGTAGAGATCATAATAGAGAAACAT |
| | | TCAAGAATTGGTTTTACAGTTTTGAGGATGAGGAATTTGAGCC |
| | | AACTTTGAAAAGAAACATGTTGCCCTATTTAATGGATGCATTT |
| | | GGTGAGCAAGCTCGTTATGATTTGGAGAAATTGAAAATAACGC |
| | | CAATTTCGATGAAGAAAGTAGTCGTTTATTGCCACGAGATACA |
| | | TATTGAAAGCTCAGTCGGGATTTTTGAATTGGAGGCTGAGGCT |
| | | TATTTGCAGAAGTATTTGGTGGAGAATGGTATTGGGACTATGA |
| | | CAGGATCTGGATTTGGCATGATAGAGCAGTTTTAGACTGGAGG |
| | | TGGAAGAATGCGGACAGAAATAGAAGTAAGGGCAAATGATTG |
| | | GTTAATAAATGCTGGTTTAACTGGTTTTCTAAATATCGTTGGAA |
| | | AAGAAAATGTAAGAATTGATGGACAAGCTATTTATTTTACAGC |
| | | AGATTTATTAGAGGACTTTGAAACGAAATACTTTAATTTCTTTA |
| | | TTCAAGAATATAAAGAAACGTTATCGTGGTATAAAATCGTTTC |
| | | TTATAAAGAGACAATGGAACAGTTTAGAATAGATGAATTTGCT |
| | | TCTTTTGACGAGGTTGCATTGGGAAATTTAAATAAATATATGA |
| | | AAGATGTAGTAAAGTTTTATTTAAAAAAAGCTAATTATATCAA |
| | | GGTGTTTCCGCTAATTGATCCTAGTGCTAATGTAACAAAATGG |
| | | TTGGAAAAATTAAATACAATTAATTTAACTAAAAAGCAAAAAT |
| | | GGGATGAAGTAAAAGCCGATATTATTGAAGAAGTAAAGCAAA |
| | | CATATACTCAACTAGATCTGATTATTGATTTTTGTGCAAGTGAA |
| | | AAGGGGCTTCAATATCTAGGAGCAAAAAATCTGATATATTCTG |
| | | TTATAAATAAAGGTTGGTCTGGTGTATCTTTCCTTCTTAAACAA |
| | | ACAAAATTTATTGATCCCTATTTAGATTACAAAACTAGTTTTTT |
| | | GGACCCAGTAATAGATTATTTAGACACCGATTTATCTAAAGCA |
| | | AAATATAATTGTTTCAATTGTAATCAACCAATTAAGAATTTGA |
| | | AACTAGATCTCAGTTTTATGAACGATGTTGGGTTTGATACTGCA |
| | | AGAAAAACAGCTCACGTATGGGATTTTAACAATGATGTTGCGA |
| | | CTTGCCCGATTTGTCGTTTGATTTATTCATGTGTTCCAGCTGGA |
| | | TTTACGTATGTGTACGGAGAAGGTATGTTTGTCAACGATTCAT |
| | | ATGGAGTAGAGGAATTACACCGTGTTAATGAACGTATGCGGAA |
| | | TTCTATCCTGCGCTTTAACAAAGATGGAATTAATTCAACTAATA |
| | | CTTACCGAGCGCTTGTGGAGTCTATTACGATGGAGCATGAAAA |
| | | TAATCGACGCTATGAACTTGCAGACATTCAGTTAGTTAGATAC |
| | | GAAAATGAGCATTATCGTTTTAATTTACTTTCTAAAAAAATGTT |
| | | GCATATTGTAAATGATTCAAAGGGAATATTGAAAAGTTTAATT |
| | | CGCTGTGGTTATAAAGAAGGTAATTTGAATGTTAATTTGTACA |
| | | AAGAAGTTATTCAGCATTTAATGAACAATGAAAATTTATTTAC |
| | | GCTTATTCATAAATTAATCTACTATAAACAAACTAGTGTAAAT |
| | | GGTTTGTATTACAATATGGGACACGTTTCAGGGATTTTAGATAT |
| | | AAATACAAAATTTTTGAAGGAGATGGAAGTGATGACAAATATT |
| | | TCTCAAAATCAATTATGGTTCGTTCAAAATTGTGGAAAGGAAT |
| | | TTAAGGAAGGTTATGTTAAGAAGAAGTCAGAAAATAAAATTTC |
| | | GGGAATAACATACAAGTTATTAAATTCGTTAAAGGTCAATGAT |
| | | AAAGATGGATTTATGGATACTTTATTGAATAGTTATTCCTATTT |
| | | ATCTATGCCCATTCCAGATGTATTCATAAAAATGTTTTCGAATA |
| | | ATGAAGCTTTTAAATCAGTAGGTTATGCATTTATGCTTGGAGTA |
| | | GGCGGAGAGAAAACTAAAAAAGAAGACGGGGGAAACACAGA |
| | | TGAAAAATAAAGGACTAGCAATGACAATTATTTTCCAAGCAGA |
| | | AAGTGCCAACTACGGGGAGTCTCTTGGTAATATTTCTTCACTTA |
| | | AAAAGATTTCTAGAAATAACGGTGACCAATATACATATATTTC |
| | | TCGCCAAGCAATTCGCTACAACTTAATGGATCAAATTGGAGAG |
| | | AAAGAAGCTCCTGTAAAGGCGGAAGGTGGCGGCGATAAGAAA |
| | | GTTATTCAATTCTTGAGTGAAGCGACTATTACAGATTTCCCAGA |
| | | ACTTGATTTCTTTGGTTACCTTAAAACAGAAAAAGGAAGCGGT |
| | | GGGCAAAAACGTTCTGCAAAAGTCCGTTTATCTAATGCCATTT |
| | | CGCTTGAAACATTTAAAGGTGATTTGGATTTCCTAACGAATAA |
| | | AGGTCAAGCTGATAAATTAAATGAAAACATGAATATCGCACA |
| | | AGCTGAAATTCATAAATCTTATTATCGTTATACGATTACGATTG |
| | | ATTTAGATCAAATTGGTATTGACGGTGAAATTGCACTTGATAA |
| | | TAAAGAAAAAGCTCGTCGTGTAAAAAAATTGATGGATACAGT |
| | | AGCATTCTTGTACCGTGACATTCGCGGGCGCCGAGAAGATTTA |
| | | AAACCACTTTTCGTCATTGGCGGAGTTTATGACGTGAAAAATC |
| | | CTGTTTTCCAAAATATTGTAGATGTAGCAGATAACAATATTGTT |
| | | ATTAAAAATATTAAAGATTTACTTACGTATGAAGATATCAAAG |
| | | AAAATACGAGAGTGGGTATTATTGACGGGCAATTTGCTAATTC |
| | | TGATGAAGTGAAAGTCGAGCTAAAAGCAGAATCTGTGCCGGC |
| | | ATTCTTTAACGATATTAAAGGAAAGATTGATGCTTATTATGAA |
| | | AGCAATTAGAGTAAGACTTTGGCAAGACTTGGTGAATTTTAAG |
| | | AAACCAACTAGTTTTCAATTAAAAGAAACGTATCCTTTGCCGC |
| | | CTTATTCAACTGTAATTGGGATGGTTCATACGCTTTGTGGTTTT |
| | | ACAAGCTATCATGAAATGAAAATTAGTATTCAAGGGAAGTACT |
| | | TTTCGAAGGTGAACGATTTAGCGACGAGGTATGAGTTTAAAAA |
| | | TGGGATGACTTATGATGCATCACGACACCAAATTAAAGTAGAT |
| | | AAGTATGGTGTTAGTCGTGGTGTATCAACGGTTGAGCTTCTTGT |
| | | GGATTTAGAACTACTGCTGCATATCATTCCGGAAGACCCATCA |
| | | CTCGTACCAATCATCGAAAAAGCATTTAGAGAGCCTATTGAGT |
| | | TCCCATCGCTAGGTCGTCGCGAGGATATTGCGACTATTCAAAA |
| | | GGTGGAAGTIGTGGATGTGGAGAAACGGCAACCTAAAAAAAG |
| | | CACAGAAATATCAGATGGTTACAATGCCTATGTGCCAATCTCG |
| | | CTCACTGAAAATAATTCTGTTCATTTCAAATCACATGAAAGCTC |
| | | AGTCGGTCGTGATAAGTIGTTAGGTACAAGATATTTGCTAACA |
| | | GAAAAATATGAAAGAGTTAACCACGGAACAGAAAAAGCACCT |
| | | AAGTTTTTCCGTAAGTGGCGAAAGAAAGATGTTATTTACTCAA |
| | | GTAGAATTTTTGTCTCTAAGAAAGATGTTTTCTTTTTTGATGAA |
| | | GATGATTACTTAGTCTTTATTGAAGAAGAGGAGTAATAATAAC |
| | | AAACAATTACCCCTTGAGTGTATAGTGTTCAAGGGGATTTTGT |
| | | ATAAGGAGCGGCTTGAGATGCATAAATATTTAGCGAAATCTAA |
| | | TCCAGAAGAAACAATTCAAGAACATACGGATAATTTATTAAAA |
| | | AACTATCAAAGACTAAAAAAACTATACCCAGCTGTGGAAATA |
| | | GATTGGTATTTACTGGAATTAGTTTGTTTACTACATGATTTAGG |
| | | GAAAATGAATCTATTATTCCAAACGAAATTAGGTAATGCTTCT |
| | | GGTAAAGGTAAAGAAATTCCGCATGGTTATTTGTCTGTTGCAT |
| | | TTGTTCCAGATAGTAAACTTGAAGAAATGGGATACTCGGAGGA |
| | | TGAAATTAAAGTTGTTTATCAGGCGATTGCGCGACATCATGAA |
| | | AGGAAATATGATTTTTCTGATTCAGAGTGGAAAAAAGAAATAA |
| | | TCAAATTAGAAGAGCAGTGGGCGGGTTTTATATACAGTCAGCT |
| | | AGGAGGAAATGCGGAATATAGTAGTGAGGTAGAAATGTTGTA |
| | | CTTCTACCCAGGCTCCCGAATTTTCGAAGGTAACTCCGCAGCG |
| | | GACGCAGAAACATTCAAAAAATACGTTCTACTAAAAGGCTTAC |
| | | TCAATCGAATTGACTTTGCAGCGAGTGGGGGAATTGATGTAGA |
| | | ACTTGAAAATGACTTCTTGATGGATTCGATGGAAGTGCAACTA |
| | | GCCCATTTTAAAGCAGAAAATCCAGAAGCTGATTGGAACGCTT |
| | | TGCAAAAATATATGATGCAGCACCAAAATGAAAATATTGTCGT |
| | | TATTGCTGAAACTGGTATGGGGAAAACGGAGGCAGGGCTGCT |
| | | ATGGCTTGGGAATAATAAGGGTTTTTTCACGCTACCATTGCGG |
| | | GCAGCAATCAATGCAATTTATGAGCGAATCACAAAAAATATTG |
| | | TTACGACCAATCAAGCCGAGCGAGTGGGTTTACTTCATTCGGA |
| | | AACATATAGTCAGTATTTGCTGCATGAAGGAAATGCGGAGATG |
| | | GATATTGATGAATATTATACGAGGACGAGGCAAATGTCTTTAC |
| | | CTATAACTATTTGCACGCTCGATCAATTGTTTGATTTTGTCTTTC |
| | | GTTATGCAGGTTTTGAACATAAATTAGCCACTTTATCTTATTCC |
| | | AAAGTGATTATTGATGAAATCCAGATGTACTCACCCGACTTAC |
| | | TGGCTTACTTGATTTTAGGGTTATCTTATATTGATAAATTTGGT |
| | | GGGAAGTTCTGCATTATGACAGCGACTTTACCGGGAATAGTGA |
| | | CAGATTTACTTTTAGAAAATGGAGTGGATTTTGTCCAACCAGA |
| | | AGAGAAATTTGTATCGTCGCGAATTCGCCACAGCATGGAGATG |
| | | GTTCATACAGAGATTGAAAGCGAATTTATTAAACCATTTTTTA |
| | | ACAATAATCGAATTTTAGTTATTTGTAACACTATTAGTAAAGC |
| | | GAAAAAAATCTATTCGGAGTTAAAAGAGCATTTTCAAGGTGAA |
| | | GAAATTCATCTCATTCACAGTCAATTTATTAAAAGAGACCGCT |
| | | CAGCTAAAGAAAAAGCAATTTTTAAAGATGGGCAAAAAGATA |
| | | GTACGAAAAAATGTATTTGGGTAGCAACGCAAGTCGTAGAGG |
| | | CTTCATTGGATATTGATTTTGATTTACTATTCACGGAGCTATCA |
| | | GATGTGAATGGGCTGTTTCAACGGATGGGCAGATGCTATCGAA |
| | | ACCGGGCGCTGGATGTGGATACGAATGTCTATGTTTTTGATGG |
| | | TGGAGCGAAAGTTTGCTCAGGAATTGGAACTTTTATCGATAAG |
| | | TTGATATTTGTAAATTCTAAGACGATTTTAAATGAGCATGCTGG |
| | | CGTTTTGACAGAAGAGAAGAAAATGGAGCTAGTGGAGCGGGT |
| | | TTATTCAACAGAGGCTTTACGTGGGAGTGAATTTTATAACGAT |
| | | TTGAGAAAGACGATAAACTATGTGAAAGCTTTTGATAGTTATG |
| | | AATTGGATAAAGCGGATGTTCGTAGGAGATTTAGAAACATTAA |
| | | TTCGGTTTCTGTTATCCCAGAAGAAGTTTGGCAAGATAATGCG |
| | | GAAGAAATCAATTCCTATTTTGTTACTCTTCGTAAAACCTCTAA |
| | | GGAAATATCAACAAAAGAGAAAATCATTGCAAGAACCAATTT |
| | | AGCAGAATTTATGGTGAGTATTCCAGACTATCTTTATAAAAAA |
| | | GGTGAGAGCGTTGTACGCGAAATAAACCGTTATGAGAGTGTTA |
| | | TTGAGTTTAAATGTGGTTATTCGAGTGATGTAGGGGTTTTTATG |
| | | AGGTGA |
| 23 | LMIB | ATGAGATTAAAGATAAATTGTGATTTTGATTCTAAAATAATTTC |
| | (optimized) | AAAAGATTTTCAAAGTAAAGTTGTTAGTCTGTTCAAAGCGGGC |
| | | ATTATGAAATCGAGTCCAGAGAGATATGAGAATTTATTTGGCG |
| | | GGAATAAGCATAAGCAATATACATTTTCAGTGTACCTTCCCAA |
| | | ACCTCAAAATAAAGGTGCGGAAATTCAGTTAAATGAAGCAAA |
| | | TTGTATTATTAATTTTTCAACGGGAGATGCGGAAACGGGAATC |
| | | ATTTTTTATAATGCATTGATGAGTTTGAGAGGTAGCAAAGTTTT |
| | | ATTTGGAGCTGGGAATCATATTACGGTAAAAAACATTCAAATA |
| | | GTCCCGGAGAAAAAAATTATCGGCAAACGAACAATTTTGAGA |
| | | ACGCTTTCACCTGTGGTTAGTAGAGACCATAATAGAGAAACAT |
| | | TCAAGAATTGGTTTTACAGTTTTGAGGATGAGGAATTTGAGCC |
| | | AACTTTGAAAAGAAACATGTTGCCCTATTTAATGGATGCATTT |
| | | GGTGAGCAAGCTCGTTATGATTTGGAGAAATTGAAAATAACGC |
| | | CAATTTCGATGAAGAAAGTAGTCGTTTATTGCCACGAGATACA |
| | | TATTGAAAGCTCAGTCGGGATTTTTGAATTGGAGGCTGAGGCT |
| | | TATTTGCAGAAGTATTTGGTGGAGAATGGTATTGGGACTATGA |
| | | CAGGTTCTGGATTTGGCATGATAGAGCAGTTTTAGACTGGAGG |
| | | TGGAAGAATGCGGACAGAAATAGAAGTAAGGGCAAATGATTG |
| | | GTTAATAAATGCTGGTTTAACTGGTTTTCTAAATATCGTTGGAA |
| | | AAGAAAATGTAAGAATTGATGGACAAGCTATTTATTTTACAGC |
| | | AGATTTATTAGAGGACTTTGAAACGAAATACTTTAATTTCTTTA |
| | | TTCAAGAATATAAAGAAACGTTATCGTGGTATAAAATCGTTTC |
| | | TTATAAAGAGACAATGGAACAGTTTAGAATAGATGAATTTGCT |
| | | TCTTTTGACGAGGTTGCATTGGGAAATTTAAATAAATATATGA |
| | | AAGATGTAGTAAAGTTTTATTTAAAAAAAGCTAATTATATCAA |
| | | GGTGTTTCCGCTAATTGACCCTAGTGCTAATGTAACAAAATGG |
| | | TTGGAAAAATTAAATACAATTAATTTAACTAAAAAGCAAAAAT |
| | | GGGATGAAGTAAAAGCCGATATTATTGAAGAAGTAAAGCAAA |
| | | CATATACTCAACTAGACCTGATTATTGATTTTTGTGCAAGTGAA |
| | | AAGGGGCTTCAATATCTAGGAGCAAAAAATCTGATATATTCTG |
| | | TTATAAATAAAGGTTGGTCTGGTGTATCTTTCCTTCTTAAACAA |
| | | ACAAAATTTATTGACCCCTATTTAGATTACAAAACTAGTTTTTT |
| | | GGACCCAGTAATAGATTATTTAGACACCGATTTATCTAAAGCA |
| | | AAATATAATTGTTTCAATTGTAATCAACCAATTAAGAATTTGA |
| | | AACTAGACCTCAGTTTTATGAACGATGTTGGGTTTGATACTGC |
| | | AAGAAAAACAGCTCACGTATGGGATTTTAACAATGATGTTGCG |
| | | ACTTGCCCGATTTGTCGTTTGATTTATTCATGTGTTCCAGCTGG |
| | | ATTTACGTATGTGTACGGAGAAGGTATGTTTGTCAACGATTCA |
| | | TATGGAGTAGAGGAATTACACCGTGTTAATGAACGTATGCGGA |
| | | ATTCTATCCTGCGCTTTAACAAAGATGGAATTAATTCAACTAAT |
| | | ACTTACCGAGCGCTTGTGGAGTCTATTACGATGGAGCATGAAA |
| | | ATAATCGACGCTATGAACTTGCAGACATTCAGTTAGTTAGATA |
| | | CGAAAATGAGCATTATCGTTTTAATTTACTTTCTAAAAAAATGT |
| | | TGCATATTGTAAATGATTCAAAGGGAATATTGAAAAGTTTAAT |
| | | TCGCTGTGGTTATAAAGAAGGTAATTTGAATGTTAATTTGTAC |
| | | AAAGAAGTTATTCAGCATTTAATGAACAATGAAAATTTATTTA |
| | | CGCTTATTCATAAATTAATCTACTATAAACAAACTAGTGTAAA |
| | | TGGTTTGTATTACAATATGGGACACGTTTCAGGGATTTTAGATA |
| | | TAAATACAAAATTTTTGAAGGAGATGGAAGTGATGACAAATAT |
| | | TTCTCAAAATCAATTATGGTTCGTTCAAAATTGTGGAAAGGAA |
| | | TTTAAGGAAGGTTATGTTAAGAAGAAGTCAGAAAATAAAATTT |
| | | CGGGAATAACATACAAGTTATTAAATTCGTTAAAGGTCAATGA |
| | | TAAAGATGGATTTATGGATACTTTATTGAATAGTTATTCCTATT |
| | | TATCTATGCCCATTCCAGATGTATTCATAAAAATGTTTTCGAAT |
| | | AATGAAGCTTTTAAATCAGTAGGTTATGCATTTATGCTTGGAGT |
| | | AGGCGGAGAGAAAACTAAAAAAGAAGACGGGGGAAACACAG |
| | | ATGAAAAATAAAGGACTAGCAATGACAATTATTTTCCAAGCAG |
| | | AAAGTGCCAACTACGGGGAGTCTCTAGGTAATATTTCTTCACT |
| | | TAAAAAGATTTCTAGAAATAATGGAGACCAATATACATATATT |
| | | AGCAGACAAGCAATTAGATACAACCTCATGGACCAAATTGGA |
| | | GAAAAAGAAGCTCCTGTTAAAGCAGAAGGTGGTGGAGATAAG |
| | | AAAGTTATTCAATTCTTAAGTGAAGCTACTATTACAGATTTCCC |
| | | TGAACTTGATTTCTTTGGTTACCTTAAAACAGAAAAAGGAAGT |
| | | GGCGGACAAAAGAGAAGTGCAAAAGTACGTTTATCTAATGCT |
| | | ATTTCTCTTGAAACATTTAAAGGTGATTTGGATTTCCTAACAAA |
| | | TAAAGGTCAAGCAGATAAATTAAATGAAAATATGAATATTGCA |
| | | CAAGCAGAAATTCATAAATCTTATTATCGTTATACGATTACGA |
| | | TTGATTTAGACCAAATTGGTATTGACGGTGAAATTGCACTTGA |
| | | TAATAAAGAAAAAGCTCGTCGTGTAAAAAAATTGATGGATAC |
| | | AGTAGCATTCTTGTACCGTGACATTCGCGGGCGCCGAGAAGAT |
| | | TTAAAACCACTTTTCGTCATTGGCGGAGTTTATGACGTGAAAA |
| | | ATCCTGTTTTCCAAAATATTGTAGATGTAGCAGATAACAATATT |
| | | GTTATTAAAAATATTAAAGATTTACTTACGTATGAAGATATCA |
| | | AAGAAAATACGAGAGTGGGTATTATTGACGGGCAATTTGCTAA |
| | | TTCTGATGAAGTGAAAGTCGAGCTAAAAGCAGAATCTGTGCCG |
| | | GCATTCTTTAACGATATTAAAGGAAAGATTGATGCTTATTATG |
| | | AAAGCAATTAGAGTAAGATTATGGCAAGATTTGGTAAATTTTA |
| | | AGAAACCAACTAGTTTTCAATTAAAAGAAACATATCCTTTACC |
| | | TCCTTATTCAACTGTAATTGGGATGGTACATACACTATGTGGAT |
| | | TTACAAGCTATCATGAAATGAAAATTAGTATACAAGGTAAATA |
| | | TTTTTCTAAAGTAAATGATTTAGCTACAAGATATGAATTTAAA |
| | | AATGGTATGACATATGATGCTTCTCGTCATCAAATTAAAGTAG |
| | | ATAAGTATGGTGTAAGTAGAGGTGTATCAACAGTAGAATTATT |
| | | AGTAGATTTAGAATTATTATTACATATTATTCCTGAAGATCCAT |
| | | CATTAGTACCAATCATAGAAAAAGCTTTTAGAGAGCCTATTGA |
| | | GTTTCCTAGTCTAGGTAGAAGAGAGGATATTGCTACAATTCAA |
| | | AAGGTAGAAGTTGTAGATGTAGAAAAAAGACAACCTAAAAAA |
| | | AGTACAGAAATATCAGATGGTTACAATGCTTATGTACCAATCT |
| | | CTTTAACTGAAAATAATTCTGTTCATTTCAAATCTCATGAATCA |
| | | TCAGTAGGTAGAGATAAGTIGTTAGGTACAAGATATTTGCTAA |
| | | CAGAAAAATATGAAAGAGTTAATCATGGAACAGAAAAAGCAC |
| | | CTAAGTTTTTCAGAAAATGGAGAAAGAAAGATGTTATTTACTC |
| | | AAGTAGAATTTTTGTCTCTAAGAAAGATGTTTTCTTTTTTGATG |
| | | AAGATGATTACTTAGTCTTTATTGAAGAAGAGGAGTAATAATA |
| | | ACAAACAATTACCCCTTGAGTGTATAGTGTTCAAGGGGATTTT |
| | | GTATAAGGAGCGGCTTGAGATGCATAAATATTTAGCTAAATCT |
| | | AATCCTGAAGAAACAATTCAAGAGCATACCGATAATTTATTAA |
| | | AAAACTATCAAAGACTAAAAAAACTATATCCTGCTGTAGAAAT |
| | | AGATTGGTATTTATTAGAATTAGTTTGTTTACTACATGATTTAG |
| | | GGAAAATGAATCTATTATTCCAAACTAAATTAGGTAATGCTTC |
| | | AGGTAAAGGTAAAGAAATACCACATGGTTATTTGTCTGTTGCA |
| | | TTTGTTCCTGATAGTAAACTTGAAGAAATGGGATATTCTGAAG |
| | | ATGAAATTAAAGTTGTTTATCAAGCTATAGCTAGGCATCATGA |
| | | AAGGAAATATGATTTTTCTGATTCAGAATGGAAAAAAGAAATA |
| | | ATCAAATTAGAAGAACAATGGGCAGGTTTTATATATTCTCAAT |
| | | TAGGTGGAAATGCAGAATATAGTAGTGAGGTAGAAATGTTATA |
| | | TTTCTATCCTGGTTCAAGAATTTTTGAAGGTAACTCTGCAGCTG |
| | | ATGCAGAAACATTCAAAAAATACGTTTTATTAAAAGGTTTATT |
| | | AAATAGAATTGACTTTGCAGCTAGTGGTGGTATTGATGTAGAA |
| | | CTTGAAAATGATTTCTTAATGGATAGTATGGAAGTACAATTAG |
| | | CACATTTTAAAGCAGAAAATCCTGAAGCGGATTGGAACGCATT |
| | | ACAAAAATATATGATGCAACATCAAAATGAAAATATTGTAGTT |
| | | ATTGCAGAAACAGGTATGGGTAAAACAGAAGCAGGTTTATTAT |
| | | GGTTAGGAAATAATAAAGGTTTTTTCACATTACCTTTACGTGCT |
| | | GCAATTAATGCAATTTATGAAAGAATTACAAAAAATATTGTAA |
| | | CAACAAATCAAGCAGAAAGAGTAGGTCTACTACATTCAGAAA |
| | | CATATTCTCAATATTTATTACATGAAGGTAATGCTGAAATGGA |
| | | TATTGATGAATATTATACAAGAACAAGACAAATGTCTTTACCT |
| | | ATAACAATATGTACATTAGACCAATTATTTGATTTTGTATTTAG |
| | | ATATGCAGGTTTTGAACATAAATTAGCTACATTATCATATTCTA |
| | | AAGTAATTATTGATGAAATTCAAATGTATTCACCCGATTTATTA |
| | | GCTTACTTGATTTTAGGGTTATCTTATATTGATAAATTTGGTGG |
| | | AAAGTTCTGCATTATGACAGCAACATTACCTGGTATAGTAACA |
| | | GATTTACTTTTAGAAAATGGAGTTGATTTTGTACAACCTGAAG |
| | | AGAAATTTGTATCTTCTAGAATTAGACATAGCATGGAGATGGT |
| | | TCATACAGAAATTGAATCAGAATTTATTAAACCATTTTTTAACA |
| | | ATAATAGAATTTTAGTTATTTGTAACACTATTAGTAAAGCTAA |
| | | AAAAATTTATTCTGAGTTAAAAGAGCATTTTCAAGGTGAAGAA |
| | | ATACATTTAATTCACAGTCAATTTATTAAAAGAGATAGAAGTG |
| | | CTAAAGAAAAAGCAATATTTAAAGATGGTCAAAAAGATAGTA |
| | | CAAAAAAATGTATTTGGGTAGCAACACAAGTAGTAGAAGCTTC |
| | | ATTGGATATTGATTTTGATTTACTATTCACGGAGCTATCAGATG |
| | | TGAATGGGCTGTTTCAACGGATGGGCAGATGCTATCGAAACCG |
| | | GGCGCTGGATGTGGATACGAATGTCTATGTTTTTGATGGTGGA |
| | | GCGAAAGTTTGCTCAGGAATTGGAACTTTTATCGATAAGTTGA |
| | | TATTTGTAAATTCTAAGACGATTTTAAATGAGCATGCTGGCGTT |
| | | TTGACAGAAGAGAAGAAAATGGAGCTAGTGGAGCGGGTTTAT |
| | | TCAACAGAGGCTTTACGTGGGAGTGAATTTTATAACGATTTGA |
| | | GAAAGACGATAAACTATGTGAAAGCTTTTGATAGTTATGAATT |
| | | GGATAAAGCGGATGTTCGTAGGAGATTTAGAAACATTAATTCG |
| | | GTTTCTGTTATCCCAGAAGAAGTTTGGCAAGATAATGCGGAAG |
| | | AAATCAATTCCTATTTTGTTACTCTTCGTAAAACCTCTAAGGAA |
| | | ATATCAACAAAAGAGAAAATCATTGCAAGAACCAATTTAGCA |
| | | GAATTTATGGTGAGTATTCCAGACTATCTTTATAAAAAAGGTG |
| | | AGAGCGTTGTACGCGAAATAAACCGTTATGAGAGTGTTATTGA |
| | | GTTTAAATGTGGTTATTCGAGTGATGTAGGGGTTTTTATGAGGT |
| | | GA |
| 25 | Cas6 | MRLKINCDFDSKIISKDFQSKVVSLFKAGIMKSSPERYENLFGGNK |
| | | HKQYTFSVYLPKPQNKGAEIQLNEANCIINFSTGDAETGIIFYNAL |
| | | MSLRGSKVLFGAGNHITVKNIQIVPEKKIIGKRTILRTLSPVVSRDH |
| | | NRETFKNWFYSFEDEEFEPTLKRNMLPYLMDAFGEQARYDLEKL |
| | | KITPISMKKVVVYCHEIHIESSVGIFELEAEAYLQKYLVENGIGTM |
| | | TGSGFGMIEQF* |
| 26 | Cas7 | MKNKGLAMTIIFQAESANYGESLGNISSLKKISRNNGDQYTYISRQ |
| | | AIRYNLMDQIGEKEAPVKAEGGGDKKVIQFLSEATITDFPELDFFG |
| | | YLKTEKGSGGQKRSAKVRLSNAISLETFKGDLDFLTNKGQADKL |
| | | NENMNIAQAEIHKSYYRYTITIDLDQIGIDGEIALDNKEKARRVKK |
| | | LMDTVAFLYRDIRGRREDLKPLFVIGGVYDVKNPVFQNIVDVAD |
| | | NNIVIKNIKDLLTYEDIKENTRVGIIDGQFANSDEVKVELKAESVP |
| | | AFFNDIKGKIDAYYESN* |
| 27 | Cas5 | MKAIRVRLWQDLVNFKKPTSFQLKETYPLPPYSTVIGMVHTLCGF |
| | | TSYHEMKISIQGKYFSKVNDLATRYEFKNGMTYDASRHQIKVDK |
| | | YGVSRGVSTVELLVDLELLLHIIPEDPSLVPIIEKAFREPIEFPSLGR |
| | | REDIATIQKVEVVDVEKRQPKKSTEISDGYNAYVPISLTENNSVHF |
| | | KSHESSVGRDKLLGTRYLLTEKYERVNHGTEKAPKFFRKWRKKD |
| | | VIYSSRIFVSKKDVFFFDEDDYLVFIEEEE* |
| 28 | Cas3 | MHKYLAKSNPEETIQEHTDNLLKNYQRLKKLYPAVEIDWYLLEL |
| | | VCLLHDLGKMNLLFQTKLGNASGKGKEIPHGYLSVAFVPDSKLE |
| | | EMGYSEDEIKVVYQAIARHHERKYDFSDSEWKKEIIKLEEQWAGF |
| | | IYSQLGGNAEYSSEVEMLYFYPGSRIFEGNSAADAETFKKYVLLK |
| | | GLLNRIDFAASGGIDVELENDFLMDSMEVQLAHFKAENPEADWN |
| | | ALQKYMMQHQNENIVVIAETGMGKTEAGLLWLGNNKGFFTLPL |
| | | RAAINAIYERITKNIVTTNQAERVGLLHSETYSQYLLHEGNAEMDI |
| | | DEYYTRTRQMSLPITICTLDQLFDFVFRYAGFEHKLATLSYSKVIID |
| | | EIQMYSPDLLAYLILGLSYIDKFGGKFCIMTATLPGIVTDLLLENG |
| | | VDFVQPEEKFVSSRIRHSMEMVHTEIESEFIKPFFNNNRILVICNTIS |
| | | KAKKIYSELKEHFQGEEIHLIHSQFIKRDRSAKEKAIFKDGQKDST |
| | | KKCIWVATQVVEASLDIDFDLLFTELSDVNGLFQRMGRCYRNRA |
| | | LDVDTNVYVFDGGAKVCSGIGTFIDKLIFVNSKTILNEHAGVLTEE |
| | | KKMELVERVYSTEALRGSEFYNDLRKTINYVKAFDSYELDKADV |
| | | RRRFRNINSVSVIPEEVWQDNAEEINSYFVTLRKTSKEISTKEKIIA |
| | | RTNLAEFMVSIPDYLYKKGESVVREINRYESVIEFKCGYSSDVGVF |
| | | MR* |
| 29 | Cas8 | MRTEIEVRANDWLINAGLTGFLNIVGKENVRIDGQAIYFTADLLE |
| | | DFETKYFNFFIQEYKETLSWYKIVSYKETMEQFRIDEFASFDEVAL |
| | | GNLNKYMKDVVKFYLKKANYIKVFPLIDPSANVTKWLEKLNTIN |
| | | LTKKQKWDEVKADIIEEVKQTYTQLDLIIDFCASEKGLQYLGAKN |
| | | LIYSVINKGWSGVSFLLKQTKFIDPYLDYKTSFLDPVIDYLDTDLS |
| | | KAKYNCFNCNQPIKNLKLDLSFMNDVGFDTARKTAHVWDENND |
| | | VATCPICRLIYSCVPAGFTYVYGEGMFVNDSYGVEELHRVNERM |
| | | RNSILRENKDGINSTNTYRALVESITMEHENNRRYELADIQLVRYE |
| | | NEHYRFNLLSKKMLHIVNDSKGILKSLIRCGYKEGNLNVNLYKEV |
| | | IQHLMNNENLFTLIHKLIYYKQTSVNGLYYNMGHVSGILDINTKF |
| | | LKEMEVMTNISQNQLWFVQNCGKEFKEGYVKKKSENKISGITYK |
| | | LLNSLKVNDKDGFMDTLLNSYSYLSMPIPDVFIKMFSNNEAFKSV |
| | | GYAFMLGVGGEKTKKEDGGNTDEK* |
| 33 | LMIB (natural) | ATGAGATTAAAGATAAATTGTGATTTTGATTCTAAAATAATTTC |
| | Cas6 | AAAAGATTTTCAAAGTAAAGTTGTTAGTCTGTTCAAAGCGGGC |
| | | ATTATGAAATCGAGTCCAGAGAGATATGAGAATTTATTTGGCG |
| | | GGAATAAGCATAAGCAATATACATTTTCAGTGTACCTTCCCAA |
| | | ACCTCAAAATAAAGGTGCGGAAATTCAGTTAAATGAAGCAAA |
| | | TTGTATTATTAATTTTTCAACGGGAGATGCGGAAACGGGAATC |
| | | ATTTTTTATAATGCATTGATGAGTTTGAGAGGTAGCAAAGTTTT |
| | | ATTTGGAGCTGGGAATCATATTACGGTAAAAAACATTCAAATA |
| | | GTCCCGGAGAAAAAAATTATCGGCAAACGAACAATTTTGAGA |
| | | ACGCTTTCACCTGTGGTTAGTAGAGATCATAATAGAGAAACAT |
| | | TCAAGAATTGGTTTTACAGTTTTGAGGATGAGGAATTTGAGCC |
| | | AACTTTGAAAAGAAACATGTTGCCCTATTTAATGGATGCATTT |
| | | GGTGAGCAAGCTCGTTATGATTTGGAGAAATTGAAAATAACGC |
| | | CAATTTCGATGAAGAAAGTAGTCGTTTATTGCCACGAGATACA |
| | | TATTGAAAGCTCAGTCGGGATTTTTGAATTGGAGGCTGAGGCT |
| | | TATTTGCAGAAGTATTTGGTGGAGAATGGTATTGGGACTATGA |
| | | CAGGATCTGGATTTGGCATGATAGAGCAGTTTTAG |
| 34 | LMIB (natural) | ATGCGGACAGAAATAGAAGTAAGGGCAAATGATTGGTTAATA |
| | Cas8 | AATGCTGGTTTAACTGGTTTTCTAAATATCGTTGGAAAAGAAA |
| | | ATGTAAGAATTGATGGACAAGCTATTTATTTTACAGCAGATTT |
| | | ATTAGAGGACTTTGAAACGAAATACTTTAATTTCTTTATTCAAG |
| | | AATATAAAGAAACGTTATCGTGGTATAAAATCGTTTCTTATAA |
| | | AGAGACAATGGAACAGTTTAGAATAGATGAATTTGCTTCTTTT |
| | | GACGAGGTTGCATTGGGAAATTTAAATAAATATATGAAAGATG |
| | | TAGTAAAGTTTTATTTAAAAAAAGCTAATTATATCAAGGTGTTT |
| | | CCGCTAATTGATCCTAGTGCTAATGTAACAAAATGGTTGGAAA |
| | | AATTAAATACAATTAATTTAACTAAAAAGCAAAAATGGGATGA |
| | | AGTAAAAGCCGATATTATTGAAGAAGTAAAGCAAACATATACT |
| | | CAACTAGATCTGATTATTGATTTTTGTGCAAGTGAAAAGGGGC |
| | | TTCAATATCTAGGAGCAAAAAATCTGATATATTCTGTTATAAA |
| | | TAAAGGTTGGTCTGGTGTATCTTTCCTTCTTAAACAAACAAAAT |
| | | TTATTGATCCCTATTTAGATTACAAAACTAGTTTTTTGGACCCA |
| | | GTAATAGATTATTTAGACACCGATTTATCTAAAGCAAAATATA |
| | | ATTGTTTCAATTGTAATCAACCAATTAAGAATTTGAAACTAGA |
| | | TCTCAGTTTTATGAACGATGTTGGGTTTGATACTGCAAGAAAA |
| | | ACAGCTCACGTATGGGATTTTAACAATGATGTTGCGACTTGCC |
| | | CGATTTGTCGTTTGATTTATTCATGTGTTCCAGCTGGATTTACG |
| | | TATGTGTACGGAGAAGGTATGTTTGTCAACGATTCATATGGAG |
| | | TAGAGGAATTACACCGTGTTAATGAACGTATGCGGAATTCTAT |
| | | CCTGCGCTTTAACAAAGATGGAATTAATTCAACTAATACTTAC |
| | | CGAGCGCTTGTGGAGTCTATTACGATGGAGCATGAAAATAATC |
| | | GACGCTATGAACTTGCAGACATTCAGTTAGTTAGATACGAAAA |
| | | TGAGCATTATCGTTTTAATTTACTTTCTAAAAAAATGTTGCATA |
| | | TTGTAAATGATTCAAAGGGAATATTGAAAAGTTTAATTCGCTG |
| | | TGGTTATAAAGAAGGTAATTTGAATGTTAATTTGTACAAAGAA |
| | | GTTATTCAGCATTTAATGAACAATGAAAATTTATTTACGCTTAT |
| | | TCATAAATTAATCTACTATAAACAAACTAGTGTAAATGGTTTG |
| | | TATTACAATATGGGACACGTTTCAGGGATTTTAGATATAAATA |
| | | CAAAATTTTTGAAGGAGATGGAAGTGATGACAAATATTTCTCA |
| | | AAATCAATTATGGTTCGTTCAAAATTGTGGAAAGGAATTTAAG |
| | | GAAGGTTATGTTAAGAAGAAGTCAGAAAATAAAATTTCGGGA |
| | | ATAACATACAAGTTATTAAATTCGTTAAAGGTCAATGATAAAG |
| | | ATGGATTTATGGATACTTTATTGAATAGTTATTCCTATTTATCT |
| | | ATGCCCATTCCAGATGTATTCATAAAAATGTTTTCGAATAATG |
| | | AAGCTTTTAAATCAGTAGGTTATGCATTTATGCTTGGAGTAGG |
| | | CGGAGAGAAAACTAAAAAAGAAGACGGGGGAAACACAGATG |
| | | AAAAATAA |
| 35 | LMIB (natural) | ATGAAAAATAAAGGACTAGCAATGACAATTATTTTCCAAGCAG |
| | Cas7 | AAAGTGCCAACTACGGGGAGTCTCTTGGTAATATTTCTTCACTT |
| | | AAAAAGATTTCTAGAAATAACGGTGACCAATATACATATATTT |
| | | CTCGCCAAGCAATTCGCTACAACTTAATGGATCAAATTGGAGA |
| | | GAAAGAAGCTCCTGTAAAGGCGGAAGGTGGCGGCGATAAGAA |
| | | AGTTATTCAATTCTTGAGTGAAGCGACTATTACAGATTTCCCAG |
| | | AACTTGATTTCTTTGGTTACCTTAAAACAGAAAAAGGAAGCGG |
| | | TGGGCAAAAACGTTCTGCAAAAGTCCGTTTATCTAATGCCATT |
| | | TCGCTTGAAACATTTAAAGGTGATTTGGATTTCCTAACGAATA |
| | | AAGGTCAAGCTGATAAATTAAATGAAAACATGAATATCGCAC |
| | | AAGCTGAAATTCATAAATCTTATTATCGTTATACGATTACGATT |
| | | GATTTAGATCAAATTGGTATTGACGGTGAAATTGCACTTGATA |
| | | ATAAAGAAAAAGCTCGTCGTGTAAAAAAATTGATGGATACAG |
| | | TAGCATTCTTGTACCGTGACATTCGCGGGCGCCGAGAAGATTT |
| | | AAAACCACTTTTCGTCATTGGCGGAGTTTATGACGTGAAAAAT |
| | | CCTGTTTTCCAAAATATTGTAGATGTAGCAGATAACAATATTGT |
| | | TATTAAAAATATTAAAGATTTACTTACGTATGAAGATATCAAA |
| | | GAAAATACGAGAGTGGGTATTATTGACGGGCAATTTGCTAATT |
| | | CTGATGAAGTGAAAGTCGAGCTAAAAGCAGAATCTGTGCCGG |
| | | CATTCTTTAACGATATTAAAGGAAAGATTGATGCTTATTATGA |
| | | AAGCAATTAG |
| 36 | LMIB (natural) | ATGAAAGCAATTAGAGTAAGACTTTGGCAAGACTTGGTGAATT |
| | Cas5 | TTAAGAAACCAACTAGTTTTCAATTAAAAGAAACGTATCCTTT |
| | | GCCGCCTTATTCAACTGTAATTGGGATGGTTCATACGCTTTGTG |
| | | GTTTTACAAGCTATCATGAAATGAAAATTAGTATTCAAGGGAA |
| | | GTACTTTTCGAAGGTGAACGATTTAGCGACGAGGTATGAGTTT |
| | | AAAAATGGGATGACTTATGATGCATCACGACACCAAATTAAAG |
| | | TAGATAAGTATGGTGTTAGTCGTGGTGTATCAACGGTTGAGCT |
| | | TCTTGTGGATTTAGAACTACTGCTGCATATCATTCCGGAAGACC |
| | | CATCACTCGTACCAATCATCGAAAAAGCATTTAGAGAGCCTAT |
| | | TGAGTTCCCATCGCTAGGTCGTCGCGAGGATATTGCGACTATT |
| | | CAAAAGGTGGAAGTTGTGGATGTGGAGAAACGGCAACCTAAA |
| | | AAAAGCACAGAAATATCAGATGGTTACAATGCCTATGTGCCAA |
| | | TCTCGCTCACTGAAAATAATTCTGTTCATTTCAAATCACATGAA |
| | | AGCTCAGTCGGTCGTGATAAGTTGTTAGGTACAAGATATTTGC |
| | | TAACAGAAAAATATGAAAGAGTTAACCACGGAACAGAAAAAG |
| | | CACCTAAGTTTTTCCGTAAGTGGCGAAAGAAAGATGTTATTTA |
| | | CTCAAGTAGAATTTTTGTCTCTAAGAAAGATGTTTTCTTTTTTG |
| | | ATGAAGATGATTACTTAGTCTTTATTGAAGAAGAGGAGTAA |
| 37 | LMIB (natural) | ATGCATAAATATTTAGCGAAATCTAATCCAGAAGAAACAATTC |
| | Cas3 | AAGAACATACGGATAATTTATTAAAAAACTATCAAAGACTAAA |
| | | AAAACTATACCCAGCTGTGGAAATAGATTGGTATTTACTGGAA |
| | | TTAGTTTGTTTACTACATGATTTAGGGAAAATGAATCTATTATT |
| | | CCAAACGAAATTAGGTAATGCTTCTGGTAAAGGTAAAGAAATT |
| | | CCGCATGGTTATTTGTCTGTTGCATTTGTTCCAGATAGTAAACT |
| | | TGAAGAAATGGGATACTCGGAGGATGAAATTAAAGTTGTTTAT |
| | | CAGGCGATTGCGCGACATCATGAAAGGAAATATGATTTTTCTG |
| | | ATTCAGAGTGGAAAAAAGAAATAATCAAATTAGAAGAGCAGT |
| | | GGGGGGTTTTATATACAGTCAGCTAGGAGGAAATGCGGAATA |
| | | TAGTAGTGAGGTAGAAATGTTGTACTTCTACCCAGGCTCCCGA |
| | | ATTTTCGAAGGTAACTCCGCAGCGGACGCAGAAACATTCAAAA |
| | | AATACGTTCTACTAAAAGGCTTACTCAATCGAATTGACTTTGC |
| | | AGCGAGTGGGGGAATTGATGTAGAACTTGAAAATGACTTCTTG |
| | | ATGGATTCGATGGAAGTGCAACTAGCCCATTTTAAAGCAGAAA |
| | | ATCCAGAAGCTGATTGGAACGCTTTGCAAAAATATATGATGCA |
| | | GCACCAAAATGAAAATATTGTCGTTATTGCTGAAACTGGTATG |
| | | GGGAAAACGGAGGCAGGGCTGCTATGGCTTGGGAATAATAAG |
| | | GGTTTTTTCACGCTACCATTGCGGGCAGCAATCAATGCAATTTA |
| | | TGAGCGAATCACAAAAAATATTGTTACGACCAATCAAGCCGAG |
| | | CGAGTGGGTTTACTTCATTCGGAAACATATAGTCAGTATTTGCT |
| | | GCATGAAGGAAATGCGGAGATGGATATTGATGAATATTATACG |
| | | AGGACGAGGCAAATGTCTTTACCTATAACTATTTGCACGCTCG |
| | | ATCAATTGTTTGATTTTGTCTTTCGTTATGCAGGTTTTGAACAT |
| | | AAATTAGCCACTTTATCTTATTCCAAAGTGATTATTGATGAAAT |
| | | CCAGATGTACTCACCCGACTTACTGGCTTACTTGATTTTAGGGT |
| | | TATCTTATATTGATAAATTTGGTGGGAAGTTCTGCATTATGACA |
| | | GCGACTTTACCGGGAATAGTGACAGATTTACTTTTAGAAAATG |
| | | GAGTGGATTTTGTCCAACCAGAAGAGAAATTTGTATCGTCGCG |
| | | AATTCGCCACAGCATGGAGATGGTTCATACAGAGATTGAAAGC |
| | | GAATTTATTAAACCATTTTTTAACAATAATCGAATTTTAGTTAT |
| | | TTGTAACACTATTAGTAAAGCGAAAAAAATCTATTCGGAGTTA |
| | | AAAGAGCATTTTCAAGGTGAAGAAATTCATCTCATTCACAGTC |
| | | AATTTATTAAAAGAGACCGCTCAGCTAAAGAAAAAGCAATTTT |
| | | TAAAGATGGGCAAAAAGATAGTACGAAAAAATGTATTTGGGT |
| | | AGCAACGCAAGTCGTAGAGGCTTCATTGGATATTGATTTTGAT |
| | | TTACTATTCACGGAGCTATCAGATGTGAATGGGCTGTTTCAAC |
| | | GGATGGGCAGATGCTATCGAAACCGGGCGCTGGATGTGGATA |
| | | CGAATGTCTATGTTTTTGATGGTGGAGCGAAAGTTTGCTCAGG |
| | | AATTGGAACTTTTATCGATAAGTTGATATTTGTAAATTCTAAGA |
| | | CGATTTTAAATGAGCATGCTGGCGTTTTGACAGAAGAGAAGAA |
| | | AATGGAGCTAGTGGAGCGGGTTTATTCAACAGAGGCTTTACGT |
| | | GGGAGTGAATTTTATAACGATTTGAGAAAGACGATAAACTATG |
| | | TGAAAGCTTTTGATAGTTATGAATTGGATAAAGCGGATGTTCG |
| | | TAGGAGATTTAGAAACATTAATTCGGTTTCTGTTATCCCAGAA |
| | | GAAGTTTGGCAAGATAATGCGGAAGAAATCAATTCCTATTTTG |
| | | TTACTCTTCGTAAAACCTCTAAGGAAATATCAACAAAAGAGAA |
| | | AATCATTGCAAGAACCAATTTAGCAGAATTTATGGTGAGTATT |
| | | CCAGACTATCTTTATAAAAAAGGTGAGAGCGTTGTACGCGAAA |
| | | TAAACCGTTATGAGAGTGTTATTGAGTTTAAATGTGGTTATTCG |
| | | AGTGATGTAGGGGTTTTTATGAGGTGA |
| 38 | LMIB | ATGAGATTAAAGATAAATTGTGATTTTGATTCTAAAATAATTTC |
| | (optimized) Cas6 | AAAAGATTTTCAAAGTAAAGTTGTTAGTCTGTTCAAAGCGGGC |
| | | ATTATGAAATCGAGTCCAGAGAGATATGAGAATTTATTTGGCG |
| | | GGAATAAGCATAAGCAATATACATTTTCAGTGTACCTTCCCAA |
| | | ACCTCAAAATAAAGGTGCGGAAATTCAGTTAAATGAAGCAAA |
| | | TTGTATTATTAATTTTTCAACGGGAGATGCGGAAACGGGAATC |
| | | ATTTTTTATAATGCATTGATGAGTTTGAGAGGTAGCAAAGTTTT |
| | | ATTTGGAGCTGGGAATCATATTACGGTAAAAAACATTCAAATA |
| | | GTCCCGGAGAAAAAAATTATCGGCAAACGAACAATTTTGAGA |
| | | ACGCTTTCACCTGTGGTTAGTAGAGACCATAATAGAGAAACAT |
| | | TCAAGAATTGGTTTTACAGTTTTGAGGATGAGGAATTTGAGCC |
| | | AACTTTGAAAAGAAACATGTTGCCCTATTTAATGGATGCATTT |
| | | GGTGAGCAAGCTCGTTATGATTTGGAGAAATTGAAAATAACGC |
| | | CAATTTCGATGAAGAAAGTAGTCGTTTATTGCCACGAGATACA |
| | | TATTGAAAGCTCAGTCGGGATTTTTGAATTGGAGGCTGAGGCT |
| | | TATTTGCAGAAGTATTTGGTGGAGAATGGTATTGGGACTATGA |
| | | CAGGTTCTGGATTTGGCATGATAGAGCAGTTTTAG |
| 39 | LMIB | ATGCGGACAGAAATAGAAGTAAGGGCAAATGATTGGTTAATA |
| | (optimized) Cas8 | AATGCTGGTTTAACTGGTTTTCTAAATATCGTTGGAAAAGAAA |
| | | ATGTAAGAATTGATGGACAAGCTATTTATTTTACAGCAGATTT |
| | | ATTAGAGGACTTTGAAACGAAATACTTTAATTTCTTTATTCAAG |
| | | AATATAAAGAAACGTTATCGTGGTATAAAATCGTTTCTTATAA |
| | | AGAGACAATGGAACAGTTTAGAATAGATGAATTTGCTTCTTTT |
| | | GACGAGGTTGCATTGGGAAATTTAAATAAATATATGAAAGATG |
| | | TAGTAAAGTTTTATTTAAAAAAAGCTAATTATATCAAGGTGTTT |
| | | CCGCTAATTGACCCTAGTGCTAATGTAACAAAATGGTTGGAAA |
| | | AATTAAATACAATTAATTTAACTAAAAAGCAAAAATGGGATGA |
| | | AGTAAAAGCCGATATTATTGAAGAAGTAAAGCAAACATATACT |
| | | CAACTAGACCTGATTATTGATTTTTGTGCAAGTGAAAAGGGGC |
| | | TTCAATATCTAGGAGCAAAAAATCTGATATATTCTGTTATAAA |
| | | TAAAGGTTGGTCTGGTGTATCTTTCCTTCTTAAACAAACAAAAT |
| | | TTATTGACCCCTATTTAGATTACAAAACTAGTTTTTTGGACCCA |
| | | GTAATAGATTATTTAGACACCGATTTATCTAAAGCAAAATATA |
| | | ATTGTTTCAATTGTAATCAACCAATTAAGAATTTGAAACTAGA |
| | | CCTCAGTTTTATGAACGATGTTGGGTTTGATACTGCAAGAAAA |
| | | ACAGCTCACGTATGGGATTTTAACAATGATGTTGCGACTTGCC |
| | | CGATTTGTCGTTTGATTTATTCATGTGTTCCAGCTGGATTTACG |
| | | TATGTGTACGGAGAAGGTATGTTTGTCAACGATTCATATGGAG |
| | | TAGAGGAATTACACCGTGTTAATGAACGTATGCGGAATTCTAT |
| | | CCTGCGCTTTAACAAAGATGGAATTAATTCAACTAATACTTAC |
| | | CGAGCGCTTGTGGAGTCTATTACGATGGAGCATGAAAATAATC |
| | | GACGCTATGAACTTGCAGACATTCAGTTAGTTAGATACGAAAA |
| | | TGAGCATTATCGTTTTAATTTACTTTCTAAAAAAATGTTGCATA |
| | | TTGTAAATGATTCAAAGGGAATATTGAAAAGTTTAATTCGCTG |
| | | TGGTTATAAAGAAGGTAATTTGAATGTTAATTTGTACAAAGAA |
| | | GTTATTCAGCATTTAATGAACAATGAAAATTTATTTACGCTTAT |
| | | TCATAAATTAATCTACTATAAACAAACTAGTGTAAATGGTTTG |
| | | TATTACAATATGGGACACGTTTCAGGGATTTTAGATATAAATA |
| | | CAAAATTTTTGAAGGAGATGGAAGTGATGACAAATATTTCTCA |
| | | AAATCAATTATGGTTCGTTCAAAATTGTGGAAAGGAATTTAAG |
| | | GAAGGTTATGTTAAGAAGAAGTCAGAAAATAAAATTTCGGGA |
| | | ATAACATACAAGTTATTAAATTCGTTAAAGGTCAATGATAAAG |
| | | ATGGATTTATGGATACTTTATTGAATAGTTATTCCTATTTATCT |
| | | ATGCCCATTCCAGATGTATTCATAAAAATGTTTTCGAATAATG |
| | | AAGCTTTTAAATCAGTAGGTTATGCATTTATGCTTGGAGTAGG |
| | | CGGAGAGAAAACTAAAAAAGAAGACGGGGGAAACACAGATG |
| | | AAAAATAA |
| 40 | LMIB | ATGAAAAATAAAGGACTAGCAATGACAATTATTTTCCAAGCAG |
| | (optimized) Cas7 | AAAGTGCCAACTACGGGGAGTCTCTAGGTAATATTTCTTCACT |
| | | TAAAAAGATTTCTAGAAATAATGGAGACCAATATACATATATT |
| | | AGCAGACAAGCAATTAGATACAACCTCATGGACCAAATTGGA |
| | | GAAAAAGAAGCTCCTGTTAAAGCAGAAGGTGGTGGAGATAAG |
| | | AAAGTTATTCAATTCTTAAGTGAAGCTACTATTACAGATTTCCC |
| | | TGAACTTGATTTCTTTGGTTACCTTAAAACAGAAAAAGGAAGT |
| | | GGCGGACAAAAGAGAAGTGCAAAAGTACGTTTATCTAATGCT |
| | | ATTTCTCTTGAAACATTTAAAGGTGATTTGGATTTCCTAACAAA |
| | | TAAAGGTCAAGCAGATAAATTAAATGAAAATATGAATATTGCA |
| | | CAAGCAGAAATTCATAAATCTTATTATCGTTATACGATTACGA |
| | | TTGATTTAGACCAAATTGGTATTGACGGTGAAATTGCACTTGA |
| | | TAATAAAGAAAAAGCTCGTCGTGTAAAAAAATTGATGGATAC |
| | | AGTAGCATTCTTGTACCGTGACATTCGCGGGCGCCGAGAAGAT |
| | | TTAAAACCACTTTTCGTCATTGGCGGAGTTTATGACGTGAAAA |
| | | ATCCTGTTTTCCAAAATATTGTAGATGTAGCAGATAACAATATT |
| | | GTTATTAAAAATATTAAAGATTTACTTACGTATGAAGATATCA |
| | | AAGAAAATACGAGAGTGGGTATTATTGACGGGCAATTTGCTAA |
| | | TTCTGATGAAGTGAAAGTCGAGCTAAAAGCAGAATCTGTGCCG |
| | | GCATTCTTTAACGATATTAAAGGAAAGATTGATGCTTATTATG |
| | | AAAGCAATTAG |
| 41 | LMIB | ATGAAAGCAATTAGAGTAAGATTATGGCAAGATTTGGTAAATT |
| | (optimized) Cas5 | TTAAGAAACCAACTAGTTTTCAATTAAAAGAAACATATCCTTT |
| | | ACCTCCTTATTCAACTGTAATTGGGATGGTACATACACTATGTG |
| | | GATTTACAAGCTATCATGAAATGAAAATTAGTATACAAGGTAA |
| | | ATATTTTTCTAAAGTAAATGATTTAGCTACAAGATATGAATTTA |
| | | AAAATGGTATGACATATGATGCTTCTCGTCATCAAATTAAAGT |
| | | AGATAAGTATGGTGTAAGTAGAGGTGTATCAACAGTAGAATTA |
| | | TTAGTAGATTTAGAATTATTATTACATATTATTCCTGAAGATCC |
| | | ATCATTAGTACCAATCATAGAAAAAGCTTTTAGAGAGCCTATT |
| | | GAGTTTCCTAGTCTAGGTAGAAGAGAGGATATTGCTACAATTC |
| | | AAAAGGTAGAAGTTGTAGATGTAGAAAAAAGACAACCTAAAA |
| | | AAAGTACAGAAATATCAGATGGTTACAATGCTTATGTACCAAT |
| | | CTCTTTAACTGAAAATAATTCTGTTCATTTCAAATCTCATGAAT |
| | | CATCAGTAGGTAGAGATAAGTIGTTAGGTACAAGATATTTGCT |
| | | AACAGAAAAATATGAAAGAGTTAATCATGGAACAGAAAAAGC |
| | | ACCTAAGTTTTTCAGAAAATGGAGAAAGAAAGATGTTATTTAC |
| | | TCAAGTAGAATTTTTGTCTCTAAGAAAGATGTTTTCTTTTTTGA |
| | | TGAAGATGATTACTTAGTCTTTATTGAAGAAGAGGAGTAA |
| 42 | LMIB | ATGCATAAATATTTAGCTAAATCTAATCCTGAAGAAACAATTC |
| | (optimized) Cas3 | AAGAGCATACCGATAATTTATTAAAAAACTATCAAAGACTAAA |
| | | AAAACTATATCCTGCTGTAGAAATAGATTGGTATTTATTAGAA |
| | | TTAGTTTGTTTACTACATGATTTAGGGAAAATGAATCTATTATT |
| | | CCAAACTAAATTAGGTAATGCTTCAGGTAAAGGTAAAGAAATA |
| | | CCACATGGTTATTTGTCTGTTGCATTTGTTCCTGATAGTAAACT |
| | | TGAAGAAATGGGATATTCTGAAGATGAAATTAAAGTTGTTTAT |
| | | CAAGCTATAGCTAGGCATCATGAAAGGAAATATGATTTTTCTG |
| | | ATTCAGAATGGAAAAAAGAAATAATCAAATTAGAAGAACAAT |
| | | GGGCAGGTTTTATATATTCTCAATTAGGTGGAAATGCAGAATA |
| | | TAGTAGTGAGGTAGAAATGTTATATTTCTATCCTGGTTCAAGA |
| | | ATTTTTGAAGGTAACTCTGCAGCTGATGCAGAAACATTCAAAA |
| | | AATACGTTTTATTAAAAGGTTTATTAAATAGAATTGACTTTGCA |
| | | GCTAGTGGTGGTATTGATGTAGAACTTGAAAATGATTTCTTAA |
| | | TGGATAGTATGGAAGTACAATTAGCACATTTTAAAGCAGAAAA |
| | | TCCTGAAGCGGATTGGAACGCATTACAAAAATATATGATGCAA |
| | | CATCAAAATGAAAATATTGTAGTTATTGCAGAAACAGGTATGG |
| | | GTAAAACAGAAGCAGGTTTATTATGGTTAGGAAATAATAAAG |
| | | GTTTTTTCACATTACCTTTACGTGCTGCAATTAATGCAATTTAT |
| | | GAAAGAATTACAAAAAATATTGTAACAACAAATCAAGCAGAA |
| | | AGAGTAGGTCTACTACATTCAGAAACATATTCTCAATATTTATT |
| | | ACATGAAGGTAATGCTGAAATGGATATTGATGAATATTATACA |
| | | AGAACAAGACAAATGTCTTTACCTATAACAATATGTACATTAG |
| | | ACCAATTATTTGATTTTGTATTTAGATATGCAGGTTTTGAACAT |
| | | AAATTAGCTACATTATCATATTCTAAAGTAATTATTGATGAAAT |
| | | TCAAATGTATTCACCCGATTTATTAGCTTACTTGATTTTAGGGT |
| | | TATCTTATATTGATAAATTTGGTGGAAAGTTCTGCATTATGACA |
| | | GCAACATTACCTGGTATAGTAACAGATTTACTTTTAGAAAATG |
| | | GAGTTGATTTTGTACAACCTGAAGAGAAATTTGTATCTTCTAG |
| | | AATTAGACATAGCATGGAGATGGTTCATACAGAAATTGAATCA |
| | | GAATTTATTAAACCATTTTTTAACAATAATAGAATTTTAGTTAT |
| | | TTGTAACACTATTAGTAAAGCTAAAAAAATTTATTCTGAGTTA |
| | | AAAGAGCATTTTCAAGGTGAAGAAATACATTTAATTCACAGTC |
| | | AATTTATTAAAAGAGATAGAAGTGCTAAAGAAAAAGCAATAT |
| | | TTAAAGATGGTCAAAAAGATAGTACAAAAAAATGTATTTGGGT |
| | | AGCAACACAAGTAGTAGAAGCTTCATTGGATATTGATTTTGAT |
| | | TTACTATTCACGGAGCTATCAGATGTGAATGGGCTGTTTCAAC |
| | | GGATGGGCAGATGCTATCGAAACCGGGCGCTGGATGTGGATA |
| | | CGAATGTCTATGTTTTTGATGGTGGAGCGAAAGTTTGCTCAGG |
| | | AATTGGAACTTTTATCGATAAGTTGATATTTGTAAATTCTAAGA |
| | | CGATTTTAAATGAGCATGCTGGCGTTTTGACAGAAGAGAAGAA |
| | | AATGGAGCTAGTGGAGCGGGTTTATTCAACAGAGGCTTTACGT |
| | | GGGAGTGAATTTTATAACGATTTGAGAAAGACGATAAACTATG |
| | | TGAAAGCTTTTGATAGTTATGAATTGGATAAAGCGGATGTTCG |
| | | TAGGAGATTTAGAAACATTAATTCGGTTTCTGTTATCCCAGAA |
| | | GAAGTTTGGCAAGATAATGCGGAAGAAATCAATTCCTATTTTG |
| | | TTACTCTTCGTAAAACCTCTAAGGAAATATCAACAAAAGAGAA |
| | | AATCATTGCAAGAACCAATTTAGCAGAATTTATGGTGAGTATT |
| | | CCAGACTATCTTTATAAAAAAGGTGAGAGCGTTGTACGCGAAA |
| | | TAAACCGTTATGAGAGTGTTATTGAGTTTAAATGTGGTTATTCG |
| | | AGTGATGTAGGGGTTTTTATGAGGTGA |

### Example CRISPR Array and CRISPR Array Components

| | | |
|---|---|---|
| 24 | crArray | AGAGTTTGCAAAATATACAGGGGATTATATATAATGGAAAG |
| | (promoter bold | |
| | and underlined) | TTTTAACTACTTATTATGAAATGTAAATCGTTTAATACCTTGTT |
| | | CAATTTTATCAGCTATTTCTTGTTTTAACTACTTATTATGAAAT |
| | | GTAAATACTTGCATTGTCTGTAGAAATTGGGAATCCAATTTCTG |
| | | TTTTAACTACTTATTATGAAATGTAAATAAGAATTATTATTAAA |
| | | AGGTTTAGAAAAATTAGAATAGTTTTAACTACTTATTATGAAA |
| | | TGTAAAT |
| 43 | Repeat | GTTTTAACTACTTATTATGAAATGTAAAT |
| 44 | Spacer 1 | CGTTTAATACCTTGTTCAATTTTATCAGCTATTTCTT |
| 45 | Spacer 2 | ACTTGCATTGTCTGTAGAAATTGGGAATCCAATTTCT |
| 46 | Spacer 3 | AAGAATTATTATTAAAAGGTTTAGAAAAATTAGAATA |

### Example Lysogeny and Deletion Sequences

| | | |
|---|---|---|
| 30 | p1473 Var010 | GGAGGTGATAAGTATGGAATTTAATGATTTTCAAAATTTCTTTG |
| | (FIG. 6) | GTGAACTTAGTAATCAAGCCGAAAAAGAATTCGGTGGTGACA |
| | | GTGACTTTTTTAGAGATAGAATAAATAAGTTGAAAGAAGATGC |
| | | TCCTGAAAACGTATCTTACGAAATTATTTATTCAATAGCTTTAT |
| | | ACGAAAGCTTAAAAGCTCAACAAGATATGAAAATTTTGAATAC |
| | | AGTTAAATATCTTTTAGATCGTGACTAGCAATATCCAACAATG |
| | | ATTTGCTCTGAGCATTATTAATTTTTGGATAATCAAAATTTCTA |
| | | AGTTTAAATCTTGTGTTTTTCTCAATCTTTACAACCTTCCACGTC |
| | | ACAACTGCCATTGTGATGAGGAGGGTTGTTTTGTATAGTGTGTT |
| | | CATTGATAATTCCTCCTATTAAGATTTTTATTTTTCTCCTAAAA |
| | | ACTTATTAACAAAGTATTGTTGTCCTTTGCCTGTTACTTTTGGC |
| | | GTCTTACTAATTGATGTGTGACCGTCCGAATGTGTGATTGATGT |
| | | TTCTTTAATTTCGAATAACTCACGTTCCATTGAATACTGTGTAG |
| | | GCATGTTATAATCCACACCCTTGCGTTTAATAAGGAATCCGTTT |
| | | TGACGTAACCACTCAAACAATCTGCGTTGCCCGATGTTTATAC |
| | | CGTTTTGTTTAATGATCTTTGCTAACTCTCCAACTAAAATTGAT |
| | | GTCTTAGTAGTAGCTACTGCATCTGCAAATACAATTTTTGGTTT |
| | | ATCACGTTCAATCTTTGTTTCTAATTGATTGATTGTGTTGTTAG |
| | | CAATTTTTAAAGCACGTTGCATAATCATTTCTGGGCTGTTCCAT |
| | | GCTTTTTCAACTTGGATGAAATATTGTCTTGCACGTTTACCGGG |
| | | TTCACTACGTTGAATCATTGCGATTTCTTTTGCAGTGTCTAGTG |
| | | TGAGTGCGTGGTCTAAATAATTAATAGCGTTACCTTGAGCTGTT |
| | | ACTCTTTTTTGAGTAAGAGCTGTATAATCAATATTTTCTTCAAA |
| | | GCCATAATTAATCATTCTTTCAAACCAATCGTTATATCTTGTCT |
| | | TAACTTCTAATGCTTGATGAAGTTCTCGACCACTGATTGCGATT |
| | | TCTCCATTTTCTTTTTCTTGTATGTTGAACATTTCTCCGATGTTC |
| | | GATTTTGTTTGTAATGCTTGCATAATGTTTATGCTCCTTTCGTGT |
| | | ATAATGTTGTTATCAACCTAA |
| 31 | p1473 Var012 | GTACGATGATATATTACACGAAGAAGTATTTTTCAAAGAAGAT |
| | (FIG. 7) | GAAGCGCCATCAAATGCTGATTTTTGCATTTTAATTAACGGCG |
| | | ATTCAATGGAACCTATGTTAAAGCAAGGAACATACGCTTTTAT |
| | | TAAGAAAGAAGATTATATTAAAGATGGCACAATTGCACTCGTT |
| | | GTATTAGATGGAGTAAGTCTTATCAAGCGTGTAGATATATGCG |
| | | AAGACTATATTAATTTGGTATCTCTAAATCCGAAGTATGATGA |
| | | TATCAAAGTCGCTTCGTTTAGTAATATTAAAGTAATGGGCAAA |
| | | GTTGTATTGTGATTAATAGCGCCTATATGGCACTTTAATATAAA |
| | | AGACGTCTATTTCATCAGTGTTTAAAAGGAGTTTATAATGAAA |
| | | ATAACTAATTGCAAAATAAAAAAAGAAACTATAGTATATGAA |
| | | GTTTTAACTAGTGGTAATCAACCATTCACTTATGAGTTATCTAA |
| | | AGATTTATCGTCACATAATGCGCGTAAATACTTGGAATTTATTT |
| | | CACAAAAAATAGATGGCGATAAGTTAAATTAATTCAAAGAAT |
| | | AAAGTAACTTCATAAAGAGTACGAAGAAAACGATCTAATGAC |
| | | CGAACTTATTCTTGAATATTTAGTAAAAAAGTATGTTGAAGAA |
| | | GAATATAAGAAATAAACGCCTATATGGCGTGAGGAGGATGAG |
| | | GGATGGAAGAGAACGCACCTTTAGAAACAGCAGTTAATAATTT |
| | | TAAAAAGATTCAAAATAGCGAGATTTACAAATTTAAATATATG |
| | | AATTCATGGTGTCTTGAATATTCAGAGTTTTTATTGGATGAAGT |
| | | TAGATTGTTAAAAGAAAACAAAAGTTACACCAGATATAAAAA |
| | | AGGCACTATAATTTATGTAAAGTTAGGTGTTAATGTTGGCAGA |
| | | GAGTTTTCTGGAAACCATTTTTGTATGGTACTTAATAATCACGA |
| | | TTCAAATAAAAATCCAATATTAACGGTAGTTCCACTTACATCTT |
| | | CCAGAAGTAAATTCAATGTGCATATCGAAGAAGATTT |
| 32 | p1473 Var042 | TCTTTTTTTATACAATTTTCACGGGTAGCACGCCTACCCTTATT |
| | (FIG. 8) | ATTTTTTGCCAATTTTGAGGAGGGAGAAGCAAAATGCCAGTAT |
| | | ATAAGGATGATAATACAGGTAAATGGTATTTTTCCATTAGATA |
| | | TAAAGATGTATACGGTAATAACAAACGAAAAATGAAGCGTGG |
| | | GTTTGAACGTAAGAAAGATGCCAAACTAGCCGAAAGCGAATTT |
| | | ATACAAAATGTTAAATATGGATACTCGGACAATCAACCCTTTG |
| | | AATATATATTTTTTAATCGTTTAAAAAATGAAAATCTTTCTGCA |
| | | CGCTCAATAGAAAAGCGAACTACAGAATATAATACTCACATAA |
| | | AAGAAAGGTTCGGAAATATCCCTATTGGCAAAATCACTACTAC |
| | | GCAATGTACTGCTTTCAGGAATTATTTGTTAAACGATGCAGGT |
| | | CTTTCTGTTGGCTATGCACGATCTGTGTGGGCAGGTTTTAAAGC |
| | | AGTTATCAATTACGCCAAAAAGCATTACAAGCTCTTATACGAC |
| | | CCCACATTATCGGTAACTCCTATTCCCAGAACAAAACCACAAG |
| | | CTAAATTTATCACTCGTGAAGAATTTGATGAAAAAGTAGAACA |
| | | AATCACAAACGATACTTCTCGTCAGCTAACTAAACTGTTATTTT |
| | | ATTCTGGTCTTAGAATAGGCGAAGCTTTAGCTTTGCAGTGGAA |
| | | AGATTACGATAAAATAAAAGGCGAAATTGACGTAAATAAGAA |
| | | AATCAATTTAAGTAATAGAGAAATTGAATATAATCTAAAAAAA |
| | | GAAAATTCTAAAGGGATAATACCTGTACCAAAATTAATTAGAG |
| | | AGATGCTTAAAAACATGTATAATGAATCTTCTAAAAGATATAA |
| | | ATATTTTGACGAAAACTATTTTATATTCGGGGGGTTAGAACCT |
| | | ATTAGATACGTTACCTATTCGTATCATTTTAAATCTGTATTCCC |
| | | GAATCTAAAAATACACCATTTAAGACACTCGTACGCAAGCTAT |
| | | TTAATTAATAATGGTGTAGATATGTATTTATTAATGGAATTAAT |
| | | GAGGCACTCTAACATTACAGAAACAATTCAAACGTACTCTCAT |
| | | TTATATACTGATAAAAAACATCAAGCTATGAACATATTTGATT |
| | | AAATGGTATCATATCGGTATCAAATAACGATTAAGGAGTTTAT |
| | | AAAATGCGTAATAACAAGCCTAAAATAAGAATTCATAATGACC |
| | | CATGGGAAGTGAAATTTATATACATTTAAATTTCATGAGACAA |
| | | TAAACGTTGATTTAATGCGTTTTTTGCCTTTTTTATTTTCCTTAT |
| | | TTTTTCTGTTTTACAACAAAATGGTATCAAAAATGGTATCATTT |
| | | GTAGTTATTTTAGCTTCACATATTAAAACAACCACACTCCTAAA |
| | | TTAATAGGTGGTGTGGTTTGATCATTTATAATATAACATAAAA |
| | | AACAACCACCCAGTAACTAGTATGGATGGTTAAGGTGTGCCTG |
| | | CAGCACATAATAAAACCGATAATATGTTTTATT |
| 47 | Repressor | MREKVSNRLKHIMKIRNLKQVDIINKSKPYQKQLGISLSKSTLSQY |
| | | INDVQSPDQDRIYLLSKTLNVGEAWLMGYDVNSYRVPDEERQEE |
| | | TVMSKINNISSQLTPPRQSNVLNYANSQLDEQNKVTSIDEYKESKL |
| | | VSYIACGATGAGIGEELYDDILHEEVFFKEDEAPSNADFCILINGDS |
| | | MEPMLKQGTYAFIKKEDYIKDGTIALVVLDGVSLIKRVDICEDYIN |
| | | LVSLNPKYDDIKVASFSNIKVMGKVVL |
| 48 | Repressor | MDKKELAKFIGNKIRYYRTKLNLTQDQLGEKLNTKKATISNYETG |
| | | YRTPKQDDLFEIAHILNISIDDLFPTRNNKKNDITSIYNKLTPPRQE |
| | | NVLNYANEQLDEQNKVTSIDEYKESKLVSYIACGATGAGIGEELY |
| | | DDILHEEVFFKEDETPSNADFCILVNGDSMEPMLKQGTYAFIKKE |
| | | DSIKDGTIALVVLDGVSLIKRVDICEDYINLVSLNPKYDDIKVASFS |
| | | NIKVMGKVVL |
| 49 | p1378e062 | TGTATTATTGATTAAGTCACTACTTCCACATTCACTACATTCTT |
| | deletion | CAGGCTCTTCATCATGACCCCAGTCTTCAAAATGCTTACAGTCC |
| | | TCACACTCATAAGTTAATACAAAATCCTCTTCAAATTCAAAAT |
| | | CTCTCTGCTTCATATTAGTTTCCTCCTTTAGCTTGTTCAATTAAA |
| | | TCTTTTACAGCAAATACTTCAGGGTTAATTATTCTTAGGATATC |
| | | TTGGGGTATTCCCCACACTAAGTAAATAAAAGCAATGAAAGTA |
| | | ATAATACCTGTAAAAACTAAATACGTAATAATCCTCAAACAAT |
| | | AGACTATAGTCATCATCTCTATCAATATCATTTACTTTTTTATG |
| | | TTGGTTTACAAATACCTTCCATAGGAATAATGTTAAAGCTATA |
| | | ACTAAAATACCAATAATAATATCTACGATACCGTATGCTATCT |
| | | GATACTTTACTAAAGCCTCCCAAGCATGTTGGCTAATATCACCT |
| | | AACTTACCTCCAAGAGAATCAATTCTCTCTAACACTTTATCTTC |
| | | TAACTTCATTACAACCACTCCTTATCTAGTCTTCTGAGTTCCAT |
| | | CCATTCATAAATCCAAGTACAAACAGTACTGCCAATATAAAAA |
| | | TTACGAATAACATAGGTCATACCTCCTCTTTATTTGTTAACTTA |
| | | AGTATAACCTATGTATACTCAATTGTCAAGACTTATTTCTTCTT |
| | | TTTGCTTAATTTTTTTTGTTGCTCTTTTAGTGAATCCTTCTGTTG |
| | | AGGTGTTAACATTACTTATCTCTCCTTATCAAATTCTTTTGTTGT |
| | | TTTAATCTTGTATTCTTCTTTAGGATTTTGTTCTTCTAATCTATC |
| | | TTTTAAATCCTCTAATACAGATACATCCCTGTGAGCTTTAATTT |
| | | GTCTAGGTATTTTCTTTTTACCTACTTTGTCGTAATAACATAAA |
| | | GCATACCACTCATTCATTTATATAACCGCCTTACTTAATATTCT |
| | | TGATTTCTAGCTCAAACTCTACTAGTGAGTTTTTAGATAAATCT |
| | | GTGATACTGAACACATTATTATCTCTTTTAATAAAGTGATTACT |
| | | AGGTGTAATTCTTTCCGGTTCATTTTTCTTAATCAATAATTCTA |
| | | ATTGAGAGAATAATCTTTCATTAACTAAATTATTAACATCTTCT |
| | | TCAATATCTTTTCTAGTTAAGTAAGAATCATTTAATTTATAGAT |
| | | ATTTTTGTTAACATTACCTTTACCAATAAACATGTTGTCTACAT |
| | | GACAATTACTTCGGTCACCATCTTTAAAACACACATGACCGTT |
| | | CTTAAGGTCTCCTCGTGATTGACTAATCATTTCAGGGAATGTAT |
| | | GTGCAATAACTACTCCCCTAGAATAACTTGTTTTCTTAGGGTTA |
| | | ATTAAGTATACAATCTCATGAGCAGTCCACTTAGGATGGTTCTT |
| | | TCTTGTCTGTGTTCCTTTAATTAACTTTTTCGTTACTTTATTTCT |
| | | AATTAAACCTGTAGGATGTGCTTCATAATACCCATTAAAAGGT |
| | | ACTGTTCTCCATCCTTTCTCAGGCTGTAATGAGTGGTCTAAATG |
| | | TTTTCTTCTTTTAATCATTATTTTTCCTCCTTTTGTAACTTCTTAT |
| | | CTAAAGTTACGCTATAATCCTCTTTATAAAATCCTTTAATTCTA |
| | | CCTTTGCGTCCTCTAGCATGTTTAACATACTTGTAAACAGTACC |
| | | TACAAATTGAATTCTCTTTCCTTGAAGCTCCTTTAGTTTTTCGTA |
| | | GAAAGAGATACCAACTTCAATGTTAACGTGGTCAATAGGTATA |
| | | CCATTAATCTTTACATCGGTAACTGTGATTTTAACACATCCATG |
| | | TTGATTACCTTTATGTCTTCTGCGCTTTGAGTACTTAACATCAT |
| | | CTAAATACCCTTTAACATTAACTTGTTTCCTATTAAAAGGTTTT |
| | | AACTCTTCTCTCATCTTTCCACCTCCTACTATAATTGTAACTTAT |
| | | GATAGTTAAACATATCTTTAGTTAATTTCTTAGCAAAGTAATTA |
| | | ATCATATATTGTGTTGAAGTTCCTTCTAGTTTATCATAATCAAA |
| | | ATCAATGTTTGTAAAAATTACAAAGCTTCCTAAGTTTCTACTGC |
| | | CTTCTCTAACTTCAAATACAAGTGTATCATTTTTAAAAGTTTTA |
| | | ATTTTAAATTCTTTATTCGTATCAAAACTTTTAGGGAAGTTCCA |
| | | CCATTCTTTAGTTGTTGCCATAATCTCAATTGCATTTGAAAATT |
| | | GTCTTCTGATTGTTTCTTTACTCATATTAATCATCTCCTGTACTT |
| | | GTTATATACTAAGTATAATACAAAAGTACAAAGGTGTCAATAC |
| | | TTATTTTTAAGAAAATGAAAGAAGATTTATTATCATTCCTAGTG |
| | | CACATATTAATGAAGCACTAGAGTATAGTATTGACTCCTTATC |
| | | ATCTTTAACTTTCCTAGCTAATAGTAAGAAAAGTAACATACCT |
| | | GCTTGTAGTAAGAAAAACCATCCATAAAATCCTGCCCAAATAT |
| | | TTATTAAGATTGCAGTACCAGTAAGCATATAGCTCATATCATC |
| | | ATTAATCAAATCAATAATGACTACAGCTATAAAAGCAACAGAA |
| | | GCAATAGTTAATAAGTCCATAGGCATTAATCCCAGTTCCAGTC |
| | | TTCCATCACAAAATCAATATAAGCTTCATTTTCTTCATCTATAA |
| | | TTTGGTCTTTAACTACTTCTATTTCTGAAATAACAAACTGTAAA |
| | | GAATCTGTTAATCTACCTAGCGCAATACGTTCTGTTGTTTTAAT |
| | | AGGTAAACGTCCTGAGTGTGGGTGAACTAAGTTATCAAT |
| 50 | p1378e074 | TGTATTATTGATTAAGTCACTACTTCCACATTCACTACATTCTT |
| | deletion | CAGGCTCTTCATCATGACCCCAGTCTTCAAAATGCTTACAGTCC |
| | | TCACACTCATAAGTTAATACAAAATCCTCTTCAAATTCAAAAT |
| | | CTCTCTGCTTCATATTAGTTTCCTCCTTTAGCTTGTTCAATTAAA |
| | | TCTTTTACAGCAAATACTTCAGGGTTAATTATTCTTAGGATATC |
| | | TTGGGGTATTCCCCACACTAAGTAAATAAAAGCAATGAAAGTA |
| | | ATAATACCTGTAAAAACTAAATACGTAATAATCCTCAAACAAT |
| | | AGACTATAGTCATCATCTCTATCAATATCATTTACTTTTTTATG |
| | | TTGGTTTACAAATACCTTCCATAGGAATAATGTTAAAGCTATA |
| | | ACTAAAATACCAATAATAATATCTACGATACCGTATGCTATCT |
| | | GATACTTTACTAAAGCCTCCCAAGCATGTTGGCTAATATCACCT |
| | | AACTTACCTCCAAGAGAATCAATTCTCTCTAACACTTTATCTTC |
| | | TAACTTCATTACAACCACTCCTTATCTAGTCTTCTGAGTTCCAT |
| | | CCATTCATAAATCCAAGTACAAACAGTACTGCCAATATAAAAA |
| | | TTACGAATAACATAGGTCATACCTCCTCTTTATTTGTTAACTTA |
| | | AGTATAACCTATGTATACTCAATTGTCAAGACTTATTTCTTCTT |
| | | TTTGCTTAATTTTTTTTGTTGCTCTTTTAGTGAATCCTTCTGTTG |
| | | AGGTGTTAACATTACTTATCTCTCCTTATCAAATTCTTTTGTTGT |
| | | TTTAATCTTGTATTCTTCTTTAGGATTTTGTTCTTCTAATCTATC |
| | | TTTTAAATCCTCTAATACAGATACATCCCTGTGAGCTTTAATTT |
| | | GTCTAGGTATTTTCTTTTTACCTACTTTGTCGTAATAACATAAA |
| | | GCATACCACTCATTCATTTATATAACCGCCTTACTTAATATTCT |
| | | TGATTTCTAGCTCAAACTCTACTAGTGAGTTTTTAGATAAATCT |
| | | GTGATACTGAACACATTATTATCTCTTTTAATAAAGTGATTACT |
| | | AGGTGTAATTCTTTCCGGTTCATTTTTCTTAATCAATAATTCTA |
| | | ATTGAGAGAATAATCTTTCATTAACTAAATTATTAACATCTTCT |
| | | TCAATATCTTTTCTAGTTAAGTAAGAATCATTTAATTTATAGAT |
| | | ATTTTTGTTAACATTACCTTTACCAATAAACATGTTGTCTACAT |
| | | GACAATTACTTCGGTCACCATCTTTAAAACACACATGACCGTT |
| | | CTTAAGGTCTCCTCGTGATTGACTAATCATTTCAGGGAATGTAT |
| | | GTGCAATAACTACTCCCCTAGAATAACTTGTTTTCTTAGGGTTA |
| | | ATTAAGTATACAATCTCATGAGCAGTCCACTTAGGATGGTTCTT |
| | | TCTTGTCTGTGTTCCTTTAATTAACTTTTTCGTTACTTTATTTCT |
| | | AATTAAACCTGTAGGATGTGCTTCATAATACCCATTAAAAGGT |
| | | ACTGTTCTCCATCCTTTCTCAGGCTGTAATGAGTGGTCTAAATG |
| | | TTTTCTTCTTTTAATCATTATTTTTCCTCCTTTTGTAACTTCTTAT |
| | | CTAAAGTTACGCTATAATCCTCTTTATAAAATCCTTTAATTCTA |
| | | CCTTTGCGTCCTCTAGCATGTTTAACATACTTGTAAACAGTACC |
| | | TACAAATTGAATTCTCTTTCCTTGAAGCTCCTTTAGTTTTTCGTA |
| | | GAAAGAGATACCAACTTCAATGTTAACGTGGTCAATAGGTATA |
| | | CCATTAATCTTTACATCGGTAACTGTGATTTTAACACATCCATG |
| | | TTGATTACCTTTATGTCTTCTGCGCTTTGAGTACTTAACATCAT |
| | | CTAAATACCCTTTAACATTAACTTGTTTCCTATTAAAAGGTTTT |
| | | AACTCTTCTCTCATCTTTCCACCTCCTACTATAATTGTAACTTAT |
| | | GATAGTTAAACATATCTTTAGTTAATTTCTTAGCAAAGTAATTA |
| | | ATCATATATTGTGTTGAAGTTCCTTCTAGTTTATCATAATCAAA |
| | | ATCAATGTTTGTAAAAATTACAAAGCTTCCTAAGTTTCTACTGC |
| | | CTTCTCTAACTTCAAATACAAGTGTATCATTTTTAAAAGTTTTA |
| | | ATTTTAAATTCTTTATTCGTATCAAAACTTTTAGGGAAGTTCCA |
| | | CCATTCTTTAGTTGTTGCCATAATCTCAATTGCATTTGAAAATT |
| | | GTCTTCTGATTGTTTCTTTACTCATATTAATCATCTCCTGTACTT |
| | | GTTATATACTAAGTATAATACAAAAGTACAAAGGTGTCAATAC |
| | | TTATTTTTAAGAAAATGAAAGAAGATTTATTATCATTCCTAGTG |
| | | CACATATTAATGAAGCACTAGAGTATAGTATTGACTCCTTATC |
| | | ATCTTTAACTTTCCTAGCTAATAGTAAGAAAAGTAACATACCT |
| | | GCTTGTAGTAAGAAAAACCATCCATAAAATCCTGCCCAAATAT |
| | | TTATTAAGATTGCAGTACCAGTAAGCATATAGCTCATATCATC |
| | | ATTAATCAAATCAATAATGACTACAGCTATAAAAGCAACAGAA |
| | | GCAATAGTTAATAAGTCCATAGGCATTAATCCCAGTTCCAGTC |
| | | TTCCATCACAAAATCAATATAAGCTTCATTTTCTTCATCTATAA |
| | | TTTGGTCTTTAACTACTTCTATTTCTGAAATAACAAACTGTAAA |
| | | GAATCTGTTAATCTACCTAGCGCAATACGTTCTGTTGTTTTAAT |
| | | AGGTAAACGTCCTGAGTGTGGGTGAACTAAGTTATCAAT |
| 51 | p1378e074 | ACTGGAAAGGTCAGAATCTATTAGGCAAAGTAATGGAAGATG |
| | deletion | TTCGAGTACATTGTATCTACAATAAGTAGGTAATTATAAAATG |
| | | GATAAGATAAATCTCAATAAAAAACATGAGGGTTCTACCGTAG |
| | | TCAACATATCAAATAATATTATGTTAAAAATACAATGTACAGA |
| | | CCTAAGAAAAGAATGTGATGATTCAGAAGCACCTACTACCTAT |
| | | ACCCATTTTAAAGCTTATATCGTATATAATATATTCATTGTAGT |
| | | TAATGATAGAAAACAAAAGAAAAAAGTTAAGTACGATTATTA |
| | | TAATGACCATGTAGGTAGAGGTAATGTTAAAGACCTATTGAAA |
| | | GTAAAAGATGTTATCTTCCAGTTATCCACTCAATTAAATACTAA |
| | | TGAAATTATTAAAATATCGGGTGCAGATGAAAGAAGATATAA |
| | | AATATATAAATATTTTATAGAAAAAGATATAAGATTTGAAGAC |
| | | AATATGTATTATAGTAAAAGTAATATATGGATTATAAATAATT |
| | | TTAGCTTATTACAAAAGTTTCAATGGAACACTGTAGTAACTAA |
| | | AGATGGAGACTATAATAAAAAAGAACTTAAAAAGGTTGATAA |
| | | AGAATGGAAAGAATTATTAATATAAAAGGAGAGTTAAAATGA |
| | | ATATCAAATATATTGATTTAGTATTAGAAAATTGCGATGTTGTA |
| | | AGATTAGAACCTAAAGACGTAAGCAGGTTCCATATATCAGGTA |
| | | TTACAGAAGGTATAGATTACTATGGTACATATAAAGGGACTTC |
| | | AAATATAAATCGAACACGTCACTGTACTTATTTTGGTATTCTTA |
| | | TTGATAAACCTATGGAAATACCTCAAGTTGGTTTTGCTTATCCT |
| | | GATAATACGAACGCTTATGAAATGATTACAGCATATTCAGATA |
| | | TTACAGCTATAGATATTATTTATGAAAATGACGCAAATGAATA |
| | | TATTTATGTAGACTTTAATGAATACAATGATAACTATAATATCA |
| | | ATCAAAAGAATGATTATTACAATAATATGTTAGAAATTACAAT |
| | | TACAGAAAGTAATTCCATGGAGGAAGAAGATGAATAAAACAT |
| | | TTTTTAAGTTCCTAGGTAAAAACACATTAGAGTATTCAAAACA |
| | | GGGGCTAGGTTTTCTTGTAGCCCTTCCTATAATGTTAATTATAT |
| | | TTTCTGTATTCCTAGCATTCATTATAGGTATTCCTGCAGTTATT |
| | | ATTTACGCTCTACATGCATTAAATGTAGACAATGATTTTATTAT |
| | | ACAATTAGTACCTGTTATGTGGTTTATAATACTTTATGGTATTG |
| | | TAAGAACAGATGAGCACAAAAAACCATTTGTTAAACTAAGATT |
| | | AAAGGATTACTTACTATCTATATTATATCTAACTACTATTATAG |
| | | CTATTAGTGTTTTAGAGAGCTATTTGCTCTTCCAATTATTACCT |
| | | TTTACAGGAGATGTAAGAGCAGTTATAACACTATTATCATTTA |
| | | TAGTATTTGTTGTTGTAAATAGAGGTATTTGTAAAATAGCCATC |
| | | AAGAGCTATAAAGAATATAAGGAGGAATTATAATGAGATATG |
| | | ATATTAATGAAAAATGTTACGATGAGAAGGATTTTGTATTACA |
| | | AATTATATATGAAAATTATAGTGATGAAACAGATAGAGAAGTA |
| | | GAAACAATTTATAATAAAGCAGAAGCATGGGACAAACTCTGT |
| | | GAAATGCTGAAAGATAAAGATATGAGTGATGGGCATTTTGAA |
| | | GAAGAAATGTTTAAACTATTCTCTAGAACAGGTAAATCCTTTA |
| | | CAATAGATAAGGAGGAATCACAATGATAGAAATTAGTATCTCA |
| | | TGGACTTATCTAATATCATTCTTGCTACTATGGTCAGCCGGCAC |
| | | CCTATACATTAACTACCTTGTTTATAGAATAAGGTTAACAAAT |
| | | AAGGAACGTAAAGAAATGAGTAAGGAACACCACCGCAATAGG |
| | | GAAGAGATAAAACAAAGGATAGAAAATAGAAGGGATAAATA |
| | | ATAATGGAAAATTATAAAAACTTTATTATAGAGGAAATGAATA |
| | | AAGCCCGTATCTTAGTAACTAAAGCAGAACAAATTAAAAGGA |
| | | ATAGAAAATTAGCAGAAACAGAACTAGAAGAAGTATATAGAA |
| | | AAGCAGAAGCCTTCGATGAAATTGTAAATGAGTTACTTTATCA |
| | | ATTACAAAATCTAGAGAGTTGGGATACTCTAGACCAAAAAGAC |
| | | TGCCAAACATTAAAACAAATACTAGAGGAAAATATAAAGGAG |
| | | GAAAAACAGTTGAAAAGATACAAAGTAAAACGTACTATTACT |
| | | ACAGAAGAGGTAAGATATATAGATGCAGAAACAGAGGAGGAT |
| | | GCATGGTATAGTGTAGAATATGAAGATGAAGGTGCAGATACA |
| | | GCACACTATAATGCAGAATATGGTACATGGTCTTATGAAGAGG |
| | | AGGAAAAATAATGGGATTAGACTTTGAAGTAATCGGTGTGACA |
| | | TTAAGTAATAGAAAAGTGGAACAGAAAGGCTTACAGCACTTTA |
| | | TTAATAACGCTAGGTATAGACATATATTAGAAAAGAATTATTA |
| | | TAAAGGATTTAACTTTGAAGATGACTTTAGAAAACCAGGATAT |
| | | TTTATGGACTTACTTCTAAGAGACGCAGAGACATATTATGATG |
| | | AGTTTGAAGAATGGTGTGAAGGCGTATTCGTACTAACTAAAGA |
| | | TAAGTTAGTTAATCTAATGAAAAATGAGTTTAATGAAAAAACG |
| | | TTTAAAGGTACACATGATGCAGAATATTATTATAGATTAATGT |
| | | CTCATATATACAATGTAGAACAATATGAAGGTAAATTCTATGA |
| | | CTTTTACTTAATCATGAGTGTAAATGTATAAGGAGGATTAAGT |
| | | ATGAACAAAGAACAAGCCAAACTTAAACTCGAAACAAGTATT |
| | | ATTAATTATGAGAACCAAATAAAATTCTTAGACCCTGCAAGTA |
| | | TGTATACTAGAGGGCTTATAGATGCAAAAGGTTACTCAAAATT |
| | | GGCATTAAAAGAATTAGAAGATACAGGTAAACACTCTTATGAA |
| | | GATACTACATGGAAAGATAGTTATGCAAAGGTATTTACAGATG |
| | | AAGAAATACTAGAATTCTTACTATCCAAACCAAGAGTCACATT |
| | | TAAAGGTAATCAAGAAAAACTAGATGAAATTAAAAAAGAAAG |
| | | AGAAAAAATACAAAAAGAAGCTACTAAAGACTTACCTAAGGG |
| | | CAGTCCACTAGGTGACTTATCAAAAGAAAATTATGAGAAATTT |
| | | TGGGGAGCATTACAATGGTCTAGAGAAGAAAGAGAAAAGTTA |
| | | ACACAAGAATCAAGAGCTTATTATGAAAACTATCTAAAAAAA |
| | | ATCAAGGAGAATAAGTAATATGAAACTATACCAAGTAGAACA |
| | | TGATAATTGTGAACCTTATGAAGATAACTTCCATTTTAGAGAA |
| | | GATAAGATATATACAGATAAGGAGAACTTAATTAAACGTATTA |
| | | AAGAAGAAGGTTATAAAGAGGAAACAAACCATAGAGGTGAGC |
| | | AAGAGTTCATTAAAGGAGACCCAAGAGATTTCTATGGGATGGA |
| | | TATGATTACTATACATGAATTAGAGGTTGTTAATAATACCTAA |
| | | GAGGAGAGGGATTTAATTTCCCCTCTTTTTTTTATTTTAGGTAT |
| | | GTTGACTTTATATTTAGAGGTTGTCTATTTGTACTATAAATTAT |
| | | TGACTTA |
| 52 | p1498e001 | TGATTTGTTTGAAATTGCTCATATTTTAAATATCAGTATCGA |
| | deletion | |
| 53 | p3693e001 | GATGATTTGTTTGAAATTGCTCATATTTTAAATATCAGTATC |
| | deletion | |
| 54 | p3224e002 | CTTGTTTTAACATAGGTTCCATTGAATCACCATTAACTAAAATA |
| | deletion | CAAAAATCAGCATTTGATGGCGTTTCGTCTTCTTTGAAAAATAC |
| | | TTCTTCGTGTAATATATCATCGTACAATTCTTCTCCGATACCAG |
| | | CACCAGTTGCGCCACACGCAATATACGACACTAACTTAGATTC |
| | | TTTATATTCATCTATAGAAGTGACTTTATTTTGTTCATCTAACT |
| | | GACTATTCGCGTAGTTGAGTACATTGCTTTGTCTTGGAGGCGTG |
| | | AGCTGAGATGATATGTTATTAATTTTTGACATCACAGTTTCCTC |
| | | TTGGCGTTCTTCATCGGGTACGCGATAAGAATTTACATCATATC |
| | | CCATAAGCCACGCTTCACCGACATTTAAAGTTTTAGAAAGTAG |
| | | GTAAATTCTATCTTGGTCAGGAGATTGTACATCGTTAATATATT |
| | | GAGACAAAGTGCTTTTACTTAAAGATATACCTAGTTGCTTTTGA |
| | | TAAGGTTTCGATTTATTAATGATATCTA |
| 55 | p5593e001 | CTGAATTAGAGAGAACTTTAGGGTTTTCAAACGGACAAATCAG |
| | deletion | AAGATGGGAGAAAACCAAACCAGGTATTGATAAGGTGCAAAA |
| | | AATTGCCGATCACTTTGATGTATCAG |
| 56 | p1468e003 | GGAAGGTAGTATTGGATAGCTTTAAACCGCGTTGTTAAGCCAT |
| | deletion | TCTTGACTTCCGGAAATGGCTATTGATACCATTTTGATACCATT |
| | | TACTTGTCAAAAATGGCTATTGCATCGTGTTTTTTCTGAGTATA |
| | | TAAATGGCTGTAAGTGCCCATCGTTTCAGTGATTTGAGCATGTC |
| | | TCATGAGTGACTGTAAAACGAAAATATCTACACCATTATTTGC |
| | | AAGATAAGATGCATAAGAATGTCTTAACGCGTGAATGTTATAA |
| | | TGGGGGAAAGCTTTTTGGAATTTCTTTTGAACATGACTATAATG |
| | | TTTGGGAGCCATTCCTCCGAAAATAAAATAACTACGTTCATCA |
| | | AAATATTTGTTTAACTCTTTTTCACGTTGGTGTCGTTCAGTTAA |
| | | CATTGTATTGATGAATTTAGGTAAAGGAACAATATCCTCTGAA |
| | | CTATCTGTTTTTGGTCTTGGATATATAGTTCTATTAGAGATGTC |
| | | CATTGTTTTATTTATGGATATCTCTTTTTTGTATTTATTGTAGTC |
| | | TGTCCAAACAAGCGCCATAGCTTCGCCAATCCTCAAACCTGTA |
| | | TAAAACATTAATGTAAATAACTCTCTGTAATCTTGCTCTTCAAT |
| | | ATCTTTGATTCTTTCTTCAAATTCTTCACGCATCATAAACTTAG |
| | | GTTTTGGCTTTACACGCGGAATAGGTTTAATTGATATTGTTGGA |
| | | TCTGTACGTAATCCAAAGTATTTTTTAGCATAATTAATTACAAC |
| | | TTTAAAACCTGACCAAATTGTACGAGCAGAATTTGTTGACGCT |
| | | ACATTCTCTATTAGATATTTACGAAACTCTTGGCATTGATTTTG |
| | | TGTTATCTTATTCATTTTTATGTGCCCAAACTTAGCTTTAAAGT |
| | | GTTTATGATATTCATTTTGCTTGCGTCGTTTTGTTTTAGGTCTCA |
| | | AATCGCTATTTTCTAAATAGTGATGAAAAACATAATCAAAAGT |
| | | TTTTGAATCGCTATATCCTTCGTTTACGTCATTCAAAAAAATAG |
| | | CCTCTGCTCTCTTAGCTTCACGCTTAGTTGAAAAACCGCGTTGC |
| | | ATCTTACGTTTGTTATTACCGTATACATCTTTATATCTAATAGA |
| | | AAAATACCATTTACCTGTATTATCATCCTTATATACTGGCATTT |
| | | TGCTTCTCCCTCCTCAAAATTGGCAAAAAAATAATAAGGGTAG |
| | | GCGGGCTACCCGAAATTTTATTGTTGAATCACTTCGCTATTTTG |
| | | ACGTTTGAAATTGTCGAAATCATTTTGTGCTTTCTTCCATGAAT |
| | | TATAGTCTTGTCCGTCTTGTACTGCCCATGAACCACCTATACCG |
| | | GCAGTATGGCCACCATTCTGACGTTTGTTTTCTTCTGTTGCTCTT |
| | | TTAGCTTCTTGATAAGCGTTATAAGATGTGTCACTTGAAAACTC |
| | | ATCTTTCACTGGTGCATTGTTGTTTTTATTAGAAGTGGGATTAT |
| | | TTTGTGTTTGATTTTGTTTAGGTGCGTTATTAGTTTGTTGATGAT |
| | | CATTAACATTTGTGTTGTTATCGTTGTTTACTTGATTATTGTTAT |
| | | CGTTTTGATTAGCATTTTCTTTTTTCACTTCTGCTTTGTCTTTAG |
| | | TTTCTTTCTTTTTGTCTTTGTTCTCTTTCTTTGTTTCCGCTTTCTT |
| | | GCTTTCCTCTTTCTTATCGCCGTCGTTGCTACCACATGCACCTA |
| | | ATACTAACGCGCTAGCTAAAATTAAATATAATAATCTTTTCAT |
| | | GTTTTACACTCCTTTATTTGCTATTTGTTTTAATAAATCTATGAC |
| | | TTCGTTGTTTTGCTCGATAATTCTATTATTTTGCTTTATTAGTTC |
| | | GTCTCGTTGAGCTATAGAGACAAAGTTTTGTTTTAATTGCGTAT |
| | | CGTAGAATACGAATTTAGCTTGTTTATCTACGTTTGTTGTGAAT |
| | | GTACCTAAACCGTTGTAGACTTTCAATAGTGTAGGGTTAATATT |
| | | TTGCTTTTGATATGCGTAAGTCGTAACGTCGGTAGCTTCTTTAA |
| | | TACCTTGTCCGTTTAAACTTTTAGCTGATTTTGATTCGTATTCTT |
| | | CGTTAGTGTTTTTAAAATTTTCAGATTTATAAAGTTGGATATCA |
| | | AGTTCTTTTCCTTCTTTAAAATCATTTAATATCTTTCTTTTTTCA |
| | | TCTGTTGTCATTTTTTTATACATGTCAATCTTTCTATTGCTTAAC |
| | | TTACTAAACATTTTTGTTTCTGTTAGAATTTCTTTAAAAGTTAA |
| | | TTTATCTCCTGCCATTTTCAATTTCTCCTTCATTTGGTTTATATT |
| | | AAAGCGCCACATAGACGCTATTAATCAAAAATTCGATAGTTAT |
| | | AAATAACTTTGCCTATCACTTCGATTTCATCAATAGAATCTAAA |
| | | TCGTAAGAATTAGTTTTAAATTCATCTGAATAGCTTACTGGGTC |
| | | TAAATGTAGTTTTGTTTCAGTACGTCTCACACGTTTAACTGTAT |
| | | ATTCACCACCTAGACGCAATACAAGGATGTCGTTGCTGTTAAG |
| | | TTTATGATCACAAGACTTTCTATAATCATGGACAATTATATAAG |
| | | AACCGTTAGCGAGTATTTTATTCATGCTATCTCCGTTTATTTTT |
| | | AGTGCTATACATTCGCTAGGTTTACGACCGTTAAAAGCAAATG |
| | | GTGGAACTTTTAATTTTTCATTTTCAATTGCAACTTCTTCGAAA |
| | | TTTCCAGCAGAAACTTTACCGAAATATGGAACCTCGATTTCGC |
| | | TATCAAATTCGGGTAAAACAATTTCTTCAATTTCTCCTAAGAGA |
| | | TAACCTTTAGAAACATTGAACAAACTTGAAATTTTTTCGACCAT |
| | | ACCCATTCTAGGTTCAGTTCTTCCGCTTTCCCACATTCTTATAG |
| | | TACCTTCGGAAACATCTAATTTTCTAGCCATCTCAACTTTAGAC |
| | | AATCTATTGTTCAATCTGATTTCTTTTAT |
| 57 | p1478e003 | TT |
| | deletion | |
| 58 | p1494e002 | TCGATACTGATATTTAAAATATGAGCAATTTCAAACAAATCA |
| | deletion | |

1. A composition comprising at least two bacteriophage, wherein a first bacteriophage is at least 80% identical to p1378 or p4815, and a second bacteriophage is at least 80% identical to p1494e002 or p2808.

2. The composition of claim 1, wherein the first bacteriophage is at least 80% identical to p1378, and the second bacteriophage is at least 80% identical to p1494e002.

3. The composition of claim 1, wherein the first bacteriophage is at least 80% identical to p1378, and the second bacteriophage is at least 80% identical to p2808.

4. The composition of claim 1, wherein the first bacteriophage is at least 80% identical to p4815, and the second bacteriophage is at least 80% identical to p1494e002.

5. The composition of claim 1, wherein the first bacteriophage is at least 80% identical to p4815, and the second bacteriophage is at least 80% identical to p2808.

6. The composition of claim 2, comprising a third bacteriophage at least 80% identical to the p4815.

7. The composition of claim 6, comprising a fourth bacteriophage at least 80% identical to the p2808.

8. The composition of claim 3, comprising a third bacteriophage at least 80% identical to the p4815.

9. The composition of claim 4, comprising a third bacteriophage at least 80% identical to the p2808.

10. A composition comprising a plurality of bacteriophage comprising a first bacteriophage and a second bacteriophage, wherein a bacteria treated with the plurality of bacteriophage has a reduced amount of regrowth as compared to treatment with the first bacteriophage or the second bacteriophage alone, wherein the first bacteriophage comprises a Rosenblumvirus and the second bacteriophage comprises a Phietavirus or a Kayvirus.

11. The composition of claim 1, wherein the amount of regrowth is measured by optical density (OD) at a wavelength of 600 nm.

12. The composition of claim 10 or claim 11, wherein the amount of regrowth of the bacteria treated with the plurality of bacteriophage 12 hours after treatment is less than or equal to about 50%, 40%, 30%, 20%, or 10% of regrowth of the bacteria treated with the first bacteriophage.

13. The composition of claim 10 or claim 11, wherein the amount of regrowth of the bacteria treated with the plurality of bacteriophage 12 hours after treatment is less than or equal to about 50%, 40%, 30%, 20%, or 10% of regrowth of the bacteria treated with the second bacteriophage.

14. The composition of any one of claims 10-13, wherein the bacteria is a Staphylococcus bacteria.

15. The composition of claim 14, wherein the bacteria comprise Staphylococcus aureus, methicillin resistant Staphylococcus aureus, Staphylococcus epidermidis, Staphylococcus salivarius, Staphylococcus argenteus, Staphylococcus hemolyticus, or Staphylococcus schweitzeri, or any combination of two or more thereof.

16. The composition of claim 15, wherein the bacteria comprise the Staphylococcus aureus.

17. A bacteriophage engineered to render the bacteriophage lytic by removal, replacement, or inactivation of a lysogenic sequence, wherein the lysogenic sequence comprises at least 10%, 20%, 30%, 40%, 50%, 60%, 70%, 80%, 90%, or 100% of a sequence selected from SEQ ID NOS: 49-58.

18. The bacteriophage of claim 17, wherein the bacteriophage is an engineered Phietavirus.

19. A composition comprising the bacteriophage of claim 17 or 18, further comprising a Rosenblumvirus and/or a Kayvirus.

20. A method of treating a disease or condition related to Staphylococcus, the method comprising administering to a subject in need thereof the bacteriophage of any one of claims 1-9, 17-18, or the composition of any one of claim 10-13 or 19.

21. The method of claim 20, wherein Staphylococcus is causative of, and/or contributes to, the disease or condition.

22. A bacteriophage comprising at least 70%, 75%, 80%, 85%, 90%, 95%, or 100% sequence identity with p1378 (PTA-127329).

23. A bacteriophage comprising at least 70%, 75%, 80%, 85%, 90%, 95%, or 100% sequence identity with p4815 (PTA-127331).

24. A bacteriophage comprising at least 70%, 75%, 80%, 85%, 90%, 95%, or 100% sequence identity with p1494e002 (PTA-127345).

25. A bacteriophage comprising at least 70%, 75%, 80%, 85%, 90%, 95%, or 100% sequence identity with p2808 (PTA-127332).

26. A composition comprising at least two bacteriophage, wherein a first bacteriophage comprises at least 70%, 75%, 80%, 85%, 90%, 95%, or 100% sequence identity with p1378, and a second bacteriophage comprises at least 70%, 75%, 80%, 85%, 90%, 95%, or 100% sequence identity with p1381, p4815, p1378e062, p1378e074, p4815e037, p1378e075, p1381e017, p4815e053, p1498e001, p3693e001, p3224e002, p5593e001, p1468e003, p1478e003, p1494e002, p2808, or p1473.

27. A composition comprising at least two bacteriophage, wherein a first bacteriophage comprises at least 70%, 75%, 80%, 85%, 90%, 95%, or 100% sequence identity with p4815, and a second bacteriophage comprises at least 70%, 75%, 80%, 85%, 90%, 95%, or 100% sequence identity with p1381, p1378, p1378e062, p1378e074, p4815e037, p1378e075, p1381e017, p4815e053, p1498e001, p3693e001, p3224e002, p5593e001, p1468e003, p1478e003, p1494e002, p2808, or p1473.

28. The composition of claim 26 or 27, wherein the second bacteriophage comprises at least at least 70%, 75%, 80%, 85%, 90%, 95% or 100% sequence identity with p4815, 01494e002, or p2808.

29. The composition of claim 28, further comprising a third bacteriophage, wherein the third bacteriophage comprises at least 70%, 75%, 80%, 85%, 90%, 95% or 100% sequence identity with p4815, 01494e002, or p2808.

30. The composition of claim 29, further comprising a fourth bacteriophage, wherein the fourth bacteriophage comprises at least 70%, 75%, 80%, 85%, 90%, 95% or 100% sequence identity with p4815, 01494e002, or p2808.

31. A composition comprising at least two bacteriophage, wherein a first bacteriophage comprises at least 70%, 75%, 80%, 85%, 90%, 95%, or 100% sequence identity with p1494e002, and a second bacteriophage comprises at least 70%, 75%, 80%, 85%, 90%, 95%, or 100% sequence identity with p1381, p1378, p4815, p1378e062, p1378e074, p4815e037, p1378e075, p1381e017, p4815e053, p1498e001, p3693e001, p3224e002, p5593e001, p1468e003, p1478e003, p2808, or p1473.

32. A composition comprising at least two bacteriophage, wherein a first bacteriophage comprises at least 70%, 75%, 80%, 85%, 90%, 95%, or 100% sequence identity with p2808, and a second bacteriophage comprises at least 70%, 75%, 80%, 85%, 90%, 95%, or 100% sequence identity with p1381, p1378, p4815, p1378e062, p1378e074, p4815e037, p1378e075, p1381e017, p4815e053, p1498e001, p3693e001, p3224e002, p5593e001, p1468e003, p1478e003, p1494e002, or p1473.

33. The composition of any one of claims 26-32, wherein the at least two bacteriophage of the composition infect at least about 90% of a collection of at least about 30 Staphylococcus bacteria.

34. The composition of claim 33, wherein the infection of the at least about 90% is determined with a plaque assay or growth inhibition assay.

35. The composition of any one of claims 33-34, wherein the at least about 90% is at least about 95%.

36. The composition of any one of claims 33-34, wherein the at least about 90% is at least about 98%.

37. The composition of any one of claims 33-34, wherein the at least about 90% is at least about 99%.

38. The composition of any one of claims 33-38, wherein the collection of Staphylococcus bacteria comprises comprise Staphylococcus bacteria having a MLST of 8, 5, 22, 15, 1, 30, 398, 105, 45, 672, 2250, 582, 72, 97, 239, 34, 87, 101, 109, 1159, 1165, 1181, 12, 121, 152, 1750, 20, 225, 25, 291, 3628, 59, 7, 779, 88, 10, 1011, 1049, 1156, 1351, 149, 1637, 1649, 1757, 1842, 188, 1970, 2066, 256, 2867, 2945, 3149, 3182, 3510, 39, 395, 4317, 47, 4730, 50, 508, 573, 6, 630, 737, 828, 848, 923, or 93, or a combination of two or more thereof.

39. The composition of any one of claims 33-38, wherein the collection of Staphylococcus bacteria comprises bacteria isolated from a bloodstream infection.

40. The composition of any one of claims 33-39, wherein at least about 40% of the collection of Staphylococcus bacteria are multidrug resistant.


SEQUENCE LISTING (58 sequences)

SEQ ID NO: 1 - DNA, 804 bp, synthetic construct
  misc_feature 1..804: note = Description of Artificial Sequence: Syntheticpolynucleotide
  source 1..804: mol_type = other DNA
atgttaaagattgctgcttttaacattcaaacatttggtgaaacaaaaatgagtaatgctacattagtatcatatattgttcaaattttatctagatatgatattgctttagttcaagaagttagagatagtcatttaacagctgtaggtaaattattagataatttaaatcaagatgctcctgatacataccattacgtagtaagtgaacctttaggtagaaattcatataaagaaagatacttatttgtatatagacctgaccaagtaagtgctgtagattcatattattatgatgatggttgtgaaccttgtggtaatgatacatttaatagagaacctgctattgttagattcttttcaagatttacagaagtaagagaatttgctattgtacctttacatgctgctcctggtgatgctgtagctgaaattgatgctttatatgatgtatatttagatgtacaagaaaaatggggtttagaagatgtaatgttaatgggtgattttaatgctggttgtagttatgtaagaccttcacaatggtctagtattagattatggacaagtcctacatttcaatggttaattcctgattcagctgatacaacagctacacctacacattgtgcttatgatagaattgtagtagctggtatgttattaagaggtgctgtagtacctgattctgctttaccttttaattttcaagctgcttatggtttaagtgaccaattagctcaagctatttcagaccattatcctgtagaagtaatgttaaaacatcatcaccatcatcactaa

SEQ ID NO: 2 - DNA, 300 bp, synthetic construct
  misc_feature 1..300: note = Description of Artificial Sequence: Syntheticpolynucleotide
  source 1..300: mol_type = other DNA
atgattgatatatacttaggagaaggttataataaagaatacttgtctaaagcactcagattaattaatgaccatgctcctagggagttaagttatgattttaataatgtagaagcggatgttaatattcacacaatgttatatgttaaacctgaagataggtatgtatataaggatatatcttattacttcccaggtgatttaattatatgtatagttgaagatgatgctattgtgtatcaccaaggtaaacaggtttcaggtattagtattttaagaataatagaagagatattttaa

SEQ ID NO: 3 - DNA, 288 bp, synthetic construct
  misc_feature 1..288: note = Description of Artificial Sequence: Syntheticpolynucleotide
  source 1..288: mol_type = other DNA
atgaaaactttcaaagaatttacttctacgactaccccggtttctactattactgaagctactcttacttctgaagttattaaagcaaataaaggacgagaaggtaaaccgatgattagtctggttgatggtgaagaaatcaaaggtactgtttacctaggtgatgggtggtctgctaaaaaggatggtgctacaatcgttatctctcctgctgaagaaactgcgttgtttaaagctaaacacatttctgcggcacatctcaagattattgctaaaaatcttttgtaa

SEQ ID NO: 4 - DNA, 27 bp, synthetic construct
  misc_feature 1..27: note = Description of Artificial Sequence: Syntheticoligonucleotide
  source 1..27: mol_type = other DNA
atgtatgctccttggacaaatttttaa

SEQ ID NO: 5 - DNA, 27 bp, synthetic construct
  misc_feature 1..27: note = Description of Artificial Sequence: Syntheticoligonucleotide
  source 1..27: mol_type = other DNA
atgtatagtccttggacaaatttttaa

SEQ ID NO: 6 - skipped

SEQ ID NO: 7 - DNA, 192 bp, synthetic construct
  source 1..192: mol_type = other DNA
atgattgttgcaaagagaaagcgtgttaaaatggcagggtttttaaaagtagttcaattactagctaaatatggttctaaagctgtacaatgggcttgggcaaacaagggtaagattttagattggcttaatgcaggtcaggctattgattgggtagtttcgaaaattaagcaaattttaggtattaagtaa

SEQ ID NO: 8 - skipped

SEQ ID NO: 9 - protein, 267 aa, synthetic construct
  REGION 1..267: note = Description of Artificial Sequence: Syntheticpolypeptide
MLKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCGNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKHHHHHH

SEQ ID NO: 10 - protein, 99 aa, synthetic construct
  REGION 1..99: note = Description of Artificial Sequence: Syntheticpolypeptide
MIDIYLGEGYNKEYLSKALRLINDHAPRELSYDFNNVEADVNIHTMLYVKPEDRYVYKDISYYFPGDLIICIVEDDAIVYHQGKQVSGISILRIIEEIF

SEQ ID NO: 11 - protein, 95 aa, synthetic construct
  REGION 1..95: note = Description of Artificial Sequence: Syntheticpolypeptide
MKTFKEFTSTTTPVSTITEATLTSEVIKANKGREGKPMISLVDGEEIKGTVYLGDGWSAKKDGATIVISPAEETALFKAKHISAAHLKIIAKNLL

SEQ ID NO: 12 - protein, 8 aa, synthetic construct
  REGION 1..8: note = Description of Artificial Sequence: Syntheticpeptide
MYAPWTNF

SEQ ID NO: 13 - protein, 63 aa, synthetic construct
MIVAKRKRVKMAGFLKVVQLLAKYGSKAVQWAWANKGKILDWLNAGQAIDWVVSKIKQILGIK

SEQ ID NO: 14 - protein, 8 aa, synthetic construct
  REGION 1..8: note = Description of Artificial Sequence: Syntheticpeptide
MYSPWTNF

SEQ ID NO: 15 - protein, 51 aa, synthetic construct
  REGION 1..51: note = Description of Artificial Sequence: Syntheticpolypeptide
MAAFMKLIQFLATKGQKYVSLAWKHKGTILKWINAGQSFEWIYKQIKKLWA

SEQ ID NO: 16 - DNA, 150 bp, synthetic construct
  misc_feature 1..150: note = Description of Artificial Sequence: Syntheticpolynucleotide
  source 1..150: mol_type = other DNA
aaacgaaaattggataaagtgggatatttttaaaatatatatttatgttacagtaatattgacttttaaaaaaggattgattctaatgaagaaagcagacaagtaagcctcctaaattcactttagataaaaatttaggaggcatatcaa

SEQ ID NO: 17 - DNA, 29 bp, synthetic construct
  misc_feature 1..29: note = Description of Artificial Sequence: Syntheticoligonucleotide
  source 1..29: mol_type = other DNA
tttacttctcatttttaattagttataat

SEQ ID NO: 18 - DNA, 84 bp, synthetic construct
  misc_feature 1..84: note = Description of Artificial Sequence: Syntheticoligonucleotide
  source 1..84: mol_type = other DNA
taataaataagttgacagaaagttaataatatggtatacttataaagtaatatttagtgggtataccatgttatattaataaag

SEQ ID NO: 19 - DNA, 40 bp, synthetic construct
  misc_feature 1..40: note = Description of Artificial Sequence: Syntheticoligonucleotide
  source 1..40: mol_type = other DNA
agagtttgcaaaatatacaggggattatatataatggaaa

SEQ ID NO: 20 - skipped

SEQ ID NO: 21 - DNA, 24 bp, synthetic construct
  misc_feature 1..24: note = Description of Artificial Sequence: Syntheticoligonucleotide
  source 1..24: mol_type = other DNA
ggtaccttaggaggatgattattt

SEQ ID NO: 22 - DNA, 6297 bp, synthetic construct
  misc_feature 1..6297: note = Description of Artificial Sequence: Syntheticpolynucleotide
  source 1..6297: mol_type = other DNA
atgagattaaagataaattgtgattttgattctaaaataatttcaaaagattttcaaagtaaagttgttagtctgttcaaagcgggcattatgaaatcgagtccagagagatatgagaatttatttggcgggaataagcataagcaatatacattttcagtgtaccttcccaaacctcaaaataaaggtgcggaaattcagttaaatgaagcaaattgtattattaatttttcaacgggagatgcggaaacgggaatcattttttataatgcattgatgagtttgagaggtagcaaagttttatttggagctgggaatcatattacggtaaaaaacattcaaatagtcccggagaaaaaaattatcggcaaacgaacaattttgagaacgctttcacctgtggttagtagagatcataatagagaaacattcaagaattggttttacagttttgaggatgaggaatttgagccaactttgaaaagaaacatgttgccctatttaatggatgcatttggtgagcaagctcgttatgatttggagaaattgaaaataacgccaatttcgatgaagaaagtagtcgtttattgccacgagatacatattgaaagctcagtcgggatttttgaattggaggctgaggcttatttgcagaagtatttggtggagaatggtattgggactatgacaggatctggatttggcatgatagagcagttttagactggaggtggaagaatgcggacagaaatagaagtaagggcaaatgattggttaataaatgctggtttaactggttttctaaatatcgttggaaaagaaaatgtaagaattgatggacaagctatttattttacagcagatttattagaggactttgaaacgaaatactttaatttctttattcaagaatataaagaaacgttatcgtggtataaaatcgtttcttataaagagacaatggaacagtttagaatagatgaatttgcttcttttgacgaggttgcattgggaaatttaaataaatatatgaaagatgtagtaaagttttatttaaaaaaagctaattatatcaaggtgtttccgctaattgatcctagtgctaatgtaacaaaatggttggaaaaattaaatacaattaatttaactaaaaagcaaaaatgggatgaagtaaaagccgatattattgaagaagtaaagcaaacatatactcaactagatctgattattgatttttgtgcaagtgaaaaggggcttcaatatctaggagcaaaaaatctgatatattctgttataaataaaggttggtctggtgtatctttccttcttaaacaaacaaaatttattgatccctatttagattacaaaactagttttttggacccagtaatagattatttagacaccgatttatctaaagcaaaatataattgtttcaattgtaatcaaccaattaagaatttgaaactagatctcagttttatgaacgatgttgggtttgatactgcaagaaaaacagctcacgtatgggattttaacaatgatgttgcgacttgcccgatttgtcgtttgatttattcatgtgttccagctggatttacgtatgtgtacggagaaggtatgtttgtcaacgattcatatggagtagaggaattacaccgtgttaatgaacgtatgcggaattctatcctgcgctttaacaaagatggaattaattcaactaatacttaccgagcgcttgtggagtctattacgatggagcatgaaaataatcgacgctatgaacttgcagacattcagttagttagatacgaaaatgagcattatcgttttaatttactttctaaaaaaatgttgcatattgtaaatgattcaaagggaatattgaaaagtttaattcgctgtggttataaagaaggtaatttgaatgttaatttgtacaaagaagttattcagcatttaatgaacaatgaaaatttatttacgcttattcataaattaatctactataaacaaactagtgtaaatggtttgtattacaatatgggacacgtttcagggattttagatataaatacaaaatttttgaaggagatggaagtgatgacaaatatttctcaaaatcaattatggttcgttcaaaattgtggaaaggaatttaaggaaggttatgttaagaagaagtcagaaaataaaatttcgggaataacatacaagttattaaattcgttaaaggtcaatgataaagatggatttatggatactttattgaatagttattcctatttatctatgcccattccagatgtattcataaaaatgttttcgaataatgaagcttttaaatcagtaggttatgcatttatgcttggagtaggcggagagaaaactaaaaaagaagacgggggaaacacagatgaaaaataaaggactagcaatgacaattattttccaagcagaaagtgccaactacggggagtctcttggtaatatttcttcacttaaaaagatttctagaaataacggtgaccaatatacatatatttctcgccaagcaattcgctacaacttaatggatcaaattggagagaaagaagctcctgtaaaggcggaaggtggcggcgataagaaagttattcaattcttgagtgaagcgactattacagatttcccagaacttgatttctttggttaccttaaaacagaaaaaggaagcggtgggcaaaaacgttctgcaaaagtccgtttatctaatgccatttcgcttgaaacatttaaaggtgatttggatttcctaacgaataaaggtcaagctgataaattaaatgaaaacatgaatatcgcacaagctgaaattcataaatcttattatcgttatacgattacgattgatttagatcaaattggtattgacggtgaaattgcacttgataataaagaaaaagctcgtcgtgtaaaaaaattgatggatacagtagcattcttgtaccgtgacattcgcgggcgccgagaagatttaaaaccacttttcgtcattggcggagtttatgacgtgaaaaatcctgttttccaaaatattgtagatgtagcagataacaatattgttattaaaaatattaaagatttacttacgtatgaagatatcaaagaaaatacgagagtgggtattattgacgggcaatttgctaattctgatgaagtgaaagtcgagctaaaagcagaatctgtgccggcattctttaacgatattaaaggaaagattgatgcttattatgaaagcaattagagtaagactttggcaagacttggtgaattttaagaaaccaactagttttcaattaaaagaaacgtatcctttgccgccttattcaactgtaattgggatggttcatacgctttgtggttttacaagctatcatgaaatgaaaattagtattcaagggaagtacttttcgaaggtgaacgatttagcgacgaggtatgagtttaaaaatgggatgacttatgatgcatcacgacaccaaattaaagtagataagtatggtgttagtcgtggtgtatcaacggttgagcttcttgtggatttagaactactgctgcatatcattccggaagacccatcactcgtaccaatcatcgaaaaagcatttagagagcctattgagttcccatcgctaggtcgtcgcgaggatattgcgactattcaaaaggtggaagttgtggatgtggagaaacggcaacctaaaaaaagcacagaaatatcagatggttacaatgcctatgtgccaatctcgctcactgaaaataattctgttcatttcaaatcacatgaaagctcagtcggtcgtgataagttgttaggtacaagatatttgctaacagaaaaatatgaaagagttaaccacggaacagaaaaagcacctaagtttttccgtaagtggcgaaagaaagatgttatttactcaagtagaatttttgtctctaagaaagatgttttcttttttgatgaagatgattacttagtctttattgaagaagaggagtaataataacaaacaattaccccttgagtgtatagtgttcaaggggattttgtataaggagcggcttgagatgcataaatatttagcgaaatctaatccagaagaaacaattcaagaacatacggataatttattaaaaaactatcaaagactaaaaaaactatacccagctgtggaaatagattggtatttactggaattagtttgtttactacatgatttagggaaaatgaatctattattccaaacgaaattaggtaatgcttctggtaaaggtaaagaaattccgcatggttatttgtctgttgcatttgttccagatagtaaacttgaagaaatgggatactcggaggatgaaattaaagttgtttatcaggcgattgcgcgacatcatgaaaggaaatatgatttttctgattcagagtggaaaaaagaaataatcaaattagaagagcagtgggcgggttttatatacagtcagctaggaggaaatgcggaatatagtagtgaggtagaaatgttgtacttctacccaggctcccgaattttcgaaggtaactccgcagcggacgcagaaacattcaaaaaatacgttctactaaaaggcttactcaatcgaattgactttgcagcgagtgggggaattgatgtagaacttgaaaatgacttcttgatggattcgatggaagtgcaactagcccattttaaagcagaaaatccagaagctgattggaacgctttgcaaaaatatatgatgcagcaccaaaatgaaaatattgtcgttattgctgaaactggtatggggaaaacggaggcagggctgctatggcttgggaataataagggttttttcacgctaccattgcgggcagcaatcaatgcaatttatgagcgaatcacaaaaaatattgttacgaccaatcaagccgagcgagtgggtttacttcattcggaaacatatagtcagtatttgctgcatgaaggaaatgcggagatggatattgatgaatattatacgaggacgaggcaaatgtctttacctataactatttgcacgctcgatcaattgtttgattttgtctttcgttatgcaggttttgaacataaattagccactttatcttattccaaagtgattattgatgaaatccagatgtactcacccgacttactggcttacttgattttagggttatcttatattgataaatttggtgggaagttctgcattatgacagcgactttaccgggaatagtgacagatttacttttagaaaatggagtggattttgtccaaccagaagagaaatttgtatcgtcgcgaattcgccacagcatggagatggttcatacagagattgaaagcgaatttattaaaccattttttaacaataatcgaattttagttatttgtaacactattagtaaagcgaaaaaaatctattcggagttaaaagagcattttcaaggtgaagaaattcatctcattcacagtcaatttattaaaagagaccgctcagctaaagaaaaagcaatttttaaagatgggcaaaaagatagtacgaaaaaatgtatttgggtagcaacgcaagtcgtagaggcttcattggatattgattttgatttactattcacggagctatcagatgtgaatgggctgtttcaacggatgggcagatgctatcgaaaccgggcgctggatgtggatacgaatgtctatgtttttgatggtggagcgaaagtttgctcaggaattggaacttttatcgataagttgatatttgtaaattctaagacgattttaaatgagcatgctggcgttttgacagaagagaagaaaatggagctagtggagcgggtttattcaacagaggctttacgtgggagtgaattttataacgatttgagaaagacgataaactatgtgaaagcttttgatagttatgaattggataaagcggatgttcgtaggagatttagaaacattaattcggtttctgttatcccagaagaagtttggcaagataatgcggaagaaatcaattcctattttgttactcttcgtaaaacctctaaggaaatatcaacaaaagagaaaatcattgcaagaaccaatttagcagaatttatggtgagtattccagactatctttataaaaaaggtgagagcgttgtacgcgaaataaaccgttatgagagtgttattgagtttaaatgtggttattcgagtgatgtaggggtttttatgaggtga

SEQ ID NO: 23 - DNA, 6297 bp, synthetic construct
  misc_feature 1..6297: note = Description of Artificial Sequence: Syntheticpolynucleotide
  source 1..6297: mol_type = other DNA
atgagattaaagataaattgtgattttgattctaaaataatttcaaaagattttcaaagtaaagttgttagtctgttcaaagcgggcattatgaaatcgagtccagagagatatgagaatttatttggcgggaataagcataagcaatatacattttcagtgtaccttcccaaacctcaaaataaaggtgcggaaattcagttaaatgaagcaaattgtattattaatttttcaacgggagatgcggaaacgggaatcattttttataatgcattgatgagtttgagaggtagcaaagttttatttggagctgggaatcatattacggtaaaaaacattcaaatagtcccggagaaaaaaattatcggcaaacgaacaattttgagaacgctttcacctgtggttagtagagaccataatagagaaacattcaagaattggttttacagttttgaggatgaggaatttgagccaactttgaaaagaaacatgttgccctatttaatggatgcatttggtgagcaagctcgttatgatttggagaaattgaaaataacgccaatttcgatgaagaaagtagtcgtttattgccacgagatacatattgaaagctcagtcgggatttttgaattggaggctgaggcttatttgcagaagtatttggtggagaatggtattgggactatgacaggttctggatttggcatgatagagcagttttagactggaggtggaagaatgcggacagaaatagaagtaagggcaaatgattggttaataaatgctggtttaactggttttctaaatatcgttggaaaagaaaatgtaagaattgatggacaagctatttattttacagcagatttattagaggactttgaaacgaaatactttaatttctttattcaagaatataaagaaacgttatcgtggtataaaatcgtttcttataaagagacaatggaacagtttagaatagatgaatttgcttcttttgacgaggttgcattgggaaatttaaataaatatatgaaagatgtagtaaagttttatttaaaaaaagctaattatatcaaggtgtttccgctaattgaccctagtgctaatgtaacaaaatggttggaaaaattaaatacaattaatttaactaaaaagcaaaaatgggatgaagtaaaagccgatattattgaagaagtaaagcaaacatatactcaactagacctgattattgatttttgtgcaagtgaaaaggggcttcaatatctaggagcaaaaaatctgatatattctgttataaataaaggttggtctggtgtatctttccttcttaaacaaacaaaatttattgacccctatttagattacaaaactagttttttggacccagtaatagattatttagacaccgatttatctaaagcaaaatataattgtttcaattgtaatcaaccaattaagaatttgaaactagacctcagttttatgaacgatgttgggtttgatactgcaagaaaaacagctcacgtatgggattttaacaatgatgttgcgacttgcccgatttgtcgtttgatttattcatgtgttccagctggatttacgtatgtgtacggagaaggtatgtttgtcaacgattcatatggagtagaggaattacaccgtgttaatgaacgtatgcggaattctatcctgcgctttaacaaagatggaattaattcaactaatacttaccgagcgcttgtggagtctattacgatggagcatgaaaataatcgacgctatgaacttgcagacattcagttagttagatacgaaaatgagcattatcgttttaatttactttctaaaaaaatgttgcatattgtaaatgattcaaagggaatattgaaaagtttaattcgctgtggttataaagaaggtaatttgaatgttaatttgtacaaagaagttattcagcatttaatgaacaatgaaaatttatttacgcttattcataaattaatctactataaacaaactagtgtaaatggtttgtattacaatatgggacacgtttcagggattttagatataaatacaaaatttttgaaggagatggaagtgatgacaaatatttctcaaaatcaattatggttcgttcaaaattgtggaaaggaatttaaggaaggttatgttaagaagaagtcagaaaataaaatttcgggaataacatacaagttattaaattcgttaaaggtcaatgataaagatggatttatggatactttattgaatagttattcctatttatctatgcccattccagatgtattcataaaaatgttttcgaataatgaagcttttaaatcagtaggttatgcatttatgcttggagtaggcggagagaaaactaaaaaagaagacgggggaaacacagatgaaaaataaaggactagcaatgacaattattttccaagcagaaagtgccaactacggggagtctctaggtaatatttcttcacttaaaaagatttctagaaataatggagaccaatatacatatattagcagacaagcaattagatacaacctcatggaccaaattggagaaaaagaagctcctgttaaagcagaaggtggtggagataagaaagttattcaattcttaagtgaagctactattacagatttccctgaacttgatttctttggttaccttaaaacagaaaaaggaagtggcggacaaaagagaagtgcaaaagtacgtttatctaatgctatttctcttgaaacatttaaaggtgatttggatttcctaacaaataaaggtcaagcagataaattaaatgaaaatatgaatattgcacaagcagaaattcataaatcttattatcgttatacgattacgattgatttagaccaaattggtattgacggtgaaattgcacttgataataaagaaaaagctcgtcgtgtaaaaaaattgatggatacagtagcattcttgtaccgtgacattcgcgggcgccgagaagatttaaaaccacttttcgtcattggcggagtttatgacgtgaaaaatcctgttttccaaaatattgtagatgtagcagataacaatattgttattaaaaatattaaagatttacttacgtatgaagatatcaaagaaaatacgagagtgggtattattgacgggcaatttgctaattctgatgaagtgaaagtcgagctaaaagcagaatctgtgccggcattctttaacgatattaaaggaaagattgatgcttattatgaaagcaattagagtaagattatggcaagatttggtaaattttaagaaaccaactagttttcaattaaaagaaacatatcctttacctccttattcaactgtaattgggatggtacatacactatgtggatttacaagctatcatgaaatgaaaattagtatacaaggtaaatatttttctaaagtaaatgatttagctacaagatatgaatttaaaaatggtatgacatatgatgcttctcgtcatcaaattaaagtagataagtatggtgtaagtagaggtgtatcaacagtagaattattagtagatttagaattattattacatattattcctgaagatccatcattagtaccaatcatagaaaaagcttttagagagcctattgagtttcctagtctaggtagaagagaggatattgctacaattcaaaaggtagaagttgtagatgtagaaaaaagacaacctaaaaaaagtacagaaatatcagatggttacaatgcttatgtaccaatctctttaactgaaaataattctgttcatttcaaatctcatgaatcatcagtaggtagagataagttgttaggtacaagatatttgctaacagaaaaatatgaaagagttaatcatggaacagaaaaagcacctaagtttttcagaaaatggagaaagaaagatgttatttactcaagtagaatttttgtctctaagaaagatgttttcttttttgatgaagatgattacttagtctttattgaagaagaggagtaataataacaaacaattaccccttgagtgtatagtgttcaaggggattttgtataaggagcggcttgagatgcataaatatttagctaaatctaatcctgaagaaacaattcaagagcataccgataatttattaaaaaactatcaaagactaaaaaaactatatcctgctgtagaaatagattggtatttattagaattagtttgtttactacatgatttagggaaaatgaatctattattccaaactaaattaggtaatgcttcaggtaaaggtaaagaaataccacatggttatttgtctgttgcatttgttcctgatagtaaacttgaagaaatgggatattctgaagatgaaattaaagttgtttatcaagctatagctaggcatcatgaaaggaaatatgatttttctgattcagaatggaaaaaagaaataatcaaattagaagaacaatgggcaggttttatatattctcaattaggtggaaatgcagaatatagtagtgaggtagaaatgttatatttctatcctggttcaagaatttttgaaggtaactctgcagctgatgcagaaacattcaaaaaatacgttttattaaaaggtttattaaatagaattgactttgcagctagtggtggtattgatgtagaacttgaaaatgatttcttaatggatagtatggaagtacaattagcacattttaaagcagaaaatcctgaagcggattggaacgcattacaaaaatatatgatgcaacatcaaaatgaaaatattgtagttattgcagaaacaggtatgggtaaaacagaagcaggtttattatggttaggaaataataaaggttttttcacattacctttacgtgctgcaattaatgcaatttatgaaagaattacaaaaaatattgtaacaacaaatcaagcagaaagagtaggtctactacattcagaaacatattctcaatatttattacatgaaggtaatgctgaaatggatattgatgaatattatacaagaacaagacaaatgtctttacctataacaatatgtacattagaccaattatttgattttgtatttagatatgcaggttttgaacataaattagctacattatcatattctaaagtaattattgatgaaattcaaatgtattcacccgatttattagcttacttgattttagggttatcttatattgataaatttggtggaaagttctgcattatgacagcaacattacctggtatagtaacagatttacttttagaaaatggagttgattttgtacaacctgaagagaaatttgtatcttctagaattagacatagcatggagatggttcatacagaaattgaatcagaatttattaaaccattttttaacaataatagaattttagttatttgtaacactattagtaaagctaaaaaaatttattctgagttaaaagagcattttcaaggtgaagaaatacatttaattcacagtcaatttattaaaagagatagaagtgctaaagaaaaagcaatatttaaagatggtcaaaaagatagtacaaaaaaatgtatttgggtagcaacacaagtagtagaagcttcattggatattgattttgatttactattcacggagctatcagatgtgaatgggctgtttcaacggatgggcagatgctatcgaaaccgggcgctggatgtggatacgaatgtctatgtttttgatggtggagcgaaagtttgctcaggaattggaacttttatcgataagttgatatttgtaaattctaagacgattttaaatgagcatgctggcgttttgacagaagagaagaaaatggagctagtggagcgggtttattcaacagaggctttacgtgggagtgaattttataacgatttgagaaagacgataaactatgtgaaagcttttgatagttatgaattggataaagcggatgttcgtaggagatttagaaacattaattcggtttctgttatcccagaagaagtttggcaagataatgcggaagaaatcaattcctattttgttactcttcgtaaaacctctaaggaaatatcaacaaaagagaaaatcattgcaagaaccaatttagcagaatttatggtgagtattccagactatctttataaaaaaggtgagagcgttgtacgcgaaataaaccgttatgagagtgttattgagtttaaatgtggttattcgagtgatgtaggggtttttatgaggtga

SEQ ID NO: 24 - DNA, 267 bp, synthetic construct
  misc_feature 1..227: note = Description of Artificial Sequence: Syntheticpolynucleotide
  source 1..267: mol_type = other DNA
agagtttgcaaaatatacaggggattatatataatggaaagttttaactacttattatgaaatgtaaatcgtttaataccttgttcaattttatcagctatttcttgttttaactacttattatgaaatgtaaatacttgcattgtctgtagaaattgggaatccaatttctgttttaactacttattatgaaatgtaaataagaattattattaaaaggtttagaaaaattagaatagttttaactacttattatgaaatgtaaat

SEQ ID NO: 25 - protein, 240 aa, synthetic construct
  REGION 1..240: note = Description of Artificial Sequence: Syntheticpolypeptide
MRLKINCDFDSKIISKDFQSKVVSLFKAGIMKSSPERYENLFGGNKHKQYTFSVYLPKPQNKGAEIQLNEANCIINFSTGDAETGIIFYNALMSLRGSKVLFGAGNHITVKNIQIVPEKKIIGKRTILRTLSPVVSRDHNRETFKNWFYSFEDEEFEPTLKRNMLPYLMDAFGEQARYDLEKLKITPISMKKVVVYCHEIHIESSVGIFELEAEAYLQKYLVENGIGTMTGSGFGMIEQF

SEQ ID NO: 26 - protein, 289 aa, synthetic construct
  REGION 1..289: note = Description of Artificial Sequence: Syntheticpolypeptide
MKNKGLAMTIIFQAESANYGESLGNISSLKKISRNNGDQYTYISRQAIRYNLMDQIGEKEAPVKAEGGGDKKVIQFLSEATITDFPELDFFGYLKTEKGSGGQKRSAKVRLSNAISLETFKGDLDFLTNKGQADKLNENMNIAQAEIHKSYYRYTITIDLDQIGIDGEIALDNKEKARRVKKLMDTVAFLYRDIRGRREDLKPLFVIGGVYDVKNPVFQNIVDVADNNIVIKNIKDLLTYEDIKENTRVGIIDGQFANSDEVKVELKAESVPAFFNDIKGKIDAYYESN

SEQ ID NO: 27 - protein, 257 aa, synthetic construct
  REGION 1..257: note = Description of Artificial Sequence: Syntheticpolypeptide
MKAIRVRLWQDLVNFKKPTSFQLKETYPLPPYSTVIGMVHTLCGFTSYHEMKISIQGKYFSKVNDLATRYEFKNGMTYDASRHQIKVDKYGVSRGVSTVELLVDLELLLHIIPEDPSLVPIIEKAFREPIEFPSLGRREDIATIQKVEVVDVEKRQPKKSTEISDGYNAYVPISLTENNSVHFKSHESSVGRDKLLGTRYLLTEKYERVNHGTEKAPKFFRKWRKKDVIYSSRIFVSKKDVFFFDEDDYLVFIEEEE

SEQ ID NO: 28 - protein, 727 aa, synthetic construct
  REGION 1..727: note = Description of Artificial Sequence: Syntheticpolypeptide
MHKYLAKSNPEETIQEHTDNLLKNYQRLKKLYPAVEIDWYLLELVCLLHDLGKMNLLFQTKLGNASGKGKEIPHGYLSVAFVPDSKLEEMGYSEDEIKVVYQAIARHHERKYDFSDSEWKKEIIKLEEQWAGFIYSQLGGNAEYSSEVEMLYFYPGSRIFEGNSAADAETFKKYVLLKGLLNRIDFAASGGIDVELENDFLMDSMEVQLAHFKAENPEADWNALQKYMMQHQNENIVVIAETGMGKTEAGLLWLGNNKGFFTLPLRAAINAIYERITKNIVTTNQAERVGLLHSETYSQYLLHEGNAEMDIDEYYTRTRQMSLPITICTLDQLFDFVFRYAGFEHKLATLSYSKVIIDEIQMYSPDLLAYLILGLSYIDKFGGKFCIMTATLPGIVTDLLLENGVDFVQPEEKFVSSRIRHSMEMVHTEIESEFIKPFFNNNRILVICNTISKAKKIYSELKEHFQGEEIHLIHSQFIKRDRSAKEKAIFKDGQKDSTKKCIWVATQVVEASLDIDFDLLFTELSDVNGLFQRMGRCYRNRALDVDTNVYVFDGGAKVCSGIGTFIDKLIFVNSKTILNEHAGVLTEEKKMELVERVYSTEALRGSEFYNDLRKTINYVKAFDSYELDKADVRRRFRNINSVSVIPEEVWQDNAEEINSYFVTLRKTSKEISTKEKIIARTNLAEFMVSIPDYLYKKGESVVREINRYESVIEFKCGYSSDVGVFMR

SEQ ID NO: 29 - protein, 562 aa, synthetic construct
  REGION 1..562: note = Description of Artificial Sequence: Syntheticpolypeptide
MRTEIEVRANDWLINAGLTGFLNIVGKENVRIDGQAIYFTADLLEDFETKYFNFFIQEYKETLSWYKIVSYKETMEQFRIDEFASFDEVALGNLNKYMKDVVKFYLKKANYIKVFPLIDPSANVTKWLEKLNTINLTKKQKWDEVKADIIEEVKQTYTQLDLIIDFCASEKGLQYLGAKNLIYSVINKGWSGVSFLLKQTKFIDPYLDYKTSFLDPVIDYLDTDLSKAKYNCFNCNQPIKNLKLDLSFMNDVGFDTARKTAHVWDFNNDVATCPICRLIYSCVPAGFTYVYGEGMFVNDSYGVEELHRVNERMRNSILRFNKDGINSTNTYRALVESITMEHENNRRYELADIQLVRYENEHYRFNLLSKKMLHIVNDSKGILKSLIRCGYKEGNLNVNLYKEVIQHLMNNENLFTLIHKLIYYKQTSVNGLYYNMGHVSGILDINTKFLKEMEVMTNISQNQLWFVQNCGKEFKEGYVKKKSENKISGITYKLLNSLKVNDKDGFMDTLLNSYSYLSMPIPDVFIKMFSNNEAFKSVGYAFMLGVGGEKTKKEDGGNTDEK

SEQ ID NO: 30 - DNA, 1206 bp, synthetic construct
  misc_feature 1..1206: note = Description of Artificial Sequence: Syntheticpolynucleotide
  source 1..1206: mol_type = other DNA
ggaggtgataagtatggaatttaatgattttcaaaatttctttggtgaacttagtaatcaagccgaaaaagaattcggtggtgacagtgacttttttagagatagaataaataagttgaaagaagatgctcctgaaaacgtatcttacgaaattatttattcaatagctttatacgaaagcttaaaagctcaacaagatatgaaaattttgaatacagttaaatatcttttagatcgtgactagcaatatccaacaatgatttgctctgagcattattaatttttggataatcaaaatttctaagtttaaatcttgtgtttttctcaatctttacaaccttccacgtcacaactgccattgtgatgaggagggttgttttgtatagtgtgttcattgataattcctcctattaagatttttatttttctcctaaaaacttattaacaaagtattgttgtcctttgcctgttacttttggcgtcttactaattgatgtgtgaccgtccgaatgtgtgattgatgtttctttaatttcgaataactcacgttccattgaatactgtgtaggcatgttataatccacacccttgcgtttaataaggaatccgttttgacgtaaccactcaaacaatctgcgttgcccgatgtttataccgttttgtttaatgatctttgctaactctccaactaaaattgatgtcttagtagtagctactgcatctgcaaatacaatttttggtttatcacgttcaatctttgtttctaattgattgattgtgttgttagcaatttttaaagcacgttgcataatcatttctgggctgttccatgctttttcaacttggatgaaatattgtcttgcacgtttaccgggttcactacgttgaatcattgcgatttcttttgcagtgtctagtgtgagtgcgtggtctaaataattaatagcgttaccttgagctgttactcttttttgagtaagagctgtataatcaatattttcttcaaagccataattaatcattctttcaaaccaatcgttatatcttgtcttaacttctaatgcttgatgaagttctcgaccactgattgcgatttctccattttctttttcttgtatgttgaacatttctccgatgttcgattttgtttgtaatgcttgcataatgtttatgctcctttcgtgtataatgttgttatcaacctaa

SEQ ID NO: 31 - DNA, 1027 bp, synthetic construct
  misc_feature 1..1027: note = Description of Artificial Sequence: Syntheticpolynucleotide
  source 1..1027: mol_type = other DNA
gtacgatgatatattacacgaagaagtatttttcaaagaagatgaagcgccatcaaatgctgatttttgcattttaattaacggcgattcaatggaacctatgttaaagcaaggaacatacgcttttattaagaaagaagattatattaaagatggcacaattgcactcgttgtattagatggagtaagtcttatcaagcgtgtagatatatgcgaagactatattaatttggtatctctaaatccgaagtatgatgatatcaaagtcgcttcgtttagtaatattaaagtaatgggcaaagttgtattgtgattaatagcgcctatatggcactttaatataaaagacgtctatttcatcagtgtttaaaaggagtttataatgaaaataactaattgcaaaataaaaaaagaaactatagtatatgaagttttaactagtggtaatcaaccattcacttatgagttatctaaagatttatcgtcacataatgcgcgtaaatacttggaatttatttcacaaaaaatagatggcgataagttaaattaattcaaagaataaagtaacttcataaagagtacgaagaaaacgatctaatgaccgaacttattcttgaatatttagtaaaaaagtatgttgaagaagaatataagaaataaacgcctatatggcgtgaggaggatgagggatggaagagaacgcacctttagaaacagcagttaataattttaaaaagattcaaaatagcgagatttacaaatttaaatatatgaattcatggtgtcttgaatattcagagtttttattggatgaagttagattgttaaaagaaaacaaaagttacaccagatataaaaaaggcactataatttatgtaaagttaggtgttaatgttggcagagagttttctggaaaccatttttgtatggtacttaataatcacgattcaaataaaaatccaatattaacggtagttccacttacatcttccagaagtaaattcaatgtgcatatcgaagaagattt

SEQ ID NO: 32 - DNA, 1503 bp, synthetic construct
  misc_feature 1..1503: note = Description of Artificial Sequence: Syntheticpolynucleotide
  source 1..1503: mol_type = other DNA
tctttttttatacaattttcacgggtagcacgcctacccttattattttttgccaattttgaggagggagaagcaaaatgccagtatataaggatgataatacaggtaaatggtatttttccattagatataaagatgtatacggtaataacaaacgaaaaatgaagcgtgggtttgaacgtaagaaagatgccaaactagccgaaagcgaatttatacaaaatgttaaatatggatactcggacaatcaaccctttgaatatatattttttaatcgtttaaaaaatgaaaatctttctgcacgctcaatagaaaagcgaactacagaatataatactcacataaaagaaaggttcggaaatatccctattggcaaaatcactactacgcaatgtactgctttcaggaattatttgttaaacgatgcaggtctttctgttggctatgcacgatctgtgtgggcaggttttaaagcagttatcaattacgccaaaaagcattacaagctcttatacgaccccacattatcggtaactcctattcccagaacaaaaccacaagctaaatttatcactcgtgaagaatttgatgaaaaagtagaacaaatcacaaacgatacttctcgtcagctaactaaactgttattttattctggtcttagaataggcgaagctttagctttgcagtggaaagattacgataaaataaaaggcgaaattgacgtaaataagaaaatcaatttaagtaatagagaaattgaatataatctaaaaaaagaaaattctaaagggataatacctgtaccaaaattaattagagagatgcttaaaaacatgtataatgaatcttctaaaagatataaatattttgacgaaaactattttatattcggggggttagaacctattagatacgttacctattcgtatcattttaaatctgtattcccgaatctaaaaatacaccatttaagacactcgtacgcaagctatttaattaataatggtgtagatatgtatttattaatggaattaatgaggcactctaacattacagaaacaattcaaacgtactctcatttatatactgataaaaaacatcaagctatgaacatatttgattaaatggtatcatatcggtatcaaataacgattaaggagtttataaaatgcgtaataacaagcctaaaataagaattcataatgacccatgggaagtgaaatttatatacatttaaatttcatgagacaataaacgttgatttaatgcgttttttgccttttttattttccttattttttctgttttacaacaaaatggtatcaaaaatggtatcatttgtagttattttagcttcacatattaaaacaaccacactcctaaattaataggtggtgtggtttgatcatttataatataacataaaaaacaaccacccagtaactagtatggatggttaaggtgtgcctgcagcacataataaaaccgataatatgttttatt

SEQ ID NO: 33 - DNA, 723 bp, Listeria monocytogenes
  source 1..723: mol_type = other DNA
atgagattaaagataaattgtgattttgattctaaaataatttcaaaagattttcaaagtaaagttgttagtctgttcaaagcgggcattatgaaatcgagtccagagagatatgagaatttatttggcgggaataagcataagcaatatacattttcagtgtaccttcccaaacctcaaaataaaggtgcggaaattcagttaaatgaagcaaattgtattattaatttttcaacgggagatgcggaaacgggaatcattttttataatgcattgatgagtttgagaggtagcaaagttttatttggagctgggaatcatattacggtaaaaaacattcaaatagtcccggagaaaaaaattatcggcaaacgaacaattttgagaacgctttcacctgtggttagtagagatcataatagagaaacattcaagaattggttttacagttttgaggatgaggaatttgagccaactttgaaaagaaacatgttgccctatttaatggatgcatttggtgagcaagctcgttatgatttggagaaattgaaaataacgccaatttcgatgaagaaagtagtcgtttattgccacgagatacatattgaaagctcagtcgggatttttgaattggaggctgaggcttatttgcagaagtatttggtggagaatggtattgggactatgacaggatctggatttggcatgatagagcagttttag

SEQ ID NO: 34 - DNA, 1689 bp, Listeria monocytogenes
  source 1..1689: mol_type = other DNA
atgcggacagaaatagaagtaagggcaaatgattggttaataaatgctggtttaactggttttctaaatatcgttggaaaagaaaatgtaagaattgatggacaagctatttattttacagcagatttattagaggactttgaaacgaaatactttaatttctttattcaagaatataaagaaacgttatcgtggtataaaatcgtttcttataaagagacaatggaacagtttagaatagatgaatttgcttcttttgacgaggttgcattgggaaatttaaataaatatatgaaagatgtagtaaagttttatttaaaaaaagctaattatatcaaggtgtttccgctaattgatcctagtgctaatgtaacaaaatggttggaaaaattaaatacaattaatttaactaaaaagcaaaaatgggatgaagtaaaagccgatattattgaagaagtaaagcaaacatatactcaactagatctgattattgatttttgtgcaagtgaaaaggggcttcaatatctaggagcaaaaaatctgatatattctgttataaataaaggttggtctggtgtatctttccttcttaaacaaacaaaatttattgatccctatttagattacaaaactagttttttggacccagtaatagattatttagacaccgatttatctaaagcaaaatataattgtttcaattgtaatcaaccaattaagaatttgaaactagatctcagttttatgaacgatgttgggtttgatactgcaagaaaaacagctcacgtatgggattttaacaatgatgttgcgacttgcccgatttgtcgtttgatttattcatgtgttccagctggatttacgtatgtgtacggagaaggtatgtttgtcaacgattcatatggagtagaggaattacaccgtgttaatgaacgtatgcggaattctatcctgcgctttaacaaagatggaattaattcaactaatacttaccgagcgcttgtggagtctattacgatggagcatgaaaataatcgacgctatgaacttgcagacattcagttagttagatacgaaaatgagcattatcgttttaatttactttctaaaaaaatgttgcatattgtaaatgattcaaagggaatattgaaaagtttaattcgctgtggttataaagaaggtaatttgaatgttaatttgtacaaagaagttattcagcatttaatgaacaatgaaaatttatttacgcttattcataaattaatctactataaacaaactagtgtaaatggtttgtattacaatatgggacacgtttcagggattttagatataaatacaaaatttttgaaggagatggaagtgatgacaaatatttctcaaaatcaattatggttcgttcaaaattgtggaaaggaatttaaggaaggttatgttaagaagaagtcagaaaataaaatttcgggaataacatacaagttattaaattcgttaaaggtcaatgataaagatggatttatggatactttattgaatagttattcctatttatctatgcccattccagatgtattcataaaaatgttttcgaataatgaagcttttaaatcagtaggttatgcatttatgcttggagtaggcggagagaaaactaaaaaagaagacgggggaaacacagatgaaaaataa

SEQ ID NO: 35 - DNA, 870 bp, Listeria monocytogenes
  source 1..870: mol_type = other DNA
atgaaaaataaaggactagcaatgacaattattttccaagcagaaagtgccaactacggggagtctcttggtaatatttcttcacttaaaaagatttctagaaataacggtgaccaatatacatatatttctcgccaagcaattcgctacaacttaatggatcaaattggagagaaagaagctcctgtaaaggcggaaggtggcggcgataagaaagttattcaattcttgagtgaagcgactattacagatttcccagaacttgatttctttggttaccttaaaacagaaaaaggaagcggtgggcaaaaacgttctgcaaaagtccgtttatctaatgccatttcgcttgaaacatttaaaggtgatttggatttcctaacgaataaaggtcaagctgataaattaaatgaaaacatgaatatcgcacaagctgaaattcataaatcttattatcgttatacgattacgattgatttagatcaaattggtattgacggtgaaattgcacttgataataaagaaaaagctcgtcgtgtaaaaaaattgatggatacagtagcattcttgtaccgtgacattcgcgggcgccgagaagatttaaaaccacttttcgtcattggcggagtttatgacgtgaaaaatcctgttttccaaaatattgtagatgtagcagataacaatattgttattaaaaatattaaagatttacttacgtatgaagatatcaaagaaaatacgagagtgggtattattgacgggcaatttgctaattctgatgaagtgaaagtcgagctaaaagcagaatctgtgccggcattctttaacgatattaaaggaaagattgatgcttattatgaaagcaattag

SEQ ID NO: 36 - DNA, 774 bp, Listeria monocytogenes
  source 1..774: mol_type = other DNA
atgaaagcaattagagtaagactttggcaagacttggtgaattttaagaaaccaactagttttcaattaaaagaaacgtatcctttgccgccttattcaactgtaattgggatggttcatacgctttgtggttttacaagctatcatgaaatgaaaattagtattcaagggaagtacttttcgaaggtgaacgatttagcgacgaggtatgagtttaaaaatgggatgacttatgatgcatcacgacaccaaattaaagtagataagtatggtgttagtcgtggtgtatcaacggttgagcttcttgtggatttagaactactgctgcatatcattccggaagacccatcactcgtaccaatcatcgaaaaagcatttagagagcctattgagttcccatcgctaggtcgtcgcgaggatattgcgactattcaaaaggtggaagttgtggatgtggagaaacggcaacctaaaaaaagcacagaaatatcagatggttacaatgcctatgtgccaatctcgctcactgaaaataattctgttcatttcaaatcacatgaaagctcagtcggtcgtgataagttgttaggtacaagatatttgctaacagaaaaatatgaaagagttaaccacggaacagaaaaagcacctaagtttttccgtaagtggcgaaagaaagatgttatttactcaagtagaatttttgtctctaagaaagatgttttcttttttgatgaagatgattacttagtctttattgaagaagaggagtaa

SEQ ID NO: 37 - DNA, 2184 bp, Listeria monocytogenes
  source 1..2184: mol_type = other DNA
atgcataaatatttagcgaaatctaatccagaagaaacaattcaagaacatacggataatttattaaaaaactatcaaagactaaaaaaactatacccagctgtggaaatagattggtatttactggaattagtttgtttactacatgatttagggaaaatgaatctattattccaaacgaaattaggtaatgcttctggtaaaggtaaagaaattccgcatggttatttgtctgttgcatttgttccagatagtaaacttgaagaaatgggatactcggaggatgaaattaaagttgtttatcaggcgattgcgcgacatcatgaaaggaaatatgatttttctgattcagagtggaaaaaagaaataatcaaattagaagagcagtgggcgggttttatatacagtcagctaggaggaaatgcggaatatagtagtgaggtagaaatgttgtacttctacccaggctcccgaattttcgaaggtaactccgcagcggacgcagaaacattcaaaaaatacgttctactaaaaggcttactcaatcgaattgactttgcagcgagtgggggaattgatgtagaacttgaaaatgacttcttgatggattcgatggaagtgcaactagcccattttaaagcagaaaatccagaagctgattggaacgctttgcaaaaatatatgatgcagcaccaaaatgaaaatattgtcgttattgctgaaactggtatggggaaaacggaggcagggctgctatggcttgggaataataagggttttttcacgctaccattgcgggcagcaatcaatgcaatttatgagcgaatcacaaaaaatattgttacgaccaatcaagccgagcgagtgggtttacttcattcggaaacatatagtcagtatttgctgcatgaaggaaatgcggagatggatattgatgaatattatacgaggacgaggcaaatgtctttacctataactatttgcacgctcgatcaattgtttgattttgtctttcgttatgcaggttttgaacataaattagccactttatcttattccaaagtgattattgatgaaatccagatgtactcacccgacttactggcttacttgattttagggttatcttatattgataaatttggtgggaagttctgcattatgacagcgactttaccgggaatagtgacagatttacttttagaaaatggagtggattttgtccaaccagaagagaaatttgtatcgtcgcgaattcgccacagcatggagatggttcatacagagattgaaagcgaatttattaaaccattttttaacaataatcgaattttagttatttgtaacactattagtaaagcgaaaaaaatctattcggagttaaaagagcattttcaaggtgaagaaattcatctcattcacagtcaatttattaaaagagaccgctcagctaaagaaaaagcaatttttaaagatgggcaaaaagatagtacgaaaaaatgtatttgggtagcaacgcaagtcgtagaggcttcattggatattgattttgatttactattcacggagctatcagatgtgaatgggctgtttcaacggatgggcagatgctatcgaaaccgggcgctggatgtggatacgaatgtctatgtttttgatggtggagcgaaagtttgctcaggaattggaacttttatcgataagttgatatttgtaaattctaagacgattttaaatgagcatgctggcgttttgacagaagagaagaaaatggagctagtggagcgggtttattcaacagaggctttacgtgggagtgaattttataacgatttgagaaagacgataaactatgtgaaagcttttgatagttatgaattggataaagcggatgttcgtaggagatttagaaacattaattcggtttctgttatcccagaagaagtttggcaagataatgcggaagaaatcaattcctattttgttactcttcgtaaaacctctaaggaaatatcaacaaaagagaaaatcattgcaagaaccaatttagcagaatttatggtgagtattccagactatctttataaaaaaggtgagagcgttgtacgcgaaataaaccgttatgagagtgttattgagtttaaatgtggttattcgagtgatgtaggggtttttatgaggtga

SEQ ID NO: 38 - DNA, 723 bp, synthetic construct
  misc_feature 1..723: note = Description of Artificial Sequence: Syntheticpolynucleotide
  source 1..723: mol_type = other DNA
atgagattaaagataaattgtgattttgattctaaaataatttcaaaagattttcaaagtaaagttgttagtctgttcaaagcgggcattatgaaatcgagtccagagagatatgagaatttatttggcgggaataagcataagcaatatacattttcagtgtaccttcccaaacctcaaaataaaggtgcggaaattcagttaaatgaagcaaattgtattattaatttttcaacgggagatgcggaaacgggaatcattttttataatgcattgatgagtttgagaggtagcaaagttttatttggagctgggaatcatattacggtaaaaaacattcaaatagtcccggagaaaaaaattatcggcaaacgaacaattttgagaacgctttcacctgtggttagtagagaccataatagagaaacattcaagaattggttttacagttttgaggatgaggaatttgagccaactttgaaaagaaacatgttgccctatttaatggatgcatttggtgagcaagctcgttatgatttggagaaattgaaaataacgccaatttcgatgaagaaagtagtcgtttattgccacgagatacatattgaaagctcagtcgggatttttgaattggaggctgaggcttatttgcagaagtatttggtggagaatggtattgggactatgacaggttctggatttggcatgatagagcagttttag

SEQ ID NO: 39 - DNA, 1689 bp, synthetic construct
  misc_feature 1..1689: note = Description of Artificial Sequence: Syntheticpolynucleotide
  source 1..1689: mol_type = other DNA
atgcggacagaaatagaagtaagggcaaatgattggttaataaatgctggtttaactggttttctaaatatcgttggaaaagaaaatgtaagaattgatggacaagctatttattttacagcagatttattagaggactttgaaacgaaatactttaatttctttattcaagaatataaagaaacgttatcgtggtataaaatcgtttcttataaagagacaatggaacagtttagaatagatgaatttgcttcttttgacgaggttgcattgggaaatttaaataaatatatgaaagatgtagtaaagttttatttaaaaaaagctaattatatcaaggtgtttccgctaattgaccctagtgctaatgtaacaaaatggttggaaaaattaaatacaattaatttaactaaaaagcaaaaatgggatgaagtaaaagccgatattattgaagaagtaaagcaaacatatactcaactagacctgattattgatttttgtgcaagtgaaaaggggcttcaatatctaggagcaaaaaatctgatatattctgttataaataaaggttggtctggtgtatctttccttcttaaacaaacaaaatttattgacccctatttagattacaaaactagttttttggacccagtaatagattatttagacaccgatttatctaaagcaaaatataattgtttcaattgtaatcaaccaattaagaatttgaaactagacctcagttttatgaacgatgttgggtttgatactgcaagaaaaacagctcacgtatgggattttaacaatgatgttgcgacttgcccgatttgtcgtttgatttattcatgtgttccagctggatttacgtatgtgtacggagaaggtatgtttgtcaacgattcatatggagtagaggaattacaccgtgttaatgaacgtatgcggaattctatcctgcgctttaacaaagatggaattaattcaactaatacttaccgagcgcttgtggagtctattacgatggagcatgaaaataatcgacgctatgaacttgcagacattcagttagttagatacgaaaatgagcattatcgttttaatttactttctaaaaaaatgttgcatattgtaaatgattcaaagggaatattgaaaagtttaattcgctgtggttataaagaaggtaatttgaatgttaatttgtacaaagaagttattcagcatttaatgaacaatgaaaatttatttacgcttattcataaattaatctactataaacaaactagtgtaaatggtttgtattacaatatgggacacgtttcagggattttagatataaatacaaaatttttgaaggagatggaagtgatgacaaatatttctcaaaatcaattatggttcgttcaaaattgtggaaaggaatttaaggaaggttatgttaagaagaagtcagaaaataaaatttcgggaataacatacaagttattaaattcgttaaaggtcaatgataaagatggatttatggatactttattgaatagttattcctatttatctatgcccattccagatgtattcataaaaatgttttcgaataatgaagcttttaaatcagtaggttatgcatttatgcttggagtaggcggagagaaaactaaaaaagaagacgggggaaacacagatgaaaaataa

SEQ ID NO: 40 - DNA, 870 bp, synthetic construct
  misc_feature 1..870: note = Description of Artificial Sequence: Syntheticpolynucleotide
  source 1..870: mol_type = other DNA
atgaaaaataaaggactagcaatgacaattattttccaagcagaaagtgccaactacggggagtctctaggtaatatttcttcacttaaaaagatttctagaaataatggagaccaatatacatatattagcagacaagcaattagatacaacctcatggaccaaattggagaaaaagaagctcctgttaaagcagaaggtggtggagataagaaagttattcaattcttaagtgaagctactattacagatttccctgaacttgatttctttggttaccttaaaacagaaaaaggaagtggcggacaaaagagaagtgcaaaagtacgtttatctaatgctatttctcttgaaacatttaaaggtgatttggatttcctaacaaataaaggtcaagcagataaattaaatgaaaatatgaatattgcacaagcagaaattcataaatcttattatcgttatacgattacgattgatttagaccaaattggtattgacggtgaaattgcacttgataataaagaaaaagctcgtcgtgtaaaaaaattgatggatacagtagcattcttgtaccgtgacattcgcgggcgccgagaagatttaaaaccacttttcgtcattggcggagtttatgacgtgaaaaatcctgttttccaaaatattgtagatgtagcagataacaatattgttattaaaaatattaaagatttacttacgtatgaagatatcaaagaaaatacgagagtgggtattattgacgggcaatttgctaattctgatgaagtgaaagtcgagctaaaagcagaatctgtgccggcattctttaacgatattaaaggaaagattgatgcttattatgaaagcaattag

SEQ ID NO: 41 - DNA, 774 bp, synthetic construct
  misc_feature 1..774: note = Description of Artificial Sequence: Syntheticpolynucleotide
  source 1..774: mol_type = other DNA
atgaaagcaattagagtaagattatggcaagatttggtaaattttaagaaaccaactagttttcaattaaaagaaacatatcctttacctccttattcaactgtaattgggatggtacatacactatgtggatttacaagctatcatgaaatgaaaattagtatacaaggtaaatatttttctaaagtaaatgatttagctacaagatatgaatttaaaaatggtatgacatatgatgcttctcgtcatcaaattaaagtagataagtatggtgtaagtagaggtgtatcaacagtagaattattagtagatttagaattattattacatattattcctgaagatccatcattagtaccaatcatagaaaaagcttttagagagcctattgagtttcctagtctaggtagaagagaggatattgctacaattcaaaaggtagaagttgtagatgtagaaaaaagacaacctaaaaaaagtacagaaatatcagatggttacaatgcttatgtaccaatctctttaactgaaaataattctgttcatttcaaatctcatgaatcatcagtaggtagagataagttgttaggtacaagatatttgctaacagaaaaatatgaaagagttaatcatggaacagaaaaagcacctaagtttttcagaaaatggagaaagaaagatgttatttactcaagtagaatttttgtctctaagaaagatgttttcttttttgatgaagatgattacttagtctttattgaagaagaggagtaa

SEQ ID NO: 42 - DNA, 2184 bp, synthetic construct
  misc_feature 1..2184: note = Description of Artificial Sequence: Syntheticpolynucleotide
  source 1..2184: mol_type = other DNA
atgcataaatatttagctaaatctaatcctgaagaaacaattcaagagcataccgataatttattaaaaaactatcaaagactaaaaaaactatatcctgctgtagaaatagattggtatttattagaattagtttgtttactacatgatttagggaaaatgaatctattattccaaactaaattaggtaatgcttcaggtaaaggtaaagaaataccacatggttatttgtctgttgcatttgttcctgatagtaaacttgaagaaatgggatattctgaagatgaaattaaagttgtttatcaagctatagctaggcatcatgaaaggaaatatgatttttctgattcagaatggaaaaaagaaataatcaaattagaagaacaatgggcaggttttatatattctcaattaggtggaaatgcagaatatagtagtgaggtagaaatgttatatttctatcctggttcaagaatttttgaaggtaactctgcagctgatgcagaaacattcaaaaaatacgttttattaaaaggtttattaaatagaattgactttgcagctagtggtggtattgatgtagaacttgaaaatgatttcttaatggatagtatggaagtacaattagcacattttaaagcagaaaatcctgaagcggattggaacgcattacaaaaatatatgatgcaacatcaaaatgaaaatattgtagttattgcagaaacaggtatgggtaaaacagaagcaggtttattatggttaggaaataataaaggttttttcacattacctttacgtgctgcaattaatgcaatttatgaaagaattacaaaaaatattgtaacaacaaatcaagcagaaagagtaggtctactacattcagaaacatattctcaatatttattacatgaaggtaatgctgaaatggatattgatgaatattatacaagaacaagacaaatgtctttacctataacaatatgtacattagaccaattatttgattttgtatttagatatgcaggttttgaacataaattagctacattatcatattctaaagtaattattgatgaaattcaaatgtattcacccgatttattagcttacttgattttagggttatcttatattgataaatttggtggaaagttctgcattatgacagcaacattacctggtatagtaacagatttacttttagaaaatggagttgattttgtacaacctgaagagaaatttgtatcttctagaattagacatagcatggagatggttcatacagaaattgaatcagaatttattaaaccattttttaacaataatagaattttagttatttgtaacactattagtaaagctaaaaaaatttattctgagttaaaagagcattttcaaggtgaagaaatacatttaattcacagtcaatttattaaaagagatagaagtgctaaagaaaaagcaatatttaaagatggtcaaaaagatagtacaaaaaaatgtatttgggtagcaacacaagtagtagaagcttcattggatattgattttgatttactattcacggagctatcagatgtgaatgggctgtttcaacggatgggcagatgctatcgaaaccgggcgctggatgtggatacgaatgtctatgtttttgatggtggagcgaaagtttgctcaggaattggaacttttatcgataagttgatatttgtaaattctaagacgattttaaatgagcatgctggcgttttgacagaagagaagaaaatggagctagtggagcgggtttattcaacagaggctttacgtgggagtgaattttataacgatttgagaaagacgataaactatgtgaaagcttttgatagttatgaattggataaagcggatgttcgtaggagatttagaaacattaattcggtttctgttatcccagaagaagtttggcaagataatgcggaagaaatcaattcctattttgttactcttcgtaaaacctctaaggaaatatcaacaaaagagaaaatcattgcaagaaccaatttagcagaatttatggtgagtattccagactatctttataaaaaaggtgagagcgttgtacgcgaaataaaccgttatgagagtgttattgagtttaaatgtggttattcgagtgatgtaggggtttttatgaggtga

SEQ ID NO: 43 - DNA, 29 bp, synthetic construct
  misc_feature 1..29: note = Description of Artificial Sequence: Syntheticoligonucleotide
  source 1..29: mol_type = other DNA
gttttaactacttattatgaaatgtaaat

SEQ ID NO: 44 - DNA, 37 bp, synthetic construct
  misc_feature 1..37: note = Description of Artificial Sequence: Syntheticoligonucleotide
  source 1..37: mol_type = other DNA
cgtttaataccttgttcaattttatcagctatttctt

SEQ ID NO: 45 - DNA, 37 bp, synthetic construct
  misc_feature 1..37: note = Description of Artificial Sequence: Syntheticoligonucleotide
  source 1..37: mol_type = other DNA
acttgcattgtctgtagaaattgggaatccaatttct

SEQ ID NO: 46 - DNA, 37 bp, synthetic construct
  source 1..37: mol_type = other DNA
aagaattattattaaaaggtttagaaaaattagaata

SEQ ID NO: 47 - protein, 256 aa, synthetic construct
MREKVSNRLKHIMKIRNLKQVDIINKSKPYQKQLGISLSKSTLSQYINDVQSPDQDRIYLLSKTLNVGEAWLMGYDVNSYRVPDEERQEETVMSKINNISSQLTPPRQSNVLNYANSQLDEQNKVTSIDEYKESKLVSYIACGATGAGIGEELYDDILHEEVFFKEDEAPSNADFCILINGDSMEPMLKQGTYAFIKKEDYIKDGTIALVVLDGVSLIKRVDICEDYINLVSLNPKYDDIKVASFSNIKVMGKVVL

SEQ ID NO: 48 - protein, 239 aa, synthetic construct
MDKKELAKFIGNKIRYYRTKLNLTQDQLGEKLNTKKATISNYETGYRTPKQDDLFEIAHILNISIDDLFPTRNNKKNDITSIYNKLTPPRQENVLNYANEQLDEQNKVTSIDEYKESKLVSYIACGATGAGIGEELYDDILHEEVFFKEDETPSNADFCILVNGDSMEPMLKQGTYAFIKKEDSIKDGTIALVVLDGVSLIKRVDICEDYINLVSLNPKYDDIKVASFSNIKVMGKVVL

SEQ ID NO: 49 - DNA, 2761 bp, synthetic construct
  source 1..2761: mol_type = other DNA
tgtattattgattaagtcactacttccacattcactacattcttcaggctcttcatcatgaccccagtcttcaaaatgcttacagtcctcacactcataagttaatacaaaatcctcttcaaattcaaaatctctctgcttcatattagtttcctcctttagcttgttcaattaaatcttttacagcaaatacttcagggttaattattcttaggatatcttggggtattccccacactaagtaaataaaagcaatgaaagtaataatacctgtaaaaactaaatacgtaataatcctcaaacaatagactatagtcatcatctctatcaatatcatttacttttttatgttggtttacaaataccttccataggaataatgttaaagctataactaaaataccaataataatatctacgataccgtatgctatctgatactttactaaagcctcccaagcatgttggctaatatcacctaacttacctccaagagaatcaattctctctaacactttatcttctaacttcattacaaccactccttatctagtcttctgagttccatccattcataaatccaagtacaaacagtactgccaatataaaaattacgaataacataggtcatacctcctctttatttgttaacttaagtataacctatgtatactcaattgtcaagacttatttcttctttttgcttaattttttttgttgctcttttagtgaatccttctgttgaggtgttaacattacttatctctccttatcaaattcttttgttgttttaatcttgtattcttctttaggattttgttcttctaatctatcttttaaatcctctaatacagatacatccctgtgagctttaatttgtctaggtattttctttttacctactttgtcgtaataacataaagcataccactcattcatttatataaccgccttacttaatattcttgatttctagctcaaactctactagtgagtttttagataaatctgtgatactgaacacattattatctcttttaataaagtgattactaggtgtaattctttccggttcatttttcttaatcaataattctaattgagagaataatctttcattaactaaattattaacatcttcttcaatatcttttctagttaagtaagaatcatttaatttatagatatttttgttaacattacctttaccaataaacatgttgtctacatgacaattacttcggtcaccatctttaaaacacacatgaccgttcttaaggtctcctcgtgattgactaatcatttcagggaatgtatgtgcaataactactcccctagaataacttgttttcttagggttaattaagtatacaatctcatgagcagtccacttaggatggttctttcttgtctgtgttcctttaattaactttttcgttactttatttctaattaaacctgtaggatgtgcttcataatacccattaaaaggtactgttctccatcctttctcaggctgtaatgagtggtctaaatgttttcttcttttaatcattatttttcctccttttgtaacttcttatctaaagttacgctataatcctctttataaaatcctttaattctacctttgcgtcctctagcatgtttaacatacttgtaaacagtacctacaaattgaattctctttccttgaagctcctttagtttttcgtagaaagagataccaacttcaatgttaacgtggtcaataggtataccattaatctttacatcggtaactgtgattttaacacatccatgttgattacctttatgtcttctgcgctttgagtacttaacatcatctaaataccctttaacattaacttgtttcctattaaaaggttttaactcttctctcatctttccacctcctactataattgtaacttatgatagttaaacatatctttagttaatttcttagcaaagtaattaatcatatattgtgttgaagttccttctagtttatcataatcaaaatcaatgtttgtaaaaattacaaagcttcctaagtttctactgccttctctaacttcaaatacaagtgtatcatttttaaaagttttaattttaaattctttattcgtatcaaaacttttagggaagttccaccattctttagttgttgccataatctcaattgcatttgaaaattgtcttctgattgtttctttactcatattaatcatctcctgtacttgttatatactaagtataatacaaaagtacaaaggtgtcaatacttatttttaagaaaatgaaagaagatttattatcattcctagtgcacatattaatgaagcactagagtatagtattgactccttatcatctttaactttcctagctaatagtaagaaaagtaacatacctgcttgtagtaagaaaaaccatccataaaatcctgcccaaatatttattaagattgcagtaccagtaagcatatagctcatatcatcattaatcaaatcaataatgactacagctataaaagcaacagaagcaatagttaataagtccataggcattaatcccagttccagtcttccatcacaaaatcaatataagcttcattttcttcatctataatttggtctttaactacttctatttctgaaataacaaactgtaaagaatctgttaatctacctagcgcaatacgttctgttgttttaataggtaaacgtcctgagtgtgggtgaactaagttatcaat

SEQ ID NO: 50 - DNA, 2761 bp, synthetic construct; identical to SEQ ID NO: 49
  source 1..2761: mol_type = other DNA

SEQ ID NO: 51 - DNA, 3653 bp, synthetic construct
  source 1..3653: mol_type = other DNA
actggaaaggtcagaatctattaggcaaagtaatggaagatgttcgagtacattgtatctacaataagtaggtaattataaaatggataagataaatctcaataaaaaacatgagggttctaccgtagtcaacatatcaaataatattatgttaaaaatacaatgtacagacctaagaaaagaatgtgatgattcagaagcacctactacctatacccattttaaagcttatatcgtatataatatattcattgtagttaatgatagaaaacaaaagaaaaaagttaagtacgattattataatgaccatgtaggtagaggtaatgttaaagacctattgaaagtaaaagatgttatcttccagttatccactcaattaaatactaatgaaattattaaaatatcgggtgcagatgaaagaagatataaaatatataaatattttatagaaaaagatataagatttgaagacaatatgtattatagtaaaagtaatatatggattataaataattttagcttattacaaaagtttcaatggaacactgtagtaactaaagatggagactataataaaaaagaacttaaaaaggttgataaagaatggaaagaattattaatataaaaggagagttaaaatgaatatcaaatatattgatttagtattagaaaattgcgatgttgtaagattagaacctaaagacgtaagcaggttccatatatcaggtattacagaaggtatagattactatggtacatataaagggacttcaaatataaatcgaacacgtcactgtacttattttggtattcttattgataaacctatggaaatacctcaagttggttttgcttatcctgataatacgaacgcttatgaaatgattacagcatattcagatattacagctatagatattatttatgaaaatgacgcaaatgaatatatttatgtagactttaatgaatacaatgataactataatatcaatcaaaagaatgattattacaataatatgttagaaattacaattacagaaagtaattccatggaggaagaagatgaataaaacattttttaagttcctaggtaaaaacacattagagtattcaaaacaggggctaggttttcttgtagcccttcctataatgttaattatattttctgtattcctagcattcattataggtattcctgcagttattatttacgctctacatgcattaaatgtagacaatgattttattatacaattagtacctgttatgtggtttataatactttatggtattgtaagaacagatgagcacaaaaaaccatttgttaaactaagattaaaggattacttactatctatattatatctaactactattatagctattagtgttttagagagctatttgctcttccaattattaccttttacaggagatgtaagagcagttataacactattatcatttatagtatttgttgttgtaaatagaggtatttgtaaaatagccatcaagagctataaagaatataaggaggaattataatgagatatgatattaatgaaaaatgttacgatgagaaggattttgtattacaaattatatatgaaaattatagtgatgaaacagatagagaagtagaaacaatttataataaagcagaagcatgggacaaactctgtgaaatgctgaaagataaagatatgagtgatgggcattttgaagaagaaatgtttaaactattctctagaacaggtaaatcctttacaatagataaggaggaatcacaatgatagaaattagtatctcatggacttatctaatatcattcttgctactatggtcagccggcaccctatacattaactaccttgtttatagaataaggttaacaaataaggaacgtaaagaaatgagtaaggaacaccaccgcaatagggaagagataaaacaaaggatagaaaatagaagggataaataataatggaaaattataaaaactttattatagaggaaatgaataaagcccgtatcttagtaactaaagcagaacaaattaaaaggaatagaaaattagcagaaacagaactagaagaagtatatagaaaagcagaagccttcgatgaaattgtaaatgagttactttatcaattacaaaatctagagagttgggatactctagaccaaaaagactgccaaacattaaaacaaatactagaggaaaatataaaggaggaaaaacagttgaaaagatacaaagtaaaacgtactattactacagaagaggtaagatatatagatgcagaaacagaggaggatgcatggtatagtgtagaatatgaagatgaaggtgcagatacagcacactataatgcagaatatggtacatggtcttatgaagaggaggaaaaataatgggattagactttgaagtaatcggtgtgacattaagtaatagaaaagtggaacagaaaggcttacagcactttattaataacgctaggtatagacatatattagaaaagaattattataaaggatttaactttgaagatgactttagaaaaccaggatattttatggacttacttctaagagacgcagagacatattatgatgagtttgaagaatggtgtgaaggcgtattcgtactaactaaagataagttagttaatctaatgaaaaatgagtttaatgaaaaaacgtttaaaggtacacatgatgcagaatattattatagattaatgtctcatatatacaatgtagaacaatatgaaggtaaattctatgacttttacttaatcatgagtgtaaatgtataaggaggattaagtatgaacaaagaacaagccaaacttaaactcgaaacaagtattattaattatgagaaccaaataaaattcttagaccctgcaagtatgtatactagagggcttatagatgcaaaaggttactcaaaattggcattaaaagaattagaagatacaggtaaacactcttatgaagatactacatggaaagatagttatgcaaaggtatttacagatgaagaaatactagaattcttactatccaaaccaagagtcacatttaaaggtaatcaagaaaaactagatgaaattaaaaaagaaagagaaaaaatacaaaaagaagctactaaagacttacctaagggcagtccactaggtgacttatcaaaagaaaattatgagaaattttggggagcattacaatggtctagagaagaaagagaaaagttaacacaagaatcaagagcttattatgaaaactatctaaaaaaaatcaaggagaataagtaatatgaaactataccaagtagaacatgataattgtgaaccttatgaagataacttccattttagagaagataagatatatacagataaggagaacttaattaaacgtattaaagaagaaggttataaagaggaaacaaaccatagaggtgagcaagagttcattaaaggagacccaagagatttctatgggatggatatgattactatacatgaattagaggttgttaataatacctaagaggagagggatttaatttcccctcttttttttattttaggtatgttgactttatatttagaggttgtctatttgtactataaattattgactta

SEQ ID NO: 52 - DNA, 42 bp, synthetic construct
  source 1..42: mol_type = other DNA
tgatttgtttgaaattgctcatattttaaatatcagtatcga

SEQ ID NO: 53 - DNA, 42 bp, synthetic construct
  source 1..42: mol_type = other DNA
gatgatttgtttgaaattgctcatattttaaatatcagtatc

SEQ ID NO: 54 - DNA, 510 bp, synthetic construct
  source 1..510: mol_type = other DNA
cttgttttaacataggttccattgaatcaccattaactaaaatacaaaaatcagcatttgatggcgtttcgtcttctttgaaaaatacttcttcgtgtaatatatcatcgtacaattcttctccgataccagcaccagttgcgccacacgcaatatacgacactaacttagattctttatattcatctatagaagtgactttattttgttcatctaactgactattcgcgtagttgagtacattgctttgtcttggaggcgtgagctgagatgatatgttattaatttttgacatcacagtttcctcttggcgttcttcatcgggtacgcgataagaatttacatcatatcccataagccacgcttcaccgacatttaaagttttagaaagtaggtaaattctatcttggtcaggagattgtacatcgttaatatattgagacaaagtgcttttacttaaagatatacctagttgcttttgataaggtttcgatttattaatgatatcta

SEQ ID NO: 55 - DNA, 111 bp, synthetic construct
  source 1..111: mol_type = other DNA
ctgaattagagagaactttagggttttcaaacggacaaatcagaagatgggagaaaaccaaaccaggtattgataaggtgcaaaaaattgccgatcactttgatgtatcag

SEQ ID NO: 56 - DNA, 2889 bp, synthetic construct
  source 1..2889: mol_type = other DNA
ggaaggtagtattggatagctttaaaccgcgttgttaagccattcttgacttccggaaatggctattgataccattttgataccatttacttgtcaaaaatggctattgcatcgtgttttttctgagtatataaatggctgtaagtgcccatcgtttcagtgatttgagcatgtctcatgagtgactgtaaaacgaaaatatctacaccattatttgcaagataagatgcataagaatgtcttaacgcgtgaatgttataatgggggaaagctttttggaatttcttttgaacatgactataatgtttgggagccattcctccgaaaataaaataactacgttcatcaaaatatttgtttaactctttttcacgttggtgtcgttcagttaacattgtattgatgaatttaggtaaaggaacaatatcctctgaactatctgtttttggtcttggatatatagttctattagagatgtccattgttttatttatggatatctcttttttgtatttattgtagtctgtccaaacaagcgccatagcttcgccaatcctcaaacctgtataaaacattaatgtaaataactctctgtaatcttgctcttcaatatctttgattctttcttcaaattcttcacgcatcataaacttaggttttggctttacacgcggaataggtttaattgatattgttggatctgtacgtaatccaaagtattttttagcataattaattacaactttaaaacctgaccaaattgtacgagcagaatttgttgacgctacattctctattagatatttacgaaactcttggcattgattttgtgttatcttattcatttttatgtgcccaaacttagctttaaagtgtttatgatattcattttgcttgcgtcgttttgttttaggtctcaaatcgctattttctaaatagtgatgaaaaacataatcaaaagtttttgaatcgctatatccttcgtttacgtcattcaaaaaaatagcctctgctctcttagcttcacgcttagttgaaaaaccgcgttgcatcttacgtttgttattaccgtatacatctttatatctaatagaaaaataccatttacctgtattatcatccttatatactggcattttgcttctccctcctcaaaattggcaaaaaaataataagggtaggcgggctacccgaaattttattgttgaatcacttcgctattttgacgtttgaaattgtcgaaatcattttgtgctttcttccatgaattatagtcttgtccgtcttgtactgcccatgaaccacctataccggcagtatggccaccattctgacgtttgttttcttctgttgctcttttagcttcttgataagcgttataagatgtgtcacttgaaaactcatctttcactggtgcattgttgtttttattagaagtgggattattttgtgtttgattttgtttaggtgcgttattagtttgttgatgatcattaacatttgtgttgttatcgttgtttacttgattattgttatcgttttgattagcattttcttttttcacttctgctttgtctttagtttctttctttttgtctttgttctctttctttgtttccgctttcttgctttcctctttcttatcgccgtcgttgctaccacatgcacctaatactaacgcgctagctaaaattaaatataataatcttttcatgttttacactcctttatttgctatttgttttaataaatctatgacttcgttgttttgctcgataattctattattttgctttattagttcgtctcgttgagctatagagacaaagttttgttttaattgcgtatcgtagaatacgaatttagcttgtttatctacgtttgttgtgaatgtacctaaaccgttgtagactttcaatagtgtagggttaatattttgcttttgatatgcgtaagtcgtaacgtcggtagcttctttaataccttgtccgtttaaacttttagctgattttgattcgtattcttcgttagtgtttttaaaattttcagatttataaagttggatatcaagttcttttccttctttaaaatcatttaatatctttcttttttcatctgttgtcatttttttatacatgtcaatctttctattgcttaacttactaaacatttttgtttctgttagaatttctttaaaagttaatttatctcctgccattttcaatttctccttcatttggtttatattaaagcgccacatagacgctattaatcaaaaattcgatagttataaataactttgcctatcacttcgatttcatcaatagaatctaaatcgtaagaattagttttaaattcatctgaatagcttactgggtctaaatgtagttttgtttcagtacgtctcacacgtttaactgtatattcaccacctagacgcaatacaaggatgtcgttgctgttaagtttatgatcacaagactttctataatcatggacaattatataagaaccgttagcgagtattttattcatgctatctccgtttatttttagtgctatacattcgctaggtttacgaccgttaaaagcaaatggtggaacttttaatttttcattttcaattgcaacttcttcgaaatttccagcagaaactttaccgaaatatggaacctcgatttcgctatcaaattcgggtaaaacaatttcttcaatttctcctaagagataacctttagaaacattgaacaaacttgaaattttttcgaccatacccattctaggttcagttcttccgctttcccacattcttatagtaccttcggaaacatctaattttctagccatctcaactttagacaatctattgttcaatctgatttcttttat

SEQ ID NO: 57 - skipped

SEQ ID NO: 58 - DNA, 42 bp, synthetic construct
  source 1..42: mol_type = other DNA
tcgatactgatatttaaaatatgagcaatttcaaacaaatca